(19) United States
(12) Patent Application Publication
(10) Pub. No.: US 2023/0312672 A1
(43) Pub. Date: Oct. 5, 2023

(54) HLA CLASS I-RESTRICTED T CELL RECEPTORS AGAINST CD20

(71) Applicant: The United States of America,as represented by the Secretary,Department of Health and Human Services, Bethesda, MD (US)

(72) Inventors: Kazusa Ishii, Bethesda, MD (US); Christian S. Hinrichs, Bethesda, MD (US)

(73) Assignee: The United States of America,as represented by the Secretary,Department of Health and Human Services, Bethesda, MD (US)

(21) Appl. No.: 18/012,056

(22) PCT Filed: Jun. 23, 2021
(86) PCT No.: PCT/US2021/038649

§ 371 Date: Dec. 21, 2022

Related U.S. Application Data
(60) Provisional application No. 63/043,520, filed on Jun. 24, 2020.

(51) Int. Cl. C07K 14/725; C07K 14/705; C12N 15/86; C12N 5/0783

(52) U.S. Cl. CPC C07K 14/7051; C07K 14/70596; C12N 15/86; C12N 5/0636; C12N 2740/13043

(57) ABSTRACT

Disclosed are isolated or purified T cell receptors (TCRs), wherein the TCRs have antigenic specificity for a CD20 amino acid sequence presented by a human leukocyte antigen (HLA) Class I molecule. Related polypeptides and proteins, as well as related nucleic acids, recombinant expression vectors, host cells, populations of cells, and pharmaceutical compositions are also provided. Also disclosed are methods of detecting the presence of cancer in a mammal and methods of treating or preventing cancer in a mammal.

### CROSS REFERENCE TO RELATED APPLICATION

This patent application claims the benefit of U.S. Provisional Patent Application No. 63/043,520, filed Jun. 24, 2020, which is incorporated by reference in its entirety herein.

### STATEMENT REGARDING FEDERALLY SPONSORED RESEARCH OR DEVELOPMENT

This invention was made with Government support under project number 1 ZIA BC011479 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 373,854 Byte ASCII (Text) file named “754474_ST25.txt” dated Jun. 22, 2021.

### BACKGROUND OF THE INVENTION

Some cancers may have very limited treatment options, particularly when the cancer becomes metastatic and unresectable. Despite advances in treatments such as, for example, surgery, chemotherapy, and radiation therapy, the prognosis for many cancers, such as, for example, lymphomas, leukemias, and epithelial cancers, including Hodgkin lymphoma, T/NK cell lymphoma, post-transplant lymphoproliferative disorders, nasopharyngeal cancer, and gastric cancer, may be poor. Accordingly, there exists an unmet need for additional treatments for cancer.

### BRIEF SUMMARY OF THE INVENTION

An embodiment of the invention provides an isolated or purified T-cell receptor (TCR) comprising the amino acid sequences of (a) SEQ ID NOs: 1-3, (b) SEQ ID NOs: 4-6, (c) SEQ ID NOs: 7-9, (d) SEQ ID NOs: 10-12, (e) SEQ ID NOs: 13-15, (f) SEQ ID NOs: 16-18, (g) SEQ ID NOs: 19-21, (h) SEQ ID NOs: 22-24, (i) SEQ ID NOs: 25-27, (j) SEQ ID NOs: 28-30, (k) SEQ ID NOs: 31-33, (1) SEQ ID NOs: 34-36, (m) SEQ ID NOs: 37-39, (n) SEQ ID NOs: 40-42, (o) SEQ ID NOs: 43-45, (p) SEQ ID NOs: 46-48, (q) SEQ ID NOs: 49-51, (r) SEQ ID NOs: 52-54, (s) SEQ ID NOs: 1-6, (t) SEQ ID NOs: 7-12, (u) SEQ ID NOs: 13-18, (v) SEQ ID NOs: 19-24, (w) SEQ ID NOs: 25-30, (x) SEQ ID NOs: 31-36, (y) SEQ ID NOs: 37-42, (z) SEQ ID NOs: 43-48, or (aa) SEQ ID NOs: 49-54, wherein the TCR has antigenic specificity for a CD20 amino acid sequence presented by a human leukocyte antigen (HLA) Class I molecule.

Another embodiment of the invention provides an isolated or purified polypeptide comprising a functional portion of the inventive TCR, wherein the functional portion comprises the amino acid sequences of (a) SEQ ID NOs: 1-3, (b) SEQ ID NOs: 4-6, (c) SEQ ID NOs: 7-9, (d) SEQ ID NOs: 10-12, (e) SEQ ID NOs: 13-15, (0 SEQ ID NOs: 16-18, (g) SEQ ID NOs: 19-21, (h) SEQ ID NOs: 22-24, (i) SEQ ID NOs: 25-27, (j) SEQ ID NOs: 28-30, (k) SEQ ID NOs: 31-33, (1) SEQ ID NOs: 34-36, (m) SEQ ID NOs: 37-39, (n) SEQ ID NOs: 40-42, (o) SEQ ID NOs: 43-45, (p) SEQ ID NOs: 46-48, (q) SEQ ID NOs: 49-51, (r) SEQ ID NOs: 52-54, (s) SEQ ID NOs: 1-6, (t) SEQ ID NOs: 7-12, (u) SEQ ID NOs: 13-18, (v) SEQ ID NOs: 19-24, (w) SEQ ID NOs: 25-30, (x) SEQ ID NOs: 31-36, (y) SEQ ID NOs: 37-42, (z) SEQ ID NOs: 43-48, or (aa) SEQ ID NOs: 49-54.

Still another embodiment of the invention provides an isolated or purified protein comprising at least one of the inventive polypeptides.

Further embodiments of the invention provide nucleic acids, recombinant expression vectors, host cells, populations of cells, and pharmaceutical compositions relating to the inventive TCRs, polypeptides, and proteins.

An embodiment of the invention provides an isolated or purified nucleic acid comprising, from 5′ to 3′, a first nucleic acid sequence and a second nucleotide sequence, wherein the first and second nucleotide sequence, respectively, encode the amino sequences of SEQ ID NOs: 56, and 57 or 58; 57 or 58, and 56; 59, and 60 or 61; 60 or 61, and 59; 62, and 63 or 64; 63 or 64, and 62; 65, and 66 or 67; 66 or 67, and 65; 68, and 69 or 70; 69 or 70, and 68; 71, and 72 or 73; 27 or 73, and 71; 74, and 75 or 76; 75 or 76, and 74; 77, and 78 or 79; 78 or 79, and 77; 80, and 81 or 82; 81 or 82, and 80; 83 and 84; 84 and 83; 85 and 86; 86 and 85; 87 and 88; 88 and 87; 89 and 90; 90 and 89; 91 and 92; 92 and 91; 93 and 94; 94 and 93; 95 and 96; 96 and 95; 97 and 98; 98 and 97; 99 and 100; or 100 and 99.

Methods of detecting the presence of cancer in a mammal, methods of treating or preventing cancer in a mammal, methods of inducing an immune response against a cancer in a mammal, methods of producing a host cell expressing a TCR that has antigenic specificity for the peptide of SEQ ID NO: 55, and methods of producing the inventive TCRs, polypeptides, and proteins, are further provided by embodiments of the invention.

Additional embodiments are as described herein.

### BRIEF DESCRIPTION OF THE DRAWINGS

FIG. 1 is a bar graph showing polyclonal mouse splenocytes, collected as described in Example 1, contain T cells that recognize CD20+/HLA-A2+ cell lines.

FIG. 2 presents flow cytometry dot plots, showing polyclonal mouse splenocytes harvested from CD20-peptide vaccinated mice contain a T cell population that binds to a CD20 p188-196 peptide-HLA-A*02:01 tetramer (top) but not to the irrelevant pMHC tetramer (bottom).

FIGS. 3A-3AB present flow cytometry dot plots, showing binding of TCRs to CD20 p-MHC tetramers, but not to irrelevant p-MHC tetramer (HPV-16, E7 p11-19), in accordance with embodiments of the invention. FIG. 3A demonstrates untransduced T cells that neither bind to CD20 p-MHC tetramer or antibodies against mTRBC; FIG. 3B demonstrates T cells transduced with irrelevant E7 TCR (TCR against HLA-A*02:01-restricted HPV-E7 p11-19 epitope) that does not bind to CD20 p-MHC tetramer but does express mTRBC; FIG. 3C demonstrates untransduced T cells that neither bind to E7 p-MHC tetramer or antibodies against mTRBC; FIG. 3D demonstrates T cells transduced with irrelevant E7 TCR (TCR against HLA-A*02:01-restricted HPV-E7 p11-19 epitope) that binds to E7 p-MHC tetramer and expresses mTRBC; FIG. 3E demonstrates T cells transduced with CD20 TCR J1A1 which expresses mTRBC and binds to CD20 p-MHC tetramer; FIG. 3F demonstrates T cells transduced with CD20 TCR J1A2 which expresses mTRBC and binds to CD20 p-MHC tetramer; FIG. 3G demonstrates T cells transduced with CD20 TCR J1A3 which expresses mTRBC and binds to CD20 p-MHC tetramer; FIG. 3H demonstrates T cells transduced with CD20 TCR 869A1 which expresses mTRBC and binds to CD20 p-MHC tetramer; FIG. 3I demonstrates T cells transduced with CD20 TCR J1A1 which expresses mTRBC and does not bind to irrelevant E7 p-MHC tetramer; FIG. 3J demonstrates T cells transduced with CD20 TCR J1A2 which expresses mTRBC and does not bind to irrelevant E7 p-MHC tetramer; FIG. 3K demonstrates T cells transduced with CD20 TCR J1A3 which expresses mTRBC and does not bind to irrelevant E7 p-MHC tetramer; FIG. 3L demonstrates T cells transduced with CD20 TCR 869A1 which expresses mTRBC and does not bind to irrelevant E7 p-MHC tetramer; FIG. 3M demonstrates untransduced T cells that neither bind to CD20 p-MHC tetramer or antibodies against mTRBC; FIG. 3N demonstrates T cells transduced with irrelevant E7 TCR (TCR against HLA-A*02:01-restricted HPV-E7 p11-19 epitope) that does not bind to CD20 p-MHC tetramer but does express mTRBC; FIG. 3O demonstrates T cells transduced with CD20 TCR J1A2 which expresses mTRBC and binds to CD20 p-MHC tetramer; FIG. 3P demonstrates T cells transduced with CD20 TCR J2B1 which expresses mTRBC and binds to CD20 p-MHC tetramer; FIG. 3Q demonstrates T cells transduced with CD20 TCR 56A22 which expresses mTRBC and binds to CD20 p-MHC tetramer; FIG. 3R demonstrates T cells transduced with CD20 TCR 56A23 which expresses mTRBC and binds to CD20 p-MHC tetramer; FIG. 3S demonstrates T cells transduced with CD20 TCR 56B2 which expresses mTRBC and binds to CD20 p-MHC tetramer; FIG. 3T demonstrates T cells transduced with CD20 TCR 79B2 which expresses mTRBC and binds to CD20 p-MHC tetramer; FIG. 3U demonstrates untransduced T cells that neither bind to E7 p-MHC tetramer or antibodies against mTRBC; FIG. 3V demonstrates T cells transduced with irrelevant E7 TCR (TCR against HLA-A*02:01-restricted HPV-E7 p11-19 epitope) that binds to E7 p-MHC tetramer and expresses mTRBC; FIG. 3W demonstrates T cells transduced with CD20 TCR J1A2 which expresses mTRBC and does not bind to irrelevant E7 p-MHC tetramer; FIG. 3X demonstrates T cells transduced with CD20 TCR J2B1 which expresses mTRBC and does not bind to irrelevant E7 p-MHC tetramer; FIG. 3Y demonstrates T cells transduced with CD20 TCR 56A22 which expresses mTRBC and does not bind to irrelevant E7 p-MHC tetramer; FIG. 3Z demonstrates T cells transduced with CD20 TCR 56A23 which expresses mTRBC and does not bind to irrelevant E7 p-MHC tetramer; FIG. 3AA demonstrates T cells transduced with CD20 TCR 56B2 which expresses mTRBC and does not bind to irrelevant E7 p-MHC tetramer; FIG. 3AB demonstrates T cells transduced with CD20 TCR 79B2 which expresses mTRBC and does not bind to irrelevant E7 p-MHC tetramer.

FIGS. 4A-4C are bar graphs showing J1A2 recognizes CD20+/HLA-A2+ cell lines, in accordance with embodiments of the invention. FIG. 4A shows results for K562-based cell lines. FIG. 4B shows results for EBV-LCL cell lines. FIG. 4C shows results for additional cell lines. Positive and negative controls are the K562 cell lines shown in FIG. 4A.

FIG. 5 is a line graph demonstrating a functional avidity of TCR J1A2 as a function of IFNγ levels in the overnight co-culture supernatant of TCR J1A2 with HLA-A*02:01+K562-based artificial antigen presenting cells loaded with different concentrations of CD20 p188-196 peptide or irrelevant peptide E7 p11-19, in accordance with embodiments of the invention.

FIGS. 6A-6C present bar graphs showing IFNγ levels measured in the overnight co-culture supernatant of inventive TCRs with cell lines, in accordance with embodiments of the invention. FIG. 6A shows results for K562-based cell lines. FIG. 6B shows results for various cell lines. FIG. 6C shows results for EBV-LCL cell lines. Positive and negative controls are the K562 cell lines shown in FIG. 6A.

FIG. 7 presents a bar graph showing IFNγ levels measured in the overnight co-culture supernatant of inventive TCRs with various cell lines, in accordance with embodiments of the invention.

FIG. 8 is a bar graph showing results of T cells transduced to express J1A2 TCR, E7TCR, or untransduced T cells that were co-cultured with cell lines as indicated on the x-axis at an E:T ratio of 1:1. Overnight co-culture supernatant was obtained and the level of IFNγ was measured using ELISA.

FIG. 9 presents bar graphs showing cell line RPMI6666 (left) and JVM2 (right), which are both HLA-A*02:01+ and CD20+, retrovirally transduced to express firefly luciferase cocultured with or without T cells. CD20TCR (J1A2) or E7TCR transduced T cells were co-cultured with RPMI6666 or JVM2 at an E:T ratios indicated on the x-axes. Six hours after co-culture, the levels of luminescence (a proxy for live lymphoma cells) were measured immediately following the addition of substrate, luciferin.

FIGS. 10A-10E are bar graphs showing IFNγ levels measured in the overnight co-culture supernatant of T cells transduced to express the anti-CD20TCR (clone J1A2) with CCRF-SB cells (10A), DG-75 cells (10B), NALM1 cells (10C), SS4050 cells (10D), and BV173 cells (10E). Each dot indicates independent PBMC donors, and the data are stratified according to the donor's endogenous HLA-A*02:01 status and whether B cells were depleted from PBMC prior to TCR transduction. ns is not significant (p>0.05), ** p<0.01.

FIG. 11 represents the result of alanine scanning assay: CD20TCR (clone J1A2)-transduced T cells or E7TCR-transduced TCRs were co-culture with K562A cells loaded with 1 μM of epitope peptide or peptides with alanine substitutions at one residue at a time as indicated on the x axis. The levels of IFNγ in the overnight co-culture supernatant was measured with ELISA.

FIG. 12 is a bar graph showing the results of CD20TCR-transduced T cells (clone J1A2) or untransduced T cells co-cultured with K562A2 loaded with 1 μM of cross-reactivity candidate peptides as indicated on the x-axis. The levels of IFNγ in the overnight co-culture supernatant was measured with ELISA. Amino acid sequences of the peptides are also provided on the x-axis for those peptides that resulted in a measurable IFNγ production by T cells.

FIG. 13 is a bar graph showing levels of IFNγ for CD20TCR-transduced T cells or untransduced T cells co-cultured with K562A2 cells loaded with titrated concentrations of peptides. IFNγ levels in overnight co-culture supernatant was measured with ELISA.

FIG. 14 is a bar graph showing levels of IFNγ for CD20TCR-transduced T cells or untransduced T cells co-cultured with K562A2 cells loaded with titrated concentrations of peptides. IFNγ levels in overnight co-culture supernatant was measured with ELISA.

### DETAILED DESCRIPTION OF THE INVENTION

An embodiment of the invention provides an isolated or purified TCR having antigenic specificity for CD20 (p188-196) amino acid sequence SLFLGILSV (SEQ ID NO: 55) presented by a human leukocyte antigen (HLA) Class I molecule. CD20 is widely expressed by mature B-lymphoid malignancies. Hereinafter, references to a “TCR” also refer to functional portions and functional variants of the TCR, unless specified otherwise.

In embodiments of the invention, the inventive TCRs are able to recognize CD20 presented by an HLA Class I molecule. In this regard, the TCR may elicit an immune response upon binding to CD20-derived peptides presented in the context of an HLA Class I molecule.

In an embodiment of the invention, the HLA Class I molecule is an HLA-A molecule. The HLA-A molecule is a heterodimer of an α chain and β2 microglobulin. The HLA-A α chain may be encoded by an HLA-A gene. β2 microglobulin binds non-covalently to the alpha chain to build the HLA-A complex. The HLA-A molecule may be any HLA-A molecule. In embodiments of the invention, the HLA Class I molecule is an HLA-A02 molecule. The HLA-A02 molecule may be any HLA-A02 molecule. Examples of HLA-A02 molecules may include, but are not limited to, those expressed by the HLA-A*02:01, HLA-A*02:02, HLA-A*02:03, HLA-A*02:05, HLA-A*02:06, HLA-A*02:07, and HLA-A*02:11 alleles. Preferably, the HLA-A02 molecule is expressed by the HLA-A*02:01 allele.

The TCRs of the invention may provide any one or more of a variety of advantages, including when expressed by cells used for adoptive cell transfer. Without being bound to a particular theory or mechanism, it is believed that the inventive TCRs advantageously target the destruction of cancer cells while minimizing or eliminating the destruction of normal, non-cancerous cells, thereby reducing, for example, by minimizing or eliminating, toxicity. Importantly, CD20 expression is limited to B-cell lineage lymphocytes, and no other normal tissues express CD20. Therefore, the TCRs of the invention are expected not to cause any tissue damage to normal tissues and organs. Moreover, the inventive TCRs may, advantageously, successfully treat or prevent CD20-positive cancers that do not respond to other types of treatment such as, for example, chemotherapy, antibody therapies, surgery, or radiation. Additionally, the inventive TCRs may provide highly avid recognition of CD20, which may provide the ability to recognize unmanipulated tumor cells (e.g., tumor cells that have not been treated with interferon (IFN)-γ). Accordingly, the inventive TCRs may provide treatment options for CD20-expressing diseases that are resistant to existing treatments. Moreover, in embodiments, the inventive TCRs, polypeptides and proteins may comprise human amino acid sequences, which may reduce the risk of rejection by the human immune system as compared to, e.g., TCRs, polypeptides and proteins comprising only mouse amino acid sequences.

The phrase “antigenic specificity,” as used herein, means that the TCR can specifically bind to and immunologically recognize CD20 with high avidity. For example, a TCR may be considered to have “antigenic specificity” for CD20 if about 1×10⁴ to about 1×10⁵ T cells expressing the TCR secrete at least about 200 pg/mL or more (e.g., 200 pg/mL or more, 300 pg/mL or more, 400 pg/mL or more, 500 pg/mL or more, 600 pg/mL or more, 700 pg/mL or more, 1000 pg/mL or more, 5,000 pg/mL or more, 7,000 pg/mL or more, 10,000 pg/mL or more, 20,000 pg/mL or more, or a range defined by any two of the foregoing values) of IFN-γ upon co-culture with (a) antigen-negative, HLA Class I molecule positive target cells pulsed with a low concentration of CD20 peptide (e.g., about 0.05 ng/mL to about 10 ng/mL, 1 ng/mL, 2 ng/mL, 5 ng/mL, 8 ng/mL, 10 ng/mL, or a range defined by any two of the foregoing values) or (b) antigen-negative, HLA Class I molecule positive target cells into which a nucleotide sequence encoding CD20 has been introduced such that the target cell expresses CD20, or (c) HLA Class I molecule positive target cells that naturally express CD20. Cells expressing the inventive TCRs may also secrete IFN-γ upon co-culture with antigen-negative, HLA Class I molecule positive target cells pulsed with higher concentrations of CD20 peptide or any peptide sequences containing the minimal epitope peptide sequence SLFLGILSV. The HLA Class I molecule may be any of the HLA Class I molecules described herein (e.g., an HLA-A*02:01 molecule).

Alternatively or additionally, a TCR may be considered to have “antigenic specificity” for CD20 if T cells expressing the TCR secrete at least twice as much IFN-γ upon co-culture with (a) antigen-negative, HLA Class I molecule positive target cells pulsed with a low concentration of CD20 peptide or (b) antigen-negative, HLA Class I molecule positive target cells into which a nucleotide sequence encoding CD20 has been introduced such that the target cell expresses CD20 or (c) HLA Class I molecule positive target cells that naturally express CD20, as compared to the amount of IFN-γ expressed by a negative control. The negative control may be, for example, (i) T cells expressing the TCR, co-cultured with (a) antigen-negative, HLA Class I molecule positive target cells pulsed with the same concentration of an irrelevant peptide (e.g., some other peptide with a different sequence from the CD20 peptide) or (b) antigen-negative, HLA Class I molecule positive target cells into which a nucleotide sequence encoding an irrelevant peptide has been introduced such that the target cell expresses the irrelevant peptide, or (ii) untransduced T cells (e.g., derived from PBMC, which do not express the TCR) co-cultured with (a) antigen-negative, HLA Class I molecule positive target cells pulsed with the same concentration of CD20 peptide or (b) antigen-negative, HLA Class I molecule positive target cells into which a nucleotide sequence encoding CD20 has been introduced such that the target cell expresses CD20 or (c) HLA Class I molecule positive target cells that naturally express CD20. The HLA Class I molecule expressed by the target cells of the negative control would be the same HLA Class I molecule expressed by the target cells that are co-cultured with the T cells being tested. The HLA Class I molecule may be any of the HLA Class I molecules described herein (e.g., an HLA-A*02:01 molecule). IFN-γ secretion may be measured by methods known in the art such as, for example, enzyme-linked immunosorbent assay (ELISA).

Alternatively or additionally, a TCR may be considered to have “antigenic specificity” for CD20 if at least twice as many of the numbers of T cells expressing the TCR secrete IFN-γ upon co-culture with (a) antigen-negative, HLA Class I molecule positive target cells pulsed with a low concentration of CD20 peptide or (b) antigen-negative, HLA Class I molecule positive target cells into which a nucleotide sequence encoding CD20 has been introduced such that the target cell expresses CD20 or (c) HLA Class I molecule positive target cells that naturally express CD20 as compared to the numbers of negative control T cells that secrete IFN-γ. The HLA Class I molecule, concentration of peptide, and the negative control may be as described herein with respect to other aspects of the invention. The numbers of cells secreting IFN-γ may be measured by methods known in the art such as, for example, ELISPOT.

Alternatively or additionally, a TCR may be considered to have “antigenic specificity” for CD20 if T cells expressing the TCR upregulate expression of one or more T-cell activation markers as measured by, for example, flow cytometry after stimulation with target cells expressing CD20 and HLA Class I molecules. Examples of T-cell activation markers include 4-1BB, OX40, CD107a, CD69, and cytokines that are upregulated upon antigen stimulation (e.g., tumor necrosis factor (TNF), interleukin (IL)-2, etc.).

An embodiment of the invention provides a TCR comprising two polypeptides (i.e., polypeptide chains), such as an alpha (α) chain of a TCR, a beta (β) chain of a TCR, a gamma (γ) chain of a TCR, a delta (δ) chain of a TCR, or a combination thereof. The polypeptides of the inventive TCR can comprise any amino acid sequence, provided that the TCR has antigenic specificity for CD20. In some embodiments, the TCR is non-naturally occurring.

In an embodiment of the invention, the TCR comprises two polypeptide chains, each of which comprises a variable region comprising a complementarity determining region (CDR)1, a CDR2, and a CDR3 of a TCR. In an embodiment of the invention, the TCR comprises a first polypeptide chain comprising a CDR1 comprising the amino acid sequence of SEQ ID NO: 1 (CDR1 of α chain), a CDR2 comprising the amino acid sequence of SEQ ID NO: 2 (CDR2 of α chain), and a CDR3 comprising the amino acid sequence of SEQ ID NO: 3 (CDR3 of α chain), and a second polypeptide chain comprising a CDR1 comprising the amino acid sequence of SEQ ID NO: 4 (CDR1 of β chain), a CDR2 comprising the amino acid sequence of SEQ ID NO: 5 (CDR2 of β chain), and a CDR3 comprising the amino acid sequence of SEQ ID NO: 6 (CDR3 of β chain).

In another embodiment of the invention, the TCR comprises a first polypeptide chain comprising a CDR1 comprising the amino acid sequence of SEQ ID NO: 7 (CDR1 of a chain), a CDR2 comprising the amino acid sequence of SEQ ID NO: 8 (CDR2 of α chain), and a CDR3 comprising the amino acid sequence of SEQ ID NO: 9 (CDR3 of α chain), and a second polypeptide chain comprising a CDR1 comprising the amino acid sequence of SEQ ID NO: 10 (CDR1 of β chain), a CDR2 comprising the amino acid sequence of SEQ ID NO: 11 (CDR2 of β chain), and a CDR3 comprising the amino acid sequence of SEQ ID NO: 12 (CDR3 of β chain).

In another embodiment of the invention, the TCR comprises a first polypeptide chain comprising a CDR1 comprising the amino acid sequence of SEQ ID NO: 13 (CDR1 of a chain), a CDR2 comprising the amino acid sequence of SEQ ID NO: 14 (CDR2 of α chain), and a CDR3 comprising the amino acid sequence of SEQ ID NO: 15 (CDR3 of α chain), and a second polypeptide chain comprising a CDR1 comprising the amino acid sequence of SEQ ID NO: 16 (CDR1 of β chain), a CDR2 comprising the amino acid sequence of SEQ ID NO: 17 (CDR2 of β chain), and a CDR3 comprising the amino acid sequence of SEQ ID NO: 18 (CDR3 of β chain).

In another embodiment of the invention, the TCR comprises a first polypeptide chain comprising a CDR1 comprising the amino acid sequence of SEQ ID NO: 19 (CDR1 of a chain), a CDR2 comprising the amino acid sequence of SEQ ID NO: 20 (CDR2 of α chain), and a CDR3 comprising the amino acid sequence of SEQ ID NO: 21 (CDR3 of α chain), and a second polypeptide chain comprising a CDR1 comprising the amino acid sequence of SEQ ID NO: 22 (CDR1 of β chain), a CDR2 comprising the amino acid sequence of SEQ ID NO: 23 (CDR2 of β chain), and a CDR3 comprising the amino acid sequence of SEQ ID NO: 24 (CDR3 of β chain).

In another embodiment of the invention, the TCR comprises a first polypeptide chain comprising a CDR1 comprising the amino acid sequence of SEQ ID NO: 25 (CDR1 of a chain), a CDR2 comprising the amino acid sequence of SEQ ID NO: 26 (CDR2 of α chain), and a CDR3 comprising the amino acid sequence of SEQ ID NO: 27 (CDR3 of α chain), and a second polypeptide chain comprising a CDR1 comprising the amino acid sequence of SEQ ID NO: 28 (CDR1 of β chain), a CDR2 comprising the amino acid sequence of SEQ ID NO: 29 (CDR2 of β chain), and a CDR3 comprising the amino acid sequence of SEQ ID NO: 30 (CDR3 of β chain).

In another embodiment of the invention, the TCR comprises a first polypeptide chain comprising a CDR1 comprising the amino acid sequence of SEQ ID NO: 31 (CDR1 of a chain), a CDR2 comprising the amino acid sequence of SEQ ID NO: 32 (CDR2 of α chain), and a CDR3 comprising the amino acid sequence of SEQ ID NO: 33 (CDR3 of α chain), and a second polypeptide chain comprising a CDR1 comprising the amino acid sequence of SEQ ID NO: 34 (CDR1 of β chain), a CDR2 comprising the amino acid sequence of SEQ ID NO: 35 (CDR2 of β chain), and a CDR3 comprising the amino acid sequence of SEQ ID NO: 36 (CDR3 of β chain).

In another embodiment of the invention, the TCR comprises a first polypeptide chain comprising a CDR1 comprising the amino acid sequence of SEQ ID NO: 37 (CDR1 of a chain), a CDR2 comprising the amino acid sequence of SEQ ID NO: 38 (CDR2 of α chain), and a CDR3 comprising the amino acid sequence of SEQ ID NO: 39 (CDR3 of α chain), and a second polypeptide chain comprising a CDR1 comprising the amino acid sequence of SEQ ID NO: 40 (CDR1 of β chain), a CDR2 comprising the amino acid sequence of SEQ ID NO: 41 (CDR2 of β chain), and a CDR3 comprising the amino acid sequence of SEQ ID NO: 42 (CDR3 of β chain).

In another embodiment of the invention, the TCR comprises a first polypeptide chain comprising a CDR1 comprising the amino acid sequence of SEQ ID NO: 43 (CDR1 of a chain), a CDR2 comprising the amino acid sequence of SEQ ID NO: 44 (CDR2 of α chain), and a CDR3 comprising the amino acid sequence of SEQ ID NO: 45 (CDR3 of α chain), and a second polypeptide chain comprising a CDR1 comprising the amino acid sequence of SEQ ID NO: 46 (CDR1 of β chain), a CDR2 comprising the amino acid sequence of SEQ ID NO: 47 (CDR2 of β chain), and a CDR3 comprising the amino acid sequence of SEQ ID NO: 48 (CDR3 of β chain).

In another embodiment of the invention, the TCR comprises a first polypeptide chain comprising a CDR1 comprising the amino acid sequence of SEQ ID NO: 49 (CDR1 of a chain), a CDR2 comprising the amino acid sequence of SEQ ID NO: 50 (CDR2 of α chain), and a CDR3 comprising the amino acid sequence of SEQ ID NO: 51 (CDR3 of α chain), and a second polypeptide chain comprising a CDR1 comprising the amino acid sequence of SEQ ID NO: 52 (CDR1 of β chain), a CDR2 comprising the amino acid sequence of SEQ ID NO: 53 (CDR2 of β chain), and a CDR3 comprising the amino acid sequence of SEQ ID NO: 54 (CDR3 of β chain).

Any CDR3 of SEQ ID NOS: 3, 6, 9, 12, 15, 18, 21, 24, 27, 30, 33, 36, 39, 42, 45, 48, 51, or 54, i.e., of the α chain or β chain or both, may further comprise a cysteine immediately N-terminal to the first amino acid of the CDR or a phenylalanine immediately C-terminal to the final amino acid or both.

In this regard, the inventive TCR can comprise any one or more of the amino acid sequences selected from SEQ ID NOs: 1-6, 7-12, 13-18, 19-24, 25-30, 31-36, 37-42, 43-48, and 49-54. In an embodiment of the invention, the TCR comprises the amino acid sequences of (a) SEQ ID NOs: 1-3, (b) SEQ ID NOs: 4-6, (c) SEQ ID NOs: 7-9, (d) SEQ ID NOs: 10-12, (e) SEQ ID NOs: 13-15, (f) SEQ ID NOs: 16-18, (g) SEQ ID NOs: 19-21, (h) SEQ ID NOs: 22-24, (i) SEQ ID NOs: 25-27, (j) SEQ ID NOs: 28-30, (k) SEQ ID NOs: 31-33, (1) SEQ ID NOs: 34-36, (m) SEQ ID NOs: 37-39, (n) SEQ ID NOs: 40-42, (o) SEQ ID NOs: 43-45, (p) SEQ ID NOs: 46-48, (q) SEQ ID NOs: 49-51, (r) SEQ ID NOs: 52-54, (s) SEQ ID NOs: 1-6, (t) SEQ ID NOs: 7-12, (u) SEQ ID NOs: 13-18, (v) SEQ ID NOs: 19-24, (w) SEQ ID NOs: 25-30, (x) SEQ ID NOs: 31-36, (y) SEQ ID NOs: 37-42, (z) SEQ ID NOs: 43-48, or (aa) SEQ ID NOs: 49-54.

In embodiments of the invention, the TCR comprises an amino acid sequence of a variable region of a TCR comprising the CDRs set forth above. In this regard, the TCR can comprise the amino acid sequence of SEQ ID NO: 56 (variable region of a chain with N-terminal signal peptide); SEQ ID NO: 57 or 58 (variable region of β chain with N-terminal signal peptide); SEQ ID NO: 59 (variable region of a chain with N-terminal signal peptide); SEQ ID NO: 60 or 61 (variable region of β chain with N-terminal signal peptide); SEQ ID NO: 62 (variable region of a chain with N-terminal signal peptide); SEQ ID NO: 63 or 64 (variable region of β chain with N-terminal signal peptide); SEQ ID NO: 65 (variable region of a chain with N-terminal signal peptide); SEQ ID NO: 66 or 67 (variable region of β chain with N-terminal signal peptide); SEQ ID NO: 68 (variable region of a chain with N-terminal signal peptide); SEQ ID NO: 69 or 70 (variable region of β chain with N-terminal signal peptide); SEQ ID NO: 71 (variable region of a chain with N-terminal signal peptide); SEQ ID NO: 72 or 73 (variable region of β chain with N-terminal signal peptide); SEQ ID NO: 74 (variable region of a chain with N-terminal signal peptide); SEQ ID NO: 75 or 76 (variable region of β chain with N-terminal signal peptide); SEQ ID NO: 77 (variable region of a chain with N-terminal signal peptide); SEQ ID NO: 78 or 79 (variable region of β chain with N-terminal signal peptide); SEQ ID NO: 80 (variable region of a chain with N-terminal signal peptide); SEQ ID NO: 81 or 82 (variable region of β chain with N-terminal signal peptide); SEQ ID NO: 83 (variable region of a chain without N-terminal signal peptide); SEQ ID NO: 84 (variable region of β chain without N-terminal signal peptide); SEQ ID NO: 85 (variable region of a chain without N-terminal signal peptide); SEQ ID NO: 86 (variable region of β chain without N-terminal signal peptide); SEQ ID NO: 87 (variable region of a chain without N-terminal signal peptide); SEQ ID NO: 88 (variable region of β chain without N-terminal signal peptide); SEQ ID NO: 89 (variable region of a chain without N-terminal signal peptide); SEQ ID NO: 90 (variable region of β chain without N-terminal signal peptide); SEQ ID NO: 91 (variable region of a chain without N-terminal signal peptide); SEQ ID NO: 92 (variable region of β chain without N-terminal signal peptide); SEQ ID NO: 93 (variable region of a chain without N-terminal signal peptide); SEQ ID NO: 94 (variable region of β chain without N-terminal signal peptide); SEQ ID NO: 95 (variable region of a chain without N-terminal signal peptide); SEQ ID NO: 96 (variable region of β chain without N-terminal signal peptide); SEQ ID NO: 97 (variable region of a chain without N-terminal signal peptide); SEQ ID NO: 98 (variable region of β chain without N-terminal signal peptide); SEQ ID NO: 99 (variable region of a chain without N-terminal signal peptide); SEQ ID NO: 100 (variable region of β chain without N-terminal signal peptide); both of SEQ ID NOs: 56, and 57 or 58; both of SEQ ID NOs: 59, and 60 or 61; both of SEQ ID NOs: 62, and 63 or 64; both of SEQ ID NOs: 65, and 66 or 67; both of SEQ ID NOs: 68, and 69 or 70; both of SEQ ID NOs: 71, and 72 or 73; both of SEQ ID NOs: 74, and 75 or 76; both of SEQ ID NOs: 77, and 78 or 79; both of SEQ ID NOs: 80, and 81 or 82; both of SEQ ID NOs: 83 and 84; both of SEQ ID NOs: 85 and 86; both of SEQ ID NOs: 87 and 88; both of SEQ ID NOs: 89 and 90; both of SEQ ID NOs: 91 and 92; both of SEQ ID NOs: 93 and 94; both of SEQ ID NOs: 95 and 96; both of SEQ ID NOs: 97 and 98; or both of SEQ ID NOs: 99 and 100. In embodiments, the TCR may comprise a human variable region, e.g., a human α chain variable region and a human β chain variable region.

The inventive TCRs may further comprise an α chain constant region and a β chain constant region. The constant region may be derived from any suitable species such as, e.g., human or mouse. In embodiments of the invention, the TCRs further comprise murine α and β chain constant regions or human α and β chain constant regions. As used herein, the term “murine” or “human,” when referring to a TCR or any component of a TCR described herein (e.g., complementarity determining region (CDR), variable region, constant region, α chain, and/or β chain), means a TCR (or component thereof) which is derived from a mouse or a human, respectively, i.e., a TCR (or component thereof) that originated from or was, at one time, expressed by a mouse T cell or a human T cell, respectively.

An embodiment of the invention provides a chimeric TCR comprising a murine variable region and a murine constant region, wherein the TCR has antigenic specificity for a CD20 amino acid sequence presented by an HLA Class I molecule. The murine constant region may provide any one or more advantages. For example, the murine constant region may diminish mispairing of the inventive TCR with the endogenous TCRs of the host cell into which the inventive TCR is introduced. Alternatively or additionally, the murine constant region may increase expression of the inventive TCR. The chimeric TCR may comprise the amino acid sequence of SEQ ID NO: 101 (wild-type (WT) murine α chain constant region), SEQ ID NO: 102 (WT murine β chain constant region), or both SEQ ID NOs: 101 and 102. Preferably, the inventive TCR comprises the amino acid sequences of both of SEQ ID NOs: 101 and 102. The chimeric TCR may comprise any of the murine constant regions described herein in combination with any of the CDR regions as described herein with respect to other aspects of the invention. In this regard, the TCR may comprise the amino acid sequences of (a) SEQ ID NOs: 1-3 and 101, (b) SEQ ID NOs: 4-6 and 102, (c) SEQ ID NOs: 7-9 and 101, (d) SEQ ID NOs: 10-12 and 102, (e) SEQ ID NOs: 13-15 and 101, (0 SEQ ID NOs: 16-18 and 102, (g) SEQ ID NOs: 19-21 and 101, (h) SEQ ID NOs: 22-24 and 102, (i) SEQ ID NOs: 25-27 and 101, (j) SEQ ID NOs: 28-30 and 102, (k) SEQ ID NOs: 31-33 and 101, (1) SEQ ID NOs: 34-36 and 102, (m) SEQ ID NOs: 37-39 and 101, (n) SEQ ID NOs: 40-42 and 102, (o) SEQ ID NOs: 43-45 and 101, (p) SEQ ID NOs: 46-48 and 102, (q) SEQ ID NOs: 49-51 and 101, (r) SEQ ID NOs: 52-54 and 102, (s) SEQ ID NOs: 1-6 and 101 and 102, (t) SEQ ID NOs: 7-12 and 101 and 102, (u) SEQ ID NOs: 13-18 and 101 and 102, (v) SEQ ID NOs: 19-24 and 101 and 102, (w) SEQ ID NOs: 25-30 and 101 and 102, (x) SEQ ID NOs: 31-36 and 101 and 102, (y) SEQ ID NOs: 37-42 and 101 and 102, (z) SEQ ID NOs: 43-48 and 101 and 102, or (aa) SEQ ID NOs: 49-54 and 101 and 102.

In another embodiment of the invention, the chimeric TCR may comprise any of the murine constant regions described herein in combination with any of the variable regions described herein with respect to other aspects of the invention. In this regard, the TCR may comprise the amino acid sequences of (a) both of SEQ ID NOS: 56 and 101; (b) both of SEQ ID NOS: 57 or 58, and 102; (c) both of SEQ ID NOS: 59 and 101; (d) both of SEQ ID NOS: 60 or 61, and 102; (e) both of SEQ ID NOS: 62 and 101; (0 both of SEQ ID NOS: 63 or 64, and 102; (g) both of SEQ ID NOS: 65 and 101; (h) both of SEQ ID NOS: 66 or 67, and 102; (i) both of SEQ ID NOS: 68 and 101; (j) both of SEQ ID NOS: 69 or 70, and 102; (k) both of SEQ ID NOS: 71 and 101; (1) both of SEQ ID NOS: 72 or 73, and 102; (m) both of SEQ ID NOS: 74 and 101; (n) both of SEQ ID NOS: 75 or 76, and 102; (o) both of SEQ ID NOS: 77 and 101; (p) both of SEQ ID NOS: 78 or 79, and 102; (q) both of SEQ ID NOS: 80 and 101; (r) both of SEQ ID NOS: 81 or 82, and 102; (s) all of SEQ ID NOS: 56 and 101, and either of 57 or 58, and 102; (t) all of SEQ ID NOS: 59 and 101, and either of 60 or 61, and 102; (u) all of SEQ ID NOS: 62 and 101, and either of 63 or 64, and 102; (v) all of SEQ ID NOS: 65 and 101, and either of 66 or 67, and 102; (w) all of SEQ ID NOS: 68 and 101, and either of 69 or 70, and 102; (x) all of SEQ ID NOS: 71 and 101, and either of 72 or 73, and 102; (y) all of SEQ ID NOS: 74 and 101, and either of 75 or 76, and 102; (z) all of SEQ ID NOS: 77 and 101, and either of 78 or 79, and 102; or (aa) all of SEQ ID NOS: 80 and 101, and either of 81 or 82, and 102.

In another embodiment of the invention, the TCR comprises the amino acid sequence(s) of SEQ ID NO: 103 (α chain with WT murine constant region and N-terminal signal peptide), SEQ ID NO: 104 or 105 (β chain with WT murine constant region and N-terminal signal peptide), SEQ ID NO: 106 (α chain with WT murine constant region and N-terminal signal peptide), SEQ ID NO: 107 or 108 (β chain with WT murine constant region and N-terminal signal peptide), SEQ ID NO: 109 (α chain with WT murine constant region and N-terminal signal peptide), SEQ ID NO: 110 or 111 (β chain with WT murine constant region and N-terminal signal peptide), SEQ ID NO: 112 (α chain with WT murine constant region and N-terminal signal peptide), SEQ ID NO: 113 or 114 (β chain with WT murine constant region and N-terminal signal peptide), SEQ ID NO: 115 (α chain with WT murine constant region and N-terminal signal peptide), SEQ ID NO: 116 or 117 (β chain with WT murine constant region and N-terminal signal peptide), SEQ ID NO: 118 (α chain with WT murine constant region and N-terminal signal peptide), SEQ ID NO: 119 or 120 (β chain with WT murine constant region and N-terminal signal peptide), SEQ ID NO: 121 (α chain with WT murine constant region and N-terminal signal peptide), SEQ ID NO: 122 or 123 (β chain with WT murine constant region and N-terminal signal peptide), SEQ ID NO: 124 (α chain with WT murine constant region and N-terminal signal peptide), SEQ ID NO: 125 or 126 (β chain with WT murine constant region and N-terminal signal peptide), SEQ ID NO: 127 (α chain with WT murine constant region and N-terminal signal peptide), SEQ ID NO: 128 or 129 (β chain with WT murine constant region and N-terminal signal peptide), SEQ ID NO: 130 (α chain with WT murine constant region and without N-terminal signal peptide), SEQ ID NO: 131 (β chain with WT murine constant region and without N-terminal signal peptide), SEQ ID NO: 132 (α chain with WT murine constant region and without N-terminal signal peptide), SEQ ID NO: 133 (β chain with WT murine constant region and without N-terminal signal peptide), SEQ ID NO: 134 (α chain with WT murine constant region and without N-terminal signal peptide), SEQ ID NO: 135 (β chain with WT murine constant region and without N-terminal signal peptide), SEQ ID NO: 136 (α chain with WT murine constant region and without N-terminal signal peptide), SEQ ID NO: 137 (β chain with WT murine constant region and without N-terminal signal peptide), SEQ ID NO: 138 (α chain with WT murine constant region and without N-terminal signal peptide), SEQ ID NO: 139 (β chain with WT murine constant region and without N-terminal signal peptide), SEQ ID NO: 140 (α chain with WT murine constant region and without N-terminal signal peptide), SEQ ID NO: 141 (β chain with WT murine constant region and without N-terminal signal peptide), SEQ ID NO: 142 (α chain with WT murine constant region and without N-terminal signal peptide), SEQ ID NO: 143 (β chain with WT murine constant region and without N-terminal signal peptide), SEQ ID NO: 144 (α chain with WT murine constant region and without N-terminal signal peptide), SEQ ID NO: 145 (β chain with WT murine constant region and without N-terminal signal peptide), SEQ ID NO: 146 (α chain with WT murine constant region and without N-terminal signal peptide), SEQ ID NO: 147 (β chain with WT murine constant region and without N-terminal signal peptide), both of SEQ ID NO: 103, and 104 or 105, both of SEQ ID NO: 106, and 107 or 108, both of SEQ ID NO: 109, and 110 or 111, both of SEQ ID NO: 112, and 113 or 114, both of SEQ ID NO: 115, and 116 or 117, both of SEQ ID NO: 118, and 119 or 120, both of SEQ ID NO: 121, and 122 or 123, both of SEQ ID NO: 124, and 125 or 126, both of SEQ ID NO: 127, and 128 or 129, both of SEQ ID NOS: 130 and 131; both of SEQ ID NOS: 132 and 133; both of SEQ ID NOS: 134 and 135; both of SEQ ID NOS: 136 and 137; both of SEQ ID NOS: 138 and 139; both of SEQ ID NOS: 140 and 141; both of SEQ ID NOS: 142 and 143; both of SEQ ID NOS: 144 and 145; or both of SEQ ID NOS: 146 and 147.

In embodiments of the invention, the TCR comprises a substituted constant region. In this regard, the TCR may comprise the amino acid sequence of any of the TCRs described herein with one, two, three, or four amino acid substitution(s) in the constant region of one or both of the α and β chain. Preferably, the TCR comprises a murine constant region with one, two, three, or four amino acid substitution(s) in the murine constant region of one or both of the α and β chains. In an especially preferred embodiment, the TCR comprises a murine constant region with one, two, three, or four amino acid substitution(s) in the murine constant region of the α chain and one amino acid substitution in the murine constant region of the β chain. In some embodiments, the TCRs comprising the substituted constant region advantageously provide one or more of increased recognition of CD20⁺ targets, increased expression by a host cell, diminished mispairing with endogenous TCRs, and increased anti-tumor activity as compared to the parent TCR comprising an unsubstituted (wild-type) constant region. In general, the substituted amino acid sequences of the murine constant regions of the TCR α and β chains, SEQ ID NOs: 148 and 149, respectively, correspond with all or portions of the unsubstituted murine constant region amino acid sequences SEQ ID NOs: 101 and 102, respectively, with SEQ ID NO: 148 having one, two, three, or four amino acid substitution(s) when compared to SEQ ID NO: 101 and SEQ ID NO: 149 having one amino acid substitution when compared to SEQ ID NO: 102. In this regard, an embodiment of the invention provides a TCR comprising the amino acid sequences of (a) SEQ ID NO: 148 (constant region of α chain), wherein (i) X at position 48 is Thr or Cys; (ii) X at position 112 is Ser, Ala, Val, Leu, Ile, Pro, Phe, Met, or Trp; (iii) X at position 114 is Met, Ala, Val, Leu, Ile, Pro, Phe, or Trp; and (iv) X at position 115 is Gly, Ala, Val, Leu, Ile, Pro, Phe, Met, or Trp; (b) SEQ ID NO: 149 (constant region of β chain), wherein X at position 57 is Ser or Cys; or (c) both of SEQ ID NOs: 148 and 149. In embodiments of the invention, the TCR comprising SEQ ID NO: 148 does not comprise SEQ ID NO: 101 (unsubstituted murine constant region of α chain). In embodiments of the invention, the TCR comprising SEQ ID NO: 149 does not comprise SEQ ID NO: 102 (unsubstituted murine constant region of β chain).

The first amino acid of any of the mouse alpha constant regions described herein may be different than N as provided in SEQ ID NOS: 101 and 148. For example, in any TCR construct, polypeptide, protein, etc., as described herein, this first amino acid can be encoded by a split codon (having nucleotides from both a variable region and a constant region) such that any of the murine alpha constant regions may have a different amino acid at that position. For example, SEQ ID NOS: 106, 132, 153, 179, and 197 have an H at the position corresponding to the first amino acid in the constant region, and any of the mouse alpha constant regions described herein may have an H at this position. Similarly, first amino acid of any of the mouse beta constant regions described herein may be different than E as provided in SEQ ID NOS: 102 and 149, e.g., this first amino acid can be encoded by a split codon.

In embodiments of the invention, the TCR comprises an α chain comprising a variable region and a constant region and a β chain comprising a variable region and a constant region. In this regard, the TCR may comprise (a) an α chain comprising the amino acid sequence of SEQ ID NO: 150 (α chain with N-terminal signal peptide), wherein: (i) X at position 178 of SEQ ID NO: 150 is Thr or Cys; (ii) X at position 242 of SEQ ID NO: 150 is Ser, Ala, Val, Leu, Ile, Pro, Phe, Met, or Trp; (iii) X at position 244 of SEQ ID NO: 150 is Met, Ala, Val, Leu, Ile, Pro, Phe, or Trp; and (iv) X at position 245 of SEQ ID NO: 150 is Gly, Ala, Val, Leu, Ile, Pro, Phe, Met, or Trp; (b) a β chain comprising the amino acid sequence of SEQ ID NO: 151 or 152 (β chain with N-terminal signal peptide), wherein X at position 188 of SEQ ID NO: 151 or 152 is Ser or Cys; (c) an α chain comprising the amino acid sequence of SEQ ID NO: 153 (α chain with N-terminal signal peptide), wherein: (i) X at position 179 of SEQ ID NO: 153 is Thr or Cys; (ii) X at position 243 of SEQ ID NO: 153 is Ser, Ala, Val, Leu, Ile, Pro, Phe, Met, or Trp; (iii) X at position 245 of SEQ ID NO: 153 is Met, Ala, Val, Leu, Ile, Pro, Phe, or Trp; and (iv) X at position 246 of SEQ ID NO: 153 is Gly, Ala, Val, Leu, Ile, Pro, Phe, Met, or Trp; (d) a β chain comprising the amino acid sequence of SEQ ID NO: 154 or 155 (β chain with N-terminal signal peptide), wherein X at position 188 of SEQ ID NO: 154 or 155 is Ser or Cys; (e) an α chain comprising the amino acid sequence of SEQ ID NO: 156 (α chain with N-terminal signal peptide), wherein: (i) X at position 178 of SEQ ID NO: 156 is Thr or Cys; (ii) X at position 242 of SEQ ID NO: 156 is Ser, Ala, Val, Leu, Ile, Pro, Phe, Met, or Trp; (iii) X at position 244 of SEQ ID NO: 156 is Met, Ala, Val, Leu, Ile, Pro, Phe, or Trp; and (iv) X at position 245 of SEQ ID NO: 156 is Gly, Ala, Val, Leu, Ile, Pro, Phe, Met, or Trp; (f) a β chain comprising the amino acid sequence of SEQ ID NO: 157 or 158 (β chain with N-terminal signal peptide), wherein X at position 188 of SEQ ID NO: 157 or 158 is Ser or Cys; (g) an α chain comprising the amino acid sequence of SEQ ID NO: 159 (α chain with N-terminal signal peptide), wherein: (i) X at position 178 of SEQ ID NO: 159 is Thr or Cys; (ii) X at position 242 of SEQ ID NO: 159 is Ser, Ala, Val, Leu, Ile, Pro, Phe, Met, or Trp; (iii) X at position 244 of SEQ ID NO: 159 is Met, Ala, Val, Leu, Ile, Pro, Phe, or Trp; and (iv) X at position 245 of SEQ ID NO: 159 is Gly, Ala, Val, Leu, Ile, Pro, Phe, Met, or Trp; (h) a β chain comprising the amino acid sequence of SEQ ID NO: 160 or 161 (β chain with N-terminal signal peptide), wherein X at position 191 of SEQ ID NO: 160 or 161 is Ser or Cys; (I) an α chain comprising the amino acid sequence of SEQ ID NO: 162 (α chain with N-terminal signal peptide), wherein: (i) X at position 191 of SEQ ID NO: 162 is Thr or Cys; (ii) X at position 255 of SEQ ID NO: 162 is Ser, Ala, Val, Leu, Ile, Pro, Phe, Met, or Trp; (iii) X at position 257 of SEQ ID NO: 162 is Met, Ala, Val, Leu, Ile, Pro, Phe, or Trp; and (iv) X at position 258 of SEQ ID NO: 162 is Gly, Ala, Val, Leu, Ile, Pro, Phe, Met, or Trp; (j) a β chain comprising the amino acid sequence of SEQ ID NO: 163 or 164 (β chain with N-terminal signal peptide), wherein X at position 188 of SEQ ID NO: 163 or 164 is Ser or Cys; (k) an α chain comprising the amino acid sequence of SEQ ID NO: 165 (α chain with N-terminal signal peptide), wherein: (i) X at position 180 of SEQ ID NO: 165 is Thr or Cys; (ii) X at position 244 of SEQ ID NO: 165 is Ser, Ala, Val, Leu, Ile, Pro, Phe, Met, or Trp; (iii) X at position 246 of SEQ ID NO: 165 is Met, Ala, Val, Leu, Ile, Pro, Phe, or Trp; and (iv) X at position 247 of SEQ ID NO: 165 is Gly, Ala, Val, Leu, Ile, Pro, Phe, Met, or Trp; (1) a β chain comprising the amino acid sequence of SEQ ID NO: 166 or 167 (β chain with N-terminal signal peptide), wherein X at position 190 of SEQ ID NO: 166 or 167 is Ser or Cys; (m) an α chain comprising the amino acid sequence of SEQ ID NO: 168 (α chain with N-terminal signal peptide), wherein: (i) X at position 180 of SEQ ID NO: 168 is Thr or Cys; (ii) X at position 244 of SEQ ID NO: 168 is Ser, Ala, Val, Leu, Ile, Pro, Phe, Met, or Trp; (iii) X at position 246 of SEQ ID NO: 168 is Met, Ala, Val, Leu, Ile, Pro, Phe, or Trp; and (iv) X at position 247 of SEQ ID NO: 168 is Gly, Ala, Val, Leu, Ile, Pro, Phe, Met, or Trp; (n) a β chain comprising the amino acid sequence of SEQ ID NO: 169 or 170 (β chain with N-terminal signal peptide), wherein X at position 188 of SEQ ID NO: 169 or 170 is Ser or Cys; (o) an α chain comprising the amino acid sequence of SEQ ID NO: 171 (α chain with N-terminal signal peptide), wherein: (i) X at position 177 of SEQ ID NO: 171 is Thr or Cys; (ii) X at position 241 of SEQ ID NO: 171 is Ser, Ala, Val, Leu, Ile, Pro, Phe, Met, or Trp; (iii) X at position 243 of SEQ ID NO: 171 is Met, Ala, Val, Leu, Ile, Pro, Phe, or Trp; and (iv) X at position 244 of SEQ ID NO: 171 is Gly, Ala, Val, Leu, Ile, Pro, Phe, Met, or Trp; (p) a β chain comprising the amino acid sequence of SEQ ID NO: 172 or 173 (β chain with N-terminal signal peptide), wherein X at position 188 of SEQ ID NO: 172 or 173 is Ser or Cys; (q) an α chain comprising the amino acid sequence of SEQ ID NO: 174 (α chain with N-terminal signal peptide), wherein: (i) X at position 184 of SEQ ID NO: 174 is Thr or Cys; (ii) X at position 248 of SEQ ID NO: 174 is Ser, Ala, Val, Leu, Ile, Pro, Phe, Met, or Trp; (iii) X at position 250 of SEQ ID NO: 174 is Met, Ala, Val, Leu, Ile, Pro, Phe, or Trp; and (iv) X at position 251 of SEQ ID NO: 174 is Gly, Ala, Val, Leu, Ile, Pro, Phe, Met, or Trp; (r) a β chain comprising the amino acid sequence of SEQ ID NO: 175 or 176 (β chain with N-terminal signal peptide), wherein X at position 189 of SEQ ID NO: 175 or 176 is Ser or Cys; (s) both (a) and (b); (t) both (c) and (d); (u) both (e) and (0; (v) both (g) and (h); (w) both (I) and (j); (x) both (k) and (1); (y) both (m) and (n); (z) both (o) and (p); (aa) both (q) and (r); (ab) an α chain comprising the amino acid sequence of SEQ ID NO: 177 (α chain without N-terminal signal peptide), wherein: (i) X at position 158 of SEQ ID NO: 177 is Thr or Cys; (ii) X at position 222 of SEQ ID NO: 177 is Ser, Ala, Val, Leu, Ile, Pro, Phe, Met, or Trp; (iii) X at position 224 of SEQ ID NO: 177 is Met, Ala, Val, Leu, Ile, Pro, Phe, or Trp; and (iv) X at position 225 of SEQ ID NO: 177 is Gly, Ala, Val, Leu, Ile, Pro, Phe, Met, or Trp; (ac) a β chain comprising the amino acid sequence of SEQ ID NO: 178 (β chain without N-terminal signal peptide), wherein X at position 167 of SEQ ID NO: 178 is Ser or Cys; (ad) an α chain comprising the amino acid sequence of SEQ ID NO: 179 (α chain without N-terminal signal peptide), wherein: (i) X at position 159 of SEQ ID NO: 179 is Thr or Cys; (ii) X at position 223 of SEQ ID NO: 179 is Ser, Ala, Val, Leu, Ile, Pro, Phe, Met, or Trp; (iii) X at position 225 of SEQ ID NO: 179 is Met, Ala, Val, Leu, Ile, Pro, Phe, or Trp; and (iv) X at position 226 of SEQ ID NO: 179 is Gly, Ala, Val, Leu, Ile, Pro, Phe, Met, or Trp; (ae) a β chain comprising the amino acid sequence of SEQ ID NO: 180 (β chain without N-terminal signal peptide), wherein X at position 169 of SEQ ID NO: 180 is Ser or Cys; (af) an α chain comprising the amino acid sequence of SEQ ID NO: 181 (α chain without N-terminal signal peptide), wherein: (i) X at position 158 of SEQ ID NO: 181 is Thr or Cys; (ii) X at position 222 of SEQ ID NO: 181 is Ser, Ala, Val, Leu, Ile, Pro, Phe, Met, or Trp; (iii) X at position 224 of SEQ ID NO: 181 is Met, Ala, Val, Leu, Ile, Pro, Phe, or Trp; and (iv) X at position 225 of SEQ ID NO: 181 is Gly, Ala, Val, Leu, Ile, Pro, Phe, Met, or Trp; (ag) a β chain comprising the amino acid sequence of SEQ ID NO: 182 (β chain without N-terminal signal peptide), wherein X at position 167 of SEQ ID NO: 182 is Ser or Cys; (ah) an α chain comprising the amino acid sequence of SEQ ID NO: 183 (α chain without N-terminal signal peptide), wherein: (i) X at position 158 of SEQ ID NO: 183 is Thr or Cys; (ii) X at position 222 of SEQ ID NO: 183 is Ser, Ala, Val, Leu, Ile, Pro, Phe, Met, or Trp; (iii) X at position 224 of SEQ ID NO: 183 is Met, Ala, Val, Leu, Ile, Pro, Phe, or Trp; and (iv) X at position 225 of SEQ ID NO: 183 is Gly, Ala, Val, Leu, Ile, Pro, Phe, Met, or Trp; (ai) a β chain comprising the amino acid sequence of SEQ ID NO: 184 (β chain without N-terminal signal peptide), wherein X at position 169 of SEQ ID NO: 184 is Ser or Cys; (aj) an α chain comprising the amino acid sequence of SEQ ID NO: 185 (α chain without N-terminal signal peptide), wherein: (i) X at position 163 of SEQ ID NO: 185 is Thr or Cys; (ii) X at position 227 of SEQ ID NO: 185 is Ser, Ala, Val, Leu, Ile, Pro, Phe, Met, or Trp; (iii) X at position 229 of SEQ ID NO: 185 is Met, Ala, Val, Leu, Ile, Pro, Phe, or Trp; and (iv) X at position 230 of SEQ ID NO: 185 is Gly, Ala, Val, Leu, Ile, Pro, Phe, Met, or Trp; (ak) a β chain comprising the amino acid sequence of SEQ ID NO: 186 (β chain without N-terminal signal peptide), wherein X at position 169 of SEQ ID NO: 186 is Ser or Cys; (al) an α chain comprising the amino acid sequence of SEQ ID NO: 187 (α chain without N-terminal signal peptide), wherein: (i) X at position 159 of SEQ ID NO: 187 is Thr or Cys; (ii) X at position 223 of SEQ ID NO: 187 is Ser, Ala, Val, Leu, Ile, Pro, Phe, Met, or Trp; (iii) X at position 225 of SEQ ID NO: 187 is Met, Ala, Val, Leu, Ile, Pro, Phe, or Trp; and (iv) X at position 226 of SEQ ID NO: 187 is Gly, Ala, Val, Leu, Ile, Pro, Phe, Met, or Trp; (am) a β chain comprising the amino acid sequence of SEQ ID NO: 188 (β chain without N-terminal signal peptide), wherein X at position 172 of SEQ ID NO: 188 is Ser or Cys; (an) an α chain comprising the amino acid sequence of SEQ ID NO: 189 (α chain without N-terminal signal peptide), wherein: (i) X at position 160 of SEQ ID NO: 189 is Thr or Cys; (ii) X at position 224 of SEQ ID NO: 189 is Ser, Ala, Val, Leu, Ile, Pro, Phe, Met, or Trp; (iii) X at position 226 of SEQ ID NO: 189 is Met, Ala, Val, Leu, Ile, Pro, Phe, or Trp; and (iv) X at position 227 of SEQ ID NO: 189 is Gly, Ala, Val, Leu, Ile, Pro, Phe, Met, or Trp; (ao) a β chain comprising the amino acid sequence of SEQ ID NO: 190 (β chain without N-terminal signal peptide), wherein X at position 169 of SEQ ID NO: 190 is Ser or Cys; (ap) an α chain comprising the amino acid sequence of SEQ ID NO: 191 (α chain without N-terminal signal peptide), wherein: (i) X at position 157 of SEQ ID NO: 191 is Thr or Cys; (ii) X at position 221 of SEQ ID NO: 191 is Ser, Ala, Val, Leu, Ile, Pro, Phe, Met, or Trp; (iii) X at position 223 of SEQ ID NO: 191 is Met, Ala, Val, Leu, Ile, Pro, Phe, or Trp; and (iv) X at position 224 of SEQ ID NO: 191 is Gly, Ala, Val, Leu, Ile, Pro, Phe, Met, or Trp; (aq) a β chain comprising the amino acid sequence of SEQ ID NO: 192 (β chain without N-terminal signal peptide), wherein X at position 169 of SEQ ID NO: 192 is Ser or Cys; (ar) an α chain comprising the amino acid sequence of SEQ ID NO: 193 (α chain without N-terminal signal peptide), wherein: (i) X at position 163 of SEQ ID NO: 193 is Thr or Cys; (ii) X at position 227 of SEQ ID NO: 193 is Ser, Ala, Val, Leu, Ile, Pro, Phe, Met, or Trp; (iii) X at position 229 of SEQ ID NO: 193 is Met, Ala, Val, Leu, Ile, Pro, Phe, or Trp; and (iv) X at position 230 of SEQ ID NO: 193 is Gly, Ala, Val, Leu, Ile, Pro, Phe, Met, or Trp; (as) a β chain comprising the amino acid sequence of SEQ ID NO: 194 (β chain without N-terminal signal peptide), wherein X at position 170 of SEQ ID NO: 194 is Ser or Cys; (at) both (ab) and (ac); (au) both (ad) and (ae); (av) both (af) and (ag); (aw) both (ah) and (ai); (ax) both (aj) and (ak); (ay) both (al) and (am); (az) both (an) and (ao); (ba) both (ap) and (aq); or (bb) both (ar) and (as).

In embodiments of the invention, the TCR comprising SEQ ID NO: 150 does not comprise SEQ ID NO: 103 (unsubstituted α chain). In embodiments of the invention, the TCR comprising SEQ ID NO: 151 or 152 does not comprise SEQ ID NO: 104 or 105 (unsubstituted β chain). In embodiments of the invention, the TCR comprising SEQ ID NO: 153 does not comprise SEQ ID NO: 106 (unsubstituted α chain). In embodiments of the invention, the TCR comprising SEQ ID NO: 154 or 155 does not comprise SEQ ID NO: 107 or 108 (unsubstituted β chain). In embodiments of the invention, the TCR comprising SEQ ID NO: 156 does not comprise SEQ ID NO: 109 (unsubstituted α chain). In embodiments of the invention, the TCR comprising SEQ ID NO: 157 or 158 does not comprise SEQ ID NO: 110 or 111 (unsubstituted β chain). In embodiments of the invention, the TCR comprising SEQ ID NO: 159 does not comprise SEQ ID NO: 112 (unsubstituted α chain). In embodiments of the invention, the TCR comprising SEQ ID NO: 160 or 161 does not comprise SEQ ID NO: 113 or 114 (unsubstituted β chain). In embodiments of the invention, the TCR comprising SEQ ID NO: 162 does not comprise SEQ ID NO: 115 (unsubstituted α chain). In embodiments of the invention, the TCR comprising SEQ ID NO: 163 or 164 does not comprise SEQ ID NO: 116 or 117 (unsubstituted β chain). In embodiments of the invention, the TCR comprising SEQ ID NO: 165 does not comprise SEQ ID NO: 118 (unsubstituted α chain). In embodiments of the invention, the TCR comprising SEQ ID NO: 166 or 167 does not comprise SEQ ID NO: 119 or 120 (unsubstituted β chain). In embodiments of the invention, the TCR comprising SEQ ID NO: 168 does not comprise SEQ ID NO: 121 (unsubstituted α chain). In embodiments of the invention, the TCR comprising SEQ ID NO: 169 or 170 does not comprise SEQ ID NO: 122 or 123 (unsubstituted β chain). In embodiments of the invention, the TCR comprising SEQ ID NO: 171 does not comprise SEQ ID NO: 124 (unsubstituted α chain). In embodiments of the invention, the TCR comprising SEQ ID NO: 172 or 173 does not comprise SEQ ID NO: 125 or 126 (unsubstituted β chain). In embodiments of the invention, the TCR comprising SEQ ID NO: 174 does not comprise SEQ ID NO: 127 (unsubstituted α chain). In embodiments of the invention, the TCR comprising SEQ ID NO: 175 or 176 does not comprise SEQ ID NO: 128 or 129 (unsubstituted β chain). In embodiments of the invention, the TCR comprising SEQ ID NO: 177 does not comprise SEQ ID NO: 130 (unsubstituted α chain). In embodiments of the invention, the TCR comprising SEQ ID NO: 178 does not comprise SEQ ID NO: 131 (unsubstituted β chain). In embodiments of the invention, the TCR comprising SEQ ID NO: 179 does not comprise SEQ ID NO: 132 (unsubstituted α chain). In embodiments of the invention, the TCR comprising SEQ ID NO: 180 does not comprise SEQ ID NO: 133 (unsubstituted β chain). In embodiments of the invention, the TCR comprising SEQ ID NO: 181 does not comprise SEQ ID NO: 134 (unsubstituted α chain). In embodiments of the invention, the TCR comprising SEQ ID NO: 182 does not comprise SEQ ID NO: 135 (unsubstituted β chain). In embodiments of the invention, the TCR comprising SEQ ID NO: 183 does not comprise SEQ ID NO: 136 (unsubstituted α chain). In embodiments of the invention, the TCR comprising SEQ ID NO: 184 does not comprise SEQ ID NO: 137 (unsubstituted β chain). In embodiments of the invention, the TCR comprising SEQ ID NO: 185 does not comprise SEQ ID NO: 138 (unsubstituted α chain). In embodiments of the invention, the TCR comprising SEQ ID NO: 186 does not comprise SEQ ID NO: 139 (unsubstituted β chain). In embodiments of the invention, the TCR comprising SEQ ID NO: 187 does not comprise SEQ ID NO: 140 (unsubstituted α chain). In embodiments of the invention, the TCR comprising SEQ ID NO: 188 does not comprise SEQ ID NO: 141 (unsubstituted β chain). In embodiments of the invention, the TCR comprising SEQ ID NO: 189 does not comprise SEQ ID NO: 142 (unsubstituted α chain). In embodiments of the invention, the TCR comprising SEQ ID NO: 190 does not comprise SEQ ID NO: 143 (unsubstituted β chain). In embodiments of the invention, the TCR comprising SEQ ID NO: 191 does not comprise SEQ ID NO: 144 (unsubstituted α chain). In embodiments of the invention, the TCR comprising SEQ ID NO: 192 does not comprise SEQ ID NO: 145 (unsubstituted β chain). In embodiments of the invention, the TCR comprising SEQ ID NO: 193 does not comprise SEQ ID NO: 146 (unsubstituted α chain). In embodiments of the invention, the TCR comprising SEQ ID NO: 194 does not comprise SEQ ID NO: 147 (unsubstituted β chain).

In embodiments of the invention, the substituted constant region includes cysteine substitutions in the constant region of one or both of the α and β chains to provide a cysteine-substituted TCR. Opposing cysteines in the α and the β chains provide a disulfide bond that links the constant regions of the α and the β chains of the substituted TCR to one another and which is not present in a TCR comprising the unsubstituted murine constant regions. In this regard, the TCR may be a cysteine-substituted TCR in which one or both of the native Thr at position 48 (Thr48) of SEQ ID NO: 101 and the native Ser at position 57 (Ser57) of SEQ ID NO: 102 may be substituted with Cys. Preferably, both of the native Thr48 of SEQ ID NO: 101 and the native Ser57 of SEQ ID NO: 102 are substituted with Cys. Examples of cysteine-substituted TCR constant regions sequences are set forth in Table 1. In embodiments of the invention, the cysteine-substituted TCR comprises (i) SEQ ID NO: 148, (ii) SEQ ID NO: 149, or (iii) both of SEQ ID NOs: 148 and 149, wherein both of SEQ ID NOs: 148 and 149 are as defined in Table 1. The cysteine-substituted TCRs of the invention may include the substituted constant region in addition to any of the CDRs or variable regions described herein.

In embodiments of the invention, the cysteine-substituted, chimeric TCR comprises a full length alpha chain and a full-length beta chain. Examples of cysteine-substituted, chimeric TCR alpha chain and beta chain sequences are set forth in Table 1. In embodiments of the invention, the TCR comprises SEQ ID NO: 150; SEQ ID NO: 151 or 152; SEQ ID NO: 153; SEQ ID NO: 154 or 155; SEQ ID NO: 156; SEQ ID NO: 157 or 158; SEQ ID NO: 159; SEQ ID NO: 160 or 161; SEQ ID NO: 162; SEQ ID NO: 163 or 164; SEQ ID NO: 165; SEQ ID NO: 166 or 167; SEQ ID NO: 168; SEQ ID NO: 169 or 170; SEQ ID NO: 171; SEQ ID NO: 172 or 173; SEQ ID NO: 174; SEQ ID NO: 175 or 176; both of SEQ ID NO: 150, and 151 or 152; both of SEQ ID NO: 153, and 154 or 155; both of SEQ ID NO: 156, and 157 or 158; both of SEQ ID NO: 159, and 160 or 161; both of SEQ ID NO: 162, and 163 or 164; both of SEQ ID NO: 165, and 166 or 167; both of SEQ ID NO: 168, and 169 or 170; both of SEQ ID NO: 171, and 172 or 173; both of SEQ ID NO: 174, and 175 or 176, SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; SEQ ID NO: 186; SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; both of SEQ ID NO: 177 and 178; both of SEQ ID NO: 179 and 180; both of SEQ ID NO: 181 and 182; both of SEQ ID NO: 183 and 184; both of SEQ ID NO: 185 and 186; both of SEQ ID NO: 187 and 188; both of SEQ ID NO: 189 and 190; both of SEQ ID NO: 191 and 192; or both of SEQ ID NO: 193 and 194; wherein all of the sequences are as defined in Table 1.

TABLE 1

| SEQ ID NO: | Definitions of “X” |
|---|---|
| SEQ ID NO: 148 | X at position 48 is Cys, |
| (constant region α chain) | X at position 112 is Ser, |
| | X at position 114 is Met, and |
| | X at position 115 is Gly. |
| SEQ ID NO: 149 | X at position 57 is Cys |
| (constant region β chain) | |
| SEQ ID NO: 150 | X at position 178 is Cys, |
| (TCR J1A1 α chain with | X at position 242 is Ser, |
| N-terminal signal peptide) | X at position 244 is Met, and |
| | X at position 245 is Gly. |
| SEQ ID NO: 151 or 152 | X at position 188 is Cys |
| (TCR J1A1 β chain with | |
| N-terminal signal peptide) | |
| SEQ ID NO: 153 | X at position 179 is Cys, |
| (TCR J1A2 α chain with | X at position 243 is Ser, |
| N-terminal signal peptide) | X at position 245 is Met, and |
| | X at position 246 is Gly. |
| SEQ ID NO: 154 or 155 | X at position 188 is Cys |
| (TCR J1A2 β chain with | |
| N-terminal signal peptide) | |
| SEQ ID NO: 156 | X at position 178 is Cys, |
| (TCR J1A3 α chain with | X at position 242 is Ser, |
| N-terminal signal peptide) | X at position 244 is Met, and |
| | X at position 245 is Gly. |
| SEQ ID NO: 157 or 158 | X at position 188 is Cys |
| (TCR J1A3 β chain with | |
| N-terminal signal peptide) | |
| SEQ ID NO: 159 | X at position 178 is Cys, |
| (TCR 869A1 α chain with | X at position 242 is Ser, |
| N-terminal signal peptide) | X at position 244 is Met, and |
| | X at position 245 is Gly. |
| SEQ ID NO: 160 or 161 | X at position 191 is Cys |
| (TCR 869A1 β chain with | |
| N-terminal signal peptide) | |
| SEQ ID NO: 162 | X at position 191 is Cys, |
| (TCR J2B1 α chain with | X at position 255 is Ser, |
| N-terminal signal peptide) | X at position 257 is Met, and |
| | X at position 258 is Gly. |
| SEQ ID NO: 163 or 164 | X at position 188 is Cys |
| (TCR J2B1 β chain with | |
| N-terminal signal peptide) | |
| SEQ ID NO: 165 | X at position 180 is Cys, |
| (TCR 56A22 α chain with | X at position 244 is Ser, |
| N-terminal signal peptide) | X at position 246 is Met, and |
| | X at position 247 is Gly. |
| SEQ ID NO: 166 or 167 | X at position 190 is Cys |
| (TCR 56A22 β chain with | |
| N-terminal signal peptide) | |
| SEQ ID NO: 168 | X at position 180 is Cys, |
| (TCR 56A23 α chain with | X at position 244 is Ser, |
| N-terminal signal peptide) | X at position 246 is Met, and |
| | X at position 247 is Gly. |
| SEQ ID NO: 169 or 170 | X at position 188 is Cys |
| (TCR 56A23 β chain with | |
| N-terminal signal peptide) | |
| SEQ ID NO: 171 | X at position 177 is Cys, |
| (TCR 56B2 α chain with | X at position 241 is Ser, |
| N-terminal signal peptide) | X at position 243 is Met, and |
| | X at position 244 is Gly. |
| SEQ ID NO: 172 or 173 | X at position 188 is Cys |
| (TCR 56B2 β chain with | |
| N-terminal signal peptide) | |
| SEQ ID NO: 174 | X at position 184 is Cys, |
| (TCR 79B2 α chain with | X at position 248 is Ser, |
| N-terminal signal peptide) | X at position 250 is Met, and |
| | X at position 251 is Gly. |
| SEQ ID NO: 175 or 176 | X at position 189 is Cys |
| (TCR 79B2 β chain with | |
| N-terminal signal peptide) | |
| SEQ ID NO: 177 | X at position 158 is Cys, |
| (TCR J1A1 α chain | X at position 222 is Ser, |
| without N-terminal signal | X at position 224 is Met, and |
| peptide) | X at position 225 is Gly. |
| SEQ ID NO: 178 | X at position 167 is Cys |
| (TCR J1A1 β chain | |
| without N-terminal signal | |
| peptide) | |
| SEQ ID NO: 179 | X at position 159 is Cys, |
| (TCR J1A2 α chain | X at position 223 is Ser, |
| without N-terminal signal | X at position 225 is Met, and |
| peptide) | X at position 226 is Gly. |
| SEQ ID NO: 180 | X at position 169 is Cys |
| (TCR J1A2 β chain | |
| without N-terminal signal | |
| peptide) | |
| SEQ ID NO: 181 | X at position 158 is Cys, |
| (TCR J1A3 α chain with | X at position 222 is Ser, |
| N-terminal signal peptide) | X at position 224 is Met, and |
| | X at position 225 is Gly. |
| SEQ ID NO: 182 | X at position 167 is Cys |
| (TCR J1A3 β chain | |
| without N-terminal signal | |
| peptide) | |
| SEQ ID NO: 183 | X at position 158 is Cys, |
| (TCR 869A1 α chain | X at position 222 is Ser, |
| without N-terminal signal | X at position 224 is Met, and |
| peptide) | X at position 225 is Gly. |
| SEQ ID NO: 184 | X at position 169 is Cys |
| (TCR 869A1 β chain | |
| without N-terminal signal | |
| peptide) | |
| SEQ ID NO: 185 | X at position 163 is Cys, |
| (TCR J2B1 α chain | X at position 227 is Ser, |
| without N-terminal signal | X at position 229 is Met, and |
| peptide) | X at position 230 is Gly. |
| SEQ ID NO: 186 | X at position 169 is Cys |
| (TCR J2B1 β chain | |
| without N-terminal signal | |
| peptide) | |
| SEQ ID NO: 187 | X at position 159 is Cys, |
| (TCR 56A22 α chain | X at position 223 is Ser, |
| without N-terminal signal | X at position 225 is Met, and |
| peptide) | X at position 226 is Gly. |
| SEQ ID NO: 188 | X at position 172 is Cys |
| (TCR 56A22 β chain | |
| without N-terminal signal | |
| peptide) | |
| SEQ ID NO: 189 | X at position 160 is Cys, |
| (TCR 56A23 α chain | X at position 224 is Ser, |
| without N-terminal signal | X at position 226 is Met, and |
| peptide) | X at position 227 is Gly. |
| SEQ ID NO: 190 | X at position 169 is Cys |
| (TCR 56A23 β chain | |
| without N-terminal signal | |
| peptide) | |
| SEQ ID NO: 191 | X at position 157 is Cys, |
| (TCR 56B2 α chain | X at position 221 is Ser, |
| without N-terminal signal | X at position 223 is Met, and |
| peptide) | X at position 224 is Gly. |
| SEQ ID NO: 192 | X at position 169 is Cys |
| (TCR 56B2 β chain | |
| without N-terminal signal | |
| peptide) | |
| SEQ ID NO: 193 | X at position 163 is Cys, |
| (TCR 79B2 α chain | X at position 227 is Ser, |
| without N-terminal signal | X at position 229 is Met, and |
| peptide) | X at position 230 is Gly. |
| SEQ ID NO: 194 | X at position 170 is Cys |
| (TCR 79B2 β chain | |
| without N-terminal signal | |
| peptide) | |

In embodiments of the invention, the substituted amino acid sequence includes substitutions of one, two, or three amino acids in the transmembrane (TM) domain of the constant region of one or both of the α and β chains with a hydrophobic amino acid to provide a hydrophobic amino acid-substituted TCR (also referred to herein as an “LVL-modified TCR”). The hydrophobic amino acid substitution(s) in the TM domain of the TCR may increase the hydrophobicity of the TM domain of the TCR as compared to a TCR that lacks the hydrophobic amino acid substitution(s) in the TM domain. In this regard, the TCR is an LVL-modified TCR in which one, two, or three of the native Ser112, Met114, and Gly115 of SEQ ID NO: 101 may, independently, be substituted with Ala, Val, Leu, Ile, Pro, Phe, Met, or Trp; preferably with Leu, Ile, or Val. Preferably, all three of the native Ser112, Met114, and Gly115 of SEQ ID NO: 101 may, independently, be substituted with Ala, Val, Leu, Ile, Pro, Phe, Met, or Trp; preferably with Leu, Ile, or Val. In embodiments of the invention, the LVL-modified TCR comprises (i) SEQ ID NO: 148, (ii) SEQ ID NO: 149, or (iii) both of SEQ ID NOs: 148 and 149, wherein both of SEQ ID NOs: 148 and 149 are as defined in Table 2. The LVL-modified TCRs of the invention may include the substituted constant region in addition to any of the CDRs or variable regions described herein.

In embodiments of the invention, the LVL-modified TCR comprises a full length alpha chain and a full-length beta chain. Examples of LVL-modified TCR alpha chain and beta chain sequences are set forth in Table 2. In embodiments of the invention, the LVL-modified TCR comprises SEQ ID NO: 150; SEQ ID NO: 151 or 152; SEQ ID NO: 153; SEQ ID NO: 154 or 155; SEQ ID NO: 156; SEQ ID NO: 157 or 158; SEQ ID NO: 159; SEQ ID NO: 160 or 161; SEQ ID NO: 162; SEQ ID NO: 163 or 164; SEQ ID NO: 165; SEQ ID NO: 166 or 167; SEQ ID NO: 168; SEQ ID NO: 169 or 170; SEQ ID NO: 171; SEQ ID NO: 172 or 173; SEQ ID NO: 174; SEQ ID NO: 175 or 176; both of SEQ ID NO: 150, and 151 or 152; both of SEQ ID NO: 153, and 154 or 155; both of SEQ ID NO: 156, and 157 or 158; both of SEQ ID NO: 159, and 160 or 161; both of SEQ ID NO: 162, and 163 or 164; both of SEQ ID NO: 165, and 166 or 167; both of SEQ ID NO: 168, and 169 or 170; both of SEQ ID NO: 171, and 172 or 173; both of SEQ ID NO: 174, and 175 or 176, SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; SEQ ID NO: 186; SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; both of SEQ ID NO: 177 and 178; both of SEQ ID NO: 179 and 180; both of SEQ ID NO: 181 and 182; both of SEQ ID NO: 183 and 184; both of SEQ ID NO: 185 and 186; both of SEQ ID NO: 187 and 188; both of SEQ ID NO: 189 and 190; both of SEQ ID NO: 191 and 192; or both of SEQ ID NO: 193 and 194; wherein all of the sequences are as defined in Table 2.

TABLE 2

| SEQ ID NO: | Definitions of “X” |
|---|---|
| SEQ ID NO: 148 | X at position 48 is Thr; |
| (constant region α chain) | X at position 112 is Ser, Ala, Val, Leu, Ile, Pro, Phe, Met, or Trp; |
| | preferably wherein X at position 112 is Leu, Ile, or Val; |
| | especially preferably wherein X at position 112 is Leu; |
| | X at position 114 is Met, Ala, Val, Leu, Ile, Pro, Phe, or Trp; |
| | preferably wherein X at position 114 is Leu, Ile, or Val; |
| | especially preferably wherein X at position 114 is Ile; and |
| | X at position 115 is Gly, Ala, Val, Leu, Ile, Pro, Phe, Met, or Trp; |
| | preferably wherein X at position 115 is Leu, Ile, or Val; |
| | especially preferably wherein X at position 115 is Val; |
| | Wherein SEQ ID NO: 148 does not comprise SEQ ID NO: 101 |
| | (unsubstituted constant region of alpha chain) |
| SEQ ID NO: 149 | X at position 57 is Ser |
| (constant region β chain) | |
| SEQ ID NO: 150 | X at position 178 is Thr; |
| (TCR J1A1 α chain with | X at position 242 is Ser, Ala, Val, Leu, Ile, Pro, Phe, Met, or Trp; |
| N-terminal signal peptide) | preferably wherein X at position 242 is Leu, Ile, or Val; |
| | especially preferably wherein X at position 242 is Leu; |
| | X at position 244 is Met, Ala, Val, Leu, Ile, Pro, Phe, or Trp; |
| | preferably wherein X at position 244 is Leu, Ile, or Val; |
| | especially preferably wherein X at position 244 is Ile; and |
| | X at position 245 is Gly, Ala, Val, Leu, Ile, Pro, Phe, Met, or Trp; |
| | preferably wherein X at position 245 is Leu, Ile, or Val; |
| | especially preferably wherein X at position 245 is Val, |
| | Wherein SEQ ID NO: 150 does not comprise SEQ ID NO: 103 |
| | (unsubstituted alpha chain) |
| SEQ ID NO: 151 or 152 | X at position 188 is Ser |
| (TCR J1A1 β chain with | |
| N-terminal signal peptide) | |
| SEQ ID NO: 153 | X at position 179 is Thr; |
| (TCR J1A2 α chain with | X at position 243 is Ser, Ala, Val, Leu, Ile, Pro, Phe, Met, or Trp; |
| N-terminal signal peptide) | preferably wherein X at position 243 is Leu, Ile, or Val; |
| | especially preferably wherein X at position 243 is Leu; |
| | X at position 245 is Met, Ala, Val, Leu, Ile, Pro, Phe, or Trp; |
| | preferably wherein X at position 245 is Leu, Ile, or Val; |
| | especially preferably wherein X at position 245 is Ile; and |
| | X at position 246 is Gly, Ala, Val, Leu, Ile, Pro, Phe, Met, or Trp; |
| | preferably wherein X at position 246 is Leu, Ile, or Val; |
| | especially preferably wherein X at position 246 is Val, |
| | Wherein SEQ ID NO: 153 does not comprise SEQ ID NO: 106 |
| | (unsubstituted alpha chain) |
| SEQ ID NO: 154 or 155 | X at position 188 is Ser |
| (TCR J1A2 β chain with | |
| N-terminal signal peptide) | |
| SEQ ID NO: 156 | X at position 178 is Thr; |
| (TCR J1A3 α chain with | X at position 242 is Ser, Ala, Val, Leu, Ile, Pro, Phe, Met, or Trp; |
| N-terminal signal peptide) | preferably wherein X at position 242 is Leu, Ile, or Val; |
| | especially preferably wherein X at position 242 is Leu; |
| | X at position 244 is Met, Ala, Val, Leu, Ile, Pro, Phe, or Trp; |
| | preferably wherein X at position 244 is Leu, Ile, or Val; |
| | especially preferably wherein X at position 244 is Ile; and |
| | X at position 245 is Gly, Ala, Val, Leu, Ile, Pro, Phe, Met, or Trp; |
| | preferably wherein X at position 245 is Leu, Ile, or Val; |
| | especially preferably wherein X at position 245 is Val, |
| | Wherein SEQ ID NO: 156 does not comprise SEQ ID NO: 109 |
| | (unsubstituted alpha chain) |
| SEQ ID NO: 157 or 158 | X at position 188 is Ser |
| (TCR J1A3 β chain with | |
| N-terminal signal peptide) | |
| SEQ ID NO: 159 | X at position 179 is Thr; |
| (TCR 869A1 α chain with | X at position 242 is Ser, Ala, Val, Leu, Ile, Pro, Phe, Met, or Trp; |
| N-terminal signal peptide) | preferably wherein X at position 242 is Leu, Ile, or Val; |
| | especially preferably wherein X at position 242 is Leu; |
| | X at position 244 is Met, Ala, Val, Leu, Ile, Pro, Phe, or Trp; |
| | preferably wherein X at position 244 is Leu, Ile, or Val; |
| | especially preferably wherein X at position 244 is Ile; and |
| | X at position 245 is Gly, Ala, Val, Leu, Ile, Pro, Phe, Met, or Trp; |
| | preferably wherein X at position 245 is Leu, Ile, or Val; |
| | especially preferably wherein X at position 245 is Val, |
| | Wherein SEQ ID NO: 159 does not comprise SEQ ID NO: 112 |
| | (unsubstituted alpha chain) |
| SEQ ID NO: 160 or 161 | X at position 191 is Ser |
| (TCR 869A1 β chain with | |
| N-terminal signal peptide) | |
| SEQ ID NO: 162 | X at position 191 is Thr; |
| (TCR J2B1 α chain with | X at position 255 is Ser, Ala, Val, Leu, Ile, Pro, Phe, Met, or Trp; |
| N-terminal signal peptide) | preferably wherein X at position 255 is Leu, Ile, or Val; |
| | especially preferably wherein X at position 255 is Leu; |
| | X at position 257 is Met, Ala, Val, Leu, Ile, Pro, Phe, or Trp; |
| | preferably wherein X at position 257 is Leu, Ile, or Val; |
| | especially preferably wherein X at position 257 is Ile; and |
| | X at position 258 is Gly, Ala, Val, Leu, Ile, Pro, Phe, Met, or Trp; |
| | preferably wherein X at position 258 is Leu, Ile, or Val; |
| | especially preferably wherein X at position 258 is Val, |
| | Wherein SEQ ID NO: 162 does not comprise SEQ ID NO: 115 |
| | (unsubstituted alpha chain) |
| SEQ ID NO: 163 or 164 | X at position 188 is Ser |
| (TCR J2B1 β chain with | |
| N-terminal signal peptide) | |
| SEQ ID NO: 165 | X at position 180 is Thr; |
| (TCR 56A22 α chain with | X at position 244 is Ser, Ala, Val, Leu, Ile, Pro, Phe, Met, or Trp; |
| N-terminal signal peptide) | preferably wherein X at position 244 is Leu, Ile, or Val; |
| | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; |
| | especially preferably wherein X at position 247 is Val, |
| | Wherein SEQ ID NO: 165 does not comprise SEQ ID NO: 118 |
| | (unsubstituted alpha chain) |
| SEQ ID NO: 166 or 167 | X at position 190 is Ser |
| (TCR 56A22 β chain with | |
| N-terminal signal peptide) | |
| SEQ ID NO: 168 | X at position 180 is Thr; |
| (TCR 56A23 α chain with | X at position 244 is Ser, Ala, Val, Leu, Ile, Pro, Phe, Met, or Trp; |
| N-terminal signal peptide) | preferably wherein X at position 244 is Leu, Ile, or Val; |
| | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; |
| | especially preferably wherein X at position 247 is Val, |
| | Wherein SEQ ID NO: 168 does not comprise SEQ ID NO: 121 |
| | (unsubstituted alpha chain) |
| SEQ ID NO: 169 or 170 | X at position 188 is Ser |
| (TCR 56A23 β chain with | |
| N-terminal signal peptide) | |
| SEQ ID NO: 171 | X at position 177 is Thr; |
| (TCR 56B2 α chain with | X at position 241 is Ser, Ala, Val, Leu, Ile, Pro, Phe, Met, or Trp; |
| N-terminal signal peptide) | preferably wherein X at position 241 is Leu, Ile, or Val; |
| | especially preferably wherein X at position 241 is Leu; |
| | X at position 243 is Met, Ala, Val, Leu, Ile, Pro, Phe, or Trp; |
| | preferably wherein X at position 243 is Leu, Ile, or Val; |
| | especially preferably wherein X at position 243 is Ile; and |
| | X at position 244 is Gly, Ala, Val, Leu, Ile, Pro, Phe, Met, or Trp; |
| | preferably wherein X at position 244 is Leu, Ile, or Val; |
| | especially preferably wherein X at position 244 is Val, |
| | Wherein SEQ ID NO: 171 does not comprise SEQ ID NO: 124 |
| | (unsubstituted alpha chain) |
| SEQ ID NO: 172 or 173 | X at position 188 is Ser |
| (TCR 56B2 β chain with | |
| N-terminal signal peptide) | |
| SEQ ID NO: 174 | X at position 184 is Thr; |
| (TCR 79B2 α chain with | X at position 248 is Ser, Ala, Val, Leu, Ile, Pro, Phe, Met, or Trp; |
| N-terminal signal peptide) | preferably wherein X at position 248 is Leu, Ile, or Val; |
| | especially preferably wherein X at position 248 is Leu; |
| | X at position 250 is Met, Ala, Val, Leu, Ile, Pro, Phe, or Trp; |
| | preferably wherein X at position 250 is Leu, Ile, or Val; |
| | especially preferably wherein X at position 250 is Ile; and |
| | X at position 251 is Gly, Ala, Val, Leu, Ile, Pro, Phe, Met, or Trp; |
| | preferably wherein X at position 251 is Leu, Ile, or Val; |
| | especially preferably wherein X at position 251 is Val, |
| | Wherein SEQ ID NO: 174 does not comprise SEQ ID NO: 127 |
| | (unsubstituted alpha chain) |
| SEQ ID NO: 175 or 176 | X at position 189 is Ser |
| (TCR 79B2 β chain with | |
| N-terminal signal peptide) | |
| SEQ ID NO: 177 | X at position 158 is Thr; |
| (TCR J1A1 α chain | X at position 222 is Ser, Ala, Val, Leu, Ile, Pro, Phe, Met, or Trp; |
| without N-terminal signal | preferably wherein X at position 222 is Leu, Ile, or Val; |
| peptide) | especially preferably wherein X at position 222 is Leu; |
| | X at position 224 is Met, Ala, Val, Leu, Ile, Pro, Phe, or Trp; |
| | preferably wherein X at position 224 is Leu, Ile, or Val; |
| | especially preferably wherein X at position 224 is Ile; and |
| | X at position 225 is Gly, Ala, Val, Leu, Ile, Pro, Phe, Met, or Trp; |
| | preferably wherein X at position 225 is Leu, Ile, or Val; |
| | especially preferably wherein X at position 225 is Val, |
| | Wherein SEQ ID NO: 177 does not comprise SEQ ID NO: 130 |
| | (unsubstituted alpha chain) |
| SEQ ID NO: 178 | X at position 167 is Ser |
| (TCR J1A1 β chain | |
| without N-terminal signal | |
| peptide) | |
| SEQ ID NO: 179 | X at position 159 is Thr; |
| (TCR J1A2 α chain | X at position 223 is Ser, Ala, Val, Leu, Ile, Pro, Phe, Met, or Trp; |
| without N-terminal signal | preferably wherein X at position 223 is Leu, Ile, or Val; |
| peptide) | especially preferably wherein X at position 223 is Leu; |
| | X at position 225 is Met, Ala, Val, Leu, Ile, Pro, Phe, or Trp; |
| | preferably wherein X at position 225 is Leu, Ile, or Val; |
| | especially preferably wherein X at position 225 is Ile; and |
| | X at position 226 is Gly, Ala, Val, Leu, Ile, Pro, Phe, Met, or Trp; |
| | preferably wherein X at position 226 is Leu, Ile, or Val; |
| | especially preferably wherein X at position 226 is Val, |
| | Wherein SEQ ID NO: 179 does not comprise SEQ ID NO: 132 |
| | (unsubstituted alpha chain) |
| SEQ ID NO: 180 | X at position 169 is Ser |
| (TCR J1A2 β chain | |
| without N-terminal signal | |
| peptide) | |
| SEQ ID NO: 181 | X at position 158 is Thr; |
| (TCR J1A3 α chain with | X at position 222 is Ser, Ala, Val, Leu, Ile, Pro, Phe, Met, or Trp; |
| N-terminal signal peptide) | preferably wherein X at position 222 is Leu, Ile, or Val; |
| | especially preferably wherein X at position 222 is Leu; |
| | X at position 224 is Met, Ala, Val, Leu, Ile, Pro, Phe, or Trp; |
| | preferably wherein X at position 224 is Leu, Ile, or Val; |
| | especially preferably wherein X at position 224 is Ile; and |
| | X at position 225 is Gly, Ala, Val, Leu, Ile, Pro, Phe, Met, or Trp; |
| | preferably wherein X at position 225 is Leu, Ile, or Val; |
| | especially preferably wherein X at position 225 is Val, |
| | Wherein SEQ ID NO: 51812 does not comprise SEQ ID NO: 134 |
| | (unsubstituted alpha chain) |
| SEQ ID NO: 182 | X at position 167 is Ser |
| (TCR J1A3 β chain | |
| without N-terminal signal | |
| peptide) | |
| SEQ ID NO: 183 | X at position 158 is Thr; |
| (TCR 869A1 α chain | X at position 222 is Ser, Ala, Val, Leu, Ile, Pro, Phe, Met, or Trp; |
| without N-terminal signal | preferably wherein X at position 222 is Leu, Ile, or Val; |
| peptide) | especially preferably wherein X at position 222 is Leu; |
| | X at position 224 is Met, Ala, Val, Leu, Ile, Pro, Phe, or Trp; |
| | preferably wherein X at position 224 is Leu, Ile, or Val; |
| | especially preferably wherein X at position 224 is Ile; and |
| | X at position 225 is Gly, Ala, Val, Leu, Ile, Pro, Phe, Met, or Trp; |
| | preferably wherein X at position 225 is Leu, Ile, or Val; |
| | especially preferably wherein X at position 225 is Val, |
| | Wherein SEQ ID NO: 183 does not comprise SEQ ID NO: 136 |
| | (unsubstituted alpha chain) |
| SEQ ID NO: 184 | X at position 169 is Ser |
| (TCR 869A1 β chain | |
| without N-terminal signal | |
| peptide) | |
| SEQ ID NO: 185 | X at position 163 is Thr; |
| (TCR J2B1 α chain | X at position 227 is Ser, Ala, Val, Leu, Ile, Pro, Phe, Met, or Trp; |
| without N-terminal signal | preferably wherein X at position 227 is Leu, Ile, or Val; |
| peptide) | especially preferably wherein X at position 227 is Leu; |
| | X at position 229 is Met, Ala, Val, Leu, Ile, Pro, Phe, or Trp; |
| | preferably wherein X at position 229 is Leu, Ile, or Val; |
| | especially preferably wherein X at position 229 is Ile; and |
| | X at position 230 is Gly, Ala, Val, Leu, Ile, Pro, Phe, Met, or Trp; |
| | preferably wherein X at position 230 is Leu, Ile, or Val; |
| | especially preferably wherein X at position 230 is Val, |
| | Wherein SEQ ID NO: 185 does not comprise SEQ ID NO: 138 |
| | (unsubstituted alpha chain) |
| SEQ ID NO: 186 | X at position 169 is Ser |
| (TCR J2B1 β chain | |
| without N-terminal signal | |
| peptide) | |
| SEQ ID NO: 187 | X at position 159 is Thr; |
| (TCR 56A22 α chain | X at position 223 is Ser, Ala, Val, Leu, Ile, Pro, Phe, Met, or Trp; |
| without N-terminal signal | preferably wherein X at position 223 is Leu, Ile, or Val; |
| peptide) | especially preferably wherein X at position 223 is Leu; |
| | X at position 225 is Met, Ala, Val, Leu, Ile, Pro, Phe, or Trp; |
| | preferably wherein X at position 225 is Leu, Ile, or Val; |
| | especially preferably wherein X at position 225 is Ile; and |
| | X at position 226 is Gly, Ala, Val, Leu, Ile, Pro, Phe, Met, or Trp; |
| | preferably wherein X at position 226 is Leu, Ile, or Val; |
| | especially preferably wherein X at position 226 is Val, |
| | Wherein SEQ ID NO: 187 does not comprise SEQ ID NO: 140 |
| | (unsubstituted alpha chain) |
| SEQ ID NO: 188 | X at position 172 is Ser |
| (TCR 56A22 β chain | |
| without N-terminal signal | |
| peptide) | |
| SEQ ID NO: 189 | X at position 160 is Thr; |
| (TCR 56A23 α chain | X at position 224 is Ser, Ala, Val, Leu, Ile, Pro, Phe, Met, or Trp; |
| without N-terminal signal | preferably wherein X at position 224 is Leu, Ile, or Val; |
| peptide) | especially preferably wherein X at position 224 is Leu; |
| | X at position 226 is Met, Ala, Val, Leu, Ile, Pro, Phe, or Trp; |
| | preferably wherein X at position 226 is Leu, Ile, or Val; |
| | especially preferably wherein X at position 226 is Ile; and |
| | X at position 227 is Gly, Ala, Val, Leu, Ile, Pro, Phe, Met, or Trp; |
| | preferably wherein X at position 227 is Leu, Ile, or Val; |
| | especially preferably wherein X at position 227 is Val, |
| | Wherein SEQ ID NO: 189 does not comprise SEQ ID NO: 142 |
| | (unsubstituted alpha chain) |
| SEQ ID NO: 190 | X at position 169 is Ser |
| (TCR 56A23 β chain | |
| without N-terminal signal | |
| peptide) | |
| SEQ ID NO: 191 | X at position 157 is Thr; |
| (TCR 56B2 α chain | X at position 221 is Ser, Ala, Val, Leu, Ile, Pro, Phe, Met, or Trp; |
| without N-terminal signal | preferably wherein X at position 221 is Leu, Ile, or Val; |
| peptide) | especially preferably wherein X at position 221 is Leu; |
| | X at position 223 is Met, Ala, Val, Leu, Ile, Pro, Phe, or Trp; |
| | preferably wherein X at position 223 is Leu, Ile, or Val; |
| | especially preferably wherein X at position 223 is Ile; and |
| | X at position 224 is Gly, Ala, Val, Leu, Ile, Pro, Phe, Met, or Trp; |
| | preferably wherein X at position 224 is Leu, Ile, or Val; |
| | especially preferably wherein X at position 224 is Val, |
| | Wherein SEQ ID NO: 191 does not comprise SEQ ID NO: 144 |
| | (unsubstituted alpha chain) |
| SEQ ID NO: 192 | X at position 169 is Ser |
| (TCR 56B2 β chain | |
| without N-terminal signal | |
| peptide) | |
| SEQ ID NO: 193 | X at position 163 is Thr; |
| (TCR 79B2 α chain | X at position 227 is Ser, Ala, Val, Leu, Ile, Pro, Phe, Met, or Trp; |
| without N-terminal signal | preferably wherein X at position 227 is Leu, Ile, or Val; |
| peptide) | especially preferably wherein X at position 227 is Leu; |
| | X at position 229 is Met, Ala, Val, Leu, Ile, Pro, Phe, or Trp; |
| | preferably wherein X at position 229 is Leu, Ile, or Val; |
| | especially preferably wherein X at position 229 is Ile; and |
| | X at position 230 is Gly, Ala, Val, Leu, Ile, Pro, Phe, Met, or Trp; |
| | preferably wherein X at position 230 is Leu, Ile, or Val; |
| | especially preferably wherein X at position 230 is Val, |
| | Wherein SEQ ID NO: 193 does not comprise SEQ ID NO: 146 |
| | (unsubstituted alpha chain) |
| SEQ ID NO: 194 | X at position 170 is Ser |
| (TCR 79B2 β chain | |
| without N-terminal signal | |
| peptide) | |

In embodiments of the invention, the substituted amino acid sequence includes the cysteine substitutions in the constant region of one or both of the α and β chains in combination with the substitution(s) of one, two, or three amino acids in the transmembrane (TM) domain of the constant region of one or both of the α and β chains with a hydrophobic amino acid (also referred to herein as “cysteine-substituted, LVL-modified TCR”). In this regard, the TCR is a cysteine-substituted, LVL-modified, chimeric TCR in which the native Thr48 of SEQ ID NO: 101 is substituted with Cys; one, two, or three of the native Ser112, Met114, and Gly115 of SEQ ID NO: 101 are, independently, substituted with Ala, Val, Leu, Ile, Pro, Phe, Met, or Trp; preferably with Leu, Ile, or Val; and the native Ser57 of SEQ ID NO: 102 is substituted with Cys. Preferably, all three of the native Ser112, Met114, and Gly115 of SEQ ID NO: 101 may, independently, be substituted with Ala, Val, Leu, Ile, Pro, Phe, Met, or Trp; preferably with Leu, Ile, or Val. In embodiments of the invention, the cysteine-substituted, LVL-modified TCR comprises (i) SEQ ID NO: 148, (ii) SEQ ID NO: 149, or (iii) both of SEQ ID NOs: 148 and 149, wherein both of SEQ ID NOs: 148 and 149 are as defined in Table 3. The cysteine-substituted, LVL-modified TCRs of the invention may include the substituted constant region in addition to any of the CDRs or variable regions described herein.

In embodiments, the cysteine-substituted, LVL-modified TCR comprises a full-length alpha chain and a full-length beta chain. In embodiments of the invention, the LVL-modified TCR comprises SEQ ID NO: 150; SEQ ID NO: 151 or 152; SEQ ID NO: 153; SEQ ID NO: 154 or 155; SEQ ID NO: 156; SEQ ID NO: 157 or 158; SEQ ID NO: 159; SEQ ID NO: 160 or 161; SEQ ID NO: 162; SEQ ID NO: 163 or 164; SEQ ID NO: 165; SEQ ID NO: 166 or 167; SEQ ID NO: 168; SEQ ID NO: 169 or 170; SEQ ID NO: 171; SEQ ID NO: 172 or 173; SEQ ID NO: 174; SEQ ID NO: 175 or 176; both of SEQ ID NO: 150, and 151 or 152; both of SEQ ID NO: 153, and 154 or 155; both of SEQ ID NO: 156, and 157 or 158; both of SEQ ID NO: 159, and 160 or 161; both of SEQ ID NO: 162, and 163 or 164; both of SEQ ID NO: 165, and 166 or 167; both of SEQ ID NO: 168, and 169 or 170; both of SEQ ID NO: 171, and 172 or 173; both of SEQ ID NO: 174, and 175 or 176, SEQ ID NO: 177; SEQ ID NO: 178; SEQ ID NO: 179; SEQ ID NO: 180; SEQ ID NO: 181; SEQ ID NO: 182; SEQ ID NO: 183; SEQ ID NO: 184; SEQ ID NO: 185; SEQ ID NO: 186; SEQ ID NO: 187; SEQ ID NO: 188; SEQ ID NO: 189; SEQ ID NO: 190; SEQ ID NO: 191; SEQ ID NO: 192; SEQ ID NO: 193; SEQ ID NO: 194; both of SEQ ID NO: 177 and 178; both of SEQ ID NO: 179 and 180; both of SEQ ID NO: 181 and 182; both of SEQ ID NO: 183 and 184; both of SEQ ID NO: 185 and 186; both of SEQ ID NO: 187 and 188; both of SEQ ID NO: 189 and 190; both of SEQ ID NO: 191 and 192; or both of SEQ ID NO: 193 and 194; wherein all of the sequences are as defined in Table 3.

TABLE 3

| SEQ ID NO: | Definitions of “X” |
|---|---|
| SEQ ID NO: 148 | X at position 48 is Cys; |
| (constant region α chain) | X at position 112 is Ser, Ala, Val, Leu, Ile, Pro, Phe, Met, or Trp; |
| | preferably wherein X at position 112 is Leu, Ile, or Val; |
| | especially preferably wherein X at position 112 is Leu; |
| | X at position 114 is Met, Ala, Val, Leu, Ile, Pro, Phe, or Trp; |
| | preferably wherein X at position 114 is Leu, Ile, or Val; |
| | especially preferably wherein X at position 114 is Ile; and |
| | X at position 115 is Gly, Ala, Val, Leu, Ile, Pro, Phe, Met, or Trp; |
| | preferably wherein X at position 115 is Leu, Ile, or Val; and |
| | especially preferably wherein X at position 115 is Val, |
| | wherein SEQ ID NO: 148 does not simultaneously comprise all of Ser at |
| | position 112, Met at position 114, and Gly at position 115. |
| SEQ ID NO: 149 | X at position 57 is Cys |
| (constant region β chain) | |
| SEQ ID NO: 150 | X at position 178 is Cys; |
| (TCR J1A1 α chain with | X at position 242 is Ser, Ala, Val, Leu, Ile, Pro, Phe, Met, or Trp; |
| N-terminal signal | preferably wherein X at position 242 is Leu, Ile, or Val; |
| peptide) | especially preferably wherein X at position 242 is Leu; |
| | X at position 244 is Met, Ala, Val, Leu, Ile, Pro, Phe, or Trp; |
| | preferably wherein X at position 244 is Leu, Ile, or Val; |
| | especially preferably wherein X at position 244 is Ile; and |
| | X at position 245 is Gly, Ala, Val, Leu, Ile, Pro, Phe, Met, or Trp; |
| | preferably wherein X at position 245 is Leu, Ile, or Val; and |
| | especially preferably wherein X at position 245 is Val, |
| | wherein SEQ ID NO: 150 does not simultaneously comprise all of Ser at |
| | position 243, Met at position 244, and Gly at position 245. |
| SEQ ID NO: 151 or 152 | X at position 188 is Cys |
| (TCR J1A1 β chain with | |
| N-terminal signal | |
| peptide) | |
| SEQ ID NO: 153 | X at position 179 is Cys; |
| (TCR J1A2 α chain with | X at position 243 is Ser, Ala, Val, Leu, Ile, Pro, Phe, Met, or Trp; |
| N-terminal signal | preferably wherein X at position 243 is Leu, Ile, or Val; |
| peptide) | especially preferably wherein X at position 243 is Leu; |
| | X at position 245 is Met, Ala, Val, Leu, Ile, Pro, Phe, or Trp; |
| | preferably wherein X at position 245 is Leu, Ile, or Val; |
| | especially preferably wherein X at position 245 is Ile; and |
| | X at position 246 is Gly, Ala, Val, Leu, Ile, Pro, Phe, Met, or Trp; |
| | preferably wherein X at position 246 is Leu, Ile, or Val; and |
| | especially preferably wherein X at position 246 is Val, |
| | wherein SEQ ID NO: 153 does not simultaneously comprise all of Ser at |
| | position 243, Met at position 245, and Gly at position 246. |
| SEQ ID NO: 154 or 155 | X at position 188 is Cys |
| (TCR J1A2 β chain with | |
| N-terminal signal | |
| peptide) | |
| SEQ ID NO: 156 | X at position 178 is Cys; |
| (TCR J1A3 α chain with | X at position 242 is Ser, Ala, Val, Leu, Ile, Pro, Phe, Met, or Trp; |
| N-terminal signal | preferably wherein X at position 242 is Leu, Ile, or Val; |
| peptide) | especially preferably wherein X at position 242 is Leu; |
| | X at position 244 is Met, Ala, Val, Leu, Ile, Pro, Phe, or Trp; |
| | preferably wherein X at position 244 is Leu, Ile, or Val; |
| | especially preferably wherein X at position 244 is Ile; and |
| | X at position 245 is Gly, Ala, Val, Leu, Ile, Pro, Phe, Met, or Trp; |
| | preferably wherein X at position 245 is Leu, Ile, or Val; and |
| | especially preferably wherein X at position 245 is Val, |
| | wherein SEQ ID NO: 156 does not simultaneously comprise all of Ser at |
| | position 242, Met at position 244 and Gly at position 245. |
| SEQ ID NO: 157 or 158 | X at position 188 is Cys |
| (TCR J1A3 β chain with | |
| N-terminal signal | |
| peptide) | |
| SEQ ID NO: 159 | X at position 178 is Cys; |
| (TCR 869A1 α chain | X at position 242 is Ser, Ala, Val, Leu, Ile, Pro, Phe, Met, or Trp; |
| with N-terminal signal | preferably wherein X at position 242 is Leu, Ile, or Val; |
| peptide) | especially preferably wherein X at position 242 is Leu; |
| | X at position 244 is Met, Ala, Val, Leu, Ile, Pro, Phe, or Trp; |
| | preferably wherein X at position 244 is Leu, Ile, or Val; |
| | especially preferably wherein X at position 244 is Ile; and |
| | X at position 245 is Gly, Ala, Val, Leu, Ile, Pro, Phe, Met, or Trp; |
| | preferably wherein X at position 245 is Leu, Ile, or Val; and |
| | especially preferably wherein X at position 245 is Val, |
| | wherein SEQ ID NO: 159 does not simultaneously comprise all of Ser at |
| | position 242, Met at position 244, and Gly at position 245. |
| SEQ ID NO: 160 or 161 | X at position 191 is Cys |
| (TCR 869A1 β chain | |
| with N-terminal signal | |
| peptide) | |
| SEQ ID NO: 162 | X at position 191 is Cys; |
| (TCR J2B1 α chain with | X at position 255 is Ser, Ala, Val, Leu, Ile, Pro, Phe, Met, or Trp; |
| N-terminal signal | preferably wherein X at position 255 is Leu, Ile, or Val; |
| peptide) | especially preferably wherein X at position 255 is Leu; |
| | X at position 257 is Met, Ala, Val, Leu, Ile, Pro, Phe, or Trp; |
| | preferably wherein X at position 257 is Leu, Ile, or Val; |
| | especially preferably wherein X at position 257 is Ile; and |
| | X at position 258 is Gly, Ala, Val, Leu, Ile, Pro, Phe, Met, or Trp; |
| | preferably wherein X at position 258 is Leu, Ile, or Val; and |
| | especially preferably wherein X at position 258 is Val, |
| | wherein SEQ ID NO: 162 does not simultaneously comprise all of Ser at |
| | position 255, Met at position 257, and Gly at position 258. |
| SEQ ID NO: 163 or 164 | X at position 188 is Cys |
| (TCR J2B1 β chain with | |
| N-terminal signal | |
| peptide) | |
| SEQ ID NO: 165 | X at position 180 is Cys; |
| (TCR 56A22 α chain | X at position 244 is Ser, Ala, Val, Leu, Ile, Pro, Phe, Met, or Trp; |
| with N-terminal signal | preferably wherein X at position 244 is Leu, Ile, or Val; |
| peptide) | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; and |
| | especially preferably wherein X at position 247 is Val, |
| | wherein SEQ ID NO: 165 does not simultaneously comprise all of Ser at |
| | position 244, Met at position 246, and Gly at position 247. |
| SEQ ID NO: 166 or 167 | X at position 190 is Cys |
| (TCR 56A22 β chain | |
| with N-terminal signal | |
| peptide) | |
| SEQ ID NO: 168 | X at position 180 is Cys; |
| (TCR 56A23 α chain | X at position 244 is Ser, Ala, Val, Leu, Ile, Pro, Phe, Met, or Trp; |
| with N-terminal signal | preferably wherein X at position 244 is Leu, Ile, or Val; |
| peptide) | especially preferably wherein X at position 244 is Leu; |
| | X at position 246 is Met, Ala, Val, Leu, Ile, Pro, Phe, or Trp; |
| | preferably wherein X at position 246 is Leu, Ile, or Val; |
| | especially preferably wherein X at position 246 is Ile; and |
| | X at position 247 is Gly, Ala, Val, Leu, Ile, Pro, Phe, Met, or Trp; |
| | preferably wherein X at position 247 is Leu, Ile, or Val; and |
| | especially preferably wherein X at position 247 is Val, |
| | wherein SEQ ID NO: 168 does not simultaneously comprise all of Ser at |
| | position 244, Met at position 246, and Gly at position 247. |
| SEQ ID NO: 169 or 170 | X at position 188 is Cys |
| (TCR 56A23 β chain | |
| with N-terminal signal | |
| peptide) | |
| SEQ ID NO: 171 | X at position 177 is Cys; |
| (TCR 56B2 α chain with | X at position 241 is Ser, Ala, Val, Leu, Ile, Pro, Phe, Met, or Trp; |
| N-terminal signal | preferably wherein X at position 241 is Leu, Ile, or Val; |
| peptide) | especially preferably wherein X at position 241 is Leu; |
| | X at position 243 is Met, Ala, Val, Leu, Ile, Pro, Phe, or Trp; |
| | preferably wherein X at position 243 is Leu, Ile, or Val; |
| | especially preferably wherein X at position 244 is Ile; and |
| | X at position 244 is Gly, Ala, Val, Leu, Ile, Pro, Phe, Met, or Trp; |
| | preferably wherein X at position 244 is Leu, Ile, or Val; and |
| | especially preferably wherein X at position 244 is Val, |
| | wherein SEQ ID NO: 171 does not simultaneously comprise all of Ser at |
| | position 241, Met at position 243, and Gly at position 244. |
| SEQ ID NO: 172 or 173 | X at position 188 is Cys |
| (TCR 56B2 β chain with | |
| N-terminal signal | |
| peptide) | |
| SEQ ID NO: 174 | X at position 184 is Cys; |
| (TCR 79B2 α chain with | X at position 248 is Ser, Ala, Val, Leu, Ile, Pro, Phe, Met, or Trp; |
| N-terminal signal | preferably wherein X at position 248 is Leu, Ile, or Val; |
| peptide) | especially preferably wherein X at position 248 is Leu; |
| | X at position 250 is Met, Ala, Val, Leu, Ile, Pro, Phe, or Trp; |
| | preferably wherein X at position 250 is Leu, Ile, or Val; |
| | especially preferably wherein X at position 250 is Ile; and |
| | X at position 251 is Gly, Ala, Val, Leu, Ile, Pro, Phe, Met, or Trp; |
| | preferably wherein X at position 251 is Leu, Ile, or Val; and |
| | especially preferably wherein X at position 251 is Val, |
| | wherein SEQ ID NO: 174 does not simultaneously comprise all of Ser at |
| | position 248, Met at position 250, and Gly at position 251. |
| SEQ ID NO: 175 or 176 | X at position 189 is Cys |
| (TCR 79B2 β chain with | |
| N-terminal signal | |
| peptide) | |
| SEQ ID NO: 177 | X at position 158 is Cys; |
| (TCR J1A1 α chain | X at position 222 is Ser, Ala, Val, Leu, Ile, Pro, Phe, Met, or Trp; |
| without N-terminal | preferably wherein X at position 222 is Leu, Ile, or Val; |
| signal peptide) | especially preferably wherein X at position 222 is Leu; |
| | X at position 224 is Met, Ala, Val, Leu, Ile, Pro, Phe, or Trp; |
| | preferably wherein X at position 224 is Leu, Ile, or Val; |
| | especially preferably wherein X at position 224 is Ile; and |
| | X at position 225 is Gly, Ala, Val, Leu, Ile, Pro, Phe, Met, or Trp; |
| | preferably wherein X at position 225 is Leu, Ile, or Val; and |
| | especially preferably wherein X at position 225 is Val, |
| | wherein SEQ ID NO: 177 does not simultaneously comprise all of Ser at |
| | position 222, Met at position 224, and Gly at position 225. |
| SEQ ID NO: 178 | X at position 167 is Cys |
| (TCR J1A1 β chain | |
| without N-terminal | |
| signal peptide) | |
| SEQ ID NO: 179 | X at position 159 is Cys; |
| (TCR J1A2 α chain | X at position 223 is Ser, Ala, Val, Leu, Ile, Pro, Phe, Met, or Trp; |
| without N-terminal | preferably wherein X at position 223 is Leu, Ile, or Val; |
| signal peptide) | especially preferably wherein X at position 223 is Leu; |
| | X at position 225 is Met, Ala, Val, Leu, Ile, Pro, Phe, or Trp; |
| | preferably wherein X at position 225 is Leu, Ile, or Val; |
| | especially preferably wherein X at position 225 is Ile; and |
| | X at position 226 is Gly, Ala, Val, Leu, Ile, Pro, Phe, Met, or Trp; |
| | preferably wherein X at position 226 is Leu, Ile, or Val; and |
| | especially preferably wherein X at position 226 is Val, |
| | wherein SEQ ID NO: 179 does not simultaneously comprise all of Ser at |
| | position 223, Met at position 225, and Gly at position 226. |
| SEQ ID NO: 180 | X at position 169 is Cys |
| (TCR J1A2 β chain | |
| without N-terminal | |
| signal peptide) | |
| SEQ ID NO: 181 | X at position 158 is Cys; |
| (TCR J1A3 α chain with | X at position 222 is Ser, Ala, Val, Leu, Ile, Pro, Phe, Met, or Trp; |
| N-terminal signal | preferably wherein X at position 222 is Leu, Ile, or Val; |
| peptide) | especially preferably wherein X at position 222 is Leu; |
| | X at position 224 is Met, Ala, Val, Leu, Ile, Pro, Phe, or Trp; |
| | preferably wherein X at position 224 is Leu, Ile, or Val; |
| | especially preferably wherein X at position 224 is Ile; and |
| | X at position 225 is Gly, Ala, Val, Leu, Ile, Pro, Phe, Met, or Trp; |
| | preferably wherein X at position 225 is Leu, Ile, or Val; and |
| | especially preferably wherein X at position 225 is Val, |
| | wherein SEQ ID NO: 181 does not simultaneously comprise all of Ser at |
| | position 222, Met at position 224, and Gly at position 225. |
| SEQ ID NO: 182 | X at position 167 is Cys |
| (TCR J1A3 β chain | |
| without N-terminal | |
| signal peptide) | |
| SEQ ID NO: 183 | X at position 158 is Cys; |
| (TCR 869A1 α chain | X at position 222 is Ser, Ala, Val, Leu, Ile, Pro, Phe, Met, or Trp; |
| without N-terminal | preferably wherein X at position 222 is Leu, Ile, or Val; |
| signal peptide) | especially preferably wherein X at position 222 is Leu; |
| | X at position 224 is Met, Ala, Val, Leu, Ile, Pro, Phe, or Trp; |
| | preferably wherein X at position 224 is Leu, Ile, or Val; |
| | especially preferably wherein X at position 224 is Ile; and |
| | X at position 225 is Gly, Ala, Val, Leu, Ile, Pro, Phe, Met, or Trp; |
| | preferably wherein X at position 225 is Leu, Ile, or Val; and |
| | especially preferably wherein X at position 225 is Val, |
| | wherein SEQ ID NO: 183 does not simultaneously comprise all of Ser at |
| | position 222, Met at position 224, and Gly at position 225. |
| SEQ ID NO: 184 | X at position 169 is Cys |
| (TCR 869A1 β chain | |
| without N-terminal | |
| signal peptide) | |
| SEQ ID NO: 185 | X at position 163 is Cys; |
| (TCR J2B1 α chain | X at position 227 is Ser, Ala, Val, Leu, Ile, Pro, Phe, Met, or Trp; |
| without N-terminal | preferably wherein X at position 227 is Leu, Ile, or Val; |
| signal peptide) | especially preferably wherein X at position 227 is Leu; |
| | X at position 229 is Met, Ala, Val, Leu, Ile, Pro, Phe, or Trp; |
| | preferably wherein X at position 229 is Leu, Ile, or Val; |
| | especially preferably wherein X at position 229 is Ile; and |
| | X at position 230 is Gly, Ala, Val, Leu, Ile, Pro, Phe, Met, or Trp; |
| | preferably wherein X at position 230 is Leu, Ile, or Val; and |
| | especially preferably wherein X at position 230 is Val, |
| | wherein SEQ ID NO: 185 does not simultaneously comprise all of Ser at |
| | position 227, Met at position 229, and Gly at position 230. |
| SEQ ID NO: 186 | X at position 169 is Cys |
| (TCR J2B1 β chain | |
| without N-terminal | |
| signal peptide) | |
| SEQ ID NO: 187 | X at position 159 is Cys; |
| (TCR 56A22 α chain | X at position 223 is Ser, Ala, Val, Leu, Ile, Pro, Phe, Met, or Trp; |
| without N-terminal | preferably wherein X at position 223 is Leu, Ile, or Val; |
| signal peptide) | especially preferably wherein X at position 223 is Leu; |
| | X at position 225 is Met, Ala, Val, Leu, Ile, Pro, Phe, or Trp; |
| | preferably wherein X at position 225 is Leu, Ile, or Val; |
| | especially preferably wherein X at position 225 is Ile; and |
| | X at position 226 is Gly, Ala, Val, Leu, Ile, Pro, Phe, Met, or Trp; |
| | preferably wherein X at position 226 is Leu, Ile, or Val; and |
| | especially preferably wherein X at position 226 is Val, |
| | wherein SEQ ID NO: 187 does not simultaneously comprise all of Ser at |
| | position 223, Met at position 225, and Gly at position 226. |
| SEQ ID NO: 188 | X at position 172 is Cys |
| (TCR 56A22 β chain | |
| without N-terminal | |
| signal peptide) | |
| SEQ ID NO: 189 | X at position 160 is Cys; |
| (TCR 56A23 α chain | X at position 224 is Ser, Ala, Val, Leu, Ile, Pro, Phe, Met, or Trp; |
| without N-terminal | preferably wherein X at position 224 is Leu, Ile, or Val; |
| signal peptide) | especially preferably wherein X at position 224 is Leu; |
| | X at position 226 is Met, Ala, Val, Leu, Ile, Pro, Phe, or Trp; |
| | preferably wherein X at position 226 is Leu, Ile, or Val; |
| | especially preferably wherein X at position 226 is Ile; and |
| | X at position 227 is Gly, Ala, Val, Leu, Ile, Pro, Phe, Met, or Trp; |
| | preferably wherein X at position 227 is Leu, Ile, or Val; and |
| | especially preferably wherein X at position 227 is Val, |
| | wherein SEQ ID NO: 189 does not simultaneously comprise all of Ser at |
| | position 224, Met at position 226, and Gly at position 227. |
| SEQ ID NO: 190 | X at position 169 is Cys |
| (TCR 56A23 β chain | |
| without N-terminal | |
| signal peptide) | |
| SEQ ID NO: 191 | X at position 157 is Cys; |
| (TCR 56B2 α chain | X at position 221 is Ser, Ala, Val, Leu, Ile, Pro, Phe, Met, or Trp; |
| without N-terminal | preferably wherein X at position 221 is Leu, Ile, or Val; |
| signal peptide) | especially preferably wherein X at position 221 is Leu; |
| | X at position 223 is Met, Ala, Val, Leu, Ile, Pro, Phe, or Trp; |
| | preferably wherein X at position 223 is Leu, Ile, or Val; |
| | especially preferably wherein X at position 223 is Ile; and |
| | X at position 224 is Gly, Ala, Val, Leu, Ile, Pro, Phe, Met, or Trp; |
| | preferably wherein X at position 224 is Leu, Ile, or Val; and |
| | especially preferably wherein X at position 224 is Val, |
| | wherein SEQ ID NO: 191 does not simultaneously comprise all of Ser at |
| | position 221, Met at position 223, and Gly at position 224. |
| SEQ ID NO: 192 | X at position 169 is Cys |
| (TCR 56B2 β chain | |
| without N-terminal | |
| signal peptide) | |
| SEQ ID NO: 193 | X at position 163 is Cys; |
| (TCR 79B2 α chain | X at position 227 is Ser, Ala, Val, Leu, Ile, Pro, Phe, Met, or Trp; |
| without N-terminal | preferably wherein X at position 227 is Leu, Ile, or Val; |
| signal peptide) | especially preferably wherein X at position 227 is Leu; |
| | X at position 229 is Met, Ala, Val, Leu, Ile, Pro, Phe, or Trp; |
| | preferably wherein X at position 229 is Leu, Ile, or Val; |
| | especially preferably wherein X at position 229 is Ile; and |
| | X at position 230 is Gly, Ala, Val, Leu, Ile, Pro, Phe, Met, or Trp; |
| | preferably wherein X at position 230 is Leu, Ile, or Val; and |
| | especially preferably wherein X at position 230 is Val, |
| | wherein SEQ ID NO: 193 does not simultaneously comprise all of Ser at |
| | position 227, Met at position 229, and Gly at position 230. |
| SEQ ID NO: 194 | X at position 170 is Cys |
| (TCR 79B2 β chain | |
| without N-terminal | |
| signal peptide) | |

Also provided by an embodiment of the invention is a polypeptide comprising a functional portion of any of the TCRs described herein. The term “polypeptide,” as used herein, includes oligopeptides and refers to a single chain of amino acids connected by one or more peptide bonds.

With respect to the inventive polypeptides, the functional portion can be any portion comprising contiguous amino acids of the TCR of which it is a part, provided that the functional portion specifically binds to CD20. The term “functional portion,” when used in reference to a TCR, refers to any part or fragment of the TCR of the invention, which part or fragment retains the biological activity of the TCR of which it is a part (the parent TCR). Functional portions encompass, for example, those parts of a TCR that retain the ability to specifically bind to CD20 (e.g., within the context of an HLA-A*02:01 molecule), or detect, treat, or prevent cancer, to a similar extent, the same extent, or to a higher extent, as the parent TCR. In reference to the parent TCR, the functional portion can comprise, for instance, about 10%, about 25%, about 30%, about 50%, about 70%, about 80%, about 90%, about 95%, or more, of the parent TCR.

The functional portion can comprise additional amino acids at the amino or carboxy terminus of the portion, or at both termini, which additional amino acids are not found in the amino acid sequence of the parent TCR. Desirably, the additional amino acids do not interfere with the biological function of the functional portion, e.g., specifically binding to CD20; and/or having the ability to detect cancer, treat or prevent cancer, etc. More desirably, the additional amino acids enhance the biological activity, as compared to the biological activity of the parent TCR.

The polypeptide can comprise a functional portion of either or both of the α and β chains of the TCRs of the invention, such as a functional portion comprising one or more of the CDR1, CDR2, and CDR3 of the variable region(s) of the α chain and/or β chain of a TCR of the invention. In an embodiment of the invention, the polypeptide can comprise the amino acid sequence of SEQ ID NO: 1 (CDR1 of α chain), SEQ ID NO: 2 (CDR2 of α chain), SEQ ID NO: 3 (CDR3 of α chain), SEQ ID NO: 4 (CDR1 of β chain), SEQ ID NO: 5 (CDR2 of β chain), SEQ ID NO: 6 (CDR3 of β chain), or a combination thereof. In another embodiment of the invention, the polypeptide can comprise the amino acid sequence of SEQ ID NO: 7 (CDR1 of α chain), SEQ ID NO: 8 (CDR2 of α chain), SEQ ID NO: 9 (CDR3 of α chain), SEQ ID NO: 10 (CDR1 of β chain), SEQ ID NO: 11 (CDR2 of β chain), SEQ ID NO: 12 (CDR3 of β chain), or a combination thereof. In another embodiment of the invention, the polypeptide can comprise the amino acid sequence of SEQ ID NO: 13 (CDR1 of α chain), SEQ ID NO: 14 (CDR2 of α chain), SEQ ID NO: 15 (CDR3 of α chain), SEQ ID NO: 16 (CDR1 of β chain), SEQ ID NO: 17 (CDR2 of β chain), SEQ ID NO: 18 (CDR3 of β chain), or a combination thereof. In an embodiment of the invention, the polypeptide can comprise the amino acid sequence of SEQ ID NO: 19 (CDR1 of α chain), SEQ ID NO: 20 (CDR2 of a chain), SEQ ID NO: 21 (CDR3 of α chain), SEQ ID NO: 22 (CDR1 of β chain), SEQ ID NO: 23 (CDR2 of β chain), SEQ ID NO: 24 (CDR3 of β chain), or a combination thereof. In another embodiment of the invention, the polypeptide can comprise the amino acid sequence of SEQ ID NO: 25 (CDR1 of α chain), SEQ ID NO: 26 (CDR2 of α chain), SEQ ID NO: 27 (CDR3 of α chain), SEQ ID NO: 28 (CDR1 of β chain), SEQ ID NO: 29 (CDR2 of β chain), SEQ ID NO: 30 (CDR3 of β chain), or a combination thereof. In another embodiment of the invention, the polypeptide can comprise the amino acid sequence of SEQ ID NO: 31 (CDR1 of α chain), SEQ ID NO: 32 (CDR2 of α chain), SEQ ID NO: 33 (CDR3 of α chain), SEQ ID NO: 34 (CDR1 of β chain), SEQ ID NO: 35 (CDR2 of β chain), SEQ ID NO: 36 (CDR3 of β chain), or a combination thereof. In another embodiment of the invention, the polypeptide can comprise the amino acid sequence of SEQ ID NO: 37 (CDR1 of α chain), SEQ ID NO: 38 (CDR2 of α chain), SEQ ID NO: 39 (CDR3 of α chain), SEQ ID NO: 40 (CDR1 of β chain), SEQ ID NO: 41 (CDR2 of β chain), SEQ ID NO: 42 (CDR3 of β chain), or a combination thereof. In another embodiment of the invention, the polypeptide can comprise the amino acid sequence of SEQ ID NO: 43 (CDR1 of α chain), SEQ ID NO: 44 (CDR2 of a chain), SEQ ID NO: 45 (CDR3 of α chain), SEQ ID NO: 46 (CDR1 of β chain), SEQ ID NO: 47 (CDR2 of β chain), SEQ ID NO: 48 (CDR3 of β chain), or a combination thereof. In another embodiment of the invention, the polypeptide can comprise the amino acid sequence of SEQ ID NO: 49 (CDR1 of α chain), SEQ ID NO: 50 (CDR2 of α chain), SEQ ID NO: 51 (CDR3 of α chain), SEQ ID NO: 52 (CDR1 of β chain), SEQ ID NO: 53 (CDR2 of β chain), SEQ ID NO: 54 (CDR3 of β chain), or a combination thereof. Any CDR3 of SEQ ID NOS: 3, 6, 9, 12, 15, 18, 21, 24, 27, 30, 33, 36, 39, 42, 45, 48, 51, or 54, i.e., of the α chain or β chain or both, may further comprise a cysteine immediately N-terminal to the first amino acid of the CDR or a phenylalanine immediately C-terminal to the final amino acid or both.

In this regard, the inventive polypeptide can comprise any one or more of the amino acid sequences selected from SEQ ID NOs: 1-6, 7-12, 13-18, 19-24, 25-30, 31-36, 37-42, 43-48, and 49-54. In an embodiment of the invention, the TCR comprises the amino acid sequences of all of (a) SEQ ID NOs: 1-3, (b) SEQ ID NOs: 4-6, (c) SEQ ID NOs: 7-9, (d) SEQ ID NOs: 10-12, (e) SEQ ID NOs: 13-15, (0 SEQ ID NOs: 16-18, (g) SEQ ID NOs: 19-21, (h) SEQ ID NOs: 22-24, (i) SEQ ID NOs: 25-27, (j) SEQ ID NOs: 28-30, (k) SEQ ID NOs: 31-33, (1) SEQ ID NOs: 34-36, (m) SEQ ID NOs: 37-39, (n) SEQ ID NOs: 40-42, (o) SEQ ID NOs: 43-45, (p) SEQ ID NOs: 46-48, (q) SEQ ID NOs: 49-51, (r) SEQ ID NOs: 52-54, (s) SEQ ID NOs: 1-6, (t) SEQ ID NOs: 7-12, (u) SEQ ID NOs: 13-18, (v) SEQ ID NOs: 19-24, (w) SEQ ID NOs: 25-30, (x) SEQ ID NOs: 31-36, (y) SEQ ID NOs: 37-42, (z) SEQ ID NOs: 43-48, or (aa) SEQ ID NOs: 49-54. In a preferred embodiment, the polypeptide comprises the amino acid sequences of (i) SEQ ID NOs: 1-6, (ii) SEQ ID NOs: 7-12, (iii) SEQ ID NOs: 13-18, (iv) SEQ ID NOs: 19-24, (v) SEQ ID NOs: 25-30, (vi) SEQ ID NOs: 31-36, (vii) SEQ ID NOs: 37-42, (viii) SEQ ID NOs: 43-48, or (ix) SEQ ID NOs: 49-54.

In an embodiment of the invention, the inventive polypeptide can comprise, for instance, the variable region of the inventive TCR comprising a combination of the CDR regions set forth above. In this regard, the polypeptide can comprise the amino acid sequence of SEQ ID NO: 56 (variable region of a chain with N-terminal signal peptide); SEQ ID NO: 57 or 58 (variable region of β chain with N-terminal signal peptide); SEQ ID NO: 59 (variable region of a chain with N-terminal signal peptide); SEQ ID NO: 60 or 61 (variable region of β chain with N-terminal signal peptide); SEQ ID NO: 62 (variable region of a chain with N-terminal signal peptide); SEQ ID NO: 63 or 64 (variable region of β chain with N-terminal signal peptide); SEQ ID NO: 65 (variable region of a chain with N-terminal signal peptide); SEQ ID NO: 66 or 67 (variable region of β chain with N-terminal signal peptide); SEQ ID NO: 68 (variable region of a chain with N-terminal signal peptide); SEQ ID NO: 69 or 70 (variable region of β chain with N-terminal signal peptide); SEQ ID NO: 71 (variable region of a chain with N-terminal signal peptide); SEQ ID NO: 72 or 73 (variable region of β chain with N-terminal signal peptide); SEQ ID NO: 74 (variable region of a chain with N-terminal signal peptide); SEQ ID NO: 75 or 76 (variable region of β chain with N-terminal signal peptide); SEQ ID NO: 77 (variable region of a chain with N-terminal signal peptide); SEQ ID NO: 78 or 79 (variable region of β chain with N-terminal signal peptide); SEQ ID NO: 80 (variable region of a chain with N-terminal signal peptide); SEQ ID NO: 81 or 82 (variable region of β chain with N-terminal signal peptide); SEQ ID NO: 83 (variable region of a chain without N-terminal signal peptide); SEQ ID NO: 84 (variable region of β chain without N-terminal signal peptide); SEQ ID NO: 85 (variable region of a chain without N-terminal signal peptide); SEQ ID NO: 86 (variable region of β chain without N-terminal signal peptide); SEQ ID NO: 87 (variable region of a chain without N-terminal signal peptide); SEQ ID NO: 88 (variable region of β chain without N-terminal signal peptide); SEQ ID NO: 89 (variable region of a chain without N-terminal signal peptide); SEQ ID NO: 90 (variable region of β chain without N-terminal signal peptide); SEQ ID NO: 91 (variable region of a chain without N-terminal signal peptide); SEQ ID NO: 92 (variable region of β chain without N-terminal signal peptide); SEQ ID NO: 93 (variable region of a chain without N-terminal signal peptide); SEQ ID NO: 94 (variable region of β chain without N-terminal signal peptide); SEQ ID NO: 95 (variable region of a chain without N-terminal signal peptide); SEQ ID NO: 96 (variable region of β chain without N-terminal signal peptide); SEQ ID NO: 97 (variable region of a chain without N-terminal signal peptide); SEQ ID NO: 98 (variable region of β chain without N-terminal signal peptide); SEQ ID NO: 99 (variable region of a chain without N-terminal signal peptide); SEQ ID NO: 100 (variable region of β chain without N-terminal signal peptide); both of SEQ ID NOs: 56, and 57 or 58; both of SEQ ID NOs: 59, and 60 or 61; both of SEQ ID NOs: 62, and 63 or 64; both of SEQ ID NOs: 65, and 66 or 67; both of SEQ ID NOs: 68, and 69 or 70; both of SEQ ID NOs: 71, and 72 or 73; both of SEQ ID NOs: 74, and 75 or 76; both of SEQ ID NOs: 77, and 78 or 79; both of SEQ ID NOs: 80, and 81 or 82; both of SEQ ID NOs: 83 and 84; both of SEQ ID NOs: 85 and 86; both of SEQ ID NOs: 87 and 88; both of SEQ ID NOs: 89 and 90; both of SEQ ID NOs: 91 and 92; both of SEQ ID NOs: 93 and 94; both of SEQ ID NOs: 95 and 96; both of SEQ ID NOs: 97 and 98; or both of SEQ ID NOs: 99 and 100.

In embodiments of the invention, the inventive polypeptide can further comprise the constant region of the inventive TCR set forth above. In this regard, the polypeptide can further comprise the amino acid sequence of SEQ ID NO: 101 (WT murine constant region of α chain), SEQ ID NO: 102 (WT murine constant region of β chain), SEQ ID NO: 148, (substituted murine constant region of α chain), SEQ ID NO: 149 (substituted murine constant region of β chain), both SEQ ID NOs: 101 and 102, or both SEQ ID NOs: 148 and 149. Preferably, the polypeptide further comprises the amino acid sequences of both of SEQ ID NOs: 101 and 102 or both of SEQ ID NO: 148 and 149 in combination with any of the CDR regions or variable regions described herein with respect to other aspects of the invention.

In embodiments of the invention, the polypeptide comprises: (a) the amino acid sequence of SEQ ID NO: 148, wherein: (i) X at position 48 of SEQ ID NO: 148 is Thr or Cys; (ii) X at position 112 of SEQ ID NO: 148 is Ser, Ala, Val, Leu, Ile, Pro, Phe, Met, or Trp; (iii) X at position 114 of SEQ ID NO: 148 is Met, Ala, Val, Leu, Ile, Pro, Phe, or Trp; and (iv) X at position 115 of SEQ ID NO: 148 is Gly, Ala, Val, Leu, Ile, Pro, Phe, Met, or Trp; (b) the amino acid sequence of SEQ ID NO: 149, wherein X at position 57 of SEQ ID NO: 149 is Ser or Cys; or (c) both (a) and (b). In embodiments of the invention, one or both of SEQ ID NOs: 148 and 149 of the polypeptide are as defined in any one of Tables 1-3.

In embodiments of the invention, the inventive polypeptide can comprise the entire length of an α or β chain of the TCR described herein. In this regard, the inventive polypeptide can comprise the amino acid sequence of both of SEQ ID NO: 103, and 104 or 105, both of SEQ ID NO: 106, and 107 or 108, both of SEQ ID NO: 109, and 110 or 111, both of SEQ ID NO: 112, and 113 or 114, both of SEQ ID NO: 115, and 116 or 117, both of SEQ ID NO: 118, and 119 or 120, both of SEQ ID NO: 121, and 122 or 123, both of SEQ ID NO: 124, and 125 or 126, both of SEQ ID NO: 127, and 128 or 129, both of SEQ ID NOS: 130 and 131; both of SEQ ID NOS: 132 and 133; both of SEQ ID NOS: 134 and 135; both of SEQ ID NOS: 136 and 137; both of SEQ ID NOS: 138 and 139; both of SEQ ID NOS: 140 and 141; both of SEQ ID NOS: 142 and 143; both of SEQ ID NOS: 144 and 145; or both of SEQ ID NOS: 146 and 147. The polypeptide of the invention can comprise both chains of the TCRs described herein.

In embodiments of the invention, the polypeptide comprises (a) an α chain comprising the amino acid sequence of SEQ ID NO: 150 (α chain with N-terminal signal peptide), wherein: (i) X at position 178 of SEQ ID NO: 150 is Thr or Cys; (ii) X at position 242 of SEQ ID NO: 150 is Ser, Ala, Val, Leu, Ile, Pro, Phe, Met, or Trp; (iii) X at position 244 of SEQ ID NO: 150 is Met, Ala, Val, Leu, Ile, Pro, Phe, or Trp; and (iv) X at position 245 of SEQ ID NO: 150 is Gly, Ala, Val, Leu, Ile, Pro, Phe, Met, or Trp; (b) a β chain comprising the amino acid sequence of SEQ ID NO: 151 or 152 (β chain with N-terminal signal peptide), wherein X at position 188 of SEQ ID NO: 151 or 152 is Ser or Cys; (c) an α chain comprising the amino acid sequence of SEQ ID NO: 153 (α chain with N-terminal signal peptide), wherein: (i) X at position 179 of SEQ ID NO: 153 is Thr or Cys; (ii) X at position 243 of SEQ ID NO: 153 is Ser, Ala, Val, Leu, Ile, Pro, Phe, Met, or Trp; (iii) X at position 245 of SEQ ID NO: 153 is Met, Ala, Val, Leu, Ile, Pro, Phe, or Trp; and (iv) X at position 246 of SEQ ID NO: 153 is Gly, Ala, Val, Leu, Ile, Pro, Phe, Met, or Trp; (d) a β chain comprising the amino acid sequence of SEQ ID NO: 154 or 155 (β chain with N-terminal signal peptide), wherein X at position 188 of SEQ ID NO: 154 or 155 is Ser or Cys; (e) an α chain comprising the amino acid sequence of SEQ ID NO: 156 (α chain with N-terminal signal peptide), wherein: (i) X at position 178 of SEQ ID NO: 156 is Thr or Cys; (ii) X at position 242 of SEQ ID NO: 156 is Ser, Ala, Val, Leu, Ile, Pro, Phe, Met, or Trp; (iii) X at position 244 of SEQ ID NO: 156 is Met, Ala, Val, Leu, Ile, Pro, Phe, or Trp; and (iv) X at position 245 of SEQ ID NO: 156 is Gly, Ala, Val, Leu, Ile, Pro, Phe, Met, or Trp; (f) a β chain comprising the amino acid sequence of SEQ ID NO: 157 or 158 (β chain with N-terminal signal peptide), wherein X at position 188 of SEQ ID NO: 157 or 158 is Ser or Cys; (g) an α chain comprising the amino acid sequence of SEQ ID NO: 159 (α chain with N-terminal signal peptide), wherein: (i) X at position 178 of SEQ ID NO: 159 is Thr or Cys; (ii) X at position 242 of SEQ ID NO: 159 is Ser, Ala, Val, Leu, Ile, Pro, Phe, Met, or Trp; (iii) X at position 244 of SEQ ID NO: 159 is Met, Ala, Val, Leu, Ile, Pro, Phe, or Trp; and (iv) X at position 245 of SEQ ID NO: 159 is Gly, Ala, Val, Leu, Ile, Pro, Phe, Met, or Trp; (h) a β chain comprising the amino acid sequence of SEQ ID NO: 160 or 161 (β chain with N-terminal signal peptide), wherein X at position 191 of SEQ ID NO: 160 or 161 is Ser or Cys; (I) an α chain comprising the amino acid sequence of SEQ ID NO: 162 (α chain with N-terminal signal peptide), wherein: (i) X at position 191 of SEQ ID NO: 162 is Thr or Cys; (ii) X at position 255 of SEQ ID NO: 162 is Ser, Ala, Val, Leu, Ile, Pro, Phe, Met, or Trp; (iii) X at position 257 of SEQ ID NO: 162 is Met, Ala, Val, Leu, Ile, Pro, Phe, or Trp; and (iv) X at position 258 of SEQ ID NO: 162 is Gly, Ala, Val, Leu, Ile, Pro, Phe, Met, or Trp; (j) a β chain comprising the amino acid sequence of SEQ ID NO: 163 or 164 (β chain with N-terminal signal peptide), wherein X at position 188 of SEQ ID NO: 163 or 164 is Ser or Cys; (k) an α chain comprising the amino acid sequence of SEQ ID NO: 165 (α chain with N-terminal signal peptide), wherein: (i) X at position 180 of SEQ ID NO: 165 is Thr or Cys; (ii) X at position 244 of SEQ ID NO: 165 is Ser, Ala, Val, Leu, Ile, Pro, Phe, Met, or Trp; (iii) X at position 246 of SEQ ID NO: 165 is Met, Ala, Val, Leu, Ile, Pro, Phe, or Trp; and (iv) X at position 247 of SEQ ID NO: 165 is Gly, Ala, Val, Leu, Ile, Pro, Phe, Met, or Trp; (l) a β chain comprising the amino acid sequence of SEQ ID NO: 166 or 167 (β chain with N-terminal signal peptide), wherein X at position 190 of SEQ ID NO: 166 or 167 is Ser or Cys; (m) an α chain comprising the amino acid sequence of SEQ ID NO: 168 (α chain with N-terminal signal peptide), wherein: (i) X at position 180 of SEQ ID NO: 168 is Thr or Cys; (ii) X at position 244 of SEQ ID NO: 168 is Ser, Ala, Val, Leu, Ile, Pro, Phe, Met, or Trp; (iii) X at position 246 of SEQ ID NO: 168 is Met, Ala, Val, Leu, Ile, Pro, Phe, or Trp; and (iv) X at position 247 of SEQ ID NO: 168 is Gly, Ala, Val, Leu, Ile, Pro, Phe, Met, or Trp; (n) a β chain comprising the amino acid sequence of SEQ ID NO: 169 or 170 (β chain with N-terminal signal peptide), wherein X at position 188 of SEQ ID NO: 169 or 170 is Ser or Cys; (o) an α chain comprising the amino acid sequence of SEQ ID NO: 171 (α chain with N-terminal signal peptide), wherein: (i) X at position 177 of SEQ ID NO: 171 is Thr or Cys; (ii) X at position 241 of SEQ ID NO: 171 is Ser, Ala, Val, Leu, Ile, Pro, Phe, Met, or Trp; (iii) X at position 243 of SEQ ID NO: 171 is Met, Ala, Val, Leu, Ile, Pro, Phe, or Trp; and (iv) X at position 244 of SEQ ID NO: 171 is Gly, Ala, Val, Leu, Ile, Pro, Phe, Met, or Trp; (p) a β chain comprising the amino acid sequence of SEQ ID NO: 172 or 173 (β chain with N-terminal signal peptide), wherein X at position 188 of SEQ ID NO: 172 or 173 is Ser or Cys; (q) an α chain comprising the amino acid sequence of SEQ ID NO: 174 (α chain with N-terminal signal peptide), wherein: (i) X at position 184 of SEQ ID NO: 174 is Thr or Cys; (ii) X at position 248 of SEQ ID NO: 174 is Ser, Ala, Val, Leu, Ile, Pro, Phe, Met, or Trp; (iii) X at position 250 of SEQ ID NO: 174 is Met, Ala, Val, Leu, Ile, Pro, Phe, or Trp; and (iv) X at position 251 of SEQ ID NO: 174 is Gly, Ala, Val, Leu, Ile, Pro, Phe, Met, or Trp; (r) a β chain comprising the amino acid sequence of SEQ ID NO: 175 or 176 (β chain with N-terminal signal peptide), wherein X at position 189 of SEQ ID NO: 175 or 176 is Ser or Cys; (s) both (a) and (b); (t) both (c) and (d); (u) both (e) and (0; (v) both (g) and (h); (w) both (I) and (j); (x) both (k) and (l); (y) both (m) and (n); (z) both (o) and (p); (aa) both (q) and (r); (ab) an α chain comprising the amino acid sequence of SEQ ID NO: 177 (α chain without N-terminal signal peptide), wherein: (i) X at position 158 of SEQ ID NO: 177 is Thr or Cys; (ii) X at position 222 of SEQ ID NO: 177 is Ser, Ala, Val, Leu, Ile, Pro, Phe, Met, or Trp; (iii) X at position 224 of SEQ ID NO: 177 is Met, Ala, Val, Leu, Ile, Pro, Phe, or Trp; and (iv) X at position 225 of SEQ ID NO: 177 is Gly, Ala, Val, Leu, Ile, Pro, Phe, Met, or Trp; (ac) a β chain comprising the amino acid sequence of SEQ ID NO: 178 (β chain without N-terminal signal peptide), wherein X at position 167 of SEQ ID NO: 178 is Ser or Cys; (ad) an α chain comprising the amino acid sequence of SEQ ID NO: 179 (α chain without N-terminal signal peptide), wherein: (i) X at position 159 of SEQ ID NO: 179 is Thr or Cys; (ii) X at position 223 of SEQ ID NO: 179 is Ser, Ala, Val, Leu, Ile, Pro, Phe, Met, or Trp; (iii) X at position 225 of SEQ ID NO: 179 is Met, Ala, Val, Leu, Ile, Pro, Phe, or Trp; and (iv) X at position 226 of SEQ ID NO: 179 is Gly, Ala, Val, Leu, Ile, Pro, Phe, Met, or Trp; (ae) a β chain comprising the amino acid sequence of SEQ ID NO: 180 (β chain without N-terminal signal peptide), wherein X at position 169 of SEQ ID NO: 180 is Ser or Cys; (af) an α chain comprising the amino acid sequence of SEQ ID NO: 181 (α chain without N-terminal signal peptide), wherein: (i) X at position 158 of SEQ ID NO: 181 is Thr or Cys; (ii) X at position 222 of SEQ ID NO: 181 is Ser, Ala, Val, Leu, Ile, Pro, Phe, Met, or Trp; (iii) X at position 224 of SEQ ID NO: 181 is Met, Ala, Val, Leu, Ile, Pro, Phe, or Trp; and (iv) X at position 225 of SEQ ID NO: 181 is Gly, Ala, Val, Leu, Ile, Pro, Phe, Met, or Trp; (ag) a β chain comprising the amino acid sequence of SEQ ID NO: 182 (β chain without N-terminal signal peptide), wherein X at position 167 of SEQ ID NO: 182 is Ser or Cys; (ah) an α chain comprising the amino acid sequence of SEQ ID NO: 183 (α chain without N-terminal signal peptide), wherein: (i) X at position 158 of SEQ ID NO: 183 is Thr or Cys; (ii) X at position 222 of SEQ ID NO: 183 is Ser, Ala, Val, Leu, Ile, Pro, Phe, Met, or Trp; (iii) X at position 224 of SEQ ID NO: 183 is Met, Ala, Val, Leu, Ile, Pro, Phe, or Trp; and (iv) X at position 225 of SEQ ID NO: 183 is Gly, Ala, Val, Leu, Ile, Pro, Phe, Met, or Trp; (ai) a β chain comprising the amino acid sequence of SEQ ID NO: 184 (β chain without N-terminal signal peptide), wherein X at position 169 of SEQ ID NO: 184 is Ser or Cys; (aj) an α chain comprising the amino acid sequence of SEQ ID NO: 185 (α chain without N-terminal signal peptide), wherein: (i) X at position 163 of SEQ ID NO: 185 is Thr or Cys; (ii) X at position 227 of SEQ ID NO: 185 is Ser, Ala, Val, Leu, Ile, Pro, Phe, Met, or Trp; (iii) X at position 229 of SEQ ID NO: 185 is Met, Ala, Val, Leu, Ile, Pro, Phe, or Trp; and (iv) X at position 230 of SEQ ID NO: 185 is Gly, Ala, Val, Leu, Ile, Pro, Phe, Met, or Trp; (ak) a β chain comprising the amino acid sequence of SEQ ID NO: 186 (β chain without N-terminal signal peptide), wherein X at position 169 of SEQ ID NO: 186 is Ser or Cys; (al) an α chain comprising the amino acid sequence of SEQ ID NO: 187 (α chain without N-terminal signal peptide), wherein: (i) X at position 159 of SEQ ID NO: 187 is Thr or Cys; (ii) X at position 223 of SEQ ID NO: 187 is Ser, Ala, Val, Leu, Ile, Pro, Phe, Met, or Trp; (iii) X at position 225 of SEQ ID NO: 187 is Met, Ala, Val, Leu, Ile, Pro, Phe, or Trp; and (iv) X at position 226 of SEQ ID NO: 187 is Gly, Ala, Val, Leu, Ile, Pro, Phe, Met, or Trp; (am) a β chain comprising the amino acid sequence of SEQ ID NO: 188 (β chain without N-terminal signal peptide), wherein X at position 172 of SEQ ID NO: 188 is Ser or Cys; (an) an α chain comprising the amino acid sequence of SEQ ID NO: 189 (α chain without N-terminal signal peptide), wherein: (i) X at position 160 of SEQ ID NO: 189 is Thr or Cys; (ii) X at position 224 of SEQ ID NO: 189 is Ser, Ala, Val, Leu, Ile, Pro, Phe, Met, or Trp; (iii) X at position 226 of SEQ ID NO: 189 is Met, Ala, Val, Leu, Ile, Pro, Phe, or Trp; and (iv) X at position 227 of SEQ ID NO: 189 is Gly, Ala, Val, Leu, Ile, Pro, Phe, Met, or Trp; (ao) a β chain comprising the amino acid sequence of SEQ ID NO: 190 (β chain without N-terminal signal peptide), wherein X at position 169 of SEQ ID NO: 190 is Ser or Cys; (ap) an α chain comprising the amino acid sequence of SEQ ID NO: 191 (α chain without N-terminal signal peptide), wherein: (i) X at position 157 of SEQ ID NO: 191 is Thr or Cys; (ii) X at position 221 of SEQ ID NO: 191 is Ser, Ala, Val, Leu, Ile, Pro, Phe, Met, or Trp; (iii) X at position 223 of SEQ ID NO: 191 is Met, Ala, Val, Leu, Ile, Pro, Phe, or Trp; and (iv) X at position 224 of SEQ ID NO: 191 is Gly, Ala, Val, Leu, Ile, Pro, Phe, Met, or Trp; (aq) a β chain comprising the amino acid sequence of SEQ ID NO: 192 (β chain without N-terminal signal peptide), wherein X at position 169 of SEQ ID NO: 192 is Ser or Cys; (ar) an α chain comprising the amino acid sequence of SEQ ID NO: 193 (α chain without N-terminal signal peptide), wherein: (i) X at position 163 of SEQ ID NO: 193 is Thr or Cys; (ii) X at position 227 of SEQ ID NO: 193 is Ser, Ala, Val, Leu, Ile, Pro, Phe, Met, or Trp; (iii) X at position 229 of SEQ ID NO: 193 is Met, Ala, Val, Leu, Ile, Pro, Phe, or Trp; and (iv) X at position 230 of SEQ ID NO: 193 is Gly, Ala, Val, Leu, Ile, Pro, Phe, Met, or Trp; (as) a β chain comprising the amino acid sequence of SEQ ID NO: 194 (β chain without N-terminal signal peptide), wherein X at position 170 of SEQ ID NO: 194 is Ser or Cys; (at) both (j) and (k); (au) both (l) and (m); (av) both (n) and (o); (aw) both (j) and (k); (ax) both (l) and (m); (ay) both (n) and (o); (az) both (j) and (k); (ba) both (1) and (m); or (bb) both (n) and (o). In an embodiment of the invention, any one or more of the sequences of this paragraph comprising the polypeptide are as defined in any one of Tables 1-3.

An embodiment of the invention further provides a protein comprising at least one of the polypeptides described herein. By “protein” is meant a molecule comprising one or more polypeptide chains.

In an embodiment, the protein of the invention can comprise (a) a first polypeptide chain comprising the amino acid sequences of SEQ ID NOs: 1-3 and a second polypeptide chain comprising the amino acid sequences of SEQ ID NOs: 4-6; (b) a first polypeptide chain comprising the amino acid sequences of SEQ ID NOs: 7-9 and a second polypeptide chain comprising the amino acid sequences of SEQ ID NOs: 10-12; (c) a first polypeptide chain comprising the amino acid sequences of SEQ ID NOs: 13-15 and a second polypeptide chain comprising the amino acid sequences of SEQ ID NOs: 16-18; (d) a first polypeptide chain comprising the amino acid sequences of SEQ ID NOs: 19-21 and a second polypeptide chain comprising the amino acid sequences of SEQ ID NOs: 22-24; (e) a first polypeptide chain comprising the amino acid sequences of SEQ ID NOs: 25-27 and a second polypeptide chain comprising the amino acid sequences of SEQ ID NOs: 28-30; (f) a first polypeptide chain comprising the amino acid sequences of SEQ ID NOs: 31-33 and a second polypeptide chain comprising the amino acid sequences of SEQ ID NOs: 34-36; (g) a first polypeptide chain comprising the amino acid sequences of SEQ ID NOs: 37-39 and a second polypeptide chain comprising the amino acid sequences of SEQ ID NOs: 40-42; (h) a first polypeptide chain comprising the amino acid sequences of SEQ ID NOs: 43-45 and a second polypeptide chain comprising the amino acid sequences of SEQ ID NOs: 46-48; or (i) a first polypeptide chain comprising the amino acid sequences of SEQ ID NOs: 49-51 and a second polypeptide chain comprising the amino acid sequences of SEQ ID NOs: 54-54. Any CDR3 of SEQ ID NOS: 3, 6, 9, 12, 15, 18, 21, 24, 27, 30, 33, 36, 39, 42, 45, 48, 51, or 54, i.e., of the α chain or β chain or both, may further comprise a cysteine immediately N-terminal to the first amino acid of the CDR or a phenylalanine immediately C-terminal to the final amino acid or both.

In another embodiment of the invention, the protein may comprise (i) a first polypeptide chain comprising the amino acid sequence of SEQ ID NO: 56 and a second polypeptide chain comprising the amino acid sequence of SEQ ID NO: 57 or 58; (ii) a first polypeptide chain comprising the amino acid sequence of SEQ ID NO: 59 and a second polypeptide chain comprising the amino acid sequence of SEQ ID NO: 60 or 61; (iii) a first polypeptide chain comprising the amino acid sequence of SEQ ID NO: 62 and a second polypeptide chain comprising the amino acid sequence of SEQ ID NO: 63 or 64; (iv) a first polypeptide chain comprising the amino acid sequence of SEQ ID NO: 65 and a second polypeptide chain comprising the amino acid sequence of SEQ ID NO: 66 or 67; (v) a first polypeptide chain comprising the amino acid sequence of SEQ ID NO: 68 and a second polypeptide chain comprising the amino acid sequence of SEQ ID NO: 69 or 70; (vi) a first polypeptide chain comprising the amino acid sequence of SEQ ID NO: 71 and a second polypeptide chain comprising the amino acid sequence of SEQ ID NO: 72 or 73; (vii) a first polypeptide chain comprising the amino acid sequence of SEQ ID NO: 74 and a second polypeptide chain comprising the amino acid sequence of SEQ ID NO: 75 or 76; (viii) a first polypeptide chain comprising the amino acid sequence of SEQ ID NO: 77 and a second polypeptide chain comprising the amino acid sequence of SEQ ID NO: 78 or 79; (ix) a first polypeptide chain comprising the amino acid sequence of SEQ ID NO: 80 and a second polypeptide chain comprising the amino acid sequence of SEQ ID NO: 81 or 82; (x) a first polypeptide chain comprising the amino acid sequence of SEQ ID NO: 83 and a second polypeptide chain comprising the amino acid sequence of SEQ ID NO: 84; (xi) a first polypeptide chain comprising the amino acid sequence of SEQ ID NO: 85 and a second polypeptide chain comprising the amino acid sequence of SEQ ID NO: 86; (xii) a first polypeptide chain comprising the amino acid sequence of SEQ ID NO: 87 and a second polypeptide chain comprising the amino acid sequence of SEQ ID NO: 88; (xiii) a first polypeptide chain comprising the amino acid sequence of SEQ ID NO: 89 and a second polypeptide chain comprising the amino acid sequence of SEQ ID NO: 90; (xiv) a first polypeptide chain comprising the amino acid sequence of SEQ ID NO: 91 and a second polypeptide chain comprising the amino acid sequence of SEQ ID NO: 92; (xv) a first polypeptide chain comprising the amino acid sequence of SEQ ID NO: 93 and a second polypeptide chain comprising the amino acid sequence of SEQ ID NO: 94; (xvi) a first polypeptide chain comprising the amino acid sequence of SEQ ID NO: 95 and a second polypeptide chain comprising the amino acid sequence of SEQ ID NO: 96; (xvii) a first polypeptide chain comprising the amino acid sequence of SEQ ID NO: 97 and a second polypeptide chain comprising the amino acid sequence of SEQ ID NO: 98; or (xviii) a first polypeptide chain comprising the amino acid sequence of SEQ ID NO: 99 and a second polypeptide chain comprising the amino acid sequence of SEQ ID NO: 100.

The inventive protein may further comprise any of the constant regions described herein with respect to other aspects of the invention. In this regard, in embodiments of the invention, the first polypeptide chain may further comprise the amino acid sequence of SEQ ID NO: 101 and the second polypeptide chain may further comprise the amino acid sequence of SEQ ID NO: 102. In embodiments of the invention, the first polypeptide chain may further comprise the amino acid sequence of SEQ ID NO: 148 and the second polypeptide chain may further comprise the amino acid sequence of SEQ ID NO: 149.

In embodiments of the invention, the protein comprises: (a) a first polypeptide chain comprising the amino acid sequence of SEQ ID NO: 148 (constant region of α chain), wherein (i) X at position 48 is Thr or Cys; (ii) X at position 112 is Ser, Ala, Val, Leu, Ile, Pro, Phe, Met, or Trp; (iii) X at position 114 is Met, Ala, Val, Leu, Ile, Pro, Phe, or Trp; and (iv) X at position 115 is Gly, Ala, Val, Leu, Ile, Pro, Phe, Met, or Trp; (b) a second polypeptide chain comprising the amino acid sequence of SEQ ID NO: 149 (constant region of β chain), wherein X at position 57 is Ser or Cys; or (c) both (a) and (b). In embodiments of the invention, one or both of SEQ ID NOs: 148 and 149 of the protein are as defined in any one of Tables 1-3.

Alternatively or additionally, the protein of an embodiment of the invention can comprise (a) a first polypeptide chain comprising the amino acid sequence of SEQ ID NO: 150 (α chain with N-terminal signal peptide), wherein: (i) X at position 178 of SEQ ID NO: 150 is Thr or Cys; (ii) X at position 242 of SEQ ID NO: 150 is Ser, Ala, Val, Leu, Ile, Pro, Phe, Met, or Trp; (iii) X at position 244 of SEQ ID NO: 150 is Met, Ala, Val, Leu, Ile, Pro, Phe, or Trp; and (iv) X at position 245 of SEQ ID NO: 150 is Gly, Ala, Val, Leu, Ile, Pro, Phe, Met, or Trp; (b) a second polypeptide chain comprising the amino acid sequence of SEQ ID NO: 151 or 152 (β chain with N-terminal signal peptide), wherein X at position 188 of SEQ ID NO: 151 or 152 is Ser or Cys; (c) a first polypeptide chain comprising the amino acid sequence of SEQ ID NO: 153 (α chain with N-terminal signal peptide), wherein: (i) X at position 179 of SEQ ID NO: 153 is Thr or Cys; (ii) X at position 243 of SEQ ID NO: 153 is Ser, Ala, Val, Leu, Ile, Pro, Phe, Met, or Trp; (iii) X at position 245 of SEQ ID NO: 153 is Met, Ala, Val, Leu, Ile, Pro, Phe, or Trp; and (iv) X at position 246 of SEQ ID NO: 153 is Gly, Ala, Val, Leu, Ile, Pro, Phe, Met, or Trp; (d) a second polypeptide chain comprising the amino acid sequence of SEQ ID NO: 154 or 155 (β chain with N-terminal signal peptide), wherein X at position 188 of SEQ ID NO: 154 or 155 is Ser or Cys; (e) a first polypeptide chain comprising the amino acid sequence of SEQ ID NO: 156 (α chain with N-terminal signal peptide), wherein: (i) X at position 178 of SEQ ID NO: 156 is Thr or Cys; (ii) X at position 242 of SEQ ID NO: 156 is Ser, Ala, Val, Leu, Ile, Pro, Phe, Met, or Trp; (iii) X at position 244 of SEQ ID NO: 156 is Met, Ala, Val, Leu, Ile, Pro, Phe, or Trp; and (iv) X at position 245 of SEQ ID NO: 156 is Gly, Ala, Val, Leu, Ile, Pro, Phe, Met, or Trp; (f) a second polypeptide chain comprising the amino acid sequence of SEQ ID NO: 157 or 158 (β chain with N-terminal signal peptide), wherein X at position 188 of SEQ ID NO: 157 or 158 is Ser or Cys; (g) a first polypeptide chain comprising the amino acid sequence of SEQ ID NO: 159 (α chain with N-terminal signal peptide), wherein: (i) X at position 178 of SEQ ID NO: 159 is Thr or Cys; (ii) X at position 242 of SEQ ID NO: 159 is Ser, Ala, Val, Leu, Ile, Pro, Phe, Met, or Trp; (iii) X at position 244 of SEQ ID NO: 159 is Met, Ala, Val, Leu, Ile, Pro, Phe, or Trp; and (iv) X at position 245 of SEQ ID NO: 159 is Gly, Ala, Val, Leu, Ile, Pro, Phe, Met, or Trp; (h) a second polypeptide chain comprising the amino acid sequence of SEQ ID NO: 160 or 161 (β chain with N-terminal signal peptide), wherein X at position 191 of SEQ ID NO: 160 or 161 is Ser or Cys; (I) a first polypeptide chain comprising the amino acid sequence of SEQ ID NO: 162 (α chain with N-terminal signal peptide), wherein: (i) X at position 191 of SEQ ID NO: 162 is Thr or Cys; (ii) X at position 255 of SEQ ID NO: 162 is Ser, Ala, Val, Leu, Ile, Pro, Phe, Met, or Trp; (iii) X at position 257 of SEQ ID NO: 162 is Met, Ala, Val, Leu, Ile, Pro, Phe, or Trp; and (iv) X at position 258 of SEQ ID NO: 162 is Gly, Ala, Val, Leu, Ile, Pro, Phe, Met, or Trp; (j) a second polypeptide chain comprising the amino acid sequence of SEQ ID NO: 163 or 164 (β chain with N-terminal signal peptide), wherein X at position 188 of SEQ ID NO: 163 or 164 is Ser or Cys; (k) a first polypeptide chain comprising the amino acid sequence of SEQ ID NO: 165 (α chain with N-terminal signal peptide), wherein: (i) X at position 180 of SEQ ID NO: 165 is Thr or Cys; (ii) X at position 244 of SEQ ID NO: 165 is Ser, Ala, Val, Leu, Ile, Pro, Phe, Met, or Trp; (iii) X at position 246 of SEQ ID NO: 165 is Met, Ala, Val, Leu, Ile, Pro, Phe, or Trp; and (iv) X at position 247 of SEQ ID NO: 165 is Gly, Ala, Val, Leu, Ile, Pro, Phe, Met, or Trp; (1) a second polypeptide chain comprising the amino acid sequence of SEQ ID NO: 166 or 167 (β chain with N-terminal signal peptide), wherein X at position 190 of SEQ ID NO: 166 or 167 is Ser or Cys; (m) a first polypeptide chain comprising the amino acid sequence of SEQ ID NO: 168 (a chain with N-terminal signal peptide), wherein: (i) X at position 180 of SEQ ID NO: 168 is Thr or Cys; (ii) X at position 244 of SEQ ID NO: 168 is Ser, Ala, Val, Leu, Ile, Pro, Phe, Met, or Trp; (iii) X at position 246 of SEQ ID NO: 168 is Met, Ala, Val, Leu, Ile, Pro, Phe, or Trp; and (iv) X at position 247 of SEQ ID NO: 168 is Gly, Ala, Val, Leu, Ile, Pro, Phe, Met, or Trp; (n) a second polypeptide chain comprising the amino acid sequence of SEQ ID NO: 169 or 170 (β chain with N-terminal signal peptide), wherein X at position 188 of SEQ ID NO: 169 or 170 is Ser or Cys; (o) a first polypeptide chain comprising the amino acid sequence of SEQ ID NO: 171 (α chain with N-terminal signal peptide), wherein: (i) X at position 177 of SEQ ID NO: 171 is Thr or Cys; (ii) X at position 241 of SEQ ID NO: 171 is Ser, Ala, Val, Leu, Ile, Pro, Phe, Met, or Trp; (iii) X at position 243 of SEQ ID NO: 171 is Met, Ala, Val, Leu, Ile, Pro, Phe, or Trp; and (iv) X at position 244 of SEQ ID NO: 171 is Gly, Ala, Val, Leu, Ile, Pro, Phe, Met, or Trp; (p) a second polypeptide chain comprising the amino acid sequence of SEQ ID NO: 172 or 173 (β chain with N-terminal signal peptide), wherein X at position 188 of SEQ ID NO: 172 or 173 is Ser or Cys; (q) a first polypeptide chain comprising the amino acid sequence of SEQ ID NO: 174 (α chain with N-terminal signal peptide), wherein: (i) X at position 184 of SEQ ID NO: 174 is Thr or Cys; (ii) X at position 248 of SEQ ID NO: 174 is Ser, Ala, Val, Leu, Ile, Pro, Phe, Met, or Trp; (iii) X at position 250 of SEQ ID NO: 174 is Met, Ala, Val, Leu, Ile, Pro, Phe, or Trp; and (iv) X at position 251 of SEQ ID NO: 174 is Gly, Ala, Val, Leu, Ile, Pro, Phe, Met, or Trp; (r) a second polypeptide chain comprising the amino acid sequence of SEQ ID NO: 175 or 176 (β chain with N-terminal signal peptide), wherein X at position 189 of SEQ ID NO: 175 or 176 is Ser or Cys; (s) both (a) and (b); (t) both (c) and (d); (u) both (e) and (0; (v) both (g) and (h); (w) both (I) and (j); (x) both (k) and (1); (y) both (m) and (n); (z) both (o) and (p); (aa) both (q) and (r); (ab) a first polypeptide chain comprising the amino acid sequence of SEQ ID NO: 177 (α chain without N-terminal signal peptide), wherein: (i) X at position 158 of SEQ ID NO: 177 is Thr or Cys; (ii) X at position 222 of SEQ ID NO: 177 is Ser, Ala, Val, Leu, Ile, Pro, Phe, Met, or Trp; (iii) X at position 224 of SEQ ID NO: 177 is Met, Ala, Val, Leu, Ile, Pro, Phe, or Trp; and (iv) X at position 225 of SEQ ID NO: 177 is Gly, Ala, Val, Leu, Ile, Pro, Phe, Met, or Trp; (ac) a second polypeptide chain comprising the amino acid sequence of SEQ ID NO: 178 (β chain without N-terminal signal peptide), wherein X at position 167 of SEQ ID NO: 178 is Ser or Cys; (ad) a first polypeptide chain comprising the amino acid sequence of SEQ ID NO: 179 (α chain without N-terminal signal peptide), wherein: (i) X at position 159 of SEQ ID NO: 179 is Thr or Cys; (ii) X at position 223 of SEQ ID NO: 179 is Ser, Ala, Val, Leu, Ile, Pro, Phe, Met, or Trp; (iii) X at position 225 of SEQ ID NO: 179 is Met, Ala, Val, Leu, Ile, Pro, Phe, or Trp; and (iv) X at position 226 of SEQ ID NO: 179 is Gly, Ala, Val, Leu, Ile, Pro, Phe, Met, or Trp; (ae) a second polypeptide chain comprising the amino acid sequence of SEQ ID NO: 180 (β chain without N-terminal signal peptide), wherein X at position 169 of SEQ ID NO: 180 is Ser or Cys; (af) a first polypeptide chain comprising the amino acid sequence of SEQ ID NO: 181 (α chain without N-terminal signal peptide), wherein: (i) X at position 158 of SEQ ID NO: 181 is Thr or Cys; (ii) X at position 222 of SEQ ID NO: 181 is Ser, Ala, Val, Leu, Ile, Pro, Phe, Met, or Trp; (iii) X at position 224 of SEQ ID NO: 181 is Met, Ala, Val, Leu, Ile, Pro, Phe, or Trp; and (iv) X at position 225 of SEQ ID NO: 181 is Gly, Ala, Val, Leu, Ile, Pro, Phe, Met, or Trp; (ag) a second polypeptide chain comprising the amino acid sequence of SEQ ID NO: 182 (β chain without N-terminal signal peptide), wherein X at position 167 of SEQ ID NO: 182 is Ser or Cys; (ah) a first polypeptide chain comprising the amino acid sequence of SEQ ID NO: 183 (α chain without N-terminal signal peptide), wherein: (i) X at position 158 of SEQ ID NO: 183 is Thr or Cys; (ii) X at position 222 of SEQ ID NO: 183 is Ser, Ala, Val, Leu, Ile, Pro, Phe, Met, or Trp; (iii) X at position 224 of SEQ ID NO: 183 is Met, Ala, Val, Leu, Ile, Pro, Phe, or Trp; and (iv) X at position 225 of SEQ ID NO: 183 is Gly, Ala, Val, Leu, Ile, Pro, Phe, Met, or Trp; (ai) a second polypeptide chain comprising the amino acid sequence of SEQ ID NO: 184 (β chain without N-terminal signal peptide), wherein X at position 169 of SEQ ID NO: 184 is Ser or Cys; (aj) a first polypeptide chain comprising the amino acid sequence of SEQ ID NO: 185 (α chain without N-terminal signal peptide), wherein: (i) X at position 163 of SEQ ID NO: 185 is Thr or Cys; (ii) X at position 227 of SEQ ID NO: 185 is Ser, Ala, Val, Leu, Ile, Pro, Phe, Met, or Trp; (iii) X at position 229 of SEQ ID NO: 185 is Met, Ala, Val, Leu, Ile, Pro, Phe, or Trp; and (iv) X at position 230 of SEQ ID NO: 185 is Gly, Ala, Val, Leu, Ile, Pro, Phe, Met, or Trp; (ak) a second polypeptide chain comprising the amino acid sequence of SEQ ID NO: 186 (β chain without N-terminal signal peptide), wherein X at position 169 of SEQ ID NO: 186 is Ser or Cys; (al) a first polypeptide chain comprising the amino acid sequence of SEQ ID NO: 187 (α chain without N-terminal signal peptide), wherein: (i) X at position 159 of SEQ ID NO: 187 is Thr or Cys; (ii) X at position 223 of SEQ ID NO: 187 is Ser, Ala, Val, Leu, Ile, Pro, Phe, Met, or Trp; (iii) X at position 225 of SEQ ID NO: 187 is Met, Ala, Val, Leu, Ile, Pro, Phe, or Trp; and (iv) X at position 226 of SEQ ID NO: 187 is Gly, Ala, Val, Leu, Ile, Pro, Phe, Met, or Trp; (am) a second polypeptide chain comprising the amino acid sequence of SEQ ID NO: 188 (β chain without N-terminal signal peptide), wherein X at position 172 of SEQ ID NO: 188 is Ser or Cys; (an) a first polypeptide chain comprising the amino acid sequence of SEQ ID NO: 189 (α chain without N-terminal signal peptide), wherein: (i) X at position 160 of SEQ ID NO: 189 is Thr or Cys; (ii) X at position 224 of SEQ ID NO: 189 is Ser, Ala, Val, Leu, Ile, Pro, Phe, Met, or Trp; (iii) X at position 226 of SEQ ID NO: 189 is Met, Ala, Val, Leu, Ile, Pro, Phe, or Trp; and (iv) X at position 227 of SEQ ID NO: 189 is Gly, Ala, Val, Leu, Ile, Pro, Phe, Met, or Trp; (ao) a second polypeptide chain comprising the amino acid sequence of SEQ ID NO: 190 (β chain without N-terminal signal peptide), wherein X at position 169 of SEQ ID NO: 190 is Ser or Cys; (ap) a first polypeptide chain comprising the amino acid sequence of SEQ ID NO: 191 (α chain without N-terminal signal peptide), wherein: (i) X at position 157 of SEQ ID NO: 191 is Thr or Cys; (ii) X at position 221 of SEQ ID NO: 191 is Ser, Ala, Val, Leu, Ile, Pro, Phe, Met, or Trp; (iii) X at position 223 of SEQ ID NO: 191 is Met, Ala, Val, Leu, Ile, Pro, Phe, or Trp; and (iv) X at position 224 of SEQ ID NO: 191 is Gly, Ala, Val, Leu, Ile, Pro, Phe, Met, or Trp; (aq) a second polypeptide chain comprising the amino acid sequence of SEQ ID NO: 192 (β chain without N-terminal signal peptide), wherein X at position 169 of SEQ ID NO: 192 is Ser or Cys; (ar) a first polypeptide chain comprising the amino acid sequence of SEQ ID NO: 193 (α chain without N-terminal signal peptide), wherein: (i) X at position 163 of SEQ ID NO: 193 is Thr or Cys; (ii) X at position 227 of SEQ ID NO: 193 is Ser, Ala, Val, Leu, Ile, Pro, Phe, Met, or Trp; (iii) X at position 229 of SEQ ID NO: 193 is Met, Ala, Val, Leu, Ile, Pro, Phe, or Trp; and (iv) X at position 230 of SEQ ID NO: 193 is Gly, Ala, Val, Leu, Ile, Pro, Phe, Met, or Trp; (as) a second polypeptide chain comprising the amino acid sequence of SEQ ID NO: 194 (β chain without N-terminal signal peptide), wherein X at position 170 of SEQ ID NO: 194 is Ser or Cys; (at) both (j) and (k); (au) both (l) and (m); (av) both (n) and (o); (aw) both (j) and (k); (ax) both (l) and (m); (ay) both (n) and (o); (az) both (j) and (k); (ba) both (1) and (m); or (bb) both (n) and (o). In an embodiment of the invention, one or more of the sequences of this paragraph are as defined in any one of Tables 1-3.

The protein of the invention can be a TCR. Alternatively, if, for example, the protein comprises a single polypeptide chain or if the first and/or second polypeptide chain(s) of the protein further comprise(s) other amino acid sequences, e.g., an amino acid sequence encoding an immunoglobulin or a portion thereof, then the inventive protein can be a fusion protein. In this regard, an embodiment of the invention also provides a fusion protein comprising at least one of the inventive polypeptides described herein along with at least one other polypeptide. The other polypeptide can exist as a separate polypeptide of the fusion protein, or can exist as a polypeptide, which is expressed in frame (in tandem) with one of the inventive polypeptides described herein. The other polypeptide can encode any peptidic or proteinaceous molecule, or a portion thereof, including, but not limited to an immunoglobulin, CD3, CD4, CD8, an MHC molecule, a CD1 molecule, e.g., CD1a, CD1b, CD1c, CD1d, etc.

The fusion protein can comprise one or more copies of the inventive polypeptide and/or one or more copies of the other polypeptide. For instance, the fusion protein can comprise 1, 2, 3, 4, 5, or more, copies of the inventive polypeptide and/or of the other polypeptide. Suitable methods of making fusion proteins are known in the art, and include, for example, recombinant methods.

In some embodiments of the invention, the TCRs, polypeptides, and proteins of the invention may be expressed as a single protein comprising a linker peptide linking the α chain and the β chain. In this regard, the TCRs, polypeptides, and proteins of the invention may further comprise a linker peptide. The linker peptide may advantageously facilitate the expression of a recombinant TCR, polypeptide, and/or protein in a host cell. The linker peptide may comprise any suitable amino acid sequence. For example, the linker peptide may be a furin-SGSG-P2A linker comprising the amino acid sequence of RAKRSGSGATNFSLLKQAGDVEENPGP (SEQ ID NO: 195). Upon expression of the construct including the linker peptide by a host cell, the linker peptide may be cleaved, resulting in separated α and β chains. In embodiments of the invention, the TCR, polypeptide, or protein may comprise an amino acid sequence comprising a full-length α chain, a full-length β chain, and a linker peptide positioned between the α and β chains, for example α chain-linker-β chain or β chain-linker-α chain. SEQ ID NOS: 196-204 are β chain-linker-α chain.

The protein of the invention can be a recombinant antibody, or an antigen binding portion thereof, comprising at least one of the inventive polypeptides described herein. As used herein, “recombinant antibody” refers to a recombinant (e.g., genetically engineered) protein comprising at least one of the polypeptides of the invention and a polypeptide chain of an antibody, or an antigen binding portion thereof. The polypeptide of an antibody, or antigen binding portion thereof, can be a heavy chain, a light chain, a variable or constant region of a heavy or light chain, a single chain variable fragment (scFv), or an Fc, Fab, or F(ab)₂′ fragment of an antibody, etc. The polypeptide chain of an antibody, or an antigen binding portion thereof, can exist as a separate polypeptide of the recombinant antibody. Alternatively, the polypeptide chain of an antibody, or an antigen binding portion thereof, can exist as a polypeptide, which is expressed in frame (in tandem) with the polypeptide of the invention. The polypeptide of an antibody, or an antigen binding portion thereof, can be a polypeptide of any antibody or any antibody fragment, including any of the antibodies and antibody fragments described herein.

Included in the scope of the invention are functional variants of the inventive TCRs, polypeptides, or proteins described herein. The term “functional variant,” as used herein, refers to a TCR, polypeptide, or protein having substantial or significant sequence identity or similarity to a parent TCR, polypeptide, or protein, which functional variant retains the biological activity of the TCR, polypeptide, or protein of which it is a variant. Functional variants encompass, for example, those variants of the TCR, polypeptide, or protein described herein (the parent TCR, polypeptide, or protein) that retain the ability to specifically bind to CD20 for which the parent TCR has antigenic specificity or to which the parent polypeptide or protein specifically binds, to a similar extent, the same extent, or to a higher extent, as the parent TCR, polypeptide, or protein. In reference to the parent TCR, polypeptide, or protein, the functional variant can, for instance, be at least about 30%, about 50%, about 75%, about 80%, about 90%, about 95%, about 96%, about 97%, about 98%, about 99% or more identical in amino acid sequence to the parent TCR, polypeptide, or protein, respectively.

The functional variant can, for example, comprise the amino acid sequence of the parent TCR, polypeptide, or protein with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the functional variants can comprise the amino acid sequence of the parent TCR, polypeptide, or protein with at least one non-conservative amino acid substitution. In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent TCR, polypeptide, or protein.

The TCR, polypeptide, or protein can consist essentially of the specified amino acid sequence or sequences described herein, such that other components of the TCR, polypeptide, or protein, e.g., other amino acids, do not materially change the biological activity of the TCR, polypeptide, or protein. In this regard, the inventive TCR, polypeptide, or protein can, for example, consist essentially of the amino acid sequence of any of SEQ ID NOS: 103-147 or both of SEQ ID NO: 103, and 104 or 105, both of SEQ ID NO: 106, and 107 or 108, both of SEQ ID NO: 109, and 110 or 111, both of SEQ ID NO: 112, and 113 or 114, both of SEQ ID NO: 115, and 116 or 117, both of SEQ ID NO: 118, and 119 or 120, both of SEQ ID NO: 121, and 122 or 123, both of SEQ ID NO: 124, and 125 or 126, both of SEQ ID NO: 127, and 128 or 129, both of SEQ ID NOS: 130 and 131; both of SEQ ID NOS: 132 and 133; both of SEQ ID NOS: 134 and 135; both of SEQ ID NOS: 136 and 137; both of SEQ ID NOS: 138 and 139; both of SEQ ID NOS: 140 and 141; both of SEQ ID NOS: 142 and 143; both of SEQ ID NOS: 144 and 145; or both of SEQ ID NOS: 146 and 147.

The TCRs, polypeptides, and proteins of the invention can be of any length, i.e., can comprise any number of amino acids, provided that the TCRs, polypeptides, or proteins retain their biological activity, e.g., the ability to specifically bind to CD20; detect cancer in a mammal; or treat or prevent cancer in a mammal, etc. For example, the polypeptide can be in the range of from about 50 to about 5000 amino acids long, such as about 50, about 70, about 75, about 100, about 125, about 150, about 175, about 200, about 300, about 400, about 500, about 600, about 700, about 800, about 900, about 1000 or more amino acids in length. In this regard, the polypeptides of the invention also include oligopeptides.

The TCRs, polypeptides, and proteins of the invention can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The TCRs, polypeptides, and proteins of the invention can be glycosylated, amidated, carboxylated, phosphorylated, esterified, N-acylated, cyclized via, e.g., a disulfide bridge, or converted into an acid addition salt and/or optionally dimerized or polymerized, or conjugated.

The TCR, polypeptide, and/or protein of the invention can be obtained by methods known in the art such as, for example, de novo synthesis. Also, polypeptides and proteins can be recombinantly produced using the nucleic acids described herein using standard recombinant methods. See, for instance, Green and Sambrook, Molecular Cloning: A Laboratory Manual, 4^{th} ed., Cold Spring Harbor Press, Cold Spring Harbor, NY (2012). Alternatively, the TCRs, polypeptides, and/or proteins described herein can be commercially synthesized by companies, such as Synpep (Dublin, CA), Peptide Technologies Corp. (Gaithersburg, MD), and Multiple Peptide Systems (San Diego, CA). In this respect, the inventive TCRs, polypeptides, and proteins can be synthetic, recombinant, isolated, and/or purified. An embodiment of the invention provides an isolated or purified TCR, polypeptide, or protein encoded by any of the nucleic acids or vectors described herein with respect to other aspects of the invention. Another embodiment of the invention provides an isolated or purified TCR, polypeptide, or protein that results from expression of any of the nucleic acids or vectors described herein with respect to other aspects of the invention in a cell. Still another embodiment of the invention provides a method of producing any of the TCRs, polypeptides, or proteins described herein, the method comprising culturing any of the host cells or populations of host cells described herein so that the TCR, polypeptide, or protein is produced.

Included in the scope of the invention are conjugates, e.g., bioconjugates, comprising any of the inventive TCRs, polypeptides, or proteins (including any of the functional portions or variants thereof), nucleic acids, recombinant expression vectors, host cells, populations of host cells, or antibodies, or antigen binding portions thereof. Conjugates, as well as methods of synthesizing conjugates in general, are known in the art.

An embodiment of the invention provides a nucleic acid comprising a nucleotide sequence encoding any of the TCRs, polypeptides, or proteins described herein. “Nucleic acid,” as used herein, includes “polynucleotide,” “oligonucleotide,” and “nucleic acid molecule,” and generally means a polymer of DNA or RNA, which can be single-stranded or double-stranded, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In embodiments, the nucleic acid comprises complementary DNA (cDNA). It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

Preferably, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Green and Sambrook et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, CO) and Synthegen (Houston, TX).

The nucleic acid can comprise any nucleotide sequence which encodes any of the TCRs, polypeptides, or proteins described herein.

TABLE 4

| TCR name | TCR chain | SEQ ID NO: |
|---|---|---|
| J1A1 | J1A1, beta chain (VDJ) | 205 |
| J1A1 | J1A1, alpha chain (VJ) | 206 |
| J1A1 | J1A1, beta chain (VDJ), codon optimized for | 207 |
| | human tissue expression, second amino acid | |
| J1A1 | J1A1, alpha chain (VJ), codon optimized for | 208 |
| | human tissue expression | |
| J1A2 | J1A2, beta chain (VDJ) | 209 |
| J1A2 | J1A2, alpha chain (VJ) | 210 |
| J1A2 | J1A2, beta chain (VDJ), codon optimized for | 211 |
| | human tissue expression, second amino acid | |
| J1A2 | J1A2, alpha chain (VJ), codon optimized for | 212 |
| | human tissue expression | |
| J1A3 | J1A3, beta chain (VDJ) | 213 |
| J1A3 | J1A3, alpha chain (VJ) | 214 |
| J1A3 | J1A3, beta chain (VDJ), codon optimized for | 215 |
| | human tissue expression, second amino acid | |
| J1A3 | J1A3, alpha chain (VJ), codon optimized for | 216 |
| | human tissue expression | |
| 869A1 | 869A1, beta chain (VDJ) | 217 |
| 869A1 | 869A1, alpha chain (VJ) | 218 |
| 869A1 | 869A1, beta chain (VDJ), codon optimized for | 219 |
| | human tissue expression, second amino acid | |
| 869A1 | 869A1, alpha chain (VJ), codon optimized for | 220 |
| | human tissue expression | |
| J2B1 | J2B1, beta chain (VDJ) | 221 |
| J2B1 | J2B1, alpha chain (VJ) | 222 |
| J2B1 | J2B1, beta chain (VDJ), codon optimized for | 223 |
| | human tissue expression, second amino acid | |
| J2B1 | J2B1, alpha chain (VJ), codon optimized for | 224 |
| | human tissue expression | |
| 56A22 | 56A22, beta chain (VDJ) | 225 |
| 56A22 | 56A22, alpha chain (VJ) | 226 |
| 56A22 | 56A22, beta chain (VDJ), codon optimized for | 227 |
| | human tissue expression, second amino acid | |
| 56A22 | 56A22, alpha chain (VJ), codon optimized for | 228 |
| | human tissue expression | |
| 56A23 | 56A23, beta chain (VDJ) | 229 |
| 56A23 | 56A23, alpha chain (VJ) | 230 |
| 56A23 | 56A23, beta chain (VDJ), codon optimized for | 231 |
| | human tissue expression, second amino acid | |
| 56A23 | 56A23, alpha chain (VJ), codon optimized for | 232 |
| | human tissue expression | |
| 56B2 | 56B2, beta chain (VDJ) | 233 |
| 56B2 | 56B2, alpha chain (VJ) | 234 |
| 56B2 | 56B2, beta chain (VDJ), codon optimized for | 235 |
| | human tissue expression, second amino acid | |
| 56B2 | 56B2, alpha chain (VJ), codon optimized for | 236 |
| | human tissue expression | |
| 79B2 | 79B2, beta chain (VDJ) | 237 |
| 79B2 | 79B2, alpha chain (VJ) | 238 |
| 79B2 | 79B2, beta chain (VDJ), codon optimized for | 239 |
| | human tissue expression, second amino acid | |
| 79B2 | 79B2, alpha chain (VJ), codon optimized for | 240 |
| | human tissue expression | |

In embodiments of the invention, the nucleic acid comprises a codon-optimized nucleotide sequence encoding any of the TCRs, polypeptides, or proteins described herein. Without being bound to any particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

The invention also provides a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than α full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive TCRs. It is generally appreciated that conditions can be rendered more stringent by the addition of increasing amounts of formamide.

The invention also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein. In this regard, the nucleic acid may consist essentially of any of the nucleotide sequences described herein.

An embodiment of the invention provides an isolated or purified nucleic acid comprising, from 5′ to 3′, a first nucleic acid sequence and a second nucleotide sequence, wherein the first and second nucleotide sequence, respectively, encode the amino sequences of SEQ ID NOs: 56, and 57 or 58; 57 or 58, and 56; 59, and 60 or 61; 60 or 61, and 59; 62, and 63 or 64; 63 or 64, and 62; 65, and 66 or 67; 66 or 67, and 65; 68, and 69 or 70; 69 or 70, and 68; 71, and 72 or 73; 27 or 73, and 71; 74, and 75 or 76; 75 or 76, and 74; 77, and 78 or 79; 78 or 79, and 77; 80, and 81 or 82; 81 or 82, and 80; 83 and 84; 84 and 83; 85 and 86; 86 and 85; 87 and 88; 88 and 87; 89 and 90; 90 and 89; 91 and 92; 92 and 91; 93 and 94; 94 and 93; 95 and 96; 96 and 95; 97 and 98; 98 and 97; 99 and 100; or 100 and 99.

In an embodiment of the invention, the isolated or purified nucleic acid further comprises a third nucleotide sequence interposed between the first and second nucleotide sequence, wherein the third nucleotide sequence encodes a cleavable linker peptide. In an embodiment of the invention, the cleavable linker peptide comprises the amino acid sequence of SEQ ID NO: 195.

The nucleic acids of the invention can be incorporated into a recombinant expression vector. In this regard, the invention provides a recombinant expression vector comprising any of the nucleic acids of the invention. In embodiments of the invention, the recombinant expression vector comprises a nucleotide sequence encoding the α chain, the β chain, and linker peptide.

For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotide, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages do not hinder the transcription or replication of the vector.

The recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the pUC series (Fermentas Life Sciences), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM and pMAMneo (Clontech). Preferably, the recombinant expression vector is a viral vector, e.g., a retroviral vector. In an especially preferred embodiment, the recombinant expression vector is an MSGV1 vector. In an embodiment of the invention, the recombinant expression vector is a transposon or a lentiviral vector.

The recombinant expression vectors of the invention can be prepared using standard recombinant DNA techniques described in, for example, Green and Sambrook et al., supra. Constructs of expression vectors, which are circular or linear, can be prepared to contain a replication system functional in a prokaryotic or eukaryotic host cell. Replication systems can be derived, e.g., from ColE1, 2μ plasmid, λ, SV40, bovine papillomavirus, and the like.

Desirably, the recombinant expression vector comprises regulatory sequences, such as transcription and translation initiation and termination codons, which are specific to the type of host cell (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected host cells. Marker genes include biocide resistance, e.g., resistance to antibiotics, heavy metals, etc., complementation in an auxotrophic host cell to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the TCR, polypeptide, or protein, or to the nucleotide sequence which is complementary to or which hybridizes to the nucleotide sequence encoding the TCR, polypeptide, or protein. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, nitroreductase, and the inducible caspase 9 gene system.

Another embodiment of the invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human or mouse. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5α cell. For purposes of producing a recombinant TCR, polypeptide, or protein, the host cell is preferably a mammalian cell. Most preferably, the host cell is a human cell. While the host cell can be of any cell type, can originate from any type of tissue, and can be of any developmental stage, the host cell preferably is a peripheral blood lymphocyte (PBL) or a peripheral blood mononuclear cell (PBMC). More preferably, the host cell is a T cell. In an embodiment of the invention, the host cell is a human lymphocyte. In another embodiment of the invention, the host cell is selected from the group consisting of a T cell, a natural killer T (NKT) cell, an invariant natural killer T (iNKT) cell, and a natural killer (NK) cell. Still another embodiment of the invention provides a method of producing a host cell expressing a TCR that has antigenic specificity for the peptide of SEQ ID NO: 55, the method comprising contacting a cell with any of the vectors described herein under conditions that allow introduction of the vector into the cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. Preferably, the T cell is a human T cell. The T cell can be any type of T cell and can be of any developmental stage, including but not limited to, CD4⁺/CD8⁺ double positive T cells, CD4⁺ helper T cells, e.g., Th₁ and Th₂ cells, CD4⁺ T cells, CD8⁺ T cells (e.g., cytotoxic T cells), tumor infiltrating lymphocytes (TILs), memory T cells (e.g., central memory T cells and effector memory T cells), naïve T cells, and the like.

Also provided by the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cells, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

In embodiments of the invention, the numbers of cells in the population may be rapidly expanded. Expansion of the numbers of T cells can be accomplished by any of a number of methods as are known in the art as described in, for example, U.S. Pat. Nos. 8,034,334; 8,383,099; U.S. Patent Application Publication No. 2012/0244133; Dudley et al., J. Immunother., 26:332-42 (2003); and Riddell et al., J. Immunol. Methods, 128:189-201 (1990). In embodiments, expansion of the numbers of T cells is carried out by culturing the T cells with OKT3 antibody, IL-2, and feeder PBMC (e.g., irradiated allogeneic PBMC).

The inventive TCRs, polypeptides, proteins, nucleic acids, recombinant expression vectors, and host cells (including populations thereof), can be isolated and/or purified. The term “isolated,” as used herein, means having been removed from its natural environment. The term “purified,” as used herein, means having been increased in purity, wherein “purity” is a relative term, and not to be necessarily construed as absolute purity. For example, the purity can be at least about 50%, can be greater than about 60%, about 70%, about 80%, about 90%, about 95%, or can be about 100%.

The inventive TCRs, polypeptides, proteins, nucleic acids, recombinant expression vectors, and host cells (including populations thereof), all of which are collectively referred to as “inventive TCR materials” hereinafter, can be formulated into a composition, such as a pharmaceutical composition. In this regard, the invention provides a pharmaceutical composition comprising any of the TCRs, polypeptides, proteins, nucleic acids, expression vectors, and host cells (including populations thereof), described herein, and a pharmaceutically acceptable carrier. The inventive pharmaceutical compositions containing any of the inventive TCR materials can comprise more than one inventive TCR material, e.g., a polypeptide and a nucleic acid, or two or more different TCRs. Alternatively, the pharmaceutical composition can comprise an inventive TCR material in combination with another pharmaceutically active agent(s) or drug(s), such as a chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

Preferably, the carrier is a pharmaceutically acceptable carrier. With respect to pharmaceutical compositions, the carrier can be any of those conventionally used for the particular inventive TCR material under consideration. Methods for preparing administrable compositions are known or apparent to those skilled in the art and are described in more detail in, for example, Remington: The Science and Practice of Pharmacy, 22^{nd} Ed., Pharmaceutical Press (2012). It is preferred that the pharmaceutically acceptable carrier be one which has no detrimental side effects or toxicity under the conditions of use.

The choice of carrier will be determined in part by the particular inventive TCR material, as well as by the particular method used to administer the inventive TCR material. Accordingly, there are a variety of suitable formulations of the pharmaceutical composition of the invention. Suitable formulations may include any of those for parenteral, subcutaneous, intravenous, intramuscular, intraarterial, intrathecal, intratumoral, or interperitoneal administration. More than one route can be used to administer the inventive TCR materials, and in certain instances, a particular route can provide a more immediate and more effective response than another route.

Preferably, the inventive TCR material is administered by injection, e.g., intravenously. When the inventive TCR material is a host cell (or population thereof) expressing the inventive TCR, the pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In embodiments, the pharmaceutically acceptable carrier is supplemented with human serum albumen.

For purposes of the invention, the amount or dose (e.g., numbers of cells when the inventive TCR material is one or more cells) of the inventive TCR material administered should be sufficient to effect, e.g., a therapeutic or prophylactic response, in the subject or animal over a reasonable time frame. For example, the dose of the inventive TCR material should be sufficient to bind to a cancer antigen (e.g., CD20), or detect, treat or prevent cancer in a period of from about 2 hours or longer, e.g., 12 to 24 or more hours, from the time of administration. In certain embodiments, the time period could be even longer. The dose will be determined by the efficacy of the particular inventive TCR material and the condition of the animal (e.g., human), as well as the body weight of the animal (e.g., human) to be treated.

Many assays for determining an administered dose are known in the art. For purposes of the invention, an assay, which comprises comparing the extent to which target cells are lysed or IFN-γ is secreted by T cells expressing the inventive TCR, polypeptide, or protein upon administration of a given dose of such T cells to a mammal among a set of mammals of which each is given a different dose of the T cells, could be used to determine a starting dose to be administered to a mammal. The extent to which target cells are lysed or IFN-γ is secreted upon administration of a certain dose can be assayed by methods known in the art.

The dose of the inventive TCR material also will be determined by the existence, nature and extent of any adverse side effects that might accompany the administration of a particular inventive TCR material. Typically, the attending physician will decide the dosage of the inventive TCR material with which to treat each individual patient, taking into consideration a variety of factors, such as age, body weight, general health, diet, sex, inventive TCR material to be administered, route of administration, and the severity of the cancer being treated. In embodiments in which the inventive TCR material is a population of cells, the number of cells administered per infusion may vary, e.g., from about 1×10⁶ to about 1×10¹² cells or more. In certain embodiments, fewer than 1×10⁶ cells may be administered.

One of ordinary skill in the art will readily appreciate that the inventive TCR materials of the invention can be modified in any number of ways, such that the therapeutic or prophylactic efficacy of the inventive TCR materials is increased through the modification. For instance, the inventive TCR materials can be conjugated either directly or indirectly through a bridge to a chemotherapeutic agent. The practice of conjugating compounds to a chemotherapeutic agent is known in the art. One of ordinary skill in the art recognizes that sites on the inventive TCR materials, which are not necessary for the function of the inventive TCR materials, are suitable sites for attaching a bridge and/or a chemotherapeutic agent, provided that the bridge and/or chemotherapeutic agent, once attached to the inventive TCR materials, do(es) not interfere with the function of the inventive TCR materials, i.e., the ability to bind to CD20 or to detect, treat, or prevent cancer.

It is contemplated that the inventive pharmaceutical compositions, TCRs, polypeptides, proteins, nucleic acids, recombinant expression vectors, host cells, and populations of cells can be used in methods of treating or preventing cancer. Without being bound to a particular theory, the inventive TCRs are believed to bind specifically to CD20, such that the TCR (or related inventive polypeptide or protein), when expressed by a cell, is able to mediate an immune response against a target cell expressing CD20. In this regard, the invention provides a method of treating or preventing cancer in a mammal, comprising administering to the mammal any of the pharmaceutical compositions, TCRs, polypeptides, or proteins described herein, any nucleic acid or recombinant expression vector comprising a nucleotide sequence encoding any of the TCRs, polypeptides, proteins described herein, or any host cell or population of cells comprising a recombinant vector which encodes any of the TCRs, polypeptides, or proteins described herein, in an amount effective to treat or prevent cancer in the mammal.

An embodiment of the invention provides a method of inducing an immune response against a cancer in a mammal, comprising administering to the mammal any of the pharmaceutical compositions, TCRs, polypeptides, or proteins described herein, any nucleic acid or recombinant expression vector comprising a nucleotide sequence encoding any of the TCRs, polypeptides, or proteins described herein, or any host cell or population of cells comprising a recombinant vector which encodes any of the TCRs, polypeptides, or proteins described herein, in an amount effective to induce an immune response against the cancer in the mammal.

An embodiment of the invention provides any of the pharmaceutical compositions, TCRs, polypeptides, or proteins described herein, any nucleic acid or recombinant expression vector comprising a nucleotide sequence encoding any of the TCRs, polypeptides, proteins described herein, or any host cell or population of cells comprising a recombinant vector which encodes any of the TCRs, polypeptides, or proteins described herein, for use in the treatment or prevention of cancer in a mammal.

An embodiment of the invention provides any of the pharmaceutical compositions, TCRs, polypeptides, or proteins described herein, any nucleic acid or recombinant expression vector comprising a nucleotide sequence encoding any of the TCRs, polypeptides, or proteins described herein, or any host cell or population of cells comprising a recombinant vector which encodes any of the TCRs, polypeptides, or proteins described herein, for use in inducing an immune response against a cancer in a mammal.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the cancer being treated or prevented. For example, treatment or prevention can include promoting the regression of a tumor. Also, for purposes herein, “prevention” can encompass delaying the onset of the cancer, or a symptom or condition thereof. Alternatively or additionally, “prevention” may encompass preventing or delaying the recurrence of cancer, or a symptom or condition thereof.

Also provided is a method of detecting the presence of cancer in a mammal. The method comprises (i) contacting a sample comprising one or more cells from the mammal with any of the inventive TCRs, polypeptides, proteins, nucleic acids, recombinant expression vectors, host cells, populations of cells, or pharmaceutical compositions described herein, thereby forming a complex, and (ii) detecting the complex, wherein detection of the complex is indicative of the presence of cancer in the mammal.

With respect to the inventive method of detecting cancer in a mammal, the sample of cells can be a sample comprising whole cells, lysates thereof, or a fraction of the whole cell lysates, e.g., a nuclear or cytoplasmic fraction, a whole protein fraction, or a nucleic acid fraction.

For purposes of the inventive method of detecting cancer, the contacting can take place in vitro or in vivo with respect to the mammal. Preferably, the contacting is in vitro.

Also, detection of the complex can occur through any number of ways known in the art. For instance, the inventive TCRs, polypeptides, proteins, nucleic acids, recombinant expression vectors, host cells, or populations of cells, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

For purposes of the inventive methods, wherein host cells or populations of cells are administered, the cells can be cells that are allogeneic or autologous to the mammal. Preferably, the cells are autologous to the mammal.

With respect to the inventive methods, the cancer can be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, chronic lymphocytic leukemia, chronic myeloid cancer, colon cancer, colorectal cancer, endometrial cancer, esophageal cancer, uterine cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, and urinary bladder cancer. A preferred cancer is a leukemia or a lymphoma (such as Hodgkin lymphoma, T/NK cell lymphoma, or post-transplant lymphoproliferative disorders). In embodiments of the invention, the cancer expresses a CD20 amino acid sequence, wherein the CD20 amino acid sequence is SEQ ID NO: 55. The CD20 expressed by the cancer may be as described herein with respect to other aspects of the invention. Treatment may also apply to other non-cancerous diseases where depletion of CD20-expression B cells have been shown to be efficacious, such as rheumatoid arthritis and other autoimmune diseases.

The mammal referred to in the inventive methods can be any mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

It shall be noted that the preceding are merely examples of embodiments. Other exemplary embodiments are apparent from the entirety of the description herein. It will also be understood by one of ordinary skill in the art that each of these embodiments may be used in various combinations with the other embodiments provided herein.

The following examples further illustrate the invention but, of course, should not be construed as in any way limiting its scope.

### Example 1

This Example demonstrates identification of TCRs having antigenic specificity for CD20 p188-196 (the position of the epitope is based on CD20 Isoform 1—Uniprot identifier: P11836-1), in accordance with embodiments of the invention.

Polyinosinic-polycytidylic acid (poly(I:C)), a Toll-like 3 receptor (TLR3) ligand (Sigma, St. Louis, MO, USA), anti-CD40 anti-body (BioXcell, Lebanon, NH, USA; clone FGK4, agonistic antibody), and CD20 p188-196 (SEQ ID NO: 55) peptide were injected (i.p.) into 6 week-old B6.HLA-A2 transgenic mice (C57BL/6-Mcph1^{Tg(HLA-A2.1)1Enge/J}) at week 0, week 4, and week 8 or at week 0, week 2, and week 4 (poly(I:C) at 50 μg, anti-CD40 Ab at 50 μg, and CD20 peptide at 100 μg in 200 μL PBS per mouse).

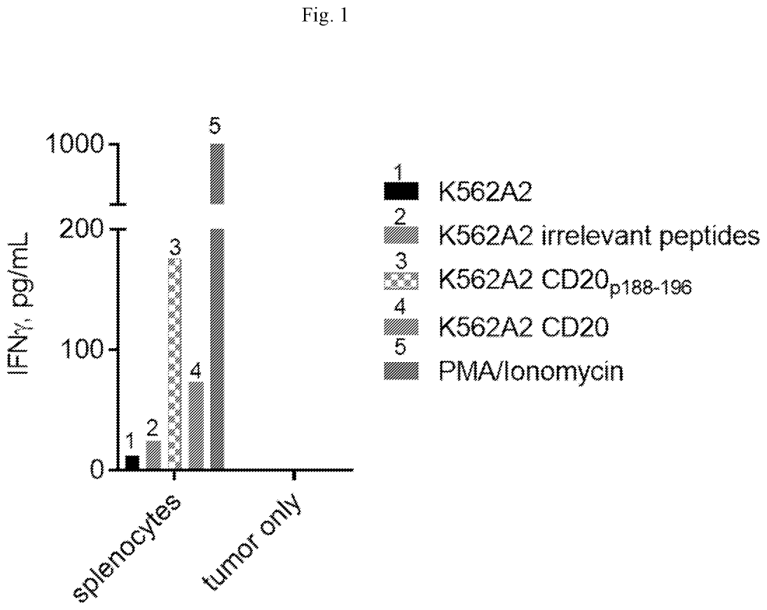

The mice were sacrificed a week after the last boost vaccination. Specimens, including peripheral blood, spleens, and lymph nodes were harvested. Screening for the presence of T cells with T cell receptors (TCRs) with desired specificity was performed with p-MHC tetramer staining and co-culture of lymphocytes with tumor cell lines (effector:tumor (E:T) ratio of 1:1, 1e5 cells each per well in 96-well U-bottom plate), followed by measurement of IFNγ in supernatant by ELISA (after overnight co-culture), intracellular staining of IFNγ (after 4-6 hours of co-culture), assessment of CD107a upregulation (after 4 hours of co-culture), or assessment of 4-1BB upregulation (after overnight co-culture). The results of IFNγ in overnight co-culture supernatant measured by ELISA are shown in FIG. 1.

If initial screening immediately following tissue harvest did not yield T cells with TCRs of desired specificity, remaining lymphocytes (derived from splenocytes or lymph nodes) were stimulated in vitro approximately 1 week apart for up to 4 times in order to increase the precursor frequency of TCRs with desired specificity. The first in vitro stimulation was performed in a tissue culture (TC)-treated 24-well plate by culturing 4e6 harvested splenocytes/lymph node single cell suspension per well in the presence of recombinant human IL-2 at 30 IU/mL and respective peptide at 1 μM. Second, third, and fourth in vitro re-stimulations were performed by adding 4e5 cells/well of irradiated T2 cells (18,000 rad) or irradiated K562-A2 (10,000 rad; K562 retrovirally transduced to express human HLA-A*02:01) pulsed with respective peptide at 1 μM for 30-60 minutes and 2e6 cells/well of irradiated C57BL/6 splenocytes (3000 rad). After repetitive in vitro stimulation, screening for the presence of TCRs with desired specificity was performed as described in the paragraph above.

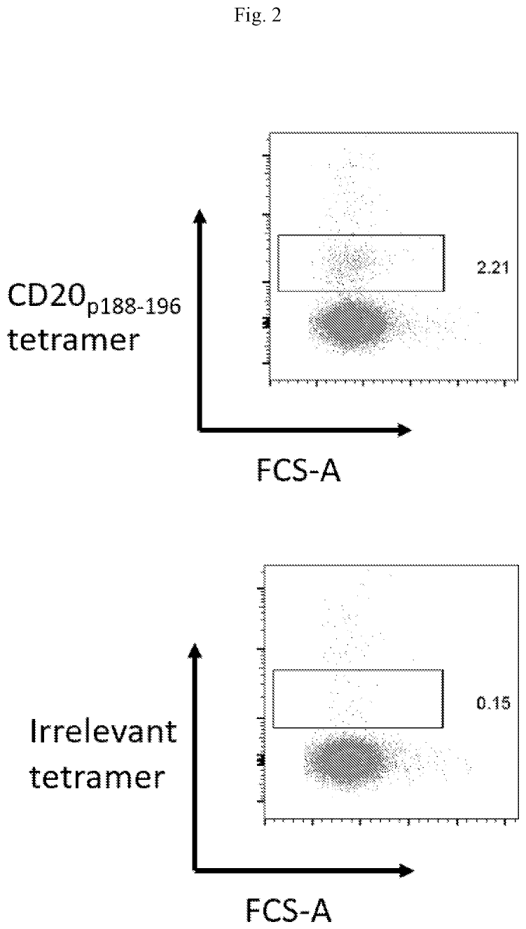

Peptide-MHC (p-MHC) tetramers were manufactured using the UV exchange technique (BioLegend, San Diego, CA, USA; UV Exchange monomer, Flex-T HLA-A*0201 monomer UVX, cat #280004). Results are presented in FIG. 2. Tetramer-bound T cell population was magnetically isolated by staining cells with PE-conjugated p-MHC tetramer followed by enrichment using Anti-PE MicroBeads (Miltenyi Biotec, Bergisch Gladbach, Germany). Alternatively, this step can be substituted with APC-conjucated p-MHC tetramer and Anti-APC Microbeads (Miltenyi Biotec, Bergisch Gladbach, Germany).

Paired single cell TCR sequencing was performed to identify paired T-cell receptor (TCR) TCRα and TCRβ chain sequences (TCRα (TCRAV) and TCRβ (TCRBV)) (10× Genomics, Pleasanton, CA, USA). Sequencing was performed on bulk lymphocytes and/or T cells enriched for tetramer-bound populations as described in the paragraph above.

### Example 2

This Example demonstrates construction of a retroviral vector encoding anti-CD20 TCRs, in accordance with embodiments of the invention.

Separate MSGV1 based-retroviral vectors were constructed which encoded TCR alpha and beta chain variable regions which were based on TCRs of Example 1 with the exception that the amino acid residue at position 2 of the N-terminal signal peptide of the beta chain was changed to an alanine in some TCRs in order to facilitate cloning into the vector. Additional modifications to the wild-type TCR were made, as described in more detail below.

Construction of CD20-specific TCRs was done as previously described (Jin et al., JCI Insight, 3(8): e99488 (2018), incorporated herein by reference in its entirety). Briefly, the TCRβ VDJ regions were fused to the mouse TCRβ constant chain, and the TCRα VJ regions were fused to the mouse TCRα constant chain. Without being bound to a particular theory or mechanism, it is believed that using the murine constant regions improves TCR expression and functionality (Cohen et al., Cancer Res., 66(17): 8878-8886 (2006)).

In addition, the murine TCRα and TCRβ constant chains were cysteine-modified, and transmembrane hydrophobic mutations were introduced into the murine TCRα constant chain. Without being bound to a particular theory or mechanism, it is believed that these modifications result in preferential pairing of the introduced TCR chains and enhanced TCR surface expression and functionality (Cohen et al., Cancer Res., 67(8):3898-903 (2007); Haga-Friedman et al., J. Immu., 188: 5538-5546 (2012)).

The TCRβ and TCRα chains were separated by a furin-recognition sequence SGSG P2A linker (RAKRSGSGATNFSLLKQAGDVEENPGP) (SEQ ID NO: 195) to ensure a comparable expression efficiency of the two chains (Szymczak et al., Nat. Biotechnol., 22(5):589-94 (2004)).

To allow cloning of the CD20-specific TCR expression cassette into the MSGV1 vector 5′NcoI site, the second amino acid in the TCRVβ chain (the second amino acid within the N-terminal signal peptide) was changed to an alanine (A) unless the second amino acid is naturally alanine (A). The expression cassette had the following configuration: 5′NcoI-VDJβ-mCβ-Furin/SGSG/P2A-VJα-mCa-SalI3′. The nucleotide sequences of each TCR were codon optimized for human tissue expression. This example describes a synthesis of bicistronic vector in 5′TCRβ to TCRα 3′ orientation, but the order of TCRβ to TCRα can be reversed.

The modifications described in this Example were made, and the TCR variable regions had sequences as in the following table.

TABLE 5

| TCR | | |
|---|---|---|
| Name | TCR chain | Amino acid sequence |
| TCR J1A1 | Alpha variable | MKSFSISLVVLWLQLNWVNSQQKVQQSPESLIVPE |
| | (TRAV7D-2_TRAJ27) | GGMASLNCTSSDRNVDYFWWYRQHSGKSPKML |
| | (with N-terminal | MSIFSNGEKEEGRFTVHLNKASLHTSLHIRDSQPSD |
| | signal peptide) | SALYLCAASTNTGKLTFGDGTVLTVKP (SEQ ID |
| | | NO: 56) |
| | Beta variable | MAQFCILCLCVLMASVATDPTVTLLEQNPRWRLV |
| | (TRBV1_TRBJ2-7) | PRGQAVNLRCILKNSQYPWMSWYQQDLQKQLQW |
| | (with N-terminal | LFTLRSPGDKEVKSLPGADYLATRVTDTELRLQVA |
| | signal peptide) | NMSQGRTLYCTCSTGDEQYFGPGTRLTVL (SEQ ID |
| | | NO: 58) |
| | Alpha variable | QQKVQQSPESLIVPEGGMASLNCTSSDRNVDYFW |
| | (TRAV7D-2_TRAJ27) | WYRQHSGKSPKMLMSIFSNGEKEEGRFTVHLNKA |
| | (without N-terminal | SLHTSLHIRDSQPSDSALYLCAASTNTGKLTFGDG |
| | signal peptide) | TVLTVKP (SEQ ID NO: 83) |
| | Beta variable | VTLLEQNPRWRLVPRGQAVNLRCILKNSQYPWMS |
| | (TRBV1_TRBJ2-7) | WYQQDLQKQLQWLFTLRSPGDKEVKSLPGADYL |
| | (without N-terminal | ATRVTDTELRLQVANMSQGRTLYCTCSTGDEQYF |
| | signal peptide) | GPGTRLTVL (SEQ ID NO: 84) |
| TCR J1A2 | Alpha variable | MHSLHVSLVFLWLQLGGVSSQEKVQQSPESLIVPE |
| | (TRAV7-6_TRAJ40) | GAMVSLNCSFSDSASQSIWWYQQHPGKGPKALISI |
| | (with N-terminal | FSNGNKKEGRLTVYLNRASLHVSLHIKDSQPSDSA |
| | signal peptide) | VYLCAVFNTGNYKYVFGAGTRLKVIA (SEQ ID |
| | | NO: 59) |
| | Beta variable | MAPRLLCCVIFCLLAATFVDTTVKQNPRYKLARV |
| | (TRBV17_TRBJ2-7) | GKPVNLICSQTMNHDTMYWYQKKPNQAPKLLLF |
| | (with N-terminal | YYDKILNREADTFEKFQSSRPNNSFCSLYIGSAGLE |
| | signal peptide) | YSAMYLCASSPGASYEQYFGPGTRLTVL (SEQ ID |
| | | NO: 61) |
| | Alpha variable | QEKVQQSPESLIVPEGAMVSLNCSFSDSASQSIWW |
| | (TRAV7-6_TRAJ40) | YQQHPGKGPKALISIFSNGNKKEGRLTVYLNRASL |
| | (without N-terminal | HVSLHIKDSQPSDSAVYLCAVFNTGNYKYVFGAG |
| | signal peptide) | TRLKVIA (SEQ ID NO: 85) |
| | Beta variable | DTTVKQNPRYKLARVGKPVNLICSQTMNHDTMY |
| | (TRBV17_TRBJ2-7) | WYQKKPNQAPKLLLFYYDKILNREADTFEKFQSS |
| | (without N-terminal | RPNNSFCSLYIGSAGLEYSAMYLCASSPGASYEQY |
| | signal peptide) | FGPGTRLTVL (SEQ ID NO: 86) |
| TCR J1A3 | Alpha variable | MKSFSISLVVLWLQLNWVNSQQKVQQSPESLIVPE |
| | (TRAV7D-2_TRAJ27) | GGMASLNCTSSDRNVDYFWWYRQHSGKSPKML |
| | (with N-terminal | MSIFSNGEKEEGRFTVHLNKASLHTSLHIRDSQPSD |
| | signal peptide) | SALYLCAASTNTGKLTFGDGTVLTVKP (SEQ ID |
| | | NO: 62) |
| | Beta variable | MAQFCILCLCVLMASVATDPTVTLLEQNPRWRLV |
| | (TRBV1_TRBJ2-7) | PRGQAVNLRCILKNSQYPWMSWYQQDLQKQLQW |
| | (with N-terminal | LFTLRSPGDKEVKSLPGADYLATRVTDTELRLQVA |
| | signal peptide) | NMSQGRTLYCTCSAGDEQYFGPGTRLTVL (SEQ |
| | | ID NO: 64) |
| | Alpha variable | QQKVQQSPESLIVPEGGMASLNCTSSDRNVDYFW |
| | (TRAV7D-2_TRAJ27) | WYRQHSGKSPKMLMSIFSNGEKEEGRFTVHLNKA |
| | (without N-terminal | SLHTSLHIRDSQPSDSALYLCAASTNTGKLTFGDG |
| | signal peptide) | TVLTVKP (SEQ ID NO: 87) |
| | Beta variable | VTLLEQNPRWRLVPRGQAVNLRCILKNSQYPWMS |
| | (TRBV1_TRBJ2-7) | WYQQDLQKQLQWLFTLRSPGDKEVKSLPGADYL |
| | (without N-terminal | ATRVTDTELRLQVANMSQGRTLYCTCSAGDEQYF |
| | signal peptide) | GPGTRLTVL (SEQ ID NO: 88) |
| TCR | Alpha variable | MKSLSVSLVVLWLQFNWVSSQQKVQQSPESLTVS |
| 869A1 | (TRAV7-5_TRAJ27) | EGAMASLNCTFSDGTSNNFRWYRQHSAKGLEVLV |
| | (with N-terminal | SIFSDGEKEEGRFTAHLNRANLHVSLHIREPQPSDS |
| | signal peptide) | AVYLCAVITNTGKLTFGDGTVLTVKP (SEQ ID |
| | | NO: 65) |
| | Beta variable | MAKWVFCWVTLCLLTVETTHGDGGIITQTPKFLIG |
| | (TRBV19_TRBJ2-7) | QEGQKLTLKCQQNFNHDTMYWYRQDSGKGLRLI |
| | (with N-terminal | YYSITENDLQKGDLSEGYDASREKKSSFSLTVTSA |
| | signal peptide) | QKNEMAVFLCASGGAFSYEQYFGPGTRLTVL |
| | | (SEQ ID NO: 67) |
| | Alpha variable | QQKVQQSPESLTVSEGAMASLNCTFSDGTSNNFR |
| | (TRAV7-5_TRAJ27) | WYRQHSAKGLEVLVSIFSDGEKEEGRFTAHLNRA |
| | (without N-terminal | NLHVSLHIREPQPSDSAVYLCAVITNTGKLTFGDG |
| | signal peptide) | TVLTVKP (SEQ ID NO: 89) |
| | Beta variable | GGIITQTPKFLIGQEGQKLTLKCQQNFNHDTMYW |
| | (TRBV19_TRBJ2-7) | YRQDSGKGLRLIYYSITENDLQKGDLSEGYDASRE |
| | (without N-terminal | KKSSFSLTVTSAQKNEMAVFLCASGGAFSYEQYF |
| | signal peptide) | GPGTRLTVL (SEQ ID NO: 90) |
| TCR J2B1 | Alpha variable | MDKILTASFLLLGLHLAGVSGQQQEKHDQQQVRQ |
| | (TRAV14-2_TRAJ52) | SPQSLTVWEGETAILNCSYEDSTFDYFPWYHQFPG |
| | (with N-terminal | ESPALLIAIRPVSNKKEDGRFTIFFNKREKKFSLHIA |
| | signal peptide) | DSQPGDSATYFCAASANTGANTGKLTFGHGTILRV |
| | | HP (SEQ ID NO: 68) |
| | Beta variable | MACRLLSCVAFCLLGIGPLETAVFQTPNYHVTQVG |
| | (TRBV10_TRBJ2-7) | NEVSFNCKQTLGHDTMYWYKQDSKKLLKIMFSY |
| | (with N-terminal | NNKQLIVNETVPRRFSPQSSDKAHLNLRIKSVEPED |
| | signal peptide) | SAVYLCASSWGLGYEQYFGPGTRLTVL (SEQ ID |
| | | NO: 70) |
| | Alpha variable | QQQVRQSPQSLTVWEGETAILNCSYEDSTFDYFPW |
| | (TRAV14-2_TRAJ52) | YHQFPGESPALLIAIRPVSNKKEDGRFTIFFNKREK |
| | (without N-terminal | KFSLHIADSQPGDSATYFCAASANTGANTGKLTFG |
| | signal peptide) | HGTILRVHP (SEQ ID NO: 91) |
| | Beta variable | ETAVFQTPNYHVTQVGNEVSFNCKQTLGHDTMY |
| | (TRBV10_TRBJ2-7) | WYKQDSKKLLKIMFSYNNKQLIVNETVPRRFSPQS |
| | (without N-terminal | SDKAHLNLRIKSVEPEDSAVYLCASSWGLGYEQY |
| | signal peptide) | FGPGTRLTVL (SEQ ID NO: 92) |
| TCR | Alpha variable | MKTVTGPLLLCFWLQLNCVSRGEQVEQRPPHLSV |
| 56A22 | (TRAV3-1_TRAJ27) | REGDSAIIICTYTDSATAYFSWYKQEAGAGLQLLM |
| | (with N-terminal | SVLSNVDRKEEQGLTVLLNKKDKRLSLNLTAAHP |
| | signal peptide) | GDSAVYFCAVDTNTGKLTFGDGTVLTVKP (SEQ |
| | | ID NO: 71) |
| | Beta variable | MASRLFFVVLILLCAKHMEAAVTQSPRSKVAVTG |
| | (TRBV13-3_TRBJ2-3) | GKVTLSCHQTNNHDYMYWYRQDTGHGLRLIHYS |
| | (with N-terminal | YVADSTEKGDIPDGYKASRPSQENFSLILELASLSQ |
| | signal peptide) | TAVYFCASSDRLGGSAETLYFGSGTRLTVL (SEQ |
| | | ID NO: 73) |
| | Alpha variable | GEQVEQRPPHLSVREGDSAIIICTYTDSATAYFSWY |
| | (TRAV3-1_TRAJ27) | KQEAGAGLQLLMSVLSNVDRKEEQGLTVLLNKK |
| | (without N-terminal | DKRLSLNLTAAHPGDSAVYFCAVDTNTGKLTFGD |
| | signal peptide) | GTVLTVKP (SEQ ID NO: 93) |
| | Beta variable | EAAVTQSPRSKVAVTGGKVTLSCHQTNNHDYMY |
| | (TRBV13-3_TRBJ2-3) | WYRQDTGHGLRLIHYSYVADSTEKGDIPDGYKAS |
| | (without N-terminal | RPSQENFSLILELASLSQTAVYFCASSDRLGGSAET |
| | signal peptide) | LYFGSGTRLTVL (SEQ ID NO: 94) |
| TCR | Alpha variable | MQRNLGAVLGILWVQICWVSGDKVKQSPSALSLQ |
| 56A23 | (TRAV4-3_TRAJ52) | EGTNSALRCNFSIAATTVQWFLQNPRGSLINLFYL |
| | (with N-terminal | VPGTKENGRLKSAFDSKESYSTLHIRDAQLEDSGT |
| | signal peptide) | YFCAALANTGANTGKLTFGHGTILRVHP (SEQ ID |
| | | NO: 74) |
| | Beta variable | MAVRLISAVVLCFLGTGLVDMKVTQMPRYLIKRM |
| | (TRBV29_TRBJ2-7) | GENVLLECGQDMSHETMYWYRQDPGLGLQLIYIS |
| | (with N-terminal | YDVDSNSEGDIPKGYRVSRKKREHFSLILDSAKTN |
| | signal peptide) | QTSVYFCASSWGQGYEQYFGPGTRLTVL (SEQ ID |
| | | NO: 76) |
| | Alpha variable | GDKVKQSPSALSLQEGTNSALRCNFSIAATTVQWF |
| | (TRAV4-3_TRAJ52) | LQNPRGSLINLFYLVPGTKENGRLKSAFDSKESYST |
| | (without N-terminal | LHIRDAQLEDSGTYFCAALANTGANTGKLTFGHG |
| | signal peptide) | TILRVHP (SEQ ID NO: 95) |
| | Beta variable | DMKVTQMPRYLIKRMGENVLLECGQDMSHETMY |
| | (TRBV29_TRBJ2-7) | WYRQDPGLGLQLIYISYDVDSNSEGDIPKGYRVSR |
| | (without N-terminal | KKREHFSLILDSAKTNQTSVYFCASSWGQGYEQYF |
| | signal peptide) | GPGTRLTVL (SEQ ID NO: 96) |
| TCR | Alpha variable | MKRLLCSLLGLLCTQVCWVKGQQVQQSPTSLVLQ |
| 56B2 | (TRAV13N-1_TRAJ9) | EGENAELQCNFSTSLNSMQWFYQRPGGSLISVFYN |
| | (with N-terminal | PSGTKQSGRLTSTTVIKERRSSLHISSSQITDSGTYL |
| | signal peptide) | CAMMSNMGYKLTFGTGTSLLVDP (SEQ ID NO: |
| | | 77) |
| | Beta variable | MATRLLGWAVFCLLDTVLSEAGVTQSPRYAVLQE |
| | (TRBV14_TRBJ2-7) | GQAVSFWCDPISGHDTLYWYQQPRDQGPQLLVYF |
| | (with N-terminal | RDEAVIDNSQLPSDRFSAVRPKGTNSTLKIQSAKQ |
| | signal peptide) | GDTATYLCASSLSSYEQYFGPGTRLTVL (SEQ ID |
| | | NO: 79) |
| | Alpha variable | GQQVQQSPTSLVLQEGENAELQCNFSTSLNSMQW |
| | (TRAV13N-1_TRAJ9) | FYQRPGGSLISVFYNPSGTKQSGRLTSTTVIKERRS |
| | (without N-terminal | SLHISSSQITDSGTYLCAMMSNMGYKLTFGTGTSL |
| | signal peptide) | LVDP (SEQ ID NO: 97) |
| | Beta variable | EAGVTQSPRYAVLQEGQAVSFWCDPISGHDTLYW |
| | (TRBV14_TRBJ2-7) | YQQPRDQGPQLLVYFRDEAVIDNSQLPSDRFSAVR |
| | (without N-terminal | PKGTNSTLKIQSAKQGDTATYLCASSLSSYEQYFG |
| | signal peptide) | PGTRLTVL (SEQ ID NO: 98) |
| TCR | Alpha variable | MKTYAPTLFMFLWLQLDGMSQGEQVEQLPSILRV |
| 79B2 | (TRAV5D-4_TRAJ52) | QEGSSASINCSYEDSASNYFPWYKQEPGENPKLIID |
| | (with N-terminal | IRSNMERKQIQELIVLLDKKAKRFSLHITDTQPGDS |
| | signal peptide) | AMYFCAASANTGANTGKLTFGHGTILRVHP (SEQ |
| | | ID NO: 80) |
| | Beta variable | MACRLLSCVAFCLLGIGPLETAVFQTPNYHVTQVG |
| | (TRBV10_TRBJ2-7) | NEVSFNCKQTLGHDTMYWYKQDSKKLLKIMFSY |
| | (with N-terminal | NNKQLIVNETVPRRFSPQSSDKAHLNLRIKSVEPED |
| | signal peptide) | SAVYLCASSWGLGSYEQYFGPGTRLTVL (SEQ ID |
| | | NO: 82) |
| | Alpha variable | GEQVEQLPSILRVQEGSSASINCSYEDSASNYFPWY |
| | (TRAV5D-4_TRAJ52) | KQEPGENPKLIIDIRSNMERKQIQELIVLLDKKAKR |
| | (without N-terminal | FSLHITDTQPGDSAMYFCAASANTGANTGKLTFG |
| | signal peptide) | HGTILRVHP (SEQ ID NO: 99) |
| | Beta variable | ETAVFQTPNYHVTQVGNEVSFNCKQTLGHDTMY |
| | (TRBV10_TRBJ2-7) | WYKQDSKKLLKIMFSYNNKQLIVNETVPRRFSPQS |
| | (without N-terminal | SDKAHLNLRIKSVEPEDSAVYLCASSWGLGSYEQ |
| | signal peptide) | YFGPGTRLTVL (SEQ ID NO: 100) |

### Example 3

This Example demonstrates characterization of TCRs of Example 2, in accordance with embodiments of the invention.

Each of the TCRs identified in Example 2 was retrovirally transduced into human peripheral blood mononuclear cells (PBMC). First, in order to make gammaretoviral supernatant, 293GP was transfected with TCR vector plasmid (1.5 ng/well in 6-well plate) and RD114 envelope plasmid (0.75 μg/well in 6-well plate) using Lipofectamine 3000 Reagent (Thermo Fisher Scientific/Invitrogen, Carlsbad, CA, USA) and Opti-MEM (Gibco Laboratories, Gaithersburg, MD, USA) following manufacturer's instructions. Supernatant of transfected 293GP was harvested and frozen down 48 hours and 72 hours following transfection. Human PBMCs were isolated from healthy donors using ficoll separation. On day 1, human PBMCs (either freshly isolated or cryopreserved PBMCs thawed and rested 0-18 hours in 37° C. prior to use) were stimulated with OKT3 50 ng/mL (CD3 Antibody, anti-human, pure-functional grade, Miltenyi Biotech, Bergisch Gladbach, Germany) and recombinant human IL-2 300 IU/mL in AIM-V medium containing 5% human serum, L-glutamate, penicillin/streptomycin, non-essential amino acid, and HEPES (Gibco Laboratories, Gaithersburg, MD, USA). On day 2, non-tissue culture treated 24 well plate was coated with retronectin (Takara Bio Inc., Kusatsu, Shiga, Japan) 10 μg/mL in PBS, 500 μL/well overnight in 2-8° C. On day 3, viral supernatant (either freshly harvested or freeze-thawed supernatant) was applied to the retronectin-coated plates (500 μL/well). Plates were spun in a centrifuge at 2000 g at 32° C. for 2 hours. Then supernatant was removed, and activated PBMCs were placed into each well at 2.5e5 cells/well in media containing IL-2 300 IU/mL in a final volume of 2 mL/well. On day 4, cells were transferred to tissue-culture treated 24-well plates. On day 5-10, cells were expanded and split as necessary before use for in vitro experiments.

Peptide-MHC tetramers were synthesized using the UV exchange technique (BioLegend, San Diego, CA, USA; UV Exchange monomer, Flex-T HLA-A*0201 monomer UVX, cat #280004). PBMCs transduced with each TCR or untransduced PBMCs were stained with respective p-MHC tetramers, an antibody against murine TCR beta constant region (mTRBC, clone H57-597), other surface antibodies such as CD3, CD8, and CD4, and live/dead discriminating dyes for 20 minutes in the dark at 4° C.

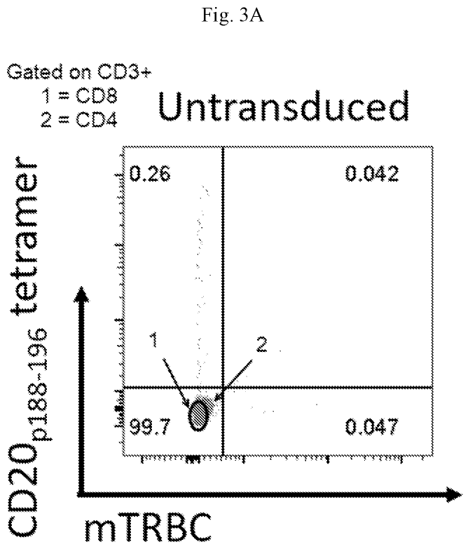

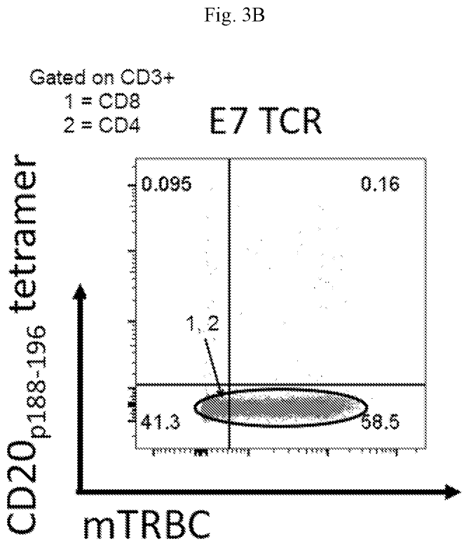

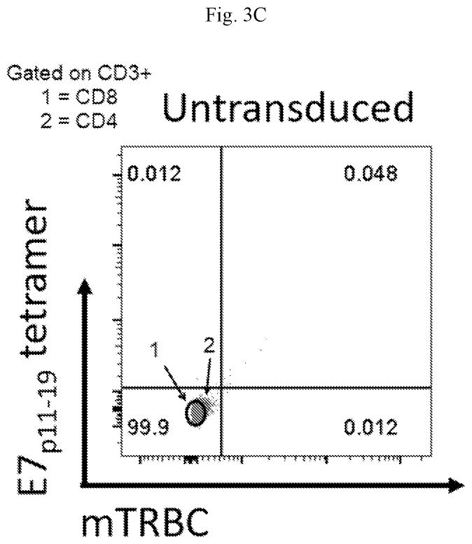

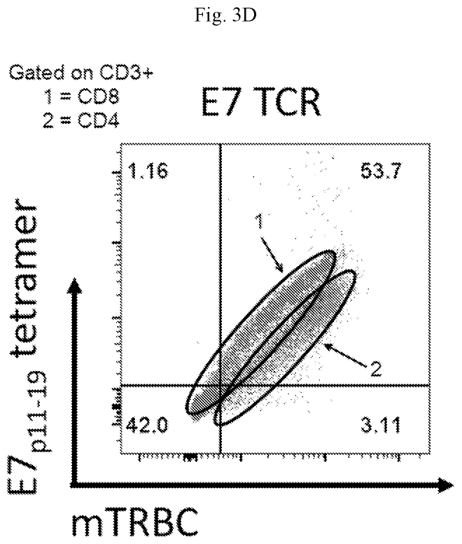

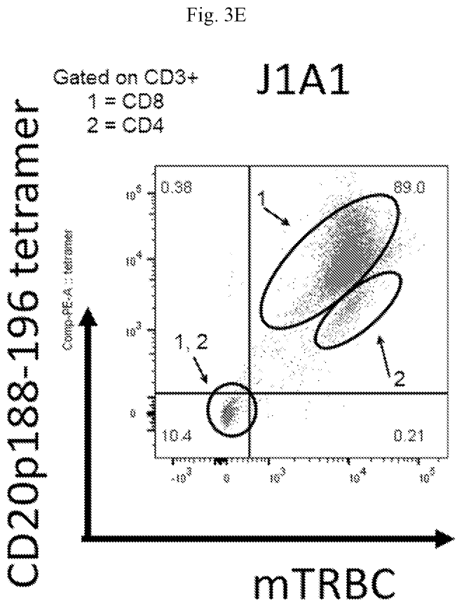

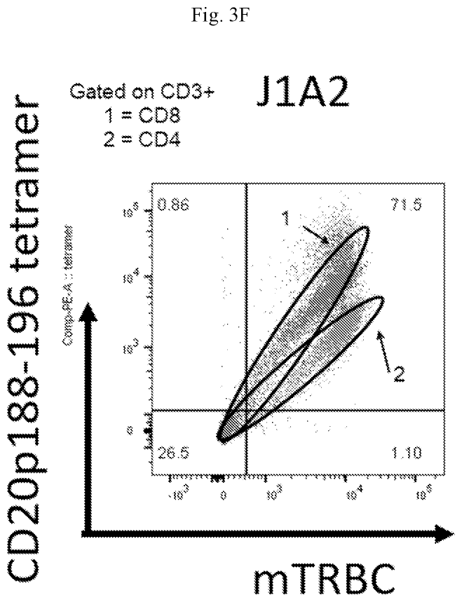

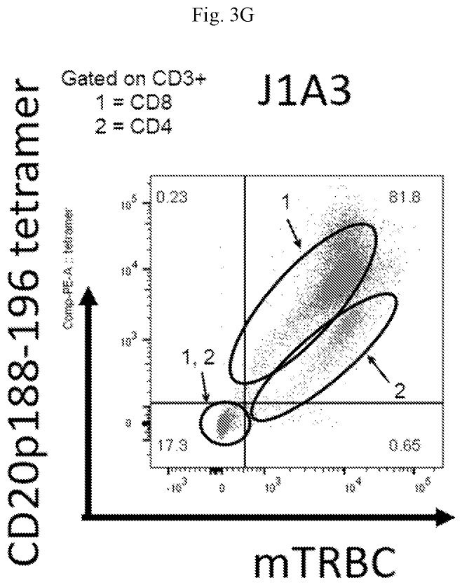

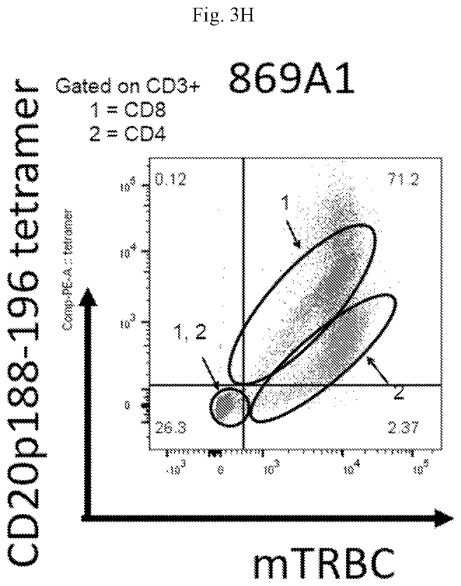

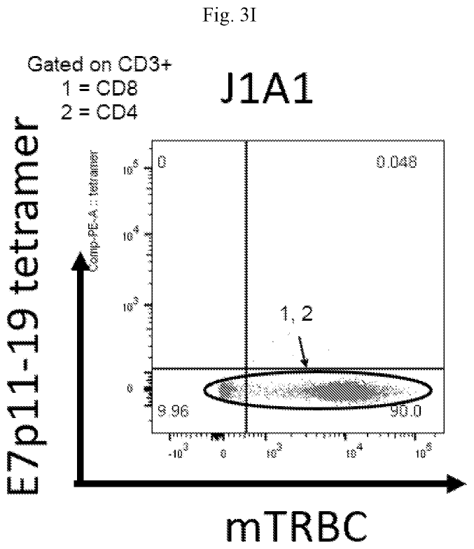

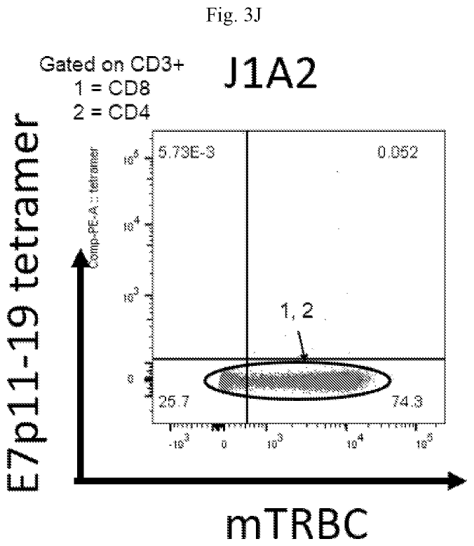

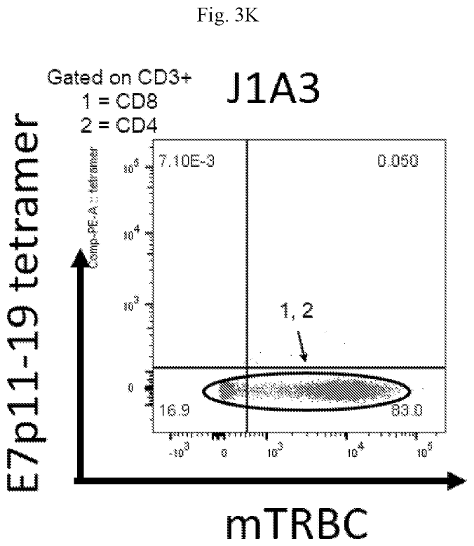

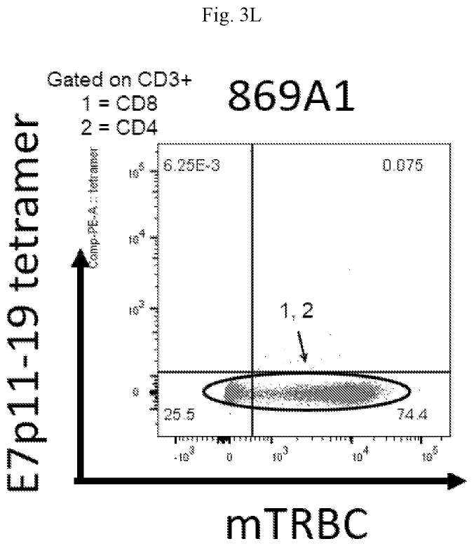

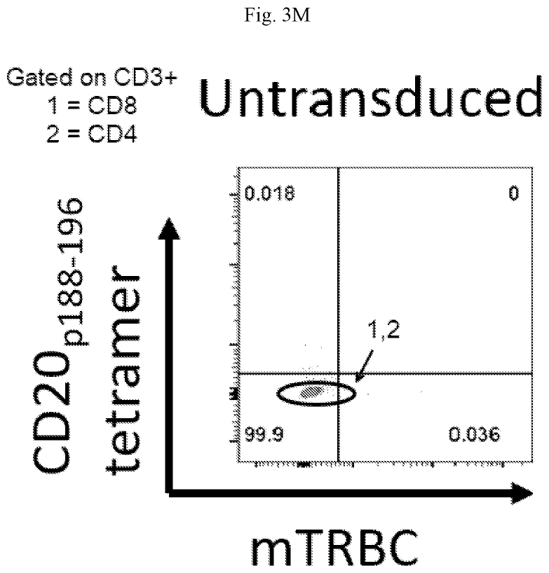

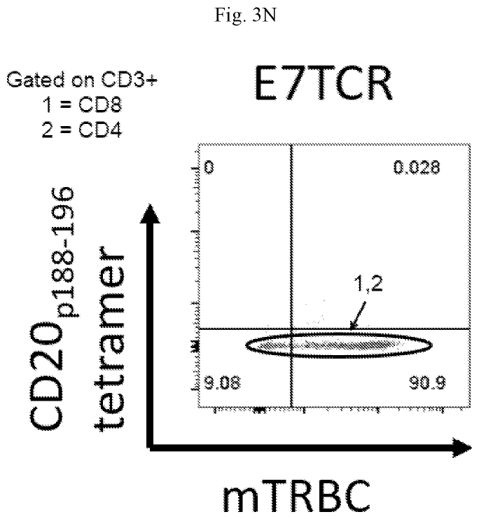

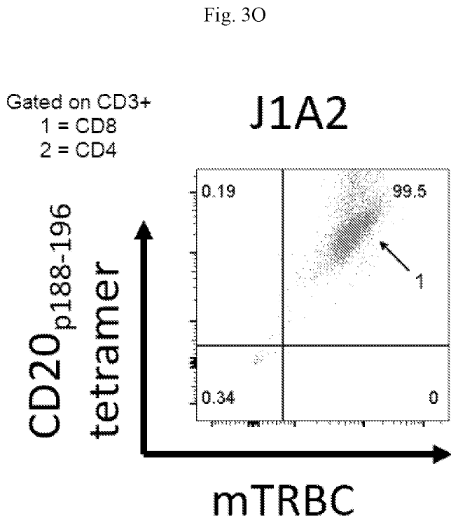

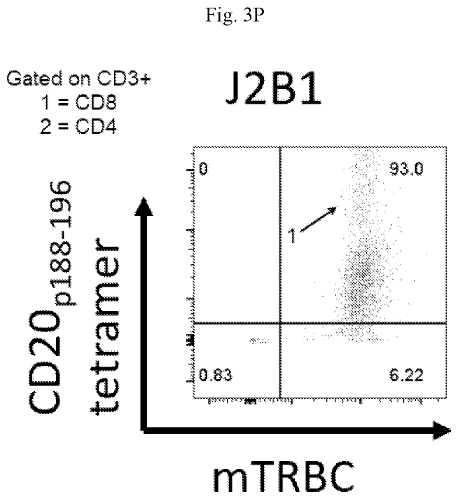

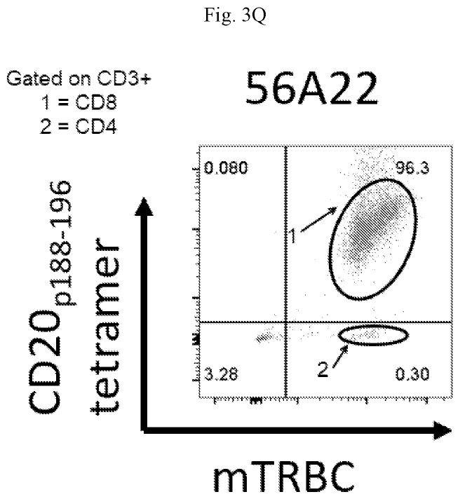

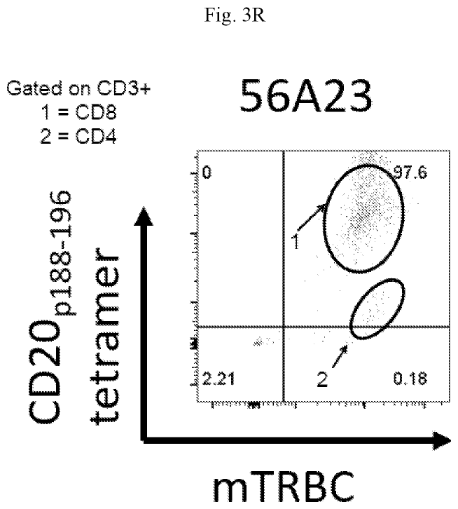

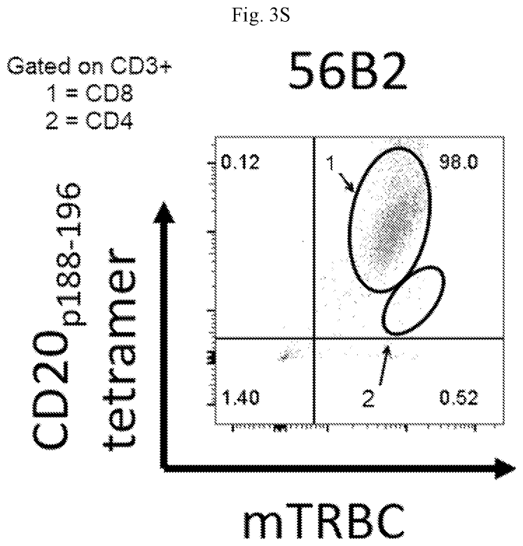

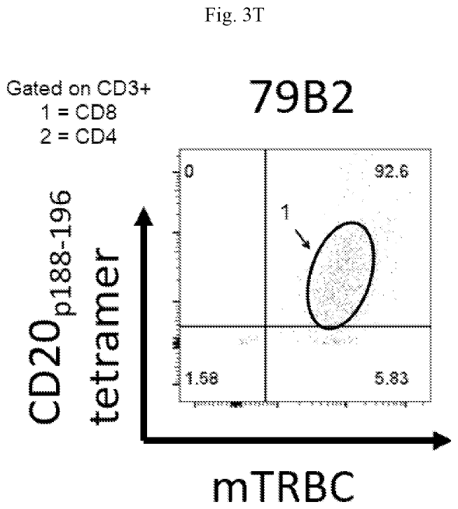

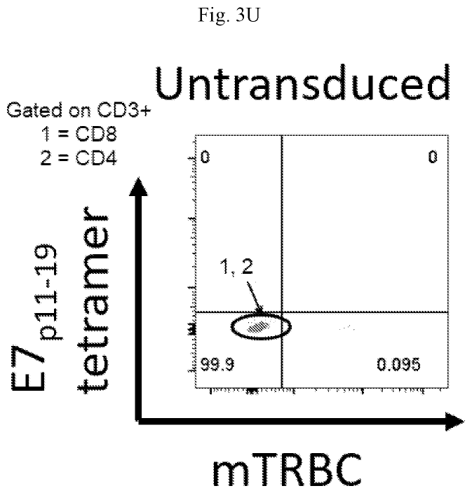

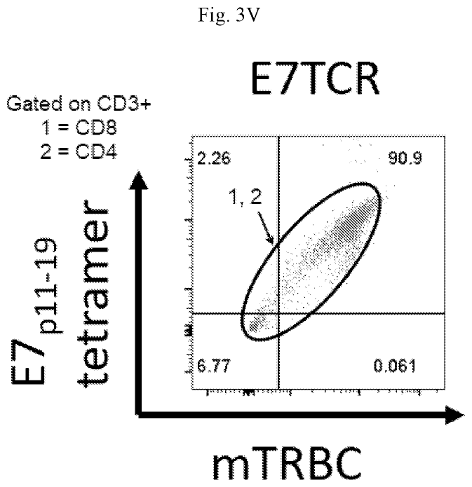

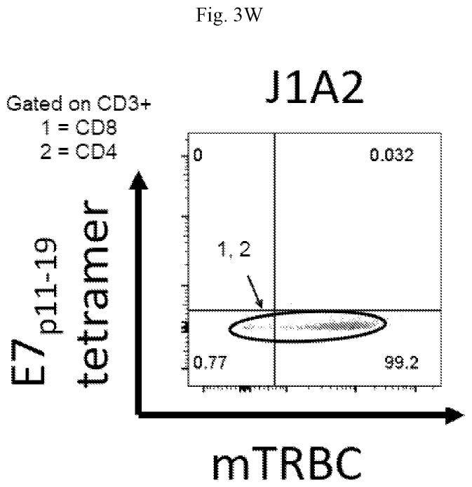

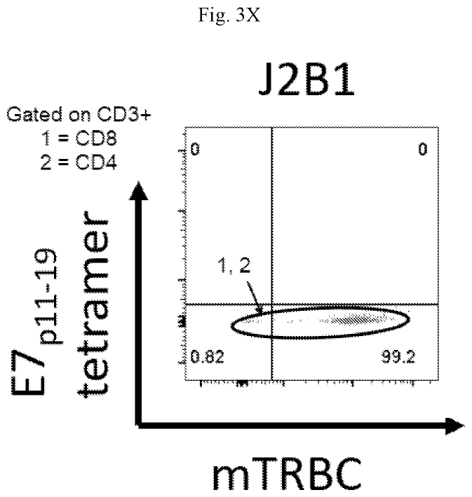

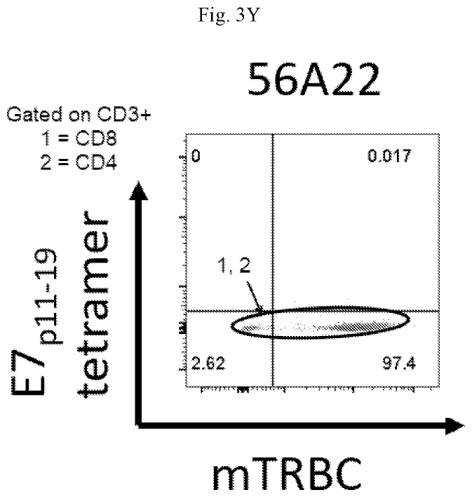

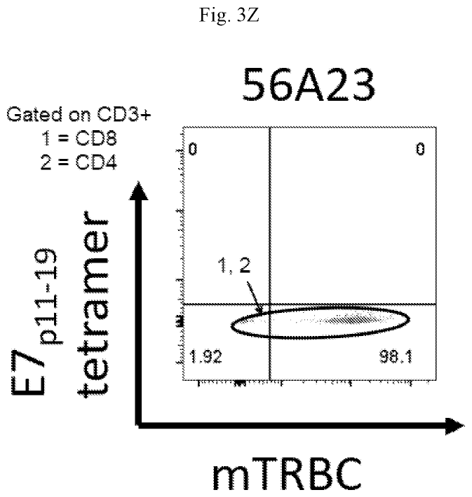

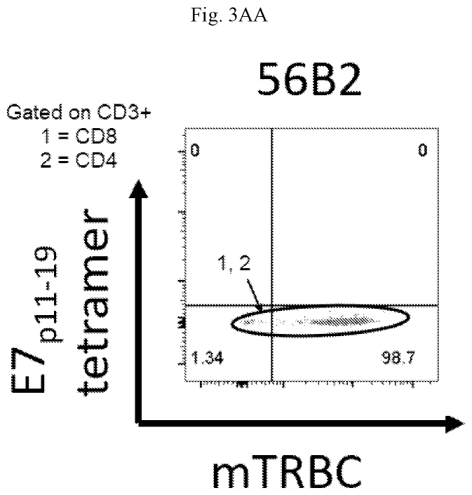

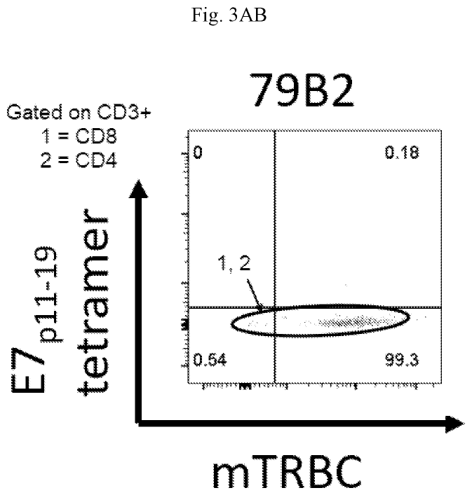

Representative flow cytometry plots are shown in FIGS. 3A-3AB. Each of the TCRs of Example 2 were found to bind specifically to CD20 p-MHC tetramers but not to irrelevant E7 p-MHC tetramer.

### Example 4

This Example demonstrates T cells engineered to express each of inventive TCRs recognized CD20+/HLA-A2+ cell lines, in accordance with embodiments of the invention.

Each target cell line and TCR-transduced T cells or untransduced T cells were co-cultured overnight at an E:T ratio of 1:1 (5e4 cells each per well in 96-well U-bottom plate), and IFNγ levels in the supernatants were measured using ELISA. The target cell lines included:
B cell acute lymphoblastic leukemia: NALM6, CCRF-SB
Burkitt lymphoma: Raji, Ramos, DG75, CA46
Chronic myeloid leukemia, blast phase: NALM1, BV173
Diffuse large B-cell lymphoma: SU-DHL-4, DB
Mantle cell lymphoma: JeKo-1
EBV-LCL: RPMI1788, TM3777, JK4156, SS4050, DC3809, SS3853
T cell acute lymphoblastic leukemia: MOLT4
Acute myeloid leukemia: THP1
HPV-related cervical cancer (HPV-16, E7+): CaSki
K562
K562 CD20: K562 retrovirally transduced with human CD20
K562A2: K562 retrovirally transduced with HLA-A*02:01
K562A2 CD20: K562 retrovirally transduced with human CD20 and HLA-A*02:01
K562A2 CD22: K562 retrovirally transduced with human CD22 and HLA-A*02:01
K562A2 loaded with respective peptide, either CD20 p188-196 or E7 p11-19, at 1 μM

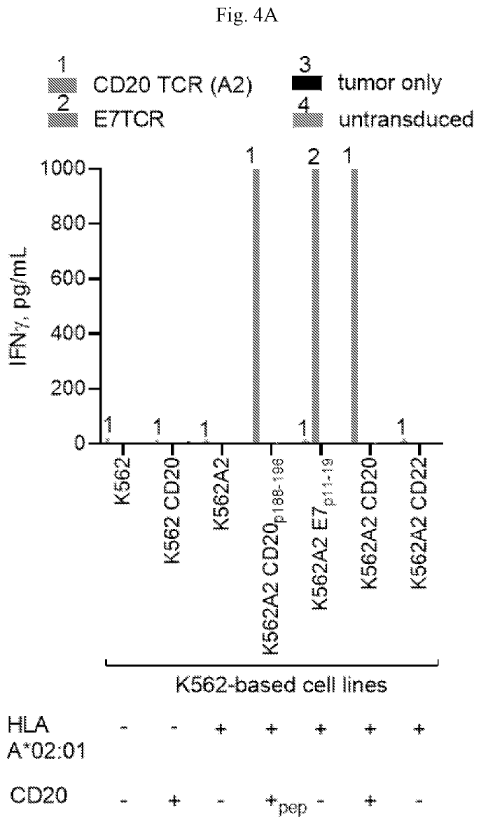

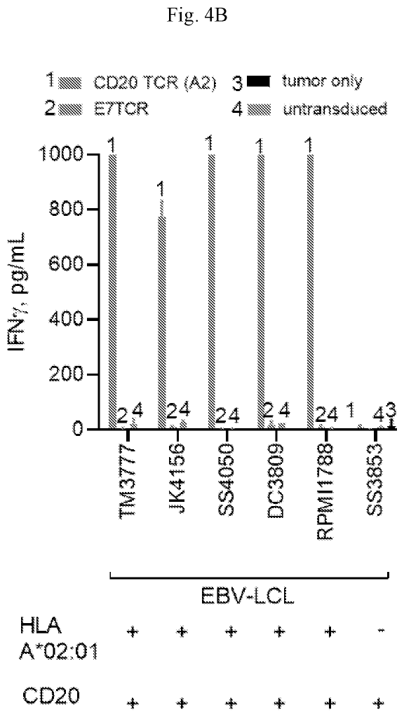

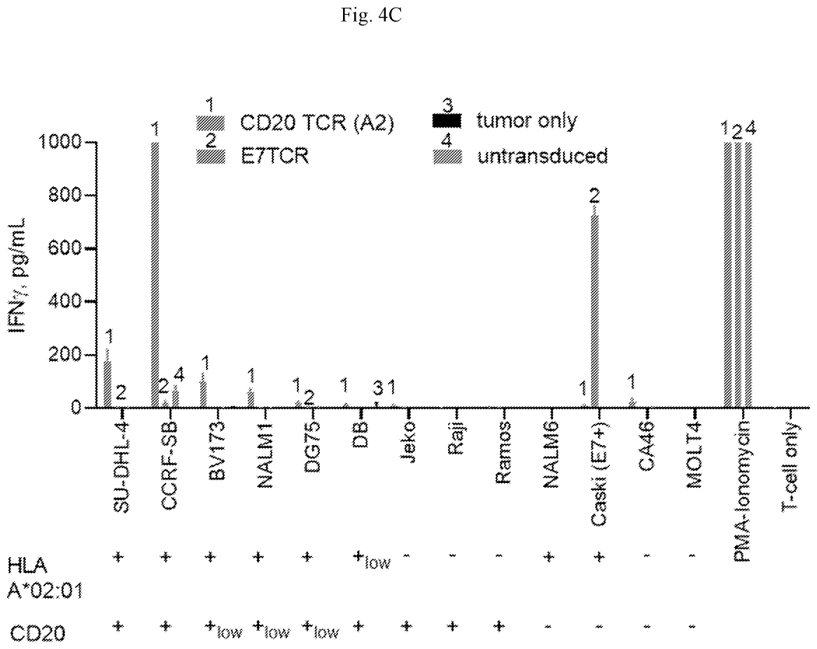

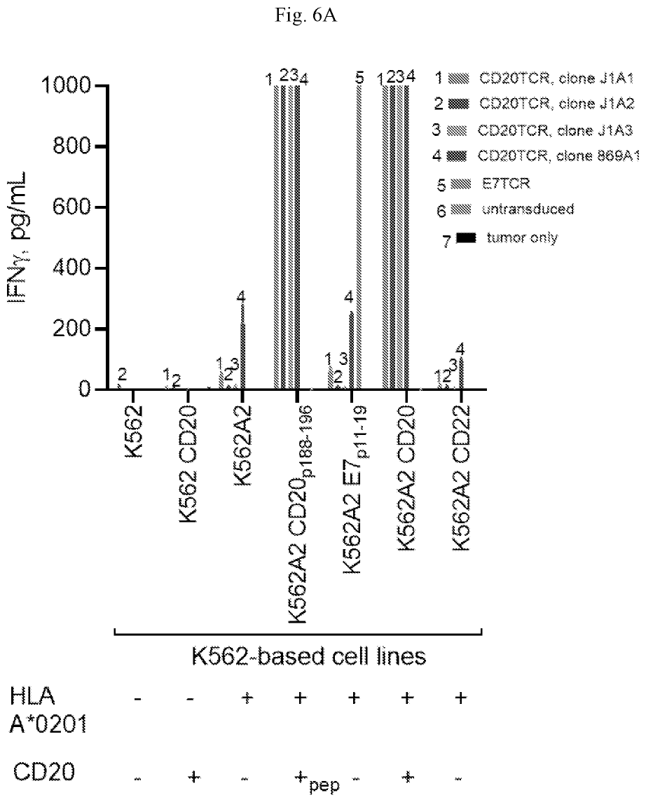

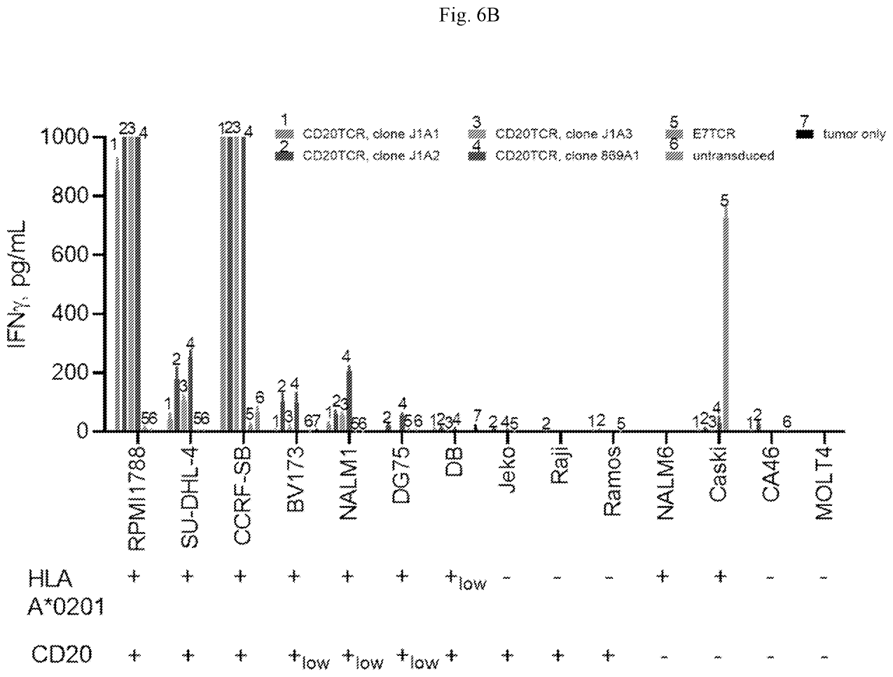

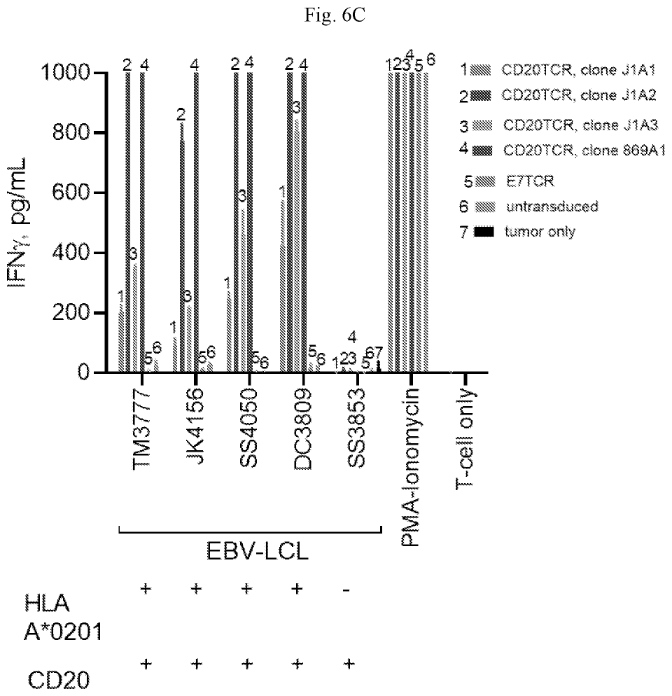

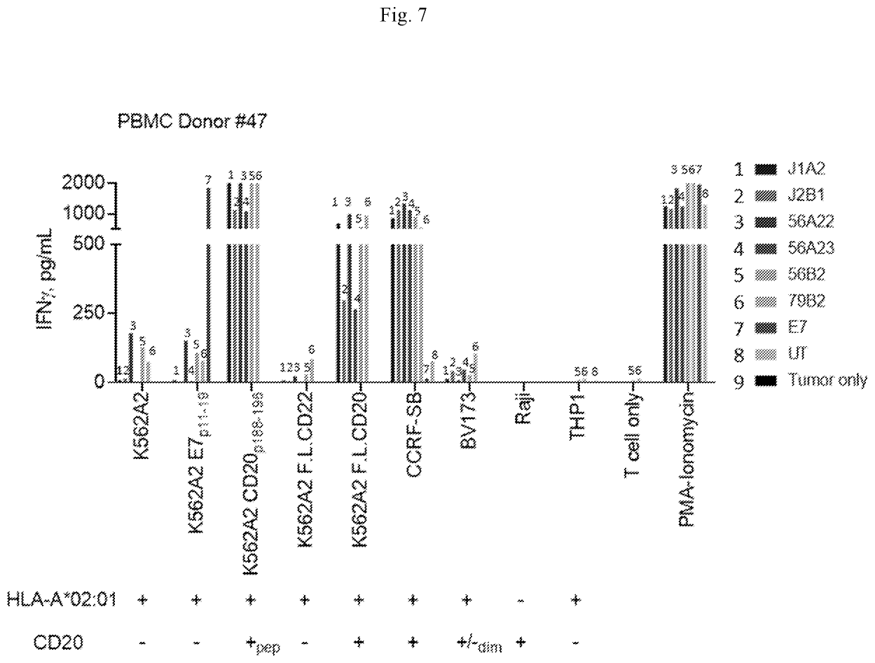

The results are shown in FIGS. 4A-4C, 6A-6C, and 7.

### Example 5

This Example demonstrates the inventive TCRs have high functional avidity, in accordance with embodiments of the invention.

HLA-A2+CD20 negative cells, K562A2 cell line, were loaded with respective peptide at a concentration indicated in the x-axis for 60 minutes at 37° C. Subsequently, TCR-transduced T cells and peptide-loaded K562A2 cells were co-cultured at an E:T=1:1, 5e4 cells each per well in 96-well U-bottom plate overnight. IFNγ levels in overnight co-culture supernatant were measured using ELISA.

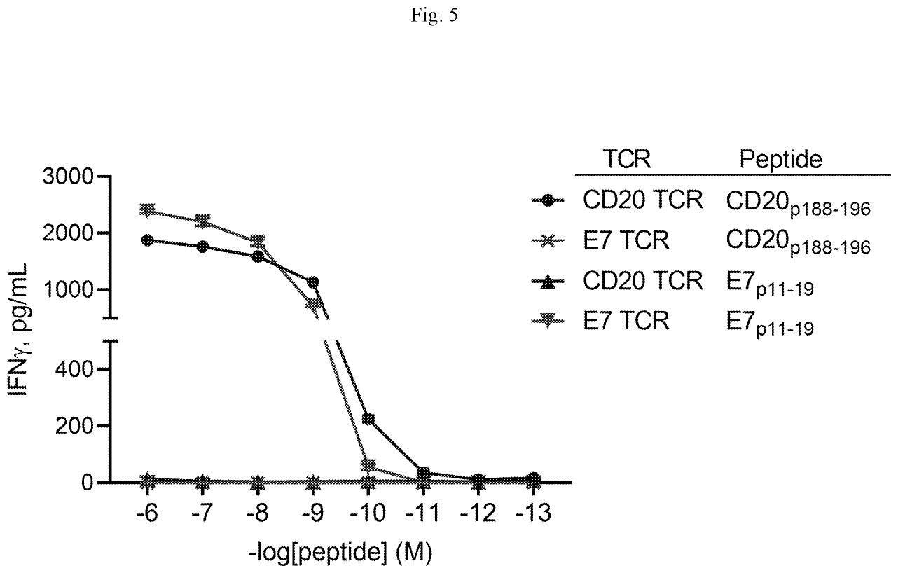

The results of functional avidity assay for TCR J1A2, compared with E7-TCR, are shown in FIG. 5.

### Example 6

This Example demonstrates activity of the J1A2 TCR, in accordance with embodiments of the invention.

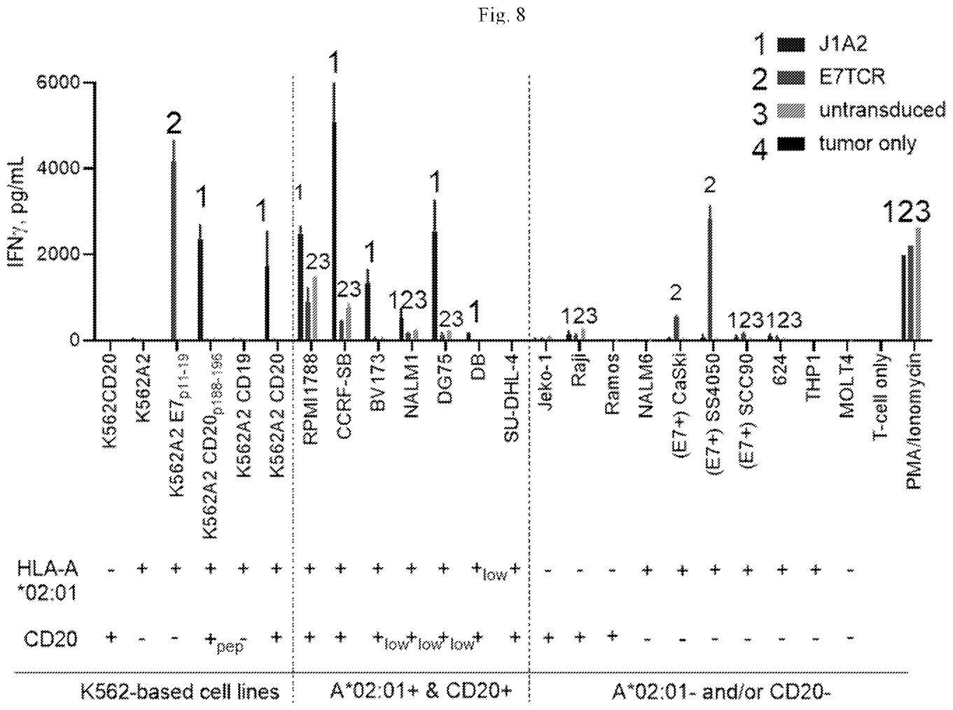

T cells transduced to express the anti-CD20 TCR (clone J1A2) do not produce IFNγ upon co-culturing with cell lines that are HLA-A*02:01+ but CD20-negative. This finding supports that the anti-CD20 TCR does not non-specifically recognize HLA-A*02:01. Results are shown in FIG. 8.

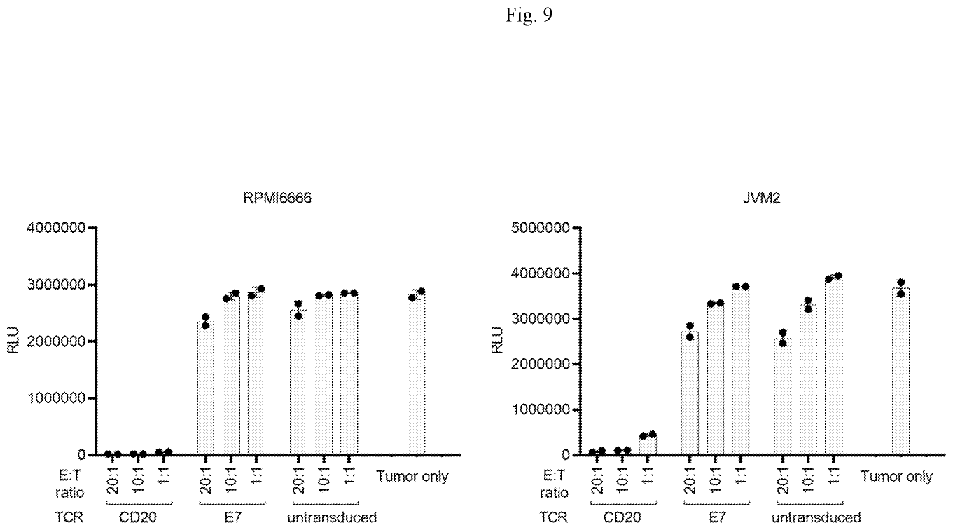

T cells transduced to express the anti-CD20 TCR (clone J1A2) mediate cytotoxicity against CD20+ lymphoid malignancy cell lines. See FIG. 9.

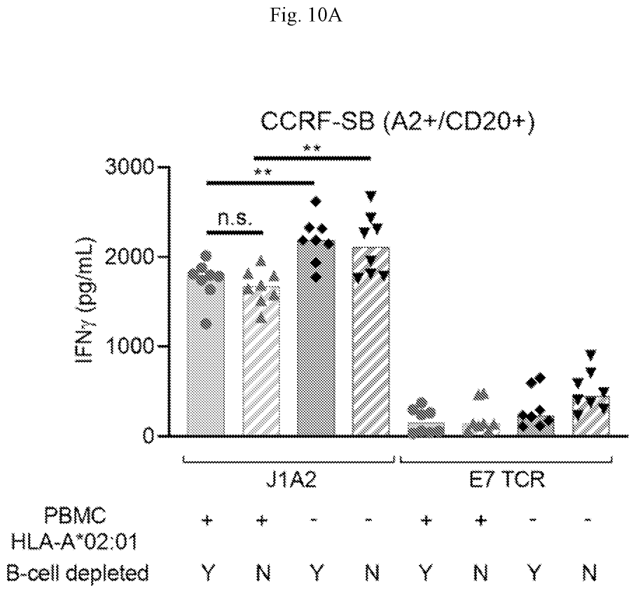

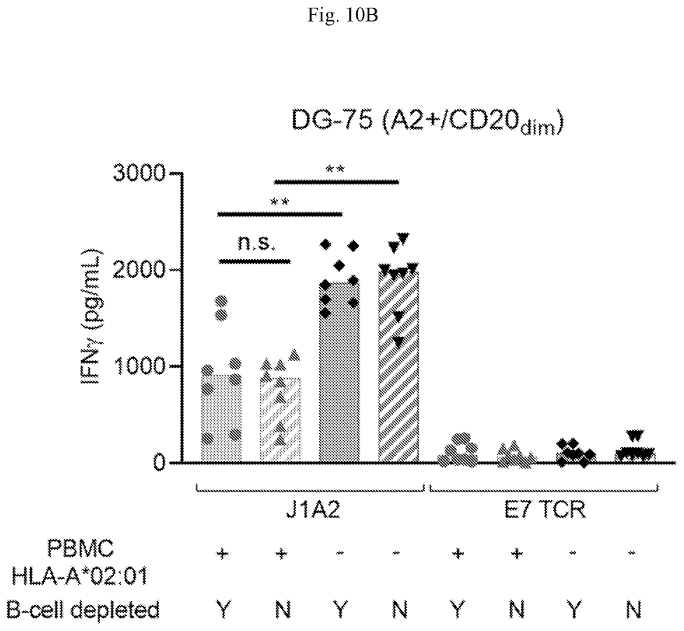

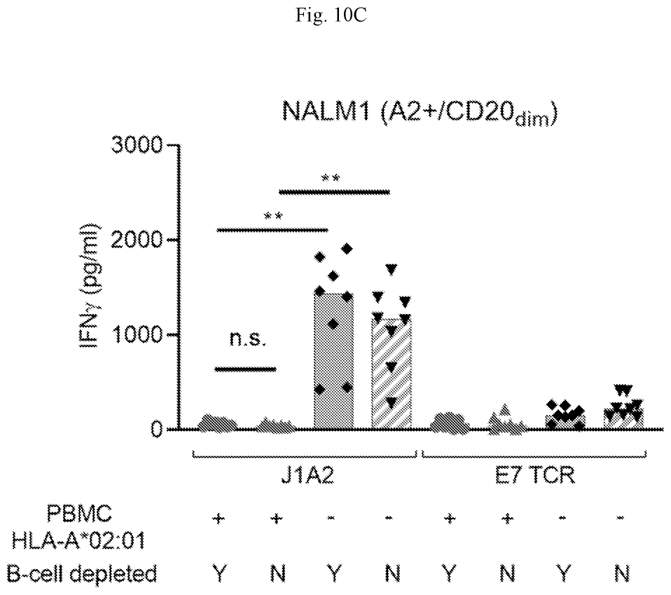

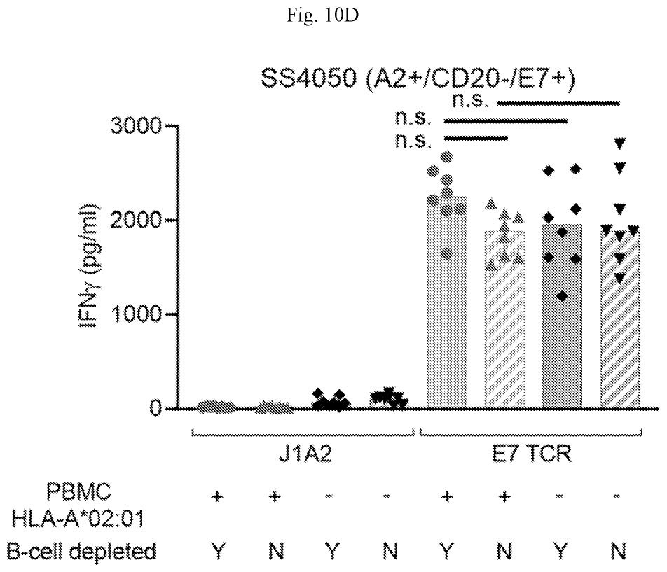

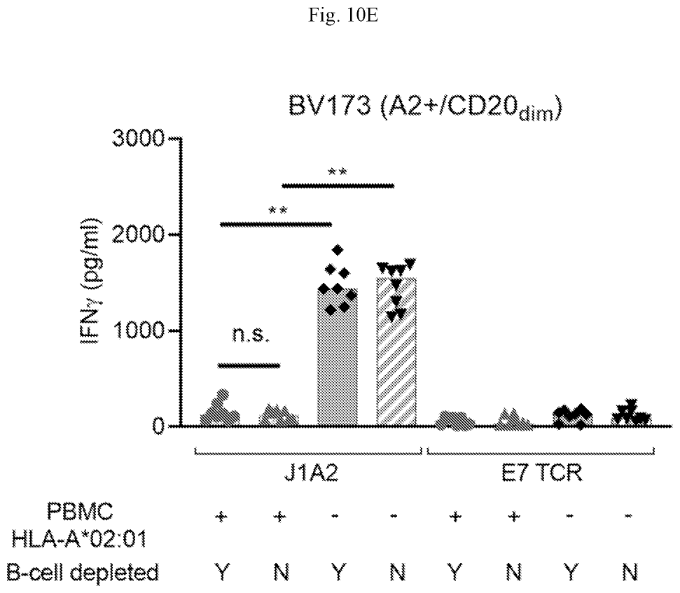

HLA-A2+ T cells transduced to express the anti-CD20TCR (clone J1A2) are functionally less avid compared to HLA-A2-negative counterparts. Depletion of autologous B cells prior to T cell activation and TCR transduction did not reverse this finding. One of the potential explanations of this finding may be that the anti-CD20TCR is cross-reacting against an unknown HLA-A2-restricted epitope expressed by T cells or other cell populations in the PBMC, causing the T cells to terminally differentiate or “exhaust” due to repetitive stimulation. See FIGS. 10A-10E.

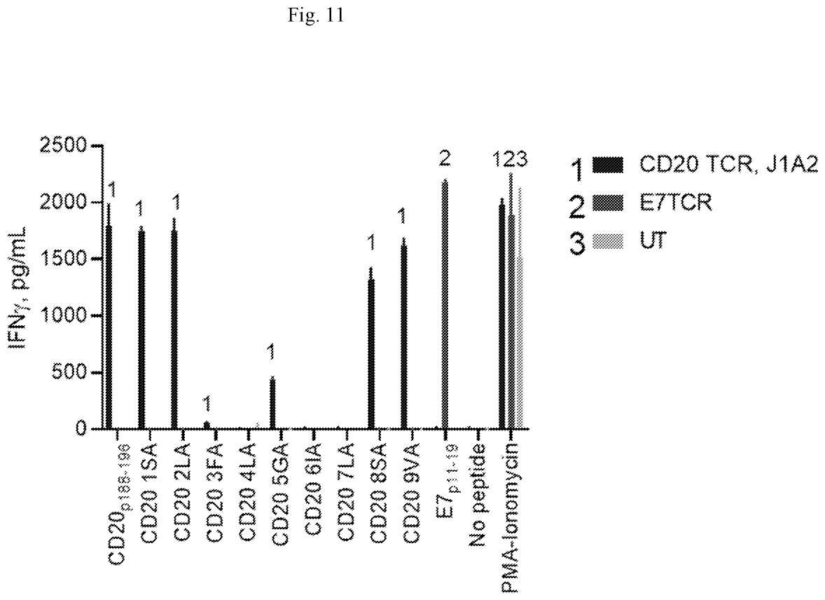

Alanine scanning was performed to determine the peptide residues that are important for TCR-pMHC interaction. K562A2 cells were loaded with 1 μM of minimal epitope peptide or peptides with alanine substitutions. Peptide-loaded K562A2 cells were co-cultured with CD20TCR- or E7TCR-transduced T cells at an E:T ratio of 1:1, and the levels of IFNγ in the overnight co-culture supernatant was measured with ELISA. See FIG. 11. The table below shows predicted IC50 values of each peptide for HLA-A*02:01 (IMGT website.

TABLE 6

| | | Predicted MHC |
|---|---|---|
| Peptide | SEQ ID NO: | IC50 [nM] |
| SLFLGILSV | 55 | 7.7 |
| ALFLGILSV | 241 | 8.6 |
| SAFLGILSV | 242 | 142.7 |
| SLALGILSV | 243 | 14.2 |
| SLFAGILSV | 244 | 3.3 |
| SLFLAILSV | 245 | 13.8 |
| SLFLGALSV | 246 | 11.8 |
| SLFLGIASV | 247 | 8.1 |
| SLFLGILAV | 248 | 8.3 |
| SLFLGILSA | 249 | 26.1 |

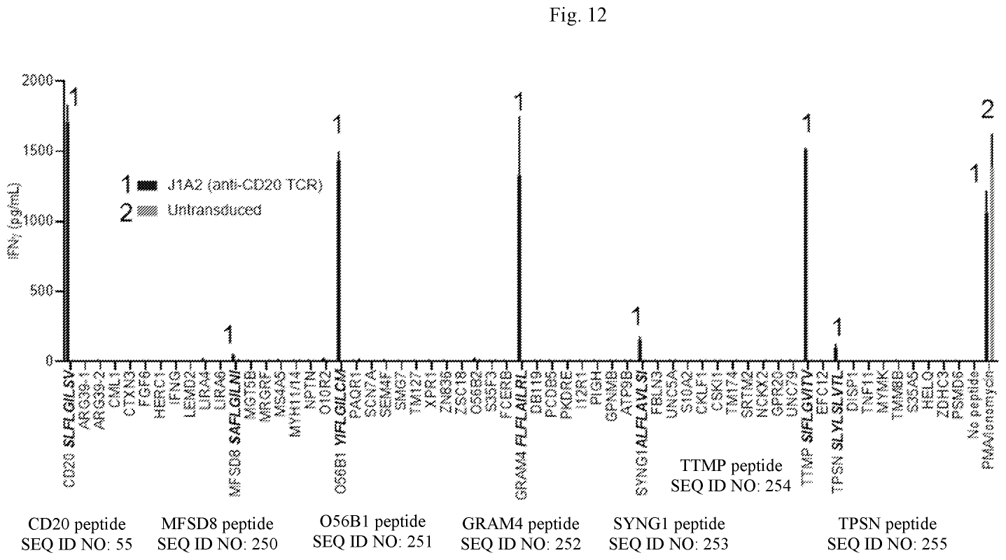

An in silico search was performed for human peptides with amino acid sequences that are similar to the TCR recognition motif and/or human peptides with high levels of sequence homology (ScanProsite tool, NCBI protein BLAST). Whether the CD20TCR (clone J1A2) cross-reacts with these peptides was evaluated by co-culturing the J1A2-transduced T cells with K562A2 (K562 transduced to express HLA-A*02:01) loaded with 1 μM of each peptide. The results of co-culture experiments are shown in FIG. 12.

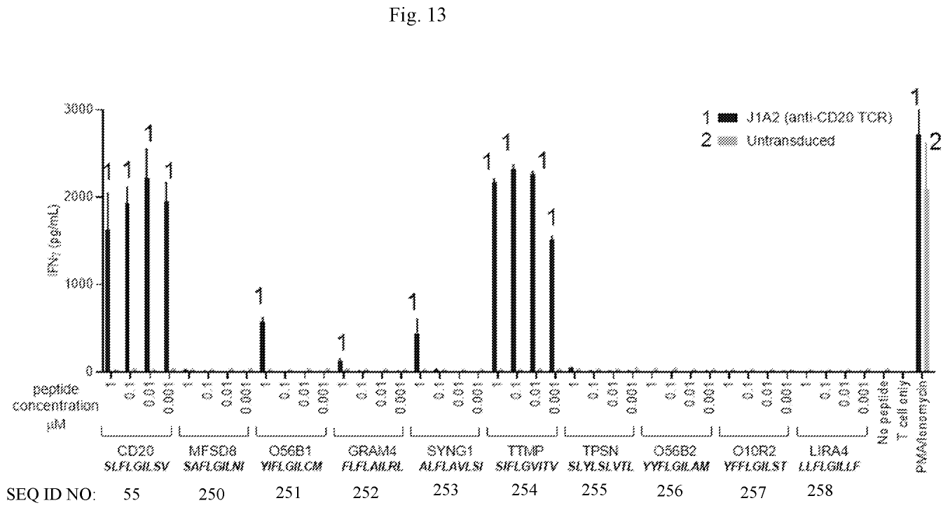

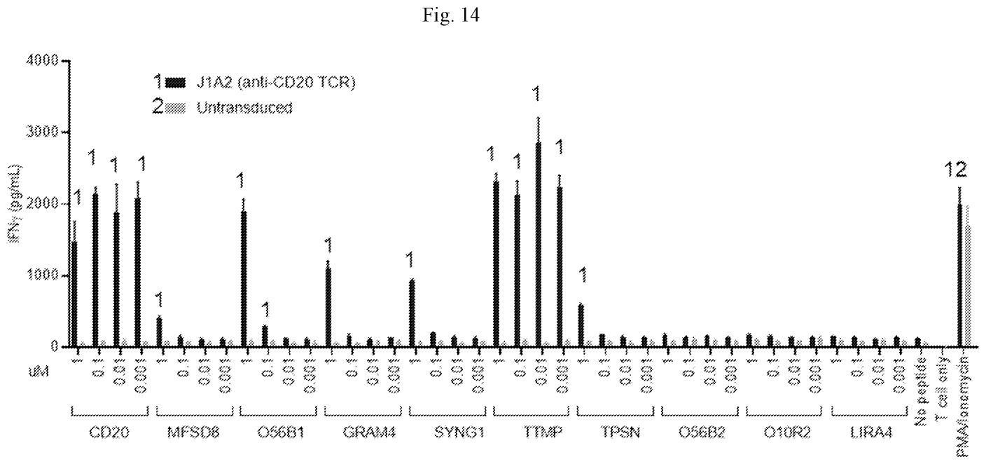

CD20TCR-transduced T cells or untransduced T cells were co-cultured with K562A2 cells loaded with titrated concentrations of peptides as indicated on the x-axis of FIG. 13. The levels of IFNγ in the overnight co-culture supernatant was measured with ELISA. Of 9 possible cross-reactive peptides shown in FIG. 12, the relative functional avidity of the CD20TCR for each irrelevant target was much lower compared to the intended epitope (CD20) with an exception of TTMP. FIG. 14 shows results for another PBMC donor.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated or purified T-cell receptor (TCR) comprising the amino acid sequences of
(a) SEQ ID NOs: 1-3,
(b) SEQ ID NOs: 4-6,
(c) SEQ ID NOs: 7-9,
(d) SEQ ID NOs: 10-12,
(e) SEQ ID NOs: 13-15,
(f) SEQ ID NOs: 16-18,
(g) SEQ ID NOs: 19-21,
(h) SEQ ID NOs: 22-24,
(i) SEQ ID NOs: 25-27,
(j) SEQ ID NOs: 28-30,
(k) SEQ ID NOs: 31-33,
(l) SEQ ID NOs: 34-36,
(m) SEQ ID NOs: 37-39,
(n) SEQ ID NOs: 40-42,
(o) SEQ ID NOs: 43-45,
(p) SEQ ID NOs: 46-48,
(q) SEQ ID NOs: 49-51,
(r) SEQ ID NOs: 52-54,
(s) all of SEQ ID NOs: 1-6,
(t) all of SEQ ID NOs: 7-12,
(u) all of SEQ ID NOs: 13-18,
(v) all of SEQ ID NOs: 19-24,
(w) all of SEQ ID NOs: 25-30,
(x) all of SEQ ID NOs: 31-36,
(y) all of SEQ ID NOs: 37-42,
(z) all of SEQ ID NOs: 43-48, or
(aa) all of SEQ ID NOs: 49-54,
wherein the TCR has antigenic specificity for a CD20 amino acid sequence presented by a human leukocyte antigen (HLA) Class I molecule.

2.-6. (canceled)

7. The TCR according to claim 1, comprising the amino acid sequence(s) of:
(i) SEQ ID NO: 56;
(ii) SEQ ID NO: 57 or 58;
(iii) SEQ ID NO: 59;
(iv) SEQ ID NO: 60 or 61;
(v) SEQ ID NO: 62;
(vi) SEQ ID NO: 63 or 64;
(vii) SEQ ID NO: 65;
(viii) SEQ ID NO: 66 or 67;
(ix) SEQ ID NO: 68;
(x) SEQ ID NO: 69 or 70;
(xi) SEQ ID NO: 71;
(xii) SEQ ID NO: 72 or 73;
(xiii) SEQ ID NO: 74;
(xiv) SEQ ID NO: 75 or 76;
(xv) SEQ ID NO: 77;
(xvi) SEQ ID NO: 78 or 79;
(xvii) SEQ ID NO: 80;
(xviii) SEQ ID NO: 81 or 82;
(xix) SEQ ID NO: 83;
(xx) SEQ ID NO: 84;
(xxi) SEQ ID NO: 85;
(xxii) SEQ ID NO: 86;
(xxiii) SEQ ID NO: 87;
(xxiv) SEQ ID NO: 88;
(xxv) SEQ ID NO: 89;
(xxvi) SEQ ID NO: 90;
(xxvii) SEQ ID NO: 91;
(xxviii) SEQ ID NO: 92;
(xxix) SEQ ID NO: 93;
(xxx) SEQ ID NO: 94;
(xxxi) SEQ ID NO: 95;
(xxxii) SEQ ID NO: 96;
(xxxiii) SEQ ID NO: 97;
(xxxiv) SEQ ID NO: 98;
(xxxv) SEQ ID NO: 99;
(xxxvi) SEQ ID NO: 100;
(xxxvii) both of SEQ ID NO: 56, and SEQ ID NO: 57 or 58;
(xxxviii) both of SEQ ID NO: 59, and SEQ ID NO: 60 or 61;
(xxxix) both of SEQ ID NO: 62, and SEQ ID NO: 63 or 64;
(xl) both of SEQ ID NO: 65, and SEQ ID NO: 66 or 67;
(xli) both of SEQ ID NO: 68, and SEQ ID NO: 69 or 70;
(xlii) both of SEQ ID NO: 71, and SEQ ID NO: 72 or 73;
(xliii) both of SEQ ID NO: 74, and SEQ ID NO: 75 or 76;
(xliv) both of SEQ ID NO: 77, and SEQ ID NO: 78 or 79;
(xlv) both of SEQ ID NO: 80, and SEQ ID NO: 81 or 82;
(xlvi) both of SEQ ID NO: 83 and SEQ ID NO: 84;
(xlvii) both of SEQ ID NO: 85 and SEQ ID NO: 86;
(xlviii) both of SEQ ID NO: 87 and SEQ ID NO: 88;
(xlix) both of SEQ ID NO: 89 and SEQ ID NO: 90;
(l) both of SEQ ID NO: 91 and SEQ ID NO: 92;
(li) both of SEQ ID NO: 93 and SEQ ID NO: 94;
(lii) both of SEQ ID NO: 95 and SEQ ID NO: 96;
(liii) both of SEQ ID NO: 97 and SEQ ID NO: 98; or
(liv) both of SEQ ID NO: 99 and SEQ ID NO: 100.

8. The TCR of claim 1, further comprising:
(a) an α chain constant region comprising the amino acid sequence of SEQ ID NO: 148, wherein:
(i) X at position 48 of SEQ ID NO: 148 is Thr or Cys;
(ii) X at position 112 of SEQ ID NO: 148 is Ser, Ala, Val, Leu, Ile, Pro, Phe, Met, or Trp;
(iii) X at position 114 of SEQ ID NO: 148 is Met, Ala, Val, Leu, Ile, Pro, Phe, or Trp; and
(iv) X at position 115 of SEQ ID NO: 148 is Gly, Ala, Val, Leu, Ile, Pro, Phe, Met, or Trp;
(b) a β chain constant region comprising the amino acid sequence of SEQ ID NO: 149, wherein X at position 57 of SEQ ID NO: 149 is Ser or Cys; or
(c) both (a) and (b).

9. The isolated or purified TCR of claim 1, comprising:
(a) an α chain comprising the amino acid sequence of SEQ ID NO: 150, wherein: (i) X at position 178 of SEQ ID NO: 150 is Thr or Cys; (ii) X at position 242 of SEQ ID NO: 150 is Ser, Ala, Val, Leu, Ile, Pro, Phe, Met, or Trp; (iii) X at position 244 of SEQ ID NO: 150 is Met, Ala, Val, Leu, Ile, Pro, Phe, or Trp; and (iv) X at position 245 of SEQ ID NO: 150 is Gly, Ala, Val, Leu, Ile, Pro, Phe, Met, or Trp;
(b) a β chain comprising the amino acid sequence of SEQ ID NO: 151 or 152, wherein X at position 188 of SEQ ID NO: 151 or 152 is Ser or Cys;
(c) an α chain comprising the amino acid sequence of SEQ ID NO: 153, wherein: (i) X at position 179 of SEQ ID NO: 153 is Thr or Cys; (ii) X at position 243 of SEQ ID NO: 153 is Ser, Ala, Val, Leu, Ile, Pro, Phe, Met, or Trp; (iii) X at position 245 of SEQ ID NO: 153 is Met, Ala, Val, Leu, Ile, Pro, Phe, or Trp; and (iv) X at position 246 of SEQ ID NO: 153 is Gly, Ala, Val, Leu, Ile, Pro, Phe, Met, or Trp;
(d) a β chain comprising the amino acid sequence of SEQ ID NO: 154 or 155, wherein X at position 188 of SEQ ID NO: 154 or 155 is Ser or Cys;
(e) an α chain comprising the amino acid sequence of SEQ ID NO: 156, wherein: (i) X at position 178 of SEQ ID NO: 156 is Thr or Cys; (ii) X at position 242 of SEQ ID NO: 156 is Ser, Ala, Val, Leu, Ile, Pro, Phe, Met, or Trp; (iii) X at position 244 of SEQ ID NO: 156 is Met, Ala, Val, Leu, Ile, Pro, Phe, or Trp; and (iv) X at position 245 of SEQ ID NO: 156 is Gly, Ala, Val, Leu, Ile, Pro, Phe, Met, or Trp;
(f) a β chain comprising the amino acid sequence of SEQ ID NO: 157 or 158, wherein X at position 188 of SEQ ID NO: 157 or 158 is Ser or Cys;
(g) an α chain comprising the amino acid sequence of SEQ ID NO: 159, wherein: (i) X at position 178 of SEQ ID NO: 159 is Thr or Cys; (ii) X at position 242 of SEQ ID NO: 159 is Ser, Ala, Val, Leu, Ile, Pro, Phe, Met, or Trp; (iii) X at position 244 of SEQ ID NO: 159 is Met, Ala, Val, Leu, Ile, Pro, Phe, or Trp; and (iv) X at position 245 of SEQ ID NO: 159 is Gly, Ala, Val, Leu, Ile, Pro, Phe, Met, or Trp;
(h) a β chain comprising the amino acid sequence of SEQ ID NO: 160 or 161, wherein X at position 191 of SEQ ID NO: 160 or 161 is Ser or Cys;
(I) an α chain comprising the amino acid sequence of SEQ ID NO: 162, wherein: (i) X at position 191 of SEQ ID NO: 162 is Thr or Cys; (ii) X at position 255 of SEQ ID NO: 162 is Ser, Ala, Val, Leu, Ile, Pro, Phe, Met, or Trp; (iii) X at position 257 of SEQ ID NO: 162 is Met, Ala, Val, Leu, Ile, Pro, Phe, or Trp; and (iv) X at position 258 of SEQ ID NO: 162 is Gly, Ala, Val, Leu, Ile, Pro, Phe, Met, or Trp;
(j) a β chain comprising the amino acid sequence of SEQ ID NO: 163 or 164, wherein X at position 188 of SEQ ID NO: 163 or 164 is Ser or Cys;
(k) an α chain comprising the amino acid sequence of SEQ ID NO: 165, wherein: (i) X at position 180 of SEQ ID NO: 165 is Thr or Cys; (ii) X at position 244 of SEQ ID NO: 165 is Ser, Ala, Val, Leu, Ile, Pro, Phe, Met, or Trp; (iii) X at position 246 of SEQ ID NO: 165 is Met, Ala, Val, Leu, Ile, Pro, Phe, or Trp; and (iv) X at position 247 of SEQ ID NO: 165 is Gly, Ala, Val, Leu, Ile, Pro, Phe, Met, or Trp;
(l) a β chain comprising the amino acid sequence of SEQ ID NO: 166 or 167, wherein X at position 190 of SEQ ID NO: 166 or 167 is Ser or Cys;
(m) an α chain comprising the amino acid sequence of SEQ ID NO: 168, wherein: (i) X at position 180 of SEQ ID NO: 168 is Thr or Cys; (ii) X at position 244 of SEQ ID NO: 168 is Ser, Ala, Val, Leu, Ile, Pro, Phe, Met, or Trp; (iii) X at position 246 of SEQ ID NO: 168 is Met, Ala, Val, Leu, Ile, Pro, Phe, or Trp; and (iv) X at position 247 of SEQ ID NO: 168 is Gly, Ala, Val, Leu, Ile, Pro, Phe, Met, or Trp;
(n) a β chain comprising the amino acid sequence of SEQ ID NO: 169 or 170, wherein X at position 188 of SEQ ID NO: 169 or 170 is Ser or Cys;
(o) an α chain comprising the amino acid sequence of SEQ ID NO: 171, wherein: (i) X at position 177 of SEQ ID NO: 171 is Thr or Cys; (ii) X at position 241 of SEQ ID NO: 171 is Ser, Ala, Val, Leu, Ile, Pro, Phe, Met, or Trp; (iii) X at position 243 of SEQ ID NO: 171 is Met, Ala, Val, Leu, Ile, Pro, Phe, or Trp; and (iv) X at position 244 of SEQ ID NO: 171 is Gly, Ala, Val, Leu, Ile, Pro, Phe, Met, or Trp;
(p) a β chain comprising the amino acid sequence of SEQ ID NO: 172 or 173, wherein X at position 188 of SEQ ID NO: 172 or 173 is Ser or Cys;
(q) an α chain comprising the amino acid sequence of SEQ ID NO: 174, wherein: (i) X at position 184 of SEQ ID NO: 174 is Thr or Cys; (ii) X at position 248 of SEQ ID NO: 174 is Ser, Ala, Val, Leu, Ile, Pro, Phe, Met, or Trp; (iii) X at position 250 of SEQ ID NO: 174 is Met, Ala, Val, Leu, Ile, Pro, Phe, or Trp; and (iv) X at position 251 of SEQ ID NO: 174 is Gly, Ala, Val, Leu, Ile, Pro, Phe, Met, or Trp;
(r) a β chain comprising the amino acid sequence of SEQ ID NO: 175 or 176, wherein X at position 189 of SEQ ID NO: 175 or 176 is Ser or Cys;
(s) both (a) and (b);
(t) both (c) and (d);
(u) both (e) and (f);
(v) both (g) and (h);
(w) both (I) and (j);
(x) both (k) and (l);
(y) both (m) and (n);
(z) both (o) and (p);
(aa) both (q) and (r);
(ab) an α chain comprising the amino acid sequence of SEQ ID NO: 177, wherein: (i) X at position 158 of SEQ ID NO: 177 is Thr or Cys; (ii) X at position 222 of SEQ ID NO: 177 is Ser, Ala, Val, Leu, Ile, Pro, Phe, Met, or Trp; (iii) X at position 224 of SEQ ID NO: 177 is Met, Ala, Val, Leu, Ile, Pro, Phe, or Trp; and (iv) X at position 225 of SEQ ID NO: 177 is Gly, Ala, Val, Leu, Ile, Pro, Phe, Met, or Trp;
(ac) a β chain comprising the amino acid sequence of SEQ ID NO: 178, wherein X at position 167 of SEQ ID NO: 178 is Ser or Cys;
(ad) an α chain comprising the amino acid sequence of SEQ ID NO: 179, wherein: (i) X at position 159 of SEQ ID NO: 179 is Thr or Cys; (ii) X at position 223 of SEQ ID NO: 179 is Ser, Ala, Val, Leu, Ile, Pro, Phe, Met, or Trp; (iii) X at position 225 of SEQ ID NO: 179 is Met, Ala, Val, Leu, Ile, Pro, Phe, or Trp; and (iv) X at position 226 of SEQ ID NO: 179 is Gly, Ala, Val, Leu, Ile, Pro, Phe, Met, or Trp;
(ae) a β chain comprising the amino acid sequence of SEQ ID NO: 180, wherein X at position 169 of SEQ ID NO: 180 is Ser or Cys;
(af) an α chain comprising the amino acid sequence of SEQ ID NO: 181, wherein: (i) X at position 158 of SEQ ID NO: 181 is Thr or Cys; (ii) X at position 222 of SEQ ID NO: 181 is Ser, Ala, Val, Leu, Ile, Pro, Phe, Met, or Trp; (iii) X at position 224 of SEQ ID NO: 181 is Met, Ala, Val, Leu, Ile, Pro, Phe, or Trp; and (iv) X at position 225 of SEQ ID NO: 181 is Gly, Ala, Val, Leu, Ile, Pro, Phe, Met, or Trp;
(ag) a β chain comprising the amino acid sequence of SEQ ID NO: 182, wherein X at position 167 of SEQ ID NO: 182 is Ser or Cys;
(ah) an α chain comprising the amino acid sequence of SEQ ID NO: 183, wherein: (i) X at position 158 of SEQ ID NO: 183 is Thr or Cys; (ii) X at position 222 of SEQ ID NO: 183 is Ser, Ala, Val, Leu, Ile, Pro, Phe, Met, or Trp; (iii) X at position 224 of SEQ ID NO: 183 is Met, Ala, Val, Leu, Ile, Pro, Phe, or Trp; and (iv) X at position 225 of SEQ ID NO: 183 is Gly, Ala, Val, Leu, Ile, Pro, Phe, Met, or Trp;
(ai) a β chain comprising the amino acid sequence of SEQ ID NO: 184, wherein X at position 169 of SEQ ID NO: 184 is Ser or Cys;
(aj) an α chain comprising the amino acid sequence of SEQ ID NO: 185, wherein: (i) X at position 163 of SEQ ID NO: 185 is Thr or Cys; (ii) X at position 227 of SEQ ID NO: 185 is Ser, Ala, Val, Leu, Ile, Pro, Phe, Met, or Trp; (iii) X at position 229 of SEQ ID NO: 185 is Met, Ala, Val, Leu, Ile, Pro, Phe, or Trp; and (iv) X at position 230 of SEQ ID NO: 185 is Gly, Ala, Val, Leu, Ile, Pro, Phe, Met, or Trp;
(ak) a β chain comprising the amino acid sequence of SEQ ID NO: 186, wherein X at position 169 of SEQ ID NO: 186 is Ser or Cys;
(al) an α chain comprising the amino acid sequence of SEQ ID NO: 187, wherein: (i) X at position 159 of SEQ ID NO: 187 is Thr or Cys; (ii) X at position 223 of SEQ ID NO: 187 is Ser, Ala, Val, Leu, Ile, Pro, Phe, Met, or Trp; (iii) X at position 225 of SEQ ID NO: 187 is Met, Ala, Val, Leu, Ile, Pro, Phe, or Trp; and (iv) X at position 226 of SEQ ID NO: 187 is Gly, Ala, Val, Leu, Ile, Pro, Phe, Met, or Trp;
(am) a β chain comprising the amino acid sequence of SEQ ID NO: 188, wherein X at position 172 of SEQ ID NO: 188 is Ser or Cys;
(an) an α chain comprising the amino acid sequence of SEQ ID NO: 189, wherein: (i) X at position 160 of SEQ ID NO: 189 is Thr or Cys; (ii) X at position 224 of SEQ ID NO: 189 is Ser, Ala, Val, Leu, Ile, Pro, Phe, Met, or Trp; (iii) X at position 226 of SEQ ID NO: 189 is Met, Ala, Val, Leu, Ile, Pro, Phe, or Trp; and (iv) X at position 227 of SEQ ID NO: 189 is Gly, Ala, Val, Leu, Ile, Pro, Phe, Met, or Trp;
(ao) a β chain comprising the amino acid sequence of SEQ ID NO: 190, wherein X at position 169 of SEQ ID NO: 190 is Ser or Cys;
(ap) an α chain comprising the amino acid sequence of SEQ ID NO: 191, wherein: (i) X at position 157 of SEQ ID NO: 191 is Thr or Cys; (ii) X at position 221 of SEQ ID NO: 191 is Ser, Ala, Val, Leu, Ile, Pro, Phe, Met, or Trp; (iii) X at position 223 of SEQ ID NO: 191 is Met, Ala, Val, Leu, Ile, Pro, Phe, or Trp; and (iv) X at position 224 of SEQ ID NO: 191 is Gly, Ala, Val, Leu, Ile, Pro, Phe, Met, or Trp;
(aq) a β chain comprising the amino acid sequence of SEQ ID NO: 192, wherein X at position 169 of SEQ ID NO: 192 is Ser or Cys;
(ar) an α chain comprising the amino acid sequence of SEQ ID NO: 193, wherein: (i) X at position 163 of SEQ ID NO: 193 is Thr or Cys; (ii) X at position 227 of SEQ ID NO: 193 is Ser, Ala, Val, Leu, Ile, Pro, Phe, Met, or Trp; (iii) X at position 229 of SEQ ID NO: 193 is Met, Ala, Val, Leu, Ile, Pro, Phe, or Trp; and (iv) X at position 230 of SEQ ID NO: 193 is Gly, Ala, Val, Leu, Ile, Pro, Phe, Met, or Trp;
(as) a β chain comprising the amino acid sequence of SEQ ID NO: 194, wherein X at position 170 of SEQ ID NO: 194 is Ser or Cys;
(at) both (ab) and (ac);
(au) both (ad) and (ae);
(av) both (af) and (ag);
(aw) both (ah) and (ai);
(ax) both (aj) and (ak);
(ay) both (al) and (am);
(az) both (an) and (ao);
(ba) both (ap) and (aq); or
(bb) both (ar) and (as).

10. An isolated or purified polypeptide comprising a functional portion of the TCR of claim 1, wherein the functional portion comprises the amino acid sequences of:
(a) all of SEQ ID NOs: 1-3,
(b) all of SEQ ID NOs: 4-6,
(c) all of SEQ ID NOs: 7-9,
(d) all of SEQ ID NOs: 10-12,
(e) all of SEQ ID NOs: 13-15,
(f) all of SEQ ID NOs: 16-18,
(g) all of SEQ ID NOs: 19-21,
(h) all of SEQ ID NOs: 22-24,
(i) all of SEQ ID NOs: 25-27,
(j) all of SEQ ID NOs: 28-30,
(k) all of SEQ ID NOs: 31-33,
(l) all of SEQ ID NOs: 34-36,
(m) all of SEQ ID NOs: 37-39,
(n) all of SEQ ID NOs: 40-42,
(o) all of SEQ ID NOs: 43-45,
(p) all of SEQ ID NOs: 46-48,
(q) all of SEQ ID NOs: 49-51,
(r) all of SEQ ID NOs: 52-54,
(s) all of SEQ ID NOs: 1-6,
(t) all of SEQ ID NOs: 7-12,
(u) all of SEQ ID NOs: 13-18,
(v) all of SEQ ID NOs: 19-24,
(w) all of SEQ ID NOs: 25-30,
(x) all of SEQ ID NOs: 31-36,
(y) all of SEQ ID NOs: 37-42,
(z) all of SEQ ID NOs: 43-48, or
(aa) all of SEQ ID NOs: 49-54.

11. The isolated or purified polypeptide according to claim 10, wherein the functional portion comprises the amino acid sequence(s) of:
(i) SEQ ID NO: 56;
(ii) SEQ ID NO: 57 or 58;
(iii) SEQ ID NO: 59;
(iv) SEQ ID NO: 60 or 61;
(v) SEQ ID NO: 62;
(vi) SEQ ID NO: 63 or 64;
(vii) SEQ ID NO: 65;
(viii) SEQ ID NO: 66 or 67;
(ix) SEQ ID NO: 68;
(x) SEQ ID NO: 69 or 70;
(xi) SEQ ID NO: 71;
(xii) SEQ ID NO: 72 or 73;
(xiii) SEQ ID NO: 74;
(xiv) SEQ ID NO: 75 or 76;
(xv) SEQ ID NO: 77;
(xvi) SEQ ID NO: 78 or 79;
(xvii) SEQ ID NO: 80;
(xviii) SEQ ID NO: 81 or 82;
(xix) SEQ ID NO: 83;
(xx) SEQ ID NO: 84;
(xxi) SEQ ID NO: 85;
(xxii) SEQ ID NO: 86;
(xxiii) SEQ ID NO: 87;
(xxiv) SEQ ID NO: 88;
(xxv) SEQ ID NO: 89;
(xxvi) SEQ ID NO: 90;
(xxvii) SEQ ID NO: 91;
(xxviii) SEQ ID NO: 92;
(xxix) SEQ ID NO: 93;
(xxx) SEQ ID NO: 94;
(xxxi) SEQ ID NO: 95;
(xxxii) SEQ ID NO: 96;
(xxxiii) SEQ ID NO: 97;
(xxxiv) SEQ ID NO: 98;
(xxxv) SEQ ID NO: 99;
(xxxvi) SEQ ID NO: 100;
(xxxvii) both of SEQ ID NO: 56, and SEQ ID NO: 57 or 58;
(xxxviii) both of SEQ ID NO: 59, and SEQ ID NO: 60 or 61;
(xxxix) both of SEQ ID NO: 62, and SEQ ID NO: 63 or 64;
(xl) both of SEQ ID NO: 65, and SEQ ID NO: 66 or 67;
(xli) both of SEQ ID NO: 68, and SEQ ID NO: 69 or 70;
(xlii) both of SEQ ID NO: 71, and SEQ ID NO: 72 or 73;
(xliii) both of SEQ ID NO: 74, and SEQ ID NO: 75 or 76;
(xliv) both of SEQ ID NO: 77, and SEQ ID NO: 78 or 79;
(xlv) both of SEQ ID NO: 80, and SEQ ID NO: 81 or 82;
(xlvi) both of SEQ ID NO: 83 and SEQ ID NO: 84;
(xlvii) both of SEQ ID NO: 85 and SEQ ID NO: 86;
(xlviii) both of SEQ ID NO: 87 and SEQ ID NO: 88;
(xlix) both of SEQ ID NO: 89 and SEQ ID NO: 90;
(l) both of SEQ ID NO: 91 and SEQ ID NO: 92;
(li) both of SEQ ID NO: 93 and SEQ ID NO: 94;
(lii) both of SEQ ID NO: 95 and SEQ ID NO: 96;
(liii) both of SEQ ID NO: 97 and SEQ ID NO: 98; or
(liv) both of SEQ ID NO: 99 and SEQ ID NO: 100.

12. The isolated or purified polypeptide of claim 10, further comprising:
(a) the amino acid sequence of SEQ ID NO: 148, wherein:
(i) X at position 48 of SEQ ID NO: 148 is Thr or Cys;
(ii) X at position 112 of SEQ ID NO: 148 is Ser, Ala, Val, Leu, Ile, Pro, Phe, Met, or Trp;
(iii) X at position 114 of SEQ ID NO: 148 is Met, Ala, Val, Leu, Ile, Pro, Phe, or Trp; and
(iv) X at position 115 of SEQ ID NO: 148 is Gly, Ala, Val, Leu, Ile, Pro, Phe, Met, or Trp;
(b) the amino acid sequence of SEQ ID NO: 149, wherein X at position 57 of SEQ ID NO: 149 is Ser or Cys; or
(c) both (a) and (b).

13. The isolated or purified polypeptide of claim 10, comprising:
(a) the amino acid sequence of SEQ ID NO: 150, wherein: (i) X at position 178 of SEQ ID NO: 150 is Thr or Cys; (ii) X at position 242 of SEQ ID NO: 150 is Ser, Ala, Val, Leu, Ile, Pro, Phe, Met, or Trp; (iii) X at position 244 of SEQ ID NO: 150 is Met, Ala, Val, Leu, Ile, Pro, Phe, or Trp; and (iv) X at position 245 of SEQ ID NO: 150 is Gly, Ala, Val, Leu, Ile, Pro, Phe, Met, or Trp;
(b) the amino acid sequence of SEQ ID NO: 151 or 152, wherein X at position 188 of SEQ ID NO: 151 or 152 is Ser or Cys;
(c) the amino acid sequence of SEQ ID NO: 153, wherein: (i) X at position 179 of SEQ ID NO: 153 is Thr or Cys; (ii) X at position 243 of SEQ ID NO: 153 is Ser, Ala, Val, Leu, Ile, Pro, Phe, Met, or Trp; (iii) X at position 245 of SEQ ID NO: 153 is Met, Ala, Val, Leu, Ile, Pro, Phe, or Trp; and (iv) X at position 246 of SEQ ID NO: 153 is Gly, Ala, Val, Leu, Ile, Pro, Phe, Met, or Trp;
(d) the amino acid sequence of SEQ ID NO: 154 or 155, wherein X at position 188 of SEQ ID NO: 154 or 155 is Ser or Cys;
(e) the amino acid sequence of SEQ ID NO: 156, wherein: (i) X at position 178 of SEQ ID NO: 156 is Thr or Cys; (ii) X at position 242 of SEQ ID NO: 156 is Ser, Ala, Val, Leu, Ile, Pro, Phe, Met, or Trp; (iii) X at position 244 of SEQ ID NO: 156 is Met, Ala, Val, Leu, Ile, Pro, Phe, or Trp; and (iv) X at position 245 of SEQ ID NO: 156 is Gly, Ala, Val, Leu, Ile, Pro, Phe, Met, or Trp;
(f) the amino acid sequence of SEQ ID NO: 157 or 158, wherein X at position 188 of SEQ ID NO: 157 or 158 is Ser or Cys;
(g) the amino acid sequence of SEQ ID NO: 159, wherein: (i) X at position 178 of SEQ ID NO: 159 is Thr or Cys; (ii) X at position 242 of SEQ ID NO: 159 is Ser, Ala, Val, Leu, Ile, Pro, Phe, Met, or Trp; (iii) X at position 244 of SEQ ID NO: 159 is Met, Ala, Val, Leu, Ile, Pro, Phe, or Trp; and (iv) X at position 245 of SEQ ID NO: 159 is Gly, Ala, Val, Leu, Ile, Pro, Phe, Met, or Trp;
(h) the amino acid sequence of SEQ ID NO: 160 or 161, wherein X at position 191 of SEQ ID NO: 160 or 161 is Ser or Cys;
(I) the amino acid sequence of SEQ ID NO: 162, wherein: (i) X at position 191 of SEQ ID NO: 162 is Thr or Cys; (ii) X at position 255 of SEQ ID NO: 162 is Ser, Ala, Val, Leu, Ile, Pro, Phe, Met, or Trp; (iii) X at position 257 of SEQ ID NO: 162 is Met, Ala, Val, Leu, Ile, Pro, Phe, or Trp; and (iv) X at position 258 of SEQ ID NO: 162 is Gly, Ala, Val, Leu, Ile, Pro, Phe, Met, or Trp;
(j) the amino acid sequence of SEQ ID NO: 163 or 164, wherein X at position 188 of SEQ ID NO: 163 or 164 is Ser or Cys;
(k) the amino acid sequence of SEQ ID NO: 165, wherein: (i) X at position 180 of SEQ ID NO: 165 is Thr or Cys; (ii) X at position 244 of SEQ ID NO: 165 is Ser, Ala, Val, Leu, Ile, Pro, Phe, Met, or Trp; (iii) X at position 246 of SEQ ID NO: 165 is Met, Ala, Val, Leu, Ile, Pro, Phe, or Trp; and (iv) X at position 247 of SEQ ID NO: 165 is Gly, Ala, Val, Leu, Ile, Pro, Phe, Met, or Trp;
(l) the amino acid sequence of SEQ ID NO: 166 or 167, wherein X at position 190 of SEQ ID NO: 166 or 167 is Ser or Cys;
(m) the amino acid sequence of SEQ ID NO: 168, wherein: (i) X at position 180 of SEQ ID NO: 168 is Thr or Cys; (ii) X at position 244 of SEQ ID NO: 168 is Ser, Ala, Val, Leu, Ile, Pro, Phe, Met, or Trp; (iii) X at position 246 of SEQ ID NO: 168 is Met, Ala, Val, Leu, Ile, Pro, Phe, or Trp; and (iv) X at position 247 of SEQ ID NO: 168 is Gly, Ala, Val, Leu, Ile, Pro, Phe, Met, or Trp;
(n) the amino acid sequence of SEQ ID NO: 169 or 170, wherein X at position 188 of SEQ ID NO: 169 or 170 is Ser or Cys;
(o) the amino acid sequence of SEQ ID NO: 171, wherein: (i) X at position 177 of SEQ ID NO: 171 is Thr or Cys; (ii) X at position 241 of SEQ ID NO: 171 is Ser, Ala, Val, Leu, Ile, Pro, Phe, Met, or Trp; (iii) X at position 243 of SEQ ID NO: 171 is Met, Ala, Val, Leu, Ile, Pro, Phe, or Trp; and (iv) X at position 244 of SEQ ID NO: 171 is Gly, Ala, Val, Leu, Ile, Pro, Phe, Met, or Trp;
(p) the amino acid sequence of SEQ ID NO: 172 or 173, wherein X at position 188 of SEQ ID NO: 172 or 173 is Ser or Cys;
(q) the amino acid sequence of SEQ ID NO: 174, wherein: (i) X at position 184 of SEQ ID NO: 174 is Thr or Cys; (ii) X at position 248 of SEQ ID NO: 174 is Ser, Ala, Val, Leu, Ile, Pro, Phe, Met, or Trp; (iii) X at position 250 of SEQ ID NO: 174 is Met, Ala, Val, Leu, Ile, Pro, Phe, or Trp; and (iv) X at position 251 of SEQ ID NO: 174 is Gly, Ala, Val, Leu, Ile, Pro, Phe, Met, or Trp;
(r) the amino acid sequence of SEQ ID NO: 175 or 176, wherein X at position 189 of SEQ ID NO: 175 or 176 is Ser or Cys;
(s) both (a) and (b);
(t) both (c) and (d);
(u) both (e) and (0;
(v) both (g) and (h);
(w) both (I) and (j);
(x) both (k) and (1);
(y) both (m) and (n);
(z) both (o) and (p);
(aa) both (q) and (r);
(ab) the amino acid sequence of SEQ ID NO: 177, wherein: (i) X at position 158 of SEQ ID NO: 177 is Thr or Cys; (ii) X at position 222 of SEQ ID NO: 177 is Ser, Ala, Val, Leu, Ile, Pro, Phe, Met, or Trp; (iii) X at position 224 of SEQ ID NO: 177 is Met, Ala, Val, Leu, Ile, Pro, Phe, or Trp; and (iv) X at position 225 of SEQ ID NO: 177 is Gly, Ala, Val, Leu, Ile, Pro, Phe, Met, or Trp;
(ac) the amino acid sequence of SEQ ID NO: 178, wherein X at position 167 of SEQ ID NO: 178 is Ser or Cys;
(ad) the amino acid sequence of SEQ ID NO: 179, wherein: (i) X at position 159 of SEQ ID NO: 179 is Thr or Cys; (ii) X at position 223 of SEQ ID NO: 179 is Ser, Ala, Val, Leu, Ile, Pro, Phe, Met, or Trp; (iii) X at position 225 of SEQ ID NO: 179 is Met, Ala, Val, Leu, Ile, Pro, Phe, or Trp; and (iv) X at position 226 of SEQ ID NO: 179 is Gly, Ala, Val, Leu, Ile, Pro, Phe, Met, or Trp;
(ae) the amino acid sequence of SEQ ID NO: 180, wherein X at position 169 of SEQ ID NO: 180 is Ser or Cys;
(af) the amino acid sequence of SEQ ID NO: 181, wherein: (i) X at position 158 of SEQ ID NO: 181 is Thr or Cys; (ii) X at position 222 of SEQ ID NO: 181 is Ser, Ala, Val, Leu, Ile, Pro, Phe, Met, or Trp; (iii) X at position 224 of SEQ ID NO: 181 is Met, Ala, Val, Leu, Ile, Pro, Phe, or Trp; and (iv) X at position 225 of SEQ ID NO: 181 is Gly, Ala, Val, Leu, Ile, Pro, Phe, Met, or Trp;
(ag) the amino acid sequence of SEQ ID NO: 182, wherein X at position 167 of SEQ ID NO: 182 is Ser or Cys;
(ah) the amino acid sequence of SEQ ID NO: 183, wherein: (i) X at position 158 of SEQ ID NO: 183 is Thr or Cys; (ii) X at position 222 of SEQ ID NO: 183 is Ser, Ala, Val, Leu, Ile, Pro, Phe, Met, or Trp; (iii) X at position 224 of SEQ ID NO: 183 is Met, Ala, Val, Leu, Ile, Pro, Phe, or Trp; and (iv) X at position 225 of SEQ ID NO: 183 is Gly, Ala, Val, Leu, Ile, Pro, Phe, Met, or Trp;
(ai) the amino acid sequence of SEQ ID NO: 184, wherein X at position 169 of SEQ ID NO: 184 is Ser or Cys;
(aj) the amino acid sequence of SEQ ID NO: 185, wherein: (i) X at position 163 of SEQ ID NO: 185 is Thr or Cys; (ii) X at position 227 of SEQ ID NO: 185 is Ser, Ala, Val, Leu, Ile, Pro, Phe, Met, or Trp; (iii) X at position 229 of SEQ ID NO: 185 is Met, Ala, Val, Leu, Ile, Pro, Phe, or Trp; and (iv) X at position 230 of SEQ ID NO: 185 is Gly, Ala, Val, Leu, Ile, Pro, Phe, Met, or Trp;
(ak) the amino acid sequence of SEQ ID NO: 186, wherein X at position 169 of SEQ ID NO: 186 is Ser or Cys;
(al) the amino acid sequence of SEQ ID NO: 187, wherein: (i) X at position 159 of SEQ ID NO: 187 is Thr or Cys; (ii) X at position 223 of SEQ ID NO: 187 is Ser, Ala, Val, Leu, Ile, Pro, Phe, Met, or Trp; (iii) X at position 225 of SEQ ID NO: 187 is Met, Ala, Val, Leu, Ile, Pro, Phe, or Trp; and (iv) X at position 226 of SEQ ID NO: 187 is Gly, Ala, Val, Leu, Ile, Pro, Phe, Met, or Trp;
(am) the amino acid sequence of SEQ ID NO: 188, wherein X at position 172 of SEQ ID NO: 188 is Ser or Cys;
(an) the amino acid sequence of SEQ ID NO: 189, wherein: (i) X at position 160 of SEQ ID NO: 189 is Thr or Cys; (ii) X at position 224 of SEQ ID NO: 189 is Ser, Ala, Val, Leu, Ile, Pro, Phe, Met, or Trp; (iii) X at position 226 of SEQ ID NO: 189 is Met, Ala, Val, Leu, Ile, Pro, Phe, or Trp; and (iv) X at position 227 of SEQ ID NO: 189 is Gly, Ala, Val, Leu, Ile, Pro, Phe, Met, or Trp;
(ao) the amino acid sequence of SEQ ID NO: 190, wherein X at position 169 of SEQ ID NO: 190 is Ser or Cys;
(ap) the amino acid sequence of SEQ ID NO: 191, wherein: (i) X at position 157 of SEQ ID NO: 191 is Thr or Cys; (ii) X at position 221 of SEQ ID NO: 191 is Ser, Ala, Val, Leu, Ile, Pro, Phe, Met, or Trp; (iii) X at position 223 of SEQ ID NO: 191 is Met, Ala, Val, Leu, Ile, Pro, Phe, or Trp; and (iv) X at position 224 of SEQ ID NO: 191 is Gly, Ala, Val, Leu, Ile, Pro, Phe, Met, or Trp;
(aq) the amino acid sequence of SEQ ID NO: 192, wherein X at position 169 of SEQ ID NO: 192 is Ser or Cys;
(ar) the amino acid sequence of SEQ ID NO: 193, wherein: (i) X at position 163 of SEQ ID NO: 193 is Thr or Cys; (ii) X at position 227 of SEQ ID NO: 193 is Ser, Ala, Val, Leu, Ile, Pro, Phe, Met, or Trp; (iii) X at position 229 of SEQ ID NO: 193 is Met, Ala, Val, Leu, Ile, Pro, Phe, or Trp; and (iv) X at position 230 of SEQ ID NO: 193 is Gly, Ala, Val, Leu, Ile, Pro, Phe, Met, or Trp;
(as) the amino acid sequence of SEQ ID NO: 194, wherein X at position 170 of SEQ ID NO: 194 is Ser or Cys;
(at) both (ab) and (ac);
(au) both (ad) and (ae);
(av) both (af) and (ag);
(aw) both (ah) and (ai);
(ax) both (aj) and (ak);
(ay) both (al) and (am);
(az) both (an) and (ao);
(ba) both (ap) and (aq); or
(bb) both (ar) and (as).

14. An isolated or purified protein comprising at least one of the polypeptides of claim 10.

15. The isolated or purified protein according to claim 14, comprising:
(a) a first polypeptide chain comprising the amino acid sequences of SEQ ID NOs: 1-3 and a second polypeptide chain comprising the amino acid sequences of SEQ ID NOs: 4-6;
(b) a first polypeptide chain comprising the amino acid sequences of SEQ ID NOs: 7-9 and a second polypeptide chain comprising the amino acid sequences of SEQ ID NOs: 10-12;
(c) a first polypeptide chain comprising the amino acid sequences of SEQ ID NOs: 13-15 and a second polypeptide chain comprising the amino acid sequences of SEQ ID NOs: 16-18;
(d) a first polypeptide chain comprising the amino acid sequences of SEQ ID NOs: 19-21 and a second polypeptide chain comprising the amino acid sequences of SEQ ID NOs: 22-24;
(e) a first polypeptide chain comprising the amino acid sequences of SEQ ID NOs: 25-27 and a second polypeptide chain comprising the amino acid sequences of SEQ ID NOs: 28-30;
(f) a first polypeptide chain comprising the amino acid sequences of SEQ ID NOs: 31-33 and a second polypeptide chain comprising the amino acid sequences of SEQ ID NOs: 34-36;
(g) a first polypeptide chain comprising the amino acid sequences of SEQ ID NOs: 37-39 and a second polypeptide chain comprising the amino acid sequences of SEQ ID NOs: 40-42;
(h) a first polypeptide chain comprising the amino acid sequences of SEQ ID NOs: 43-45 and a second polypeptide chain comprising the amino acid sequences of SEQ ID NOs: 46-48; or
(i) a first polypeptide chain comprising the amino acid sequences of SEQ ID NOs: 49-51 and a second polypeptide chain comprising the amino acid sequences of SEQ ID NOs: 52-54.

16. The isolated or purified protein according to claim 14, comprising:
(i) a first polypeptide chain comprising the amino acid sequence of SEQ ID NO: 56 and a second polypeptide chain comprising the amino acid sequence of SEQ ID NO: 57 or 58;
(ii) a first polypeptide chain comprising the amino acid sequence of SEQ ID NO: 59 and a second polypeptide chain comprising the amino acid sequence of SEQ ID NO: 60 or 61;
(iii) a first polypeptide chain comprising the amino acid sequence of SEQ ID NO: 62 and a second polypeptide chain comprising the amino acid sequence of SEQ ID NO: 63 or 64;
(iv) a first polypeptide chain comprising the amino acid sequence of SEQ ID NO: 65 and a second polypeptide chain comprising the amino acid sequence of SEQ ID NO: 66 or 67;
(v) a first polypeptide chain comprising the amino acid sequence of SEQ ID NO: 68 and a second polypeptide chain comprising the amino acid sequence of SEQ ID NO: 69 or 70;
(vi) a first polypeptide chain comprising the amino acid sequence of SEQ ID NO: 71 and a second polypeptide chain comprising the amino acid sequence of SEQ ID NO: 72 or 73;
(vii) a first polypeptide chain comprising the amino acid sequence of SEQ ID NO: 74 and a second polypeptide chain comprising the amino acid sequence of SEQ ID NO: 75 or 76;
(viii) a first polypeptide chain comprising the amino acid sequence of SEQ ID NO: 77 and a second polypeptide chain comprising the amino acid sequence of SEQ ID NO: 78 or 79;
(ix) a first polypeptide chain comprising the amino acid sequence of SEQ ID NO: 80 and a second polypeptide chain comprising the amino acid sequence of SEQ ID NO: 81 or 82;
(x) a first polypeptide chain comprising the amino acid sequence of SEQ ID NO: 83 and a second polypeptide chain comprising the amino acid sequence of SEQ ID NO: 84;
(xi) a first polypeptide chain comprising the amino acid sequence of SEQ ID NO: 85 and a second polypeptide chain comprising the amino acid sequence of SEQ ID NO: 86;
(xii) a first polypeptide chain comprising the amino acid sequence of SEQ ID NO: 87 and a second polypeptide chain comprising the amino acid sequence of SEQ ID NO: 88;
(xiii) a first polypeptide chain comprising the amino acid sequence of SEQ ID NO: 89 and a second polypeptide chain comprising the amino acid sequence of SEQ ID NO: 90;
(xiv) a first polypeptide chain comprising the amino acid sequence of SEQ ID NO: 91 and a second polypeptide chain comprising the amino acid sequence of SEQ ID NO: 92;
(xv) a first polypeptide chain comprising the amino acid sequence of SEQ ID NO: 93 and a second polypeptide chain comprising the amino acid sequence of SEQ ID NO: 94;
(xvi) a first polypeptide chain comprising the amino acid sequence of SEQ ID NO: 95 and a second polypeptide chain comprising the amino acid sequence of SEQ ID NO: 96;
(xvii) a first polypeptide chain comprising the amino acid sequence of SEQ ID NO: 97 and a second polypeptide chain comprising the amino acid sequence of SEQ ID NO: 98; or
(xviii) a first polypeptide chain comprising the amino acid sequence of SEQ ID NO: 99 and a second polypeptide chain comprising the amino acid sequence of SEQ ID NO: 100.

17. The isolated or purified protein of claim 14, further comprising:
(a) a first polypeptide chain comprising the amino acid sequence of SEQ ID NO: 148, wherein:
(i) X at position 48 of SEQ ID NO: 148 is Thr or Cys;
(ii) X at position 112 of SEQ ID NO: 148 is Ser, Ala, Val, Leu, Ile, Pro, Phe, Met, or Trp;
(iii) X at position 114 of SEQ ID NO: 148 is Met, Ala, Val, Leu, Ile, Pro, Phe, or Trp; and
(iv) X at position 115 of SEQ ID NO: 148 is Gly, Ala, Val, Leu, Ile, Pro, Phe, Met, or Trp;
(b) a second polypeptide chain constant region comprising the amino acid sequence of SEQ ID NO: 149, wherein X at position 57 of SEQ ID NO: 149 is Ser or Cys; or
(c) both (a) and (b).

18. The isolated or purified protein of claim 14, comprising:
(a) a first polypeptide chain comprising the amino acid sequence of SEQ ID NO: 150, wherein: (i) X at position 178 of SEQ ID NO: 150 is Thr or Cys; (ii) X at position 242 of SEQ ID NO: 150 is Ser, Ala, Val, Leu, Ile, Pro, Phe, Met, or Trp; (iii) X at position 244 of SEQ ID NO: 150 is Met, Ala, Val, Leu, Ile, Pro, Phe, or Trp; and (iv) X at position 245 of SEQ ID NO: 150 is Gly, Ala, Val, Leu, Ile, Pro, Phe, Met, or Trp;
(b) a second polypeptide chain comprising the amino acid sequence of SEQ ID NO: 151 or 152, wherein X at position 188 of SEQ ID NO: 151 or 152 is Ser or Cys;
(c) a first polypeptide chain comprising the amino acid sequence of SEQ ID NO: 153, wherein: (i) X at position 179 of SEQ ID NO: 153 is Thr or Cys; (ii) X at position 243 of SEQ ID NO: 153 is Ser, Ala, Val, Leu, Ile, Pro, Phe, Met, or Trp; (iii) X at position 245 of SEQ ID NO: 153 is Met, Ala, Val, Leu, Ile, Pro, Phe, or Trp; and (iv) X at position 246 of SEQ ID NO: 153 is Gly, Ala, Val, Leu, Ile, Pro, Phe, Met, or Trp;
(d) a second polypeptide chain comprising the amino acid sequence of SEQ ID NO: 154 or 155, wherein X at position 188 of SEQ ID NO: 154 or 155 is Ser or Cys;
(e) a first polypeptide chain comprising the amino acid sequence of SEQ ID NO: 156, wherein: (i) X at position 178 of SEQ ID NO: 156 is Thr or Cys; (ii) X at position 242 of SEQ ID NO: 156 is Ser, Ala, Val, Leu, Ile, Pro, Phe, Met, or Trp; (iii) X at position 244 of SEQ ID NO: 156 is Met, Ala, Val, Leu, Ile, Pro, Phe, or Trp; and (iv) X at position 245 of SEQ ID NO: 156 is Gly, Ala, Val, Leu, Ile, Pro, Phe, Met, or Trp;
(f) a second polypeptide chain comprising the amino acid sequence of SEQ ID NO: 157 or 158, wherein X at position 188 of SEQ ID NO: 157 or 158 is Ser or Cys;
(g) a first polypeptide chain comprising the amino acid sequence of SEQ ID NO: 159, wherein: (i) X at position 178 of SEQ ID NO: 159 is Thr or Cys; (ii) X at position 242 of SEQ ID NO: 159 is Ser, Ala, Val, Leu, Ile, Pro, Phe, Met, or Trp; (iii) X at position 244 of SEQ ID NO: 159 is Met, Ala, Val, Leu, Ile, Pro, Phe, or Trp; and (iv) X at position 245 of SEQ ID NO: 159 is Gly, Ala, Val, Leu, Ile, Pro, Phe, Met, or Trp;
(h) a second polypeptide chain comprising the amino acid sequence of SEQ ID NO: 160 or 161, wherein X at position 191 of SEQ ID NO: 160 or 161 is Ser or Cys;
(i) a first polypeptide chain comprising the amino acid sequence of SEQ ID NO: 162, wherein: (i) X at position 191 of SEQ ID NO: 162 is Thr or Cys; (ii) X at position 255 of SEQ ID NO: 162 is Ser, Ala, Val, Leu, Ile, Pro, Phe, Met, or Trp; (iii) X at position 257 of SEQ ID NO: 162 is Met, Ala, Val, Leu, Ile, Pro, Phe, or Trp; and (iv) X at position 258 of SEQ ID NO: 162 is Gly, Ala, Val, Leu, Ile, Pro, Phe, Met, or Trp;
(j) a second polypeptide chain comprising the amino acid sequence of SEQ ID NO: 163 or 164, wherein X at position 188 of SEQ ID NO: 163 or 164 is Ser or Cys;
(k) a first polypeptide chain comprising the amino acid sequence of SEQ ID NO: 165, wherein: (i) X at position 180 of SEQ ID NO: 165 is Thr or Cys; (ii) X at position 244 of SEQ ID NO: 165 is Ser, Ala, Val, Leu, Ile, Pro, Phe, Met, or Trp; (iii) X at position 246 of SEQ ID NO: 165 is Met, Ala, Val, Leu, Ile, Pro, Phe, or Trp; and (iv) X at position 247 of SEQ ID NO: 165 is Gly, Ala, Val, Leu, Ile, Pro, Phe, Met, or Trp;
(l) a second polypeptide chain comprising the amino acid sequence of SEQ ID NO: 166 or 167, wherein X at position 190 of SEQ ID NO: 166 or 167 is Ser or Cys;
(m) a first polypeptide chain comprising the amino acid sequence of SEQ ID NO: 168, wherein: (i) X at position 180 of SEQ ID NO: 168 is Thr or Cys; (ii) X at position 244 of SEQ ID NO: 168 is Ser, Ala, Val, Leu, Ile, Pro, Phe, Met, or Trp; (iii) X at position 246 of SEQ ID NO: 168 is Met, Ala, Val, Leu, Ile, Pro, Phe, or Trp; and (iv) X at position 247 of SEQ ID NO: 168 is Gly, Ala, Val, Leu, Ile, Pro, Phe, Met, or Trp;
(n) a second polypeptide chain comprising the amino acid sequence of SEQ ID NO: 169 or 170, wherein X at position 188 of SEQ ID NO: 169 or 170 is Ser or Cys;
(o) a first polypeptide chain comprising the amino acid sequence of SEQ ID NO: 171, wherein: (i) X at position 177 of SEQ ID NO: 171 is Thr or Cys; (ii) X at position 241 of SEQ ID NO: 171 is Ser, Ala, Val, Leu, Ile, Pro, Phe, Met, or Trp; (iii) X at position 243 of SEQ ID NO: 171 is Met, Ala, Val, Leu, Ile, Pro, Phe, or Trp; and (iv) X at position 244 of SEQ ID NO: 171 is Gly, Ala, Val, Leu, Ile, Pro, Phe, Met, or Trp;
(p) a second polypeptide chain comprising the amino acid sequence of SEQ ID NO: 172 or 173, wherein X at position 188 of SEQ ID NO: 172 or 173 is Ser or Cys;
(q) a first polypeptide chain comprising the amino acid sequence of SEQ ID NO: 174, wherein: (i) X at position 184 of SEQ ID NO: 174 is Thr or Cys; (ii) X at position 248 of SEQ ID NO: 174 is Ser, Ala, Val, Leu, Ile, Pro, Phe, Met, or Trp; (iii) X at position 250 of SEQ ID NO: 174 is Met, Ala, Val, Leu, Ile, Pro, Phe, or Trp; and (iv) X at position 251 of SEQ ID NO: 174 is Gly, Ala, Val, Leu, Ile, Pro, Phe, Met, or Trp;
(r) a second polypeptide chain comprising the amino acid sequence of SEQ ID NO: 175 or 176, wherein X at position 189 of SEQ ID NO: 175 or 176 is Ser or Cys;
(s) both (a) and (b);
(t) both (c) and (d);
(u) both (e) and (f);
(v) both (g) and (h);
(w) both (I) and (j);
(x) both (k) and (l);
(y) both (m) and (n);
(z) both (o) and (p);
(aa) both (q) and (r);
(ab) a first polypeptide chain comprising the amino acid sequence of SEQ ID NO: 177, wherein: (i) X at position 158 of SEQ ID NO: 177 is Thr or Cys; (ii) X at position 222 of SEQ ID NO: 177 is Ser, Ala, Val, Leu, Ile, Pro, Phe, Met, or Trp; (iii) X at position 224 of SEQ ID NO: 177 is Met, Ala, Val, Leu, Ile, Pro, Phe, or Trp; and (iv) X at position 225 of SEQ ID NO: 177 is Gly, Ala, Val, Leu, Ile, Pro, Phe, Met, or Trp;
(ac) a second polypeptide chain comprising the amino acid sequence of SEQ ID NO: 178, wherein X at position 167 of SEQ ID NO: 178 is Ser or Cys;
(ad) a first polypeptide chain comprising the amino acid sequence of SEQ ID NO: 179, wherein: (i) X at position 159 of SEQ ID NO: 179 is Thr or Cys; (ii) X at position 223 of SEQ ID NO: 179 is Ser, Ala, Val, Leu, Ile, Pro, Phe, Met, or Trp; (iii) X at position 225 of SEQ ID NO: 179 is Met, Ala, Val, Leu, Ile, Pro, Phe, or Trp; and (iv) X at position 226 of SEQ ID NO: 179 is Gly, Ala, Val, Leu, Ile, Pro, Phe, Met, or Trp;
(ae) a second polypeptide chain comprising the amino acid sequence of SEQ ID NO: 180, wherein X at position 169 of SEQ ID NO: 180 is Ser or Cys;
(af) a first polypeptide chain comprising the amino acid sequence of SEQ ID NO: 181, wherein: (i) X at position 158 of SEQ ID NO: 181 is Thr or Cys; (ii) X at position 222 of SEQ ID NO: 181 is Ser, Ala, Val, Leu, Ile, Pro, Phe, Met, or Trp; (iii) X at position 224 of SEQ ID NO: 181 is Met, Ala, Val, Leu, Ile, Pro, Phe, or Trp; and (iv) X at position 225 of SEQ ID NO: 181 is Gly, Ala, Val, Leu, Ile, Pro, Phe, Met, or Trp;
(ag) a second polypeptide chain comprising the amino acid sequence of SEQ ID NO: 182, wherein X at position 167 of SEQ ID NO: 182 is Ser or Cys;
(ah) a first polypeptide chain comprising the amino acid sequence of SEQ ID NO: 183, wherein: (i) X at position 158 of SEQ ID NO: 183 is Thr or Cys; (ii) X at position 222 of SEQ ID NO: 183 is Ser, Ala, Val, Leu, Ile, Pro, Phe, Met, or Trp; (iii) X at position 224 of SEQ ID NO: 183 is Met, Ala, Val, Leu, Ile, Pro, Phe, or Trp; and (iv) X at position 225 of SEQ ID NO: 183 is Gly, Ala, Val, Leu, Ile, Pro, Phe, Met, or Trp;
(ai) a second polypeptide chain comprising the amino acid sequence of SEQ ID NO: 184, wherein X at position 169 of SEQ ID NO: 184 is Ser or Cys;
(aj) a first polypeptide chain comprising the amino acid sequence of SEQ ID NO: 185, wherein: (i) X at position 163 of SEQ ID NO: 185 is Thr or Cys; (ii) X at position 227 of SEQ ID NO: 185 is Ser, Ala, Val, Leu, Ile, Pro, Phe, Met, or Trp; (iii) X at position 229 of SEQ ID NO: 185 is Met, Ala, Val, Leu, Ile, Pro, Phe, or Trp; and (iv) X at position 230 of SEQ ID NO: 185 is Gly, Ala, Val, Leu, Ile, Pro, Phe, Met, or Trp;
(ak) a second polypeptide chain comprising the amino acid sequence of SEQ ID NO: 186, wherein X at position 169 of SEQ ID NO: 186 is Ser or Cys;
(al) a first polypeptide chain comprising the amino acid sequence of SEQ ID NO: 187, wherein: (i) X at position 159 of SEQ ID NO: 187 is Thr or Cys; (ii) X at position 223 of SEQ ID NO: 187 is Ser, Ala, Val, Leu, Ile, Pro, Phe, Met, or Trp; (iii) X at position 225 of SEQ ID NO: 187 is Met, Ala, Val, Leu, Ile, Pro, Phe, or Trp; and (iv) X at position 226 of SEQ ID NO: 187 is Gly, Ala, Val, Leu, Ile, Pro, Phe, Met, or Trp;
(am) a second polypeptide chain comprising the amino acid sequence of SEQ ID NO: 188, wherein X at position 172 of SEQ ID NO: 188 is Ser or Cys;
(an) a first polypeptide chain comprising the amino acid sequence of SEQ ID NO: 189, wherein: (i) X at position 160 of SEQ ID NO: 189 is Thr or Cys; (ii) X at position 224 of SEQ ID NO: 189 is Ser, Ala, Val, Leu, Ile, Pro, Phe, Met, or Trp; (iii) X at position 226 of SEQ ID NO: 189 is Met, Ala, Val, Leu, Ile, Pro, Phe, or Trp; and (iv) X at position 227 of SEQ ID NO: 189 is Gly, Ala, Val, Leu, Ile, Pro, Phe, Met, or Trp;
(ao) a second polypeptide chain comprising the amino acid sequence of SEQ ID NO: 190, wherein X at position 169 of SEQ ID NO: 190 is Ser or Cys;
(ap) a first polypeptide chain comprising the amino acid sequence of SEQ ID NO: 191, wherein: (i) X at position 157 of SEQ ID NO: 191 is Thr or Cys; (ii) X at position 221 of SEQ ID NO: 191 is Ser, Ala, Val, Leu, Ile, Pro, Phe, Met, or Trp; (iii) X at position 223 of SEQ ID NO: 191 is Met, Ala, Val, Leu, Ile, Pro, Phe, or Trp; and (iv) X at position 224 of SEQ ID NO: 191 is Gly, Ala, Val, Leu, Ile, Pro, Phe, Met, or Trp;
(aq) a second polypeptide chain comprising the amino acid sequence of SEQ ID NO: 192, wherein X at position 169 of SEQ ID NO: 192 is Ser or Cys;
(ar) a first polypeptide chain comprising the amino acid sequence of SEQ ID NO: 193, wherein: (i) X at position 163 of SEQ ID NO: 193 is Thr or Cys; (ii) X at position 227 of SEQ ID NO: 193 is Ser, Ala, Val, Leu, Ile, Pro, Phe, Met, or Trp; (iii) X at position 229 of SEQ ID NO: 193 is Met, Ala, Val, Leu, Ile, Pro, Phe, or Trp; and (iv) X at position 230 of SEQ ID NO: 193 is Gly, Ala, Val, Leu, Ile, Pro, Phe, Met, or Trp;
(as) a second polypeptide chain comprising the amino acid sequence of SEQ ID NO: 194, wherein X at position 170 of SEQ ID NO: 194 is Ser or Cys;
(at) both (ab) and (ac);
(au) both (ad) and (ae);
(av) both (af) and (ag);
(aw) both (ah) and (ai);
(ax) both (aj) and (ak);
(ay) both (al) and (am);
(az) both (an) and (ao);
(ba) both (ap) and (aq); or
(bb) both (ar) and (as).

19. An isolated or purified nucleic acid comprising a nucleotide sequence encoding the TCR according to claim 1.

20-22. (canceled)

23. A recombinant expression vector comprising the nucleic acid according to claim 19.

24.-27. (canceled)

28. An isolated or purified host cell comprising the nucleic acid according to claim 19.

29. (canceled)

30. The host cell according to claim 28, wherein the cell is selected from the group consisting of a T cell, a natural killer T (NKT) cell, an invariant natural killer T (iNKT) cell, and a natural killer (NK) cell.

31. An isolated or purified population of cells comprising the host cell according to claim 28.

32. (canceled)

33. A pharmaceutical composition comprising (a) the population of cells according to claim 31 and (b) a pharmaceutically acceptable carrier.

34.-35. (canceled)

36. A method of treating or preventing cancer in a mammal, the method comprising administering to a subject in need thereof the population of cells according to claim 31 in an amount effective to treat or prevent cancer in the mammal, wherein the cancer expresses a CD20 amino acid sequence.

37.-38. (canceled)


SEQUENCE LISTING (258 sequences)

SEQ ID NO: 1 - protein, 6 aa, Mus musculus
DRNVDY

SEQ ID NO: 2 - protein, 6 aa, Mus musculus
IFSNGE

SEQ ID NO: 3 - protein, 10 aa, Mus musculus
AASTNTGKLT

SEQ ID NO: 4 - protein, 6 aa, Mus musculus
NSQYPW

SEQ ID NO: 5 - protein, 6 aa, Mus musculus
LRSPGD

SEQ ID NO: 6 - protein, 9 aa, Mus musculus
TCSTGDEQY

SEQ ID NO: 7 - protein, 6 aa, Mus musculus
DSASQS

SEQ ID NO: 8 - protein, 6 aa, Mus musculus
IFSNGN

SEQ ID NO: 9 - protein, 11 aa, Mus musculus
AVFNTGNYKYV

SEQ ID NO: 10 - protein, 5 aa, Mus musculus
MNHDT

SEQ ID NO: 11 - protein, 6 aa, Mus musculus
YYDKIL

SEQ ID NO: 12 - protein, 11 aa, Mus musculus
ASSPGASYEQY

SEQ ID NO: 13 - protein, 6 aa, Mus musculus
DRNVDY

SEQ ID NO: 14 - protein, 6 aa, Mus musculus
IFSNGE

SEQ ID NO: 15 - protein, 10 aa, Mus musculus
AASTNTGKLT

SEQ ID NO: 16 - protein, 6 aa, Mus musculus
NSQYPW

SEQ ID NO: 17 - protein, 6 aa, Mus musculus
LRSPGD

SEQ ID NO: 18 - protein, 9 aa, Mus musculus
TCSAGDEQY

SEQ ID NO: 19 - protein, 6 aa, Mus musculus
DGTSNN

SEQ ID NO: 20 - protein, 6 aa, Mus musculus
IFSDGE

SEQ ID NO: 21 - protein, 10 aa, Mus musculus
AVITNTGKLT

SEQ ID NO: 22 - protein, 5 aa, Mus musculus
FNHDT

SEQ ID NO: 23 - protein, 6 aa, Mus musculus
SITEND

SEQ ID NO: 24 - protein, 11 aa, Mus musculus
ASGGAFSYEQY

SEQ ID NO: 25 - protein, 6 aa, Mus musculus
DSTFDY

SEQ ID NO: 26 - protein, 7 aa, Mus musculus
IRPVSNK

SEQ ID NO: 27 - protein, 14 aa, Mus musculus
AASANTGANTGKLT

SEQ ID NO: 28 - protein, 5 aa, Mus musculus
LGHDT

SEQ ID NO: 29 - protein, 6 aa, Mus musculus
YNNKQL

SEQ ID NO: 30 - protein, 11 aa, Mus musculus
ASSWGLGYEQY

SEQ ID NO: 31 - protein, 6 aa, Mus musculus
DSATAY

SEQ ID NO: 32 - protein, 7 aa, Mus musculus
VLSNVDR

SEQ ID NO: 33 - protein, 10 aa, Mus musculus
AVDTNTGKLT

SEQ ID NO: 34 - protein, 5 aa, Mus musculus
NNHDY

SEQ ID NO: 35 - protein, 6 aa, Mus musculus
SYVADS

SEQ ID NO: 36 - protein, 14 aa, Mus musculus
ASSDRLGGSAETLY

SEQ ID NO: 37 - protein, 5 aa, Mus musculus
IAATT

SEQ ID NO: 38 - protein, 5 aa, Mus musculus
LVPGT

SEQ ID NO: 39 - protein, 14 aa, Mus musculus
AALANTGANTGKLT

SEQ ID NO: 40 - protein, 5 aa, Mus musculus
MSHET

SEQ ID NO: 41 - protein, 6 aa, Mus musculus
SYDVDS

SEQ ID NO: 42 - protein, 11 aa, Mus musculus
ASSWGQGYEQY

SEQ ID NO: 43 - protein, 5 aa, Mus musculus
TSLNS

SEQ ID NO: 44 - protein, 5 aa, Mus musculus
NPSGT

SEQ ID NO: 45 - protein, 11 aa, Mus musculus
AMMSNMGYKLT

SEQ ID NO: 46 - protein, 5 aa, Mus musculus
SGHDT

SEQ ID NO: 47 - protein, 6 aa, Mus musculus
FRDEAV

SEQ ID NO: 48 - protein, 10 aa, Mus musculus
ASSLSSYEQY

SEQ ID NO: 49 - protein, 6 aa, Mus musculus
DSASNY

SEQ ID NO: 50 - protein, 7 aa, Mus musculus
IRSNMER

SEQ ID NO: 51 - protein, 14 aa, Mus musculus
AASANTGANTGKLT

SEQ ID NO: 52 - protein, 5 aa, Mus musculus
LGHDT

SEQ ID NO: 53 - protein, 6 aa, Mus musculus
YNNKQL

SEQ ID NO: 54 - protein, 12 aa, Mus musculus
ASSWGLGSYEQY

SEQ ID NO: 55 - protein, 9 aa, Homo sapiens
SLFLGILSV

SEQ ID NO: 56 - protein, 130 aa, Mus musculus
MKSFSISLVVLWLQLNWVNSQQKVQQSPESLIVPEGGMASLNCTSSDRNVDYFWWYRQHSGKSPKMLMSIFSNGEKEEGRFTVHLNKASLHTSLHIRDSQPSDSALYLCAASTNTGKLTFGDGTVLTVKP

SEQ ID NO: 57 - protein, 131 aa, Mus musculus
MWQFCILCLCVLMASVATDPTVTLLEQNPRWRLVPRGQAVNLRCILKNSQYPWMSWYQQDLQKQLQWLFTLRSPGDKEVKSLPGADYLATRVTDTELRLQVANMSQGRTLYCTCSTGDEQYFGPGTRLTVL

SEQ ID NO: 58 - protein, 131 aa, Artificial Sequence
  synthetic
MAQFCILCLCVLMASVATDPTVTLLEQNPRWRLVPRGQAVNLRCILKNSQYPWMSWYQQDLQKQLQWLFTLRSPGDKEVKSLPGADYLATRVTDTELRLQVANMSQGRTLYCTCSTGDEQYFGPGTRLTVL

SEQ ID NO: 59 - protein, 131 aa, Mus musculus
MHSLHVSLVFLWLQLGGVSSQEKVQQSPESLIVPEGAMVSLNCSFSDSASQSIWWYQQHPGKGPKALISIFSNGNKKEGRLTVYLNRASLHVSLHIKDSQPSDSAVYLCAVFNTGNYKYVFGAGTRLKVIA

SEQ ID NO: 60 - protein, 131 aa, Mus musculus
MDPRLLCCVIFCLLAATFVDTTVKQNPRYKLARVGKPVNLICSQTMNHDTMYWYQKKPNQAPKLLLFYYDKILNREADTFEKFQSSRPNNSFCSLYIGSAGLEYSAMYLCASSPGASYEQYFGPGTRLTVL

SEQ ID NO: 61 - protein, 131 aa, Artificial Sequence
  synthetic
MAPRLLCCVIFCLLAATFVDTTVKQNPRYKLARVGKPVNLICSQTMNHDTMYWYQKKPNQAPKLLLFYYDKILNREADTFEKFQSSRPNNSFCSLYIGSAGLEYSAMYLCASSPGASYEQYFGPGTRLTVL

SEQ ID NO: 62 - protein, 130 aa, Mus musculus; identical to SEQ ID NO: 56

SEQ ID NO: 63 - protein, 131 aa, Mus musculus
MWQFCILCLCVLMASVATDPTVTLLEQNPRWRLVPRGQAVNLRCILKNSQYPWMSWYQQDLQKQLQWLFTLRSPGDKEVKSLPGADYLATRVTDTELRLQVANMSQGRTLYCTCSAGDEQYFGPGTRLTVL

SEQ ID NO: 64 - protein, 131 aa, Artificial Sequence
  synthetic
MAQFCILCLCVLMASVATDPTVTLLEQNPRWRLVPRGQAVNLRCILKNSQYPWMSWYQQDLQKQLQWLFTLRSPGDKEVKSLPGADYLATRVTDTELRLQVANMSQGRTLYCTCSAGDEQYFGPGTRLTVL

SEQ ID NO: 65 - protein, 130 aa, Mus musculus
MKSLSVSLVVLWLQFNWVSSQQKVQQSPESLTVSEGAMASLNCTFSDGTSNNFRWYRQHSAKGLEVLVSIFSDGEKEEGRFTAHLNRANLHVSLHIREPQPSDSAVYLCAVITNTGKLTFGDGTVLTVKP

SEQ ID NO: 66 - protein, 134 aa, Mus musculus
MNKWVFCWVTLCLLTVETTHGDGGIITQTPKFLIGQEGQKLTLKCQQNFNHDTMYWYRQDSGKGLRLIYYSITENDLQKGDLSEGYDASREKKSSFSLTVTSAQKNEMAVFLCASGGAFSYEQYFGPGTRLTVL

SEQ ID NO: 67 - protein, 134 aa, Artificial Sequence
  synthetic
MAKWVFCWVTLCLLTVETTHGDGGIITQTPKFLIGQEGQKLTLKCQQNFNHDTMYWYRQDSGKGLRLIYYSITENDLQKGDLSEGYDASREKKSSFSLTVTSAQKNEMAVFLCASGGAFSYEQYFGPGTRLTVL

SEQ ID NO: 68 - protein, 143 aa, Mus musculus
MDKILTASFLLLGLHLAGVSGQQQEKHDQQQVRQSPQSLTVWEGETAILNCSYEDSTFDYFPWYHQFPGESPALLIAIRPVSNKKEDGRFTIFFNKREKKFSLHIADSQPGDSATYFCAASANTGANTGKLTFGHGTILRVHP

SEQ ID NO: 69 - protein, 131 aa, Mus musculus
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGYEQYFGPGTRLTVL

SEQ ID NO: 70 - protein, 131 aa, Artificial Sequence
  synthetic
MACRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGYEQYFGPGTRLTVL

SEQ ID NO: 71 - protein, 132 aa, Mus musculus
MKTVTGPLLLCFWLQLNCVSRGEQVEQRPPHLSVREGDSAIIICTYTDSATAYFSWYKQEAGAGLQLLMSVLSNVDRKEEQGLTVLLNKKDKRLSLNLTAAHPGDSAVYFCAVDTNTGKLTFGDGTVLTVKP

SEQ ID NO: 72 - protein, 133 aa, Mus musculus
MGSRLFFVVLILLCAKHMEAAVTQSPRSKVAVTGGKVTLSCHQTNNHDYMYWYRQDTGHGLRLIHYSYVADSTEKGDIPDGYKASRPSQENFSLILELASLSQTAVYFCASSDRLGGSAETLYFGSGTRLTVL

SEQ ID NO: 73 - protein, 133 aa, Artificial Sequence
  synthetic
MASRLFFVVLILLCAKHMEAAVTQSPRSKVAVTGGKVTLSCHQTNNHDYMYWYRQDTGHGLRLIHYSYVADSTEKGDIPDGYKASRPSQENFSLILELASLSQTAVYFCASSDRLGGSAETLYFGSGTRLTVL

SEQ ID NO: 74 - protein, 132 aa, Mus musculus
MQRNLGAVLGILWVQICWVSGDKVKQSPSALSLQEGTNSALRCNFSIAATTVQWFLQNPRGSLINLFYLVPGTKENGRLKSAFDSKESYSTLHIRDAQLEDSGTYFCAALANTGANTGKLTFGHGTILRVHP

SEQ ID NO: 75 - protein, 131 aa, Mus musculus
MRVRLISAVVLCFLGTGLVDMKVTQMPRYLIKRMGENVLLECGQDMSHETMYWYRQDPGLGLQLIYISYDVDSNSEGDIPKGYRVSRKKREHFSLILDSAKTNQTSVYFCASSWGQGYEQYFGPGTRLTVL

SEQ ID NO: 76 - protein, 131 aa, Artificial Sequence
  synthetic
MAVRLISAVVLCFLGTGLVDMKVTQMPRYLIKRMGENVLLECGQDMSHETMYWYRQDPGLGLQLIYISYDVDSNSEGDIPKGYRVSRKKREHFSLILDSAKTNQTSVYFCASSWGQGYEQYFGPGTRLTVL

SEQ ID NO: 77 - protein, 129 aa, Mus musculus
MKRLLCSLLGLLCTQVCWVKGQQVQQSPTSLVLQEGENAELQCNFSTSLNSMQWFYQRPGGSLISVFYNPSGTKQSGRLTSTTVIKERRSSLHISSSQITDSGTYLCAMMSNMGYKLTFGTGTSLLVDP

SEQ ID NO: 78 - protein, 131 aa, Mus musculus
MGTRLLGWAVFCLLDTVLSEAGVTQSPRYAVLQEGQAVSFWCDPISGHDTLYWYQQPRDQGPQLLVYFRDEAVIDNSQLPSDRFSAVRPKGTNSTLKIQSAKQGDTATYLCASSLSSYEQYFGPGTRLTVL

SEQ ID NO: 79 - protein, 131 aa, Artificial Sequence
  synthetic
MATRLLGWAVFCLLDTVLSEAGVTQSPRYAVLQEGQAVSFWCDPISGHDTLYWYQQPRDQGPQLLVYFRDEAVIDNSQLPSDRFSAVRPKGTNSTLKIQSAKQGDTATYLCASSLSSYEQYFGPGTRLTVL

SEQ ID NO: 80 - protein, 136 aa, Mus musculus
MKTYAPTLFMFLWLQLDGMSQGEQVEQLPSILRVQEGSSASINCSYEDSASNYFPWYKQEPGENPKLIIDIRSNMERKQIQELIVLLDKKAKRFSLHITDTQPGDSAMYFCAASANTGANTGKLTFGHGTILRVHP

SEQ ID NO: 81 - protein, 132 aa, Mus musculus
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGSYEQYFGPGTRLTVL

SEQ ID NO: 82 - protein, 132 aa, Artificial Sequence
  synthetic
MACRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGSYEQYFGPGTRLTVL

SEQ ID NO: 83 - protein, 110 aa, Mus musculus
QQKVQQSPESLIVPEGGMASLNCTSSDRNVDYFWWYRQHSGKSPKMLMSIFSNGEKEEGRFTVHLNKASLHTSLHIRDSQPSDSALYLCAASTNTGKLTFGDGTVLTVKP

SEQ ID NO: 84 - protein, 110 aa, Mus musculus
VTLLEQNPRWRLVPRGQAVNLRCILKNSQYPWMSWYQQDLQKQLQWLFTLRSPGDKEVKSLPGADYLATRVTDTELRLQVANMSQGRTLYCTCSTGDEQYFGPGTRLTVL

SEQ ID NO: 85 - protein, 111 aa, Mus musculus
QEKVQQSPESLIVPEGAMVSLNCSFSDSASQSIWWYQQHPGKGPKALISIFSNGNKKEGRLTVYLNRASLHVSLHIKDSQPSDSAVYLCAVFNTGNYKYVFGAGTRLKVIA

SEQ ID NO: 86 - protein, 112 aa, Mus musculus
DTTVKQNPRYKLARVGKPVNLICSQTMNHDTMYWYQKKPNQAPKLLLFYYDKILNREADTFEKFQSSRPNNSFCSLYIGSAGLEYSAMYLCASSPGASYEQYFGPGTRLTVL

SEQ ID NO: 87 - protein, 110 aa, Mus musculus; identical to SEQ ID NO: 83

SEQ ID NO: 88 - protein, 110 aa, Mus musculus
VTLLEQNPRWRLVPRGQAVNLRCILKNSQYPWMSWYQQDLQKQLQWLFTLRSPGDKEVKSLPGADYLATRVTDTELRLQVANMSQGRTLYCTCSAGDEQYFGPGTRLTVL

SEQ ID NO: 89 - protein, 110 aa, Mus musculus
QQKVQQSPESLTVSEGAMASLNCTFSDGTSNNFRWYRQHSAKGLEVLVSIFSDGEKEEGRFTAHLNRANLHVSLHIREPQPSDSAVYLCAVITNTGKLTFGDGTVLTVKP

SEQ ID NO: 90 - protein, 112 aa, Mus musculus
GGIITQTPKFLIGQEGQKLTLKCQQNFNHDTMYWYRQDSGKGLRLIYYSITENDLQKGDLSEGYDASREKKSSFSLTVTSAQKNEMAVFLCASGGAFSYEQYFGPGTRLTVL

SEQ ID NO: 91 - protein, 115 aa, Mus musculus
QQQVRQSPQSLTVWEGETAILNCSYEDSTFDYFPWYHQFPGESPALLIAIRPVSNKKEDGRFTIFFNKREKKFSLHIADSQPGDSATYFCAASANTGANTGKLTFGHGTILRVHP

SEQ ID NO: 92 - protein, 112 aa, Mus musculus
ETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGYEQYFGPGTRLTVL

SEQ ID NO: 93 - protein, 111 aa, Mus musculus
GEQVEQRPPHLSVREGDSAIIICTYTDSATAYFSWYKQEAGAGLQLLMSVLSNVDRKEEQGLTVLLNKKDKRLSLNLTAAHPGDSAVYFCAVDTNTGKLTFGDGTVLTVKP

SEQ ID NO: 94 - protein, 115 aa, Mus musculus
EAAVTQSPRSKVAVTGGKVTLSCHQTNNHDYMYWYRQDTGHGLRLIHYSYVADSTEKGDIPDGYKASRPSQENFSLILELASLSQTAVYFCASSDRLGGSAETLYFGSGTRLTVL

SEQ ID NO: 95 - protein, 112 aa, Mus musculus
GDKVKQSPSALSLQEGTNSALRCNFSIAATTVQWFLQNPRGSLINLFYLVPGTKENGRLKSAFDSKESYSTLHIRDAQLEDSGTYFCAALANTGANTGKLTFGHGTILRVHP

SEQ ID NO: 96 - protein, 112 aa, Mus musculus
DMKVTQMPRYLIKRMGENVLLECGQDMSHETMYWYRQDPGLGLQLIYISYDVDSNSEGDIPKGYRVSRKKREHFSLILDSAKTNQTSVYFCASSWGQGYEQYFGPGTRLTVL

SEQ ID NO: 97 - protein, 109 aa, Mus musculus
GQQVQQSPTSLVLQEGENAELQCNFSTSLNSMQWFYQRPGGSLISVFYNPSGTKQSGRLTSTTVIKERRSSLHISSSQITDSGTYLCAMMSNMGYKLTFGTGTSLLVDP

SEQ ID NO: 98 - protein, 112 aa, Mus musculus
EAGVTQSPRYAVLQEGQAVSFWCDPISGHDTLYWYQQPRDQGPQLLVYFRDEAVIDNSQLPSDRFSAVRPKGTNSTLKIQSAKQGDTATYLCASSLSSYEQYFGPGTRLTVL

SEQ ID NO: 99 - protein, 115 aa, Mus musculus
GEQVEQLPSILRVQEGSSASINCSYEDSASNYFPWYKQEPGENPKLIIDIRSNMERKQIQELIVLLDKKAKRFSLHITDTQPGDSAMYFCAASANTGANTGKLTFGHGTILRVHP

SEQ ID NO: 100 - protein, 113 aa, Mus musculus
ETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGSYEQYFGPGTRLTVL

SEQ ID NO: 101 - protein, 137 aa, Mus musculus
NIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 102 - protein, 173 aa, Mus musculus
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 103 - protein, 267 aa, Artificial Sequence
  synthetic
MKSFSISLVVLWLQLNWVNSQQKVQQSPESLIVPEGGMASLNCTSSDRNVDYFWWYRQHSGKSPKMLMSIFSNGEKEEGRFTVHLNKASLHTSLHIRDSQPSDSALYLCAASTNTGKLTFGDGTVLTVKPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 104 - protein, 304 aa, Artificial Sequence
  synthetic
MWQFCILCLCVLMASVATDPTVTLLEQNPRWRLVPRGQAVNLRCILKNSQYPWMSWYQQDLQKQLQWLFTLRSPGDKEVKSLPGADYLATRVTDTELRLQVANMSQGRTLYCTCSTGDEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 105 - protein, 304 aa, Artificial Sequence
  synthetic
MAQFCILCLCVLMASVATDPTVTLLEQNPRWRLVPRGQAVNLRCILKNSQYPWMSWYQQDLQKQLQWLFTLRSPGDKEVKSLPGADYLATRVTDTELRLQVANMSQGRTLYCTCSTGDEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 106 - protein, 268 aa, Artificial Sequence
  synthetic
MHSLHVSLVFLWLQLGGVSSQEKVQQSPESLIVPEGAMVSLNCSFSDSASQSIWWYQQHPGKGPKALISIFSNGNKKEGRLTVYLNRASLHVSLHIKDSQPSDSAVYLCAVFNTGNYKYVFGAGTRLKVIAHIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 107 - protein, 304 aa, Artificial Sequence
  synthetic
MDPRLLCCVIFCLLAATFVDTTVKQNPRYKLARVGKPVNLICSQTMNHDTMYWYQKKPNQAPKLLLFYYDKILNREADTFEKFQSSRPNNSFCSLYIGSAGLEYSAMYLCASSPGASYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 108 - protein, 304 aa, Artificial Sequence
  synthetic
MAPRLLCCVIFCLLAATFVDTTVKQNPRYKLARVGKPVNLICSQTMNHDTMYWYQKKPNQAPKLLLFYYDKILNREADTFEKFQSSRPNNSFCSLYIGSAGLEYSAMYLCASSPGASYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 109 - protein, 267 aa, Artificial Sequence; identical to SEQ ID NO: 103
  synthetic

SEQ ID NO: 110 - protein, 304 aa, Artificial Sequence
  synthetic
MWQFCILCLCVLMASVATDPTVTLLEQNPRWRLVPRGQAVNLRCILKNSQYPWMSWYQQDLQKQLQWLFTLRSPGDKEVKSLPGADYLATRVTDTELRLQVANMSQGRTLYCTCSAGDEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 111 - protein, 304 aa, Artificial Sequence
  synthetic
MAQFCILCLCVLMASVATDPTVTLLEQNPRWRLVPRGQAVNLRCILKNSQYPWMSWYQQDLQKQLQWLFTLRSPGDKEVKSLPGADYLATRVTDTELRLQVANMSQGRTLYCTCSAGDEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 112 - protein, 267 aa, Artificial Sequence
  synthetic
MKSLSVSLVVLWLQFNWVSSQQKVQQSPESLTVSEGAMASLNCTFSDGTSNNFRWYRQHSAKGLEVLVSIFSDGEKEEGRFTAHLNRANLHVSLHIREPQPSDSAVYLCAVITNTGKLTFGDGTVLTVKPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 113 - protein, 307 aa, Artificial Sequence
  synthetic
MNKWVFCWVTLCLLTVETTHGDGGIITQTPKFLIGQEGQKLTLKCQQNFNHDTMYWYRQDSGKGLRLIYYSITENDLQKGDLSEGYDASREKKSSFSLTVTSAQKNEMAVFLCASGGAFSYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 114 - protein, 307 aa, Artificial Sequence
  synthetic
MAKWVFCWVTLCLLTVETTHGDGGIITQTPKFLIGQEGQKLTLKCQQNFNHDTMYWYRQDSGKGLRLIYYSITENDLQKGDLSEGYDASREKKSSFSLTVTSAQKNEMAVFLCASGGAFSYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 115 - protein, 280 aa, Artificial Sequence
  synthetic
MDKILTASFLLLGLHLAGVSGQQQEKHDQQQVRQSPQSLTVWEGETAILNCSYEDSTFDYFPWYHQFPGESPALLIAIRPVSNKKEDGRFTIFFNKREKKFSLHIADSQPGDSATYFCAASANTGANTGKLTFGHGTILRVHPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 116 - protein, 304 aa, Artificial Sequence
  synthetic
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 117 - protein, 304 aa, Artificial Sequence
  synthetic
MACRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 118 - protein, 269 aa, Artificial Sequence
  synthetic
MKTVTGPLLLCFWLQLNCVSRGEQVEQRPPHLSVREGDSAIIICTYTDSATAYFSWYKQEAGAGLQLLMSVLSNVDRKEEQGLTVLLNKKDKRLSLNLTAAHPGDSAVYFCAVDTNTGKLTFGDGTVLTVKPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 119 - protein, 306 aa, Artificial Sequence
  synthetic
MGSRLFFVVLILLCAKHMEAAVTQSPRSKVAVTGGKVTLSCHQTNNHDYMYWYRQDTGHGLRLIHYSYVADSTEKGDIPDGYKASRPSQENFSLILELASLSQTAVYFCASSDRLGGSAETLYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 120 - protein, 306 aa, Artificial Sequence
  synthetic
MASRLFFVVLILLCAKHMEAAVTQSPRSKVAVTGGKVTLSCHQTNNHDYMYWYRQDTGHGLRLIHYSYVADSTEKGDIPDGYKASRPSQENFSLILELASLSQTAVYFCASSDRLGGSAETLYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 121 - protein, 269 aa, Artificial Sequence
  synthetic
MQRNLGAVLGILWVQICWVSGDKVKQSPSALSLQEGTNSALRCNFSIAATTVQWFLQNPRGSLINLFYLVPGTKENGRLKSAFDSKESYSTLHIRDAQLEDSGTYFCAALANTGANTGKLTFGHGTILRVHPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 122 - protein, 304 aa, Artificial Sequence
  synthetic
MRVRLISAVVLCFLGTGLVDMKVTQMPRYLIKRMGENVLLECGQDMSHETMYWYRQDPGLGLQLIYISYDVDSNSEGDIPKGYRVSRKKREHFSLILDSAKTNQTSVYFCASSWGQGYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 123 - protein, 304 aa, Artificial Sequence
  synthetic
MAVRLISAVVLCFLGTGLVDMKVTQMPRYLIKRMGENVLLECGQDMSHETMYWYRQDPGLGLQLIYISYDVDSNSEGDIPKGYRVSRKKREHFSLILDSAKTNQTSVYFCASSWGQGYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 124 - protein, 266 aa, Artificial Sequence
  synthetic
MKRLLCSLLGLLCTQVCWVKGQQVQQSPTSLVLQEGENAELQCNFSTSLNSMQWFYQRPGGSLISVFYNPSGTKQSGRLTSTTVIKERRSSLHISSSQITDSGTYLCAMMSNMGYKLTFGTGTSLLVDPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 125 - protein, 304 aa, Artificial Sequence
  synthetic
MGTRLLGWAVFCLLDTVLSEAGVTQSPRYAVLQEGQAVSFWCDPISGHDTLYWYQQPRDQGPQLLVYFRDEAVIDNSQLPSDRFSAVRPKGTNSTLKIQSAKQGDTATYLCASSLSSYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 126 - protein, 304 aa, Artificial Sequence
  synthetic
MATRLLGWAVFCLLDTVLSEAGVTQSPRYAVLQEGQAVSFWCDPISGHDTLYWYQQPRDQGPQLLVYFRDEAVIDNSQLPSDRFSAVRPKGTNSTLKIQSAKQGDTATYLCASSLSSYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 127 - protein, 273 aa, Artificial Sequence
  synthetic
MKTYAPTLFMFLWLQLDGMSQGEQVEQLPSILRVQEGSSASINCSYEDSASNYFPWYKQEPGENPKLIIDIRSNMERKQIQELIVLLDKKAKRFSLHITDTQPGDSAMYFCAASANTGANTGKLTFGHGTILRVHPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 128 - protein, 305 aa, Artificial Sequence
  synthetic
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGSYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 129 - protein, 305 aa, Artificial Sequence
  synthetic
MACRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGSYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 130 - protein, 247 aa, Artificial Sequence
  synthetic
QQKVQQSPESLIVPEGGMASLNCTSSDRNVDYFWWYRQHSGKSPKMLMSIFSNGEKEEGRFTVHLNKASLHTSLHIRDSQPSDSALYLCAASTNTGKLTFGDGTVLTVKPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 131 - protein, 283 aa, Artificial Sequence
  synthetic
VTLLEQNPRWRLVPRGQAVNLRCILKNSQYPWMSWYQQDLQKQLQWLFTLRSPGDKEVKSLPGADYLATRVTDTELRLQVANMSQGRTLYCTCSTGDEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 132 - protein, 248 aa, Artificial Sequence
  synthetic
QEKVQQSPESLIVPEGAMVSLNCSFSDSASQSIWWYQQHPGKGPKALISIFSNGNKKEGRLTVYLNRASLHVSLHIKDSQPSDSAVYLCAVFNTGNYKYVFGAGTRLKVIAHIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 133 - protein, 285 aa, Artificial Sequence
  synthetic
DTTVKQNPRYKLARVGKPVNLICSQTMNHDTMYWYQKKPNQAPKLLLFYYDKILNREADTFEKFQSSRPNNSFCSLYIGSAGLEYSAMYLCASSPGASYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 134 - protein, 247 aa, Artificial Sequence; identical to SEQ ID NO: 130
  synthetic

SEQ ID NO: 135 - protein, 283 aa, Artificial Sequence
  synthetic
VTLLEQNPRWRLVPRGQAVNLRCILKNSQYPWMSWYQQDLQKQLQWLFTLRSPGDKEVKSLPGADYLATRVTDTELRLQVANMSQGRTLYCTCSAGDEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 136 - protein, 247 aa, Artificial Sequence
  synthetic
QQKVQQSPESLTVSEGAMASLNCTFSDGTSNNFRWYRQHSAKGLEVLVSIFSDGEKEEGRFTAHLNRANLHVSLHIREPQPSDSAVYLCAVITNTGKLTFGDGTVLTVKPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 137 - protein, 285 aa, Artificial Sequence
  synthetic
GGIITQTPKFLIGQEGQKLTLKCQQNFNHDTMYWYRQDSGKGLRLIYYSITENDLQKGDLSEGYDASREKKSSFSLTVTSAQKNEMAVFLCASGGAFSYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 138 - protein, 252 aa, Artificial Sequence
  synthetic
QQQVRQSPQSLTVWEGETAILNCSYEDSTFDYFPWYHQFPGESPALLIAIRPVSNKKEDGRFTIFFNKREKKFSLHIADSQPGDSATYFCAASANTGANTGKLTFGHGTILRVHPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 139 - protein, 285 aa, Artificial Sequence
  synthetic
ETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 140 - protein, 248 aa, Artificial Sequence
  synthetic
GEQVEQRPPHLSVREGDSAIIICTYTDSATAYFSWYKQEAGAGLQLLMSVLSNVDRKEEQGLTVLLNKKDKRLSLNLTAAHPGDSAVYFCAVDTNTGKLTFGDGTVLTVKPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 141 - protein, 288 aa, Artificial Sequence
  synthetic
EAAVTQSPRSKVAVTGGKVTLSCHQTNNHDYMYWYRQDTGHGLRLIHYSYVADSTEKGDIPDGYKASRPSQENFSLILELASLSQTAVYFCASSDRLGGSAETLYFGSGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 142 - protein, 249 aa, Artificial Sequence
  synthetic
GDKVKQSPSALSLQEGTNSALRCNFSIAATTVQWFLQNPRGSLINLFYLVPGTKENGRLKSAFDSKESYSTLHIRDAQLEDSGTYFCAALANTGANTGKLTFGHGTILRVHPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 143 - protein, 285 aa, Artificial Sequence
  synthetic
DMKVTQMPRYLIKRMGENVLLECGQDMSHETMYWYRQDPGLGLQLIYISYDVDSNSEGDIPKGYRVSRKKREHFSLILDSAKTNQTSVYFCASSWGQGYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 144 - protein, 246 aa, Artificial Sequence
  synthetic
GQQVQQSPTSLVLQEGENAELQCNFSTSLNSMQWFYQRPGGSLISVFYNPSGTKQSGRLTSTTVIKERRSSLHISSSQITDSGTYLCAMMSNMGYKLTFGTGTSLLVDPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 145 - protein, 285 aa, Artificial Sequence
  synthetic
EAGVTQSPRYAVLQEGQAVSFWCDPISGHDTLYWYQQPRDQGPQLLVYFRDEAVIDNSQLPSDRFSAVRPKGTNSTLKIQSAKQGDTATYLCASSLSSYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 146 - protein, 252 aa, Artificial Sequence
  synthetic
GEQVEQLPSILRVQEGSSASINCSYEDSASNYFPWYKQEPGENPKLIIDIRSNMERKQIQELIVLLDKKAKRFSLHITDTQPGDSAMYFCAASANTGANTGKLTFGHGTILRVHPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 147 - protein, 286 aa, Artificial Sequence
  synthetic
ETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGSYEQYFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 148 - protein, 137 aa, Artificial Sequence
  synthetic
  MISC_FEATURE (48)..(48): Xaa is Thr or Cys.
  MISC_FEATURE (112)..(112): Xaa is Ser, Ala, Val, Leu, Ile, Pro, Phe, Met, or Trp.
  MISC_FEATURE (114)..(114): Xaa is Met, Ala, Val, Leu, Ile, Pro, Phe, or Trp.
  MISC_FEATURE (115)..(115): Xaa is Gly, Ala, Val, Leu, Ile, Pro, Phe, Met, or Trp.
NIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 149 - protein, 173 aa, Artificial Sequence
  synthetic
  MISC_FEATURE (57)..(57): Xaa is Ser or Cys.
EDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 150 - protein, 267 aa, Artificial Sequence
  synthetic
  MISC_FEATURE (178)..(178): Xaa is Thr or Cys.
  MISC_FEATURE (242)..(242): Xaa is Ser, Ala, Val, Leu, Ile, Pro, Phe, Met, or Trp.
  MISC_FEATURE (244)..(244): Xaa is Met, Ala, Val, Leu, Ile, Pro, Phe, or Trp.
  MISC_FEATURE (245)..(245): Xaa is Gly, Ala, Val, Leu, Ile, Pro, Phe, Met, or Trp.
MKSFSISLVVLWLQLNWVNSQQKVQQSPESLIVPEGGMASLNCTSSDRNVDYFWWYRQHSGKSPKMLMSIFSNGEKEEGRFTVHLNKASLHTSLHIRDSQPSDSALYLCAASTNTGKLTFGDGTVLTVKPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 151 - protein, 304 aa, Artificial Sequence
  synthetic
  MISC_FEATURE (188)..(188): Xaa is Ser or Cys.
MWQFCILCLCVLMASVATDPTVTLLEQNPRWRLVPRGQAVNLRCILKNSQYPWMSWYQQDLQKQLQWLFTLRSPGDKEVKSLPGADYLATRVTDTELRLQVANMSQGRTLYCTCSTGDEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 152 - protein, 304 aa, Artificial Sequence
  synthetic
  MISC_FEATURE (188)..(188): Xaa is Ser or Cys.
MAQFCILCLCVLMASVATDPTVTLLEQNPRWRLVPRGQAVNLRCILKNSQYPWMSWYQQDLQKQLQWLFTLRSPGDKEVKSLPGADYLATRVTDTELRLQVANMSQGRTLYCTCSTGDEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 153 - protein, 268 aa, Artificial Sequence
  synthetic
  MISC_FEATURE (179)..(179): Xaa is Thr or Cys.
  MISC_FEATURE (243)..(243): Xaa is Ser, Ala, Val, Leu, Ile, Pro, Phe, Met, or Trp.
  MISC_FEATURE (245)..(245): Xaa is Met, Ala, Val, Leu, Ile, Pro, Phe, or Trp.
  MISC_FEATURE (246)..(246): Xaa is Gly, Ala, Val, Leu, Ile, Pro, Phe, Met, or Trp.
MHSLHVSLVFLWLQLGGVSSQEKVQQSPESLIVPEGAMVSLNCSFSDSASQSIWWYQQHPGKGPKALISIFSNGNKKEGRLTVYLNRASLHVSLHIKDSQPSDSAVYLCAVFNTGNYKYVFGAGTRLKVIAHIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 154 - protein, 304 aa, Artificial Sequence
  synthetic
  MISC_FEATURE (188)..(188): Xaa is Ser or Cys.
MDPRLLCCVIFCLLAATFVDTTVKQNPRYKLARVGKPVNLICSQTMNHDTMYWYQKKPNQAPKLLLFYYDKILNREADTFEKFQSSRPNNSFCSLYIGSAGLEYSAMYLCASSPGASYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 155 - protein, 304 aa, Artificial Sequence
  synthetic
  MISC_FEATURE (188)..(188): Xaa is Ser or Cys.
MAPRLLCCVIFCLLAATFVDTTVKQNPRYKLARVGKPVNLICSQTMNHDTMYWYQKKPNQAPKLLLFYYDKILNREADTFEKFQSSRPNNSFCSLYIGSAGLEYSAMYLCASSPGASYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 156 - protein, 267 aa, Artificial Sequence; identical to SEQ ID NO: 150
  synthetic
  MISC_FEATURE (178)..(178): Xaa is Thr or Cys.
  MISC_FEATURE (242)..(242): Xaa is Ser, Ala, Val, Leu, Ile, Pro, Phe, Met, or Trp.
  MISC_FEATURE (244)..(244): Xaa is Met, Ala, Val, Leu, Ile, Pro, Phe, or Trp.
  MISC_FEATURE (245)..(245): Xaa is Gly, Ala, Val, Leu, Ile, Pro, Phe, Met, or Trp.

SEQ ID NO: 157 - protein, 304 aa, Artificial Sequence
  synthetic
  MISC_FEATURE (188)..(188): Xaa is Ser or Cys.
MWQFCILCLCVLMASVATDPTVTLLEQNPRWRLVPRGQAVNLRCILKNSQYPWMSWYQQDLQKQLQWLFTLRSPGDKEVKSLPGADYLATRVTDTELRLQVANMSQGRTLYCTCSAGDEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 158 - protein, 304 aa, Artificial Sequence
  synthetic
  MISC_FEATURE (188)..(188): Xaa is Ser or Cys.
MAQFCILCLCVLMASVATDPTVTLLEQNPRWRLVPRGQAVNLRCILKNSQYPWMSWYQQDLQKQLQWLFTLRSPGDKEVKSLPGADYLATRVTDTELRLQVANMSQGRTLYCTCSAGDEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 159 - protein, 267 aa, Artificial Sequence
  synthetic
  MISC_FEATURE (178)..(178): Xaa is Thr or Cys.
  MISC_FEATURE (242)..(242): Xaa is Ser, Ala, Val, Leu, Ile, Pro, Phe, Met, or Trp.
  MISC_FEATURE (244)..(244): Xaa is Met, Ala, Val, Leu, Ile, Pro, Phe, or Trp.
  MISC_FEATURE (245)..(245): Xaa is Gly, Ala, Val, Leu, Ile, Pro, Phe, Met, or Trp.
MKSLSVSLVVLWLQFNWVSSQQKVQQSPESLTVSEGAMASLNCTFSDGTSNNFRWYRQHSAKGLEVLVSIFSDGEKEEGRFTAHLNRANLHVSLHIREPQPSDSAVYLCAVITNTGKLTFGDGTVLTVKPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 160 - protein, 307 aa, Artificial Sequence
  synthetic
  MISC_FEATURE (191)..(191): Xaa is Ser or Cys.
MNKWVFCWVTLCLLTVETTHGDGGIITQTPKFLIGQEGQKLTLKCQQNFNHDTMYWYRQDSGKGLRLIYYSITENDLQKGDLSEGYDASREKKSSFSLTVTSAQKNEMAVFLCASGGAFSYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 161 - protein, 307 aa, Artificial Sequence
  synthetic
  MISC_FEATURE (191)..(191): Xaa is Ser or Cys.
MAKWVFCWVTLCLLTVETTHGDGGIITQTPKFLIGQEGQKLTLKCQQNFNHDTMYWYRQDSGKGLRLIYYSITENDLQKGDLSEGYDASREKKSSFSLTVTSAQKNEMAVFLCASGGAFSYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 162 - protein, 280 aa, Artificial Sequence
  synthetic
  MISC_FEATURE (191)..(191): Xaa is Thr or Cys.
  MISC_FEATURE (255)..(255): Xaa is Ser, Ala, Val, Leu, Ile, Pro, Phe, Met, or Trp.
  MISC_FEATURE (257)..(257): Xaa is Met, Ala, Val, Leu, Ile, Pro, Phe, or Trp.
  MISC_FEATURE (258)..(258): Xaa is Gly, Ala, Val, Leu, Ile, Pro, Phe, Met, or Trp.
MDKILTASFLLLGLHLAGVSGQQQEKHDQQQVRQSPQSLTVWEGETAILNCSYEDSTFDYFPWYHQFPGESPALLIAIRPVSNKKEDGRFTIFFNKREKKFSLHIADSQPGDSATYFCAASANTGANTGKLTFGHGTILRVHPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 163 - protein, 304 aa, Artificial Sequence
  synthetic
  MISC_FEATURE (188)..(188): Xaa is Ser or Cys.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 164 - protein, 304 aa, Artificial Sequence
  synthetic
  MISC_FEATURE (188)..(188): Xaa is Ser or Cys.
MACRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 165 - protein, 269 aa, Artificial Sequence
  synthetic
  MISC_FEATURE (180)..(180): Xaa is Thr or Cys.
  MISC_FEATURE (244)..(244): Xaa is Ser, Ala, Val, Leu, Ile, Pro, Phe, Met, or Trp.
  MISC_FEATURE (246)..(246): Xaa is Met, Ala, Val, Leu, Ile, Pro, Phe, or Trp.
  MISC_FEATURE (247)..(247): Xaa is Gly, Ala, Val, Leu, Ile, Pro, Phe, Met, or Trp.
MKTVTGPLLLCFWLQLNCVSRGEQVEQRPPHLSVREGDSAIIICTYTDSATAYFSWYKQEAGAGLQLLMSVLSNVDRKEEQGLTVLLNKKDKRLSLNLTAAHPGDSAVYFCAVDTNTGKLTFGDGTVLTVKPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 166 - protein, 306 aa, Artificial Sequence
  synthetic
  MISC_FEATURE (190)..(190): Xaa is Ser or Cys.
MGSRLFFVVLILLCAKHMEAAVTQSPRSKVAVTGGKVTLSCHQTNNHDYMYWYRQDTGHGLRLIHYSYVADSTEKGDIPDGYKASRPSQENFSLILELASLSQTAVYFCASSDRLGGSAETLYFGSGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 167 - protein, 306 aa, Artificial Sequence
  synthetic
  MISC_FEATURE (190)..(190): Xaa is Ser or Cys.
MASRLFFVVLILLCAKHMEAAVTQSPRSKVAVTGGKVTLSCHQTNNHDYMYWYRQDTGHGLRLIHYSYVADSTEKGDIPDGYKASRPSQENFSLILELASLSQTAVYFCASSDRLGGSAETLYFGSGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 168 - protein, 269 aa, Artificial Sequence
  synthetic
  MISC_FEATURE (180)..(180): Xaa is Thr or Cys.
  MISC_FEATURE (244)..(244): Xaa is Ser, Ala, Val, Leu, Ile, Pro, Phe, Met, or Trp.
  MISC_FEATURE (246)..(246): Xaa is Met, Ala, Val, Leu, Ile, Pro, Phe, or Trp.
  MISC_FEATURE (247)..(247): Xaa is Gly, Ala, Val, Leu, Ile, Pro, Phe, Met, or Trp.
MQRNLGAVLGILWVQICWVSGDKVKQSPSALSLQEGTNSALRCNFSIAATTVQWFLQNPRGSLINLFYLVPGTKENGRLKSAFDSKESYSTLHIRDAQLEDSGTYFCAALANTGANTGKLTFGHGTILRVHPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 169 - protein, 304 aa, Artificial Sequence
  synthetic
  MISC_FEATURE (188)..(188): Xaa is Ser or Cys.
MRVRLISAVVLCFLGTGLVDMKVTQMPRYLIKRMGENVLLECGQDMSHETMYWYRQDPGLGLQLIYISYDVDSNSEGDIPKGYRVSRKKREHFSLILDSAKTNQTSVYFCASSWGQGYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 170 - protein, 304 aa, Artificial Sequence
  synthetic
  MISC_FEATURE (188)..(188): Xaa is Ser or Cys.
MAVRLISAVVLCFLGTGLVDMKVTQMPRYLIKRMGENVLLECGQDMSHETMYWYRQDPGLGLQLIYISYDVDSNSEGDIPKGYRVSRKKREHFSLILDSAKTNQTSVYFCASSWGQGYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 171 - protein, 266 aa, Artificial Sequence
  synthetic
  MISC_FEATURE (177)..(177): Xaa is Thr or Cys.
  MISC_FEATURE (241)..(241): Xaa is Ser, Ala, Val, Leu, Ile, Pro, Phe, Met, or Trp.
  MISC_FEATURE (243)..(243): Xaa is Met, Ala, Val, Leu, Ile, Pro, Phe, or Trp.
  MISC_FEATURE (244)..(244): Xaa is Gly, Ala, Val, Leu, Ile, Pro, Phe, Met, or Trp.
MKRLLCSLLGLLCTQVCWVKGQQVQQSPTSLVLQEGENAELQCNFSTSLNSMQWFYQRPGGSLISVFYNPSGTKQSGRLTSTTVIKERRSSLHISSSQITDSGTYLCAMMSNMGYKLTFGTGTSLLVDPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 172 - protein, 304 aa, Artificial Sequence
  synthetic
  MISC_FEATURE (188)..(188): Xaa is Ser or Cys.
MGTRLLGWAVFCLLDTVLSEAGVTQSPRYAVLQEGQAVSFWCDPISGHDTLYWYQQPRDQGPQLLVYFRDEAVIDNSQLPSDRFSAVRPKGTNSTLKIQSAKQGDTATYLCASSLSSYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 173 - protein, 304 aa, Artificial Sequence
  synthetic
  MISC_FEATURE (188)..(188): Xaa is Ser or Cys.
MATRLLGWAVFCLLDTVLSEAGVTQSPRYAVLQEGQAVSFWCDPISGHDTLYWYQQPRDQGPQLLVYFRDEAVIDNSQLPSDRFSAVRPKGTNSTLKIQSAKQGDTATYLCASSLSSYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 174 - protein, 273 aa, Artificial Sequence
  synthetic
  MISC_FEATURE (184)..(184): Xaa is Thr or Cys.
  MISC_FEATURE (248)..(248): Xaa is Ser, Ala, Val, Leu, Ile, Pro, Phe, Met, or Trp.
  MISC_FEATURE (250)..(250): Xaa is Met, Ala, Val, Leu, Ile, Pro, Phe, or Trp.
  MISC_FEATURE (251)..(251): Xaa is Gly, Ala, Val, Leu, Ile, Pro, Phe, Met, or Trp.
MKTYAPTLFMFLWLQLDGMSQGEQVEQLPSILRVQEGSSASINCSYEDSASNYFPWYKQEPGENPKLIIDIRSNMERKQIQELIVLLDKKAKRFSLHITDTQPGDSAMYFCAASANTGANTGKLTFGHGTILRVHPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 175 - protein, 305 aa, Artificial Sequence
  synthetic
  MISC_FEATURE (189)..(189): Xaa is Ser or Cys.
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGSYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 176 - protein, 305 aa, Artificial Sequence
  synthetic
  MISC_FEATURE (189)..(189): Xaa is Ser or Cys.
MACRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGSYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 177 - protein, 247 aa, Artificial Sequence
  synthetic
  MISC_FEATURE (158)..(158): Xaa is Thr or Cys.
  MISC_FEATURE (222)..(222): Xaa is Ser, Ala, Val, Leu, Ile, Pro, Phe, Met, or Trp.
  MISC_FEATURE (224)..(224): Xaa is Met, Ala, Val, Leu, Ile, Pro, Phe, or Trp.
  MISC_FEATURE (225)..(225): Xaa is Gly, Ala, Val, Leu, Ile, Pro, Phe, Met, or Trp.
QQKVQQSPESLIVPEGGMASLNCTSSDRNVDYFWWYRQHSGKSPKMLMSIFSNGEKEEGRFTVHLNKASLHTSLHIRDSQPSDSALYLCAASTNTGKLTFGDGTVLTVKPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 178 - protein, 283 aa, Artificial Sequence
  synthetic
  MISC_FEATURE (167)..(167): Xaa is Ser or Cys.
VTLLEQNPRWRLVPRGQAVNLRCILKNSQYPWMSWYQQDLQKQLQWLFTLRSPGDKEVKSLPGADYLATRVTDTELRLQVANMSQGRTLYCTCSTGDEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 179 - protein, 248 aa, Artificial Sequence
  synthetic
  MISC_FEATURE (159)..(159): Xaa is Thr or Cys.
  MISC_FEATURE (223)..(223): Xaa is Ser, Ala, Val, Leu, Ile, Pro, Phe, Met, or Trp.
  MISC_FEATURE (225)..(225): Xaa is Met, Ala, Val, Leu, Ile, Pro, Phe, or Trp.
  MISC_FEATURE (226)..(226): Xaa is Gly, Ala, Val, Leu, Ile, Pro, Phe, Met, or Trp.
QEKVQQSPESLIVPEGAMVSLNCSFSDSASQSIWWYQQHPGKGPKALISIFSNGNKKEGRLTVYLNRASLHVSLHIKDSQPSDSAVYLCAVFNTGNYKYVFGAGTRLKVIAHIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 180 - protein, 285 aa, Artificial Sequence
  synthetic
  MISC_FEATURE (169)..(169): Xaa is Ser or Cys.
DTTVKQNPRYKLARVGKPVNLICSQTMNHDTMYWYQKKPNQAPKLLLFYYDKILNREADTFEKFQSSRPNNSFCSLYIGSAGLEYSAMYLCASSPGASYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 181 - protein, 247 aa, Artificial Sequence; identical to SEQ ID NO: 177
  synthetic
  MISC_FEATURE (158)..(158): Xaa is Thr or Cys.
  MISC_FEATURE (222)..(222): Xaa is Ser, Ala, Val, Leu, Ile, Pro, Phe, Met, or Trp.
  MISC_FEATURE (224)..(224): Xaa is Met, Ala, Val, Leu, Ile, Pro, Phe, or Trp.
  MISC_FEATURE (225)..(225): Xaa is Gly, Ala, Val, Leu, Ile, Pro, Phe, Met, or Trp.

SEQ ID NO: 182 - protein, 283 aa, Artificial Sequence
  synthetic
  MISC_FEATURE (167)..(167): Xaa is Ser or Cys.
VTLLEQNPRWRLVPRGQAVNLRCILKNSQYPWMSWYQQDLQKQLQWLFTLRSPGDKEVKSLPGADYLATRVTDTELRLQVANMSQGRTLYCTCSAGDEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 183 - protein, 247 aa, Artificial Sequence
  synthetic
  MISC_FEATURE (158)..(158): Xaa is Thr or Cys.
  MISC_FEATURE (222)..(222): Xaa is Ser, Ala, Val, Leu, Ile, Pro, Phe, Met, or Trp.
  MISC_FEATURE (224)..(224): Xaa is Met, Ala, Val, Leu, Ile, Pro, Phe, or Trp.
  MISC_FEATURE (225)..(225): Xaa is Gly, Ala, Val, Leu, Ile, Pro, Phe, Met, or Trp.
QQKVQQSPESLTVSEGAMASLNCTFSDGTSNNFRWYRQHSAKGLEVLVSIFSDGEKEEGRFTAHLNRANLHVSLHIREPQPSDSAVYLCAVITNTGKLTFGDGTVLTVKPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 184 - protein, 285 aa, Artificial Sequence
  synthetic
  MISC_FEATURE (169)..(169): Xaa is Ser or Cys.
GGIITQTPKFLIGQEGQKLTLKCQQNFNHDTMYWYRQDSGKGLRLIYYSITENDLQKGDLSEGYDASREKKSSFSLTVTSAQKNEMAVFLCASGGAFSYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 185 - protein, 252 aa, Artificial Sequence
  synthetic
  MISC_FEATURE (163)..(163): Xaa is Thr or Cys.
  MISC_FEATURE (227)..(227): Xaa is Ser, Ala, Val, Leu, Ile, Pro, Phe, Met, or Trp.
  MISC_FEATURE (229)..(229): Xaa is Met, Ala, Val, Leu, Ile, Pro, Phe, or Trp.
  MISC_FEATURE (230)..(230): Xaa is Gly, Ala, Val, Leu, Ile, Pro, Phe, Met, or Trp.
QQQVRQSPQSLTVWEGETAILNCSYEDSTFDYFPWYHQFPGESPALLIAIRPVSNKKEDGRFTIFFNKREKKFSLHIADSQPGDSATYFCAASANTGANTGKLTFGHGTILRVHPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 186 - protein, 285 aa, Artificial Sequence
  synthetic
  MISC_FEATURE (169)..(169): Xaa is Ser or Cys.
ETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 187 - protein, 248 aa, Artificial Sequence
  synthetic
  MISC_FEATURE (159)..(159): Xaa is Thr or Cys.
  MISC_FEATURE (223)..(223): Xaa is Ser, Ala, Val, Leu, Ile, Pro, Phe, Met, or Trp.
  MISC_FEATURE (225)..(225): Xaa is Met, Ala, Val, Leu, Ile, Pro, Phe, or Trp.
  MISC_FEATURE (226)..(226): Xaa is Gly, Ala, Val, Leu, Ile, Pro, Phe, Met, or Trp.
GEQVEQRPPHLSVREGDSAIIICTYTDSATAYFSWYKQEAGAGLQLLMSVLSNVDRKEEQGLTVLLNKKDKRLSLNLTAAHPGDSAVYFCAVDTNTGKLTFGDGTVLTVKPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 188 - protein, 288 aa, Artificial Sequence
  synthetic
  MISC_FEATURE (172)..(172): Xaa is Ser or Cys.
EAAVTQSPRSKVAVTGGKVTLSCHQTNNHDYMYWYRQDTGHGLRLIHYSYVADSTEKGDIPDGYKASRPSQENFSLILELASLSQTAVYFCASSDRLGGSAETLYFGSGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 189 - protein, 249 aa, Artificial Sequence
  synthetic
  MISC_FEATURE (160)..(160): Xaa is Thr or Cys.
  MISC_FEATURE (224)..(224): Xaa is Ser, Ala, Val, Leu, Ile, Pro, Phe, Met, or Trp.
  MISC_FEATURE (226)..(226): Xaa is Met, Ala, Val, Leu, Ile, Pro, Phe, or Trp.
  MISC_FEATURE (227)..(227): Xaa is Gly, Ala, Val, Leu, Ile, Pro, Phe, Met, or Trp.
GDKVKQSPSALSLQEGTNSALRCNFSIAATTVQWFLQNPRGSLINLFYLVPGTKENGRLKSAFDSKESYSTLHIRDAQLEDSGTYFCAALANTGANTGKLTFGHGTILRVHPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 190 - protein, 285 aa, Artificial Sequence
  synthetic
  MISC_FEATURE (169)..(169): Xaa is Ser or Cys.
DMKVTQMPRYLIKRMGENVLLECGQDMSHETMYWYRQDPGLGLQLIYISYDVDSNSEGDIPKGYRVSRKKREHFSLILDSAKTNQTSVYFCASSWGQGYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 191 - protein, 246 aa, Artificial Sequence
  synthetic
  MISC_FEATURE (157)..(157): Xaa is Thr or Cys.
  MISC_FEATURE (221)..(221): Xaa is Ser, Ala, Val, Leu, Ile, Pro, Phe, Met, or Trp.
  MISC_FEATURE (223)..(223): Xaa is Met, Ala, Val, Leu, Ile, Pro, Phe, or Trp.
  MISC_FEATURE (224)..(224): Xaa is Gly, Ala, Val, Leu, Ile, Pro, Phe, Met, or Trp.
GQQVQQSPTSLVLQEGENAELQCNFSTSLNSMQWFYQRPGGSLISVFYNPSGTKQSGRLTSTTVIKERRSSLHISSSQITDSGTYLCAMMSNMGYKLTFGTGTSLLVDPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 192 - protein, 285 aa, Artificial Sequence
  synthetic
  MISC_FEATURE (169)..(169): Xaa is Ser or Cys.
EAGVTQSPRYAVLQEGQAVSFWCDPISGHDTLYWYQQPRDQGPQLLVYFRDEAVIDNSQLPSDRFSAVRPKGTNSTLKIQSAKQGDTATYLCASSLSSYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 193 - protein, 252 aa, Artificial Sequence
  synthetic
  MISC_FEATURE (163)..(163): Xaa is Thr or Cys.
  MISC_FEATURE (227)..(227): Xaa is Ser, Ala, Val, Leu, Ile, Pro, Phe, Met, or Trp.
  MISC_FEATURE (229)..(229): Xaa is Met, Ala, Val, Leu, Ile, Pro, Phe, or Trp.
  MISC_FEATURE (230)..(230): Xaa is Gly, Ala, Val, Leu, Ile, Pro, Phe, Met, or Trp.
GEQVEQLPSILRVQEGSSASINCSYEDSASNYFPWYKQEPGENPKLIIDIRSNMERKQIQELIVLLDKKAKRFSLHITDTQPGDSAMYFCAASANTGANTGKLTFGHGTILRVHPNIQNPEPAVYQLKDPRSQDSTLCLFTDFDSQINVPKTMESGTFITDKXVLDMKAMDSKSNGAIAWSNQTSFTCQDIFKETNATYPSSDVPCDATLTEKSFETDMNLNFQNLXVXXLRILLLKVAGFNLLMTLRLWSS

SEQ ID NO: 194 - protein, 286 aa, Artificial Sequence
  synthetic
  MISC_FEATURE (170)..(170): Xaa is Ser or Cys.
ETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGSYEQYFGPGTRLTVLEDLRNVTPPKVSLFEPSKAEIANKQKATLVCLARGFFPDHVELSWWVNGKEVHSGVXTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 195 - protein, 27 aa, Artificial Sequence
  synthetic
RAKRSGSGATNFSLLKQAGDVEENPGP

SEQ ID NO: 196 - protein, 598 aa, Artificial Sequence
  synthetic
MAQFCILCLCVLMASVATDPTVTLLEQNPRWRLVPRGQAVNLRCILKNSQYPWMSWYQQDLQKQLQWLFTLRSPGDKEVKSLPGADYLATRVTDTELRLQVANMSQGRTLYCTCSTGDEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMKSFSISLVVLWLQLNWVNSQQKVQQSPESLIVPEGGMASLNCTSSDRNVDYFWWYRQHSGKSPKMLMSIFSNGEKEEGRFTVHLNKASLHTSLHIRDSQPSDSALYLCAASTNTGKLTFGDGTVLTVKPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 197 - protein, 599 aa, Artificial Sequence
  synthetic
MAPRLLCCVIFCLLAATFVDTTVKQNPRYKLARVGKPVNLICSQTMNHDTMYWYQKKPNQAPKLLLFYYDKILNREADTFEKFQSSRPNNSFCSLYIGSAGLEYSAMYLCASSPGASYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMHSLHVSLVFLWLQLGGVSSQEKVQQSPESLIVPEGAMVSLNCSFSDSASQSIWWYQQHPGKGPKALISIFSNGNKKEGRLTVYLNRASLHVSLHIKDSQPSDSAVYLCAVFNTGNYKYVFGAGTRLKVIAHIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 198 - protein, 598 aa, Artificial Sequence
  synthetic
MAQFCILCLCVLMASVATDPTVTLLEQNPRWRLVPRGQAVNLRCILKNSQYPWMSWYQQDLQKQLQWLFTLRSPGDKEVKSLPGADYLATRVTDTELRLQVANMSQGRTLYCTCSAGDEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMKSFSISLVVLWLQLNWVNSQQKVQQSPESLIVPEGGMASLNCTSSDRNVDYFWWYRQHSGKSPKMLMSIFSNGEKEEGRFTVHLNKASLHTSLHIRDSQPSDSALYLCAASTNTGKLTFGDGTVLTVKPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 199 - protein, 601 aa, Artificial Sequence
  synthetic
MAKWVFCWVTLCLLTVETTHGDGGIITQTPKFLIGQEGQKLTLKCQQNFNHDTMYWYRQDSGKGLRLIYYSITENDLQKGDLSEGYDASREKKSSFSLTVTSAQKNEMAVFLCASGGAFSYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMKSLSVSLVVLWLQFNWVSSQQKVQQSPESLTVSEGAMASLNCTFSDGTSNNFRWYRQHSAKGLEVLVSIFSDGEKEEGRFTAHLNRANLHVSLHIREPQPSDSAVYLCAVITNTGKLTFGDGTVLTVKPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 200 - protein, 611 aa, Artificial Sequence
  synthetic
MACRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMDKILTASFLLLGLHLAGVSGQQQEKHDQQQVRQSPQSLTVWEGETAILNCSYEDSTFDYFPWYHQFPGESPALLIAIRPVSNKKEDGRFTIFFNKREKKFSLHIADSQPGDSATYFCAASANTGANTGKLTFGHGTILRVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 201 - protein, 602 aa, Artificial Sequence
  synthetic
MASRLFFVVLILLCAKHMEAAVTQSPRSKVAVTGGKVTLSCHQTNNHDYMYWYRQDTGHGLRLIHYSYVADSTEKGDIPDGYKASRPSQENFSLILELASLSQTAVYFCASSDRLGGSAETLYFGSGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMKTVTGPLLLCFWLQLNCVSRGEQVEQRPPHLSVREGDSAIIICTYTDSATAYFSWYKQEAGAGLQLLMSVLSNVDRKEEQGLTVLLNKKDKRLSLNLTAAHPGDSAVYFCAVDTNTGKLTFGDGTVLTVKPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 202 - protein, 600 aa, Artificial Sequence
  synthetic
MAVRLISAVVLCFLGTGLVDMKVTQMPRYLIKRMGENVLLECGQDMSHETMYWYRQDPGLGLQLIYISYDVDSNSEGDIPKGYRVSRKKREHFSLILDSAKTNQTSVYFCASSWGQGYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMQRNLGAVLGILWVQICWVSGDKVKQSPSALSLQEGTNSALRCNFSIAATTVQWFLQNPRGSLINLFYLVPGTKENGRLKSAFDSKESYSTLHIRDAQLEDSGTYFCAALANTGANTGKLTFGHGTILRVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 203 - protein, 597 aa, Artificial Sequence
  synthetic
MATRLLGWAVFCLLDTVLSEAGVTQSPRYAVLQEGQAVSFWCDPISGHDTLYWYQQPRDQGPQLLVYFRDEAVIDNSQLPSDRFSAVRPKGTNSTLKIQSAKQGDTATYLCASSLSSYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMKRLLCSLLGLLCTQVCWVKGQQVQQSPTSLVLQEGENAELQCNFSTSLNSMQWFYQRPGGSLISVFYNPSGTKQSGRLTSTTVIKERRSSLHISSSQITDSGTYLCAMMSNMGYKLTFGTGTSLLVDPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 204 - protein, 605 aa, Artificial Sequence
  synthetic
MACRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSWGLGSYEQYFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMKTYAPTLFMFLWLQLDGMSQGEQVEQLPSILRVQEGSSASINCSYEDSASNYFPWYKQEPGENPKLIIDIRSNMERKQIQELIVLLDKKAKRFSLHITDTQPGDSAMYFCAASANTGANTGKLTFGHGTILRVHPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 205 - DNA, 394 bp, Mus musculus
atgtggcagttttgcattctgtgcctctgtgtactcatggcttctgtggctacagaccccacagtgactttgctggagcaaaacccaaggtggcgtctggtaccacgtggtcaagctgtgaacctacgctgcatcttgaagaattcccagtatccctggatgagctggtatcagcaggatctccaaaagcaactacagtggctgttcactctgcggagtcctggggacaaagaggtcaaatctcttcccggtgctgattacctggccacacgggtcactgatacggagctgaggctgcaagtggccaacatgagccagggcagaaccttgtactgcacctgcagtacaggggatgaacagtacttcggtcccggcaccaggctcacggttttag

SEQ ID NO: 206 - DNA, 391 bp, Mus musculus
atgaaatcctttagtatttccctagtggtcctgtggcttcagctaaactgggtgaacagccaacagaaggtgcagcagagcccagaatccctcattgttccagagggaggcatggcctctctcaactgcacttccagtgatcgtaatgttgactacttctggtggtacagacagcactctgggaaaagccccaagatgctgatgtctatcttctccaatggtgaaaaggaagaaggcagattcacagttcacctcaataaagccagcctgcatacttccctgcacatcagagactcccagcccagtgactctgctctctacctctgtgcagcaagcaccaatacaggcaaattaacctttggggatgggaccgtgctcacagtgaagccaa

SEQ ID NO: 207 - DNA, 393 bp, Mus musculus
atggcccagttctgcatcctgtgcctgtgcgtgctgatggcaagcgtggcaaccgacccaaccgtgacactgctggagcagaaccctaggtggagactggtgccaaggggacaggcagtgaacctgagatgtatcctgaagaattctcagtacccctggatgagctggtatcagcaggatctgcagaagcagctgcagtggctgtttaccctgcggtccccaggcgacaaggaggtgaagtctctgccaggagcagattacctggcaaccagggtgaccgacacagagctgcgcctgcaggtggccaatatgagccagggcagaacactgtactgcacctgttccacaggcgatgagcagtatttcggacctggaaccaggctgacagtgctg

SEQ ID NO: 208 - DNA, 390 bp, Mus musculus
atgaagagcttttccatctctctggtggtgctgtggctgcagctgaactgggtgaattcccagcagaaggtgcagcagagcccagagtccctgatcgtgcctgagggaggcatggccagcctgaactgcacctctagcgacaggaatgtggattacttttggtggtatcgccagcactctggcaagagccctaagatgctgatgtccatcttctctaacggcgagaaggaggagggcaggtttaccgtgcacctgaataaggcctctctgcacacaagcctgcacatccgcgactcccagccaagcgattccgccctgtacctgtgcgcagccagcaccaacacaggcaagctgaccttcggcgacggcaccgtgctgacagtgaagcct

SEQ ID NO: 209 - DNA, 394 bp, Mus musculus
atggatcctagacttctttgctgtgtgatcttctgtcttcttgcagccacttttgtggatactacggttaagcagaacccaagatacaagcttgcaagagttggaaaaccagtgaatttgatctgttctcagactatgaatcatgataccatgtactggtaccaaaagaagccaaaccaagcaccaaagcttcttcttttctactatgataagattttgaacagggaagctgacacttttgagaagttccaatccagtcggcctaacaattctttctgctctctctacattggctctgcaggcctagagtattctgccatgtacctctgtgctagcagcccgggggcctcctatgaacagtacttcggtcccggcaccaggctcacggttttag

SEQ ID NO: 210 - DNA, 394 bp, Mus musculus
atgcattccttacatgtttcactagtgttcctctggcttcaactaggtggggtgagcagccaggagaaggtacagcagagcccagaatccctcattgtcccagagggagccatggtctccctcaactgcagtttcagcgacagtgcttctcagtccatctggtggtaccaacagcatcctgggaaaggccccaaagcactaatatccatcttctctaatggcaacaagaaagaaggcagattgacagtttacctcaatagagccagcctgcatgtttccctgcacatcaaagactcccagcccagtgactccgctgtctacctctgtgcagtctttaatacaggaaactacaaatacgtctttggagcaggtaccagactgaaggttatagcac

SEQ ID NO: 211 - DNA, 393 bp, Mus musculus
atggccccccggctgctgtgctgcgtgatcttctgcctgctggccgccacctttgtggataccacagtgaagcagaaccctaggtacaagctggcccgcgtgggcaagccagtgaacctgatctgttcccagaccatgaatcacgacacaatgtactggtatcagaagaagccaaaccaggcccccaagctgctgctgttctactatgataagatcctgaatcgggaggccgacacattcgagaagtttcagagctccagacctaacaattccttttgctctctgtatatcggctccgccggcctggagtactctgccatgtacctgtgcgcatctagcccaggagcaagctacgagcagtatttcggccctggcaccaggctgacagtgctg

SEQ ID NO: 212 - DNA, 393 bp, Mus musculus
atgcactctctgcacgtgagcctggtgtttctgtggctgcagctgggaggcgtgagctcccaggagaaggtgcagcagtctccagagagcctgatcgtgcccgagggcgctatggtgagcctgaactgctccttctctgacagcgcctcccagtctatctggtggtaccagcagcaccctggcaagggcccaaaggccctgatctccatcttttctaacggcaataagaaggagggccggctgaccgtgtacctgaatagagcctccctgcacgtgtctctgcacatcaaggacagccagcccagcgatagcgccgtgtacctgtgcgccgtgttcaacaccggcaattacaagtacgtgttcggcgccggcacacggctgaaagtgatcgcc

SEQ ID NO: 213 - DNA, 394 bp, Mus musculus
atgtggcagttttgcattctgtgcctctgtgtactcatggcttctgtggctacagaccccacagtgactttgctggagcaaaacccaaggtggcgtctggtaccacgtggtcaagctgtgaacctacgctgcatcttgaagaattcccagtatccctggatgagctggtatcagcaggatctccaaaagcaactacagtggctgttcactctgcggagtcctggggacaaagaggtcaaatctcttcccggtgctgattacctggccacacgggtcactgatacggagctgaggctgcaagtggccaacatgagccagggcagaaccttgtactgcacctgcagtgctggggatgaacagtacttcggtcccggcaccaggctcacggttttag

SEQ ID NO: 214 - DNA, 391 bp, Mus musculus; identical to SEQ ID NO: 206

SEQ ID NO: 215 - DNA, 393 bp, Mus musculus
atggcccagttctgcatcctgtgcctgtgcgtgctgatggcaagcgtggcaaccgacccaaccgtgacactgctggagcagaaccctaggtggagactggtgccaaggggacaggcagtgaacctgagatgtatcctgaagaattctcagtacccctggatgagctggtatcagcaggatctgcagaagcagctgcagtggctgtttaccctgcggtccccaggcgacaaggaggtgaagtctctgcccggcgccgattacctggccacaagggtgaccgacacagagctgcgcctgcaggtggccaatatgagccagggcagaaccctgtactgcacatgttccgccggcgatgagcagtatttcggacctggaaccaggctgacagtgctg

SEQ ID NO: 216 - DNA, 390 bp, Mus musculus
atgaagagcttttccatctctctggtggtgctgtggctgcagctgaactgggtgaattcccagcagaaggtgcagcagagcccagagtccctgatcgtgcctgagggaggcatggccagcctgaactgcacctctagcgacaggaatgtggattacttttggtggtatcgccagcactctggcaagagccctaagatgctgatgtccatcttctctaacggcgagaaggaggagggcaggtttaccgtgcacctgaataaggcctctctgcacacaagcctgcacatccgcgattcccagccaagcgattccgccctgtacctgtgcgcagccagcaccaacacaggcaagctgaccttcggcgacggcaccgtgctgacagtgaagcct

SEQ ID NO: 217 - DNA, 403 bp, Mus musculus
atgaacaagtgggttttctgctgggtaaccctttgtctccttactgtagagaccacacatggtgatggtggcatcattactcagacacccaaattcctgattggtcaggaagggcaaaaactgaccttgaaatgtcaacagaatttcaatcatgatacaatgtactggtaccgacaggattcagggaaaggattgagactgatctactattcaataactgaaaacgatcttcaaaaaggcgatctatctgaaggctatgatgcgtctcgagagaagaagtcatctttttctctcactgtgacatctgcccagaagaacgagatggccgtttttctctgtgccagcgggggggccttctcctatgaacagtacttcggtcccggcaccaggctcacggttttag

SEQ ID NO: 218 - DNA, 391 bp, Mus musculus
atgaaatccttgagtgtttcactagtggtcctgtggctccagtttaattgggtgagcagccagcagaaggtgcagcagagcccagaatccctcactgtctcagagggagccatggcctctctcaactgcacgttcagtgatggtacttctaacaacttcaggtggtacagacagcattctgcgaaaggccttgaggtgctagtgtccatcttctctgatggtgaaaaggaagaaggcagatttacagctcacctcaatagagccaacttgcatgtttccctacacatcagagaaccacaacccagtgactctgctgtctacctctgtgcagtgatcaccaatacaggcaaattaacctttggggatgggaccgtgctcacagtgaagccaa

SEQ ID NO: 219 - DNA, 402 bp, Mus musculus
atggccaagtgggtgttctgctgggtgaccctgtgcctgctgacagtggagaccacacacggcgatggcggcatcatcacccagacacctaagttcctgatcggccaggagggccagaagctgaccctgaagtgccagcagaactttaatcacgatacaatgtactggtataggcaggactccggcaagggactgaggctgatctactattctatcaccgagaacgacctgcagaagggcgatctgagcgagggctacgatgcctcccgggagaagaagagctccttcagcctgaccgtgacatccgcccagaagaatgagatggccgtgtttctgtgcgcatctggaggagccttcagctacgagcagtattttggaccaggaaccaggctgacagtgctg

SEQ ID NO: 220 - DNA, 390 bp, Mus musculus
atgaagtctctgagcgtgtccctggtggtgctgtggctgcagtttaattgggtgtcctctcagcagaaggtgcagcagtctcccgagagcctgaccgtgtctgagggcgctatggccagcctgaactgcaccttctctgatggcacaagcaacaattttcggtggtacagacagcactccgccaagggcctggaggtgctggtgtccatcttctctgacggcgagaaggaggagggccggtttaccgcccacctgaacagagccaatctgcacgtgagcctgcacatcagggagccccagccttctgatagcgccgtgtacctgtgcgccgtgatcaccaatacaggcaagctgacattcggcgacggcaccgtgctgacagtgaagccc

SEQ ID NO: 221 - DNA, 394 bp, Mus musculus
atgggctgtaggctcctaagctgtgtggccttctgcctcttgggaataggccctttggagacggctgttttccagactccaaactatcatgtcacacaggtgggaaatgaagtgtctttcaattgtaagcaaactctgggccacgatactatgtattggtacaagcaagactctaagaaattgctgaagattatgtttagctacaataataagcaactcattgtaaacgaaacagttccaaggcgcttctcacctcagtcttcagataaagctcatttgaatcttcgaatcaagtctgtagagccggaggactctgctgtgtatctctgtgccagcagctggggactggggtatgaacagtacttcggtcccggcaccaggctcacggttttag

SEQ ID NO: 222 - DNA, 430 bp, Mus musculus
atggacaagatcctgacagcatcgtttttactcctaggccttcacctagctggggtgagtggccagcagcaggagaaacatgaccagcagcaggtgagacaaagtccccaatctctgacagtctgggaaggagagacagcaattctgaactgcagttatgaggacagcacttttgactacttcccatggtaccatcagttccctggggaaagccctgcactcctgatagccatacgtccagtgtccaataaaaaggaagatggacgattcacaatcttcttcaataaaagggagaaaaagttctccttgcacatcgcagactctcagcctggagactcagccacctacttctgtgcagcaagtgctaacactggagctaacactggaaagctcacgtttggacacggcaccatccttagggtccatccaa

SEQ ID NO: 223 - DNA, 393 bp, Mus musculus
atggcctgcagactgctgagctgcgtggccttctgtctgctgggcatcggcccactggagacagccgtgtttcagacccccaactaccacgtgacccaagtgggcaacgaggtgtccttcaattgtaagcagacactgggccacgataccatgtactggtataagcaggactctaagaagctgctgaagatcatgttcagctataacaataagcagctgatcgtgaacgagaccgtgcctcggcggttcagcccacagagctccgataaggcccacctgaatctgcggatcaagagcgtggagcctgaggactccgccgtgtacctgtgcgcatctagctggggactgggatacgagcagtatttcggaccaggaaccaggctgacagtgctg

SEQ ID NO: 224 - DNA, 429 bp, Mus musculus
atggacaagatcctgaccgcctcctttctgctgctgggactgcacctggcaggcgtgtctggccagcagcaggagaagcacgatcagcagcaggtgaggcagagcccacagtccctgacagtgtgggagggagagaccgccatcctgaattgctcctacgaggactctacattcgattactttccctggtatcaccagttcccaggagagtctcccgccctgctgatcgcaatcaggcctgtgagcaacaagaaggaggacggcaggttcaccatcttctttaataagcgcgagaagaagtttagcctgcacatcgcagactcccagccaggcgattctgccacatacttctgtgcagcatccgccaacaccggagccaatacaggcaagctgacctttggccacggcaccatcctgagggtgcaccct

SEQ ID NO: 225 - DNA, 400 bp, Mus musculus
atgggctccagactcttctttgtggttttgattctcctgtgtgcaaaacacatggaggctgcagtcacccaaagtccaagaagcaaggtggcagtaacaggaggaaaggtgacattgagctgtcaccagactaataaccatgactatatgtactggtatcggcaggacacggggcatgggctgaggctgatccattactcatatgtcgctgacagcacggagaaaggagatatccctgatgggtacaaggcctccagaccaagccaagagaatttctctctcattctggagttggcttccctttctcagacagctgtatatttctgtgccagcagtgatcggctggggggtagtgcagaaacgctgtattttggctcaggaaccagactgactgttctcg

SEQ ID NO: 226 - DNA, 397 bp, Mus musculus
atgaagacagtgactggacctttgttgctgtgcttctggctgcagctgaactgtgtgagcagaggcgagcaggtggagcagcgccctcctcacctgagtgtccgggagggagacagcgccattatcatctgcacctacacagacagtgccactgcttacttctcctggtacaagcaagaggcgggggcaggtcttcagttgcttatgagtgtcctttcaaatgtggacagaaaagaagaacaaggactcactgtcttactgaacaagaaagacaaacgactctctctgaacctcacagctgcccatcctggggactcagccgtgtacttctgcgcagtcgacaccaatacaggcaaattaacctttggggatgggaccgtgctcacagtgaagccaa

SEQ ID NO: 227 - DNA, 399 bp, Mus musculus
atggcctctagactgttctttgtggtgctgatcctgctgtgcgcaaagcacatggaggcagcagtgacccagtccccaaggtctaaggtggccgtgaccggcggcaaggtgacactgtcctgtcaccagaccaacaatcacgattacatgtattggtacagacaggacacaggccacggcctgaggctgatccactattcctacgtggccgactctaccgagaagggcgacatccccgatggctacaaggccagccgcccttcccaggagaacttcagcctgatcctggagctggcctctctgagccagaccgccgtgtatttttgcgcaagctccgataggctgggaggaagcgccgagacactgtacttcggatccggaaccaggctgacagtgctg

SEQ ID NO: 228 - DNA, 396 bp, Mus musculus
atgaagaccgtgacaggccccctgctgctgtgcttctggctgcagctgaattgcgtgagcaggggagagcaggtggagcagaggccaccacacctgtctgtgcgggagggcgatagcgccatcatcatctgcacctatacagactccgccaccgcctatttctcttggtacaagcaggaggcaggagcaggactgcagctgctgatgtccgtgctgtctaacgtggatagaaaggaggagcagggcctgaccgtgctgctgaacaagaaggacaagaggctgagcctgaatctgacagcagcacacccaggcgattccgccgtgtatttctgtgccgtggacaccaatacaggcaagctgacctttggcgacggcaccgtgctgacagtgaagcca

SEQ ID NO: 229 - DNA, 394 bp, Mus musculus
atgagagttaggctcatctctgctgtggtgctgtgtttcctaggaacaggccttgtggacatgaaagtaacccagatgccaagatacctgatcaaaagaatgggagagaatgttttgctggaatgtggacaggacatgagccatgaaacaatgtactggtatcgacaagaccctggtctggggctacagctgatttatatctcatacgatgttgatagtaacagcgaaggagacatccctaaaggatacagggtctcacggaagaagcgggagcatttctccctgattctggattctgctaaaacaaaccagacatctgtgtacttctgtgctagcagttggggacagggctatgaacagtacttcggtcccggcaccaggctcacggttttag

SEQ ID NO: 230 - DNA, 397 bp, Mus musculus
atgcagaggaacctgggagctgtgctggggattctgtgggtgcagatttgctgggtgagcggagataaggtgaaacaaagtccctcagcgctgagtctccaagaaggaaccaattctgctctgagatgcaatttttctatcgccgcgacaactgtgcagtggttcctacagaatcccaggggcagcctcatcaatcttttttacctggttccaggaacaaaggagaatgggaggttaaagtcagcattcgattctaaggagagctacagcaccctgcacatcagggatgcccagctggaggactcaggcacttacttctgtgctgctcttgctaacactggagctaacactggaaagctcacgtttggacacggcaccatccttagggtccatccaa

SEQ ID NO: 231 - DNA, 393 bp, Mus musculus
atggccgtgcgcctgatctctgccgtggtgctgtgcttcctgggcacaggcctggtggatatgaaggtgacccagatgcccagatacctgatcaagaggatgggcgagaacgtgctgctggagtgtggccaggatatgagccacgagaccatgtactggtataggcaggaccctggactgggactgcagctgatctacatcagctatgacgtggattctaacagcgagggcgatatcccaaagggctacagggtgtcccgcaagaagcgggagcacttctccctgatcctggactctgccaagaccaatcagacaagcgtgtatttctgcgcaagctcctggggacagggatacgagcagtattttggacctggaaccaggctgacagtgctg

SEQ ID NO: 232 - DNA, 396 bp, Mus musculus
atgcagaggaatctgggagccgtgctgggaatcctgtgggtgcagatctgctgggtgtccggcgacaaggtgaagcagtctccaagcgccctgtctctgcaggagggcacaaacagcgccctgagatgtaatttctccatcgccgccaccacagtgcagtggtttctgcagaaccctaggggctccctgatcaatctgttctacctggtgccaggcaccaaggagaatggccgcctgaagagcgcctttgactccaaggagtcctactctacactgcacatcagggacgcacagctggaggatagcggaacctatttctgcgccgccctggcaaacaccggagcaaatacaggcaagctgacctttggccacggcacaatcctgagggtgcaccca

SEQ ID NO: 233 - DNA, 394 bp, Mus musculus
atgggcaccaggcttcttggctgggcagtgttctgtctccttgacacagtactgtctgaagctggagtcacccagtctcccagatatgcagtcctacaggaagggcaagctgtttccttttggtgtgaccctatttctggacatgataccctttactggtatcagcagcccagagaccaggggccccagcttctagtttactttcgggatgaggctgttatagataattcacagttgccctcggatcgattttctgctgtgaggcctaaaggaactaactccactctcaagatccagtctgcaaagcagggcgacacagccacctatctctgtgccagcagtttgtcttcctatgaacagtacttcggtcccggcaccaggctcacggttttag

SEQ ID NO: 234 - DNA, 388 bp, Mus musculus
atgaagaggctgctgtgctctctgctggggcttctgtgcacccaggtttgctgggtgaaaggacagcaagtgcagcagagtcccacgtccttggttctgcaggagggggaaaacgcagagctgcagtgtaacttttccacatctttgaacagtatgcagtggttttaccaacgtcctgggggaagtctcatcagcgtgttctacaatccttctgggacaaagcagagtgggagactgacatccacaacagtcattaaagaacgccgcagctctttgcacatttcctcctcccagatcacagactcaggcacttatctctgtgctatgatgagcaacatgggctacaaacttaccttcgggacaggaacaagcttgttggttgatccaa

SEQ ID NO: 235 - DNA, 393 bp, Mus musculus
atggccaccaggctgctgggatgggccgtgttctgcctgctggacaccgtgctgtccgaggcaggagtgacacagtctccaagatacgccgtgctgcaggagggacaggccgtgagcttttggtgtgatcctatctccggccacgacacactgtactggtatcagcagccaagggatcagggaccacagctgctggtgtatttcagagacgaggccgtgatcgataattcccagctgccctctgaccggtttagcgccgtgagacctaagggcaccaactctacactgaagatccagagcgccaagcagggcgataccgccacatacctgtgcgccagctccctgtctagctacgagcagtatttcggaccaggaaccaggctgacagtgctg

SEQ ID NO: 236 - DNA, 387 bp, Mus musculus
atgaagagactgctgtgctccctgctgggactgctgtgcacccaggtgtgctgggtgaagggccagcaggtgcagcagtcccctacatctctggtgctgcaggagggcgagaacgccgagctgcagtgtaatttctccacctctctgaacagcatgcagtggttttaccagaggccaggaggaagcctgatctccgtgttctataatccctctggcaccaagcagagcggcagactgacatccaccacagtgatcaaggagcggagaagctccctgcacatctctagctcccagatcaccgactctggcacatacctgtgcgccatgatgagcaacatgggctataagctgacctttggcaccggcacatccctgctggtggatcct

SEQ ID NO: 237 - DNA, 397 bp, Mus musculus
atgggctgtaggctcctaagctgtgtggccttctgcctcttgggaataggccctttggagacggctgttttccagactccaaactatcatgtcacacaggtgggaaatgaagtgtctttcaattgtaagcaaactctgggccacgatactatgtattggtacaagcaagactctaagaaattgctgaagattatgtttagctacaataataagcaactcattgtaaacgaaacagttccaaggcgcttctcacctcagtcttcagataaagctcatttgaatcttcgaatcaagtctgtagagccggaggactctgctgtgtatctctgtgccagcagctggggactggggtcctatgaacagtacttcggtcccggcaccaggctcacggttttag

SEQ ID NO: 238 - DNA, 409 bp, Mus musculus
atgaaaacatatgctcctacattattcatgtttctatggctgcagctggatgggatgagccaaggcgagcaggtggagcagcttccttccatcctgagagtccaggagggatccagtgccagcatcaactgcagttatgaagacagtgcctccaactacttcccttggtataagcaagaacctggagagaatcctaagctcatcattgacattcgttcaaatatggaaagaaagcagatccaagaactcatcgttttactggataagaaagccaaacgcttctccctgcacatcacagacacccagcctggagactcagccatgtacttctgtgctgcaagtgctaacactggagctaacactggaaagctcacgtttggacacggcaccatccttagggtccatccaa

SEQ ID NO: 239 - DNA, 396 bp, Mus musculus
atggcctgcagactgctgtcttgcgtggccttctgtctgctgggcatcggcccactggagacagccgtgtttcagacccccaactaccacgtgacccaagtgggcaacgaggtgtcctttaattgtaagcagacactgggccacgataccatgtactggtataagcaggactccaagaagctgctgaagatcatgttctcttacaacaataagcagctgatcgtgaacgagaccgtgcctcggagattcagcccacagagctccgataaggcccacctgaatctgcggatcaagagcgtggagccagaggactccgccgtgtacctgtgcgcatctagctggggactgggatcctacgagcagtattttggaccaggaaccaggctgacagtgctg

SEQ ID NO: 240 - DNA, 408 bp, Mus musculus
atgaagacctacgcccctacactgttcatgtttctgtggctgcagctggacggcatgtctcagggcgagcaggtggagcagctgccaagcatcctgagggtgcaggagggaagctccgccagcatcaactgctcctatgaggactccgcctctaattacttcccctggtataagcaggagccaggcgagaaccccaagctgatcatcgatatccggagcaatatggagagaaagcagatccaggagctgatcgtgctgctggacaagaaggccaagcggttcagcctgcacatcaccgacacacagcctggcgattctgccatgtacttttgtgccgccagcgccaacaccggcgccaatacaggcaagctgaccttcggccacggcacaatcctgagggtgcaccct

SEQ ID NO: 241 - protein, 9 aa, Artificial Sequence
  synthetic
ALFLGILSV

SEQ ID NO: 242 - protein, 9 aa, Artificial Sequence
  synthetic
SAFLGILSV

SEQ ID NO: 243 - protein, 9 aa, Artificial Sequence
  synthetic
SLALGILSV

SEQ ID NO: 244 - protein, 9 aa, Artificial Sequence
  synthetic
SLFAGILSV

SEQ ID NO: 245 - protein, 9 aa, Artificial Sequence
  synthetic
SLFLAILSV

SEQ ID NO: 246 - protein, 9 aa, Artificial Sequence
  synthetic
SLFLGALSV

SEQ ID NO: 247 - protein, 9 aa, Artificial Sequence
  synthetic
SLFLGIASV

SEQ ID NO: 248 - protein, 9 aa, Artificial Sequence
  synthetic
SLFLGILAV

SEQ ID NO: 249 - protein, 9 aa, Artificial Sequence
  synthetic
SLFLGILSA

SEQ ID NO: 250 - protein, 9 aa, Artificial Sequence
  synthetic
SAFLGILNI

SEQ ID NO: 251 - protein, 9 aa, Artificial Sequence
  synthetic
YIFLGILCM

SEQ ID NO: 252 - protein, 9 aa, Artificial Sequence
  synthetic
FLFLAILRL

SEQ ID NO: 253 - protein, 9 aa, Artificial Sequence
  synthetic
ALFLAVLSI

SEQ ID NO: 254 - protein, 9 aa, Artificial Sequence
  synthetic
SIFLGVITV

SEQ ID NO: 255 - protein, 9 aa, Artificial Sequence
  synthetic
SLYLSLVTL

SEQ ID NO: 256 - protein, 9 aa, Artificial Sequence
  synthetic
YYFLGILAM

SEQ ID NO: 257 - protein, 9 aa, Artificial Sequence
  synthetic
YFFLGILST

SEQ ID NO: 258 - protein, 9 aa, Artificial Sequence
  synthetic
LLFLGILLF